(19) United States
(12) Patent Application Publication
(10) Pub. No.: US 2015/0059023 A1
(43) Pub. Date: Feb. 26, 2015

(54) BIOMASS YIELD GENES

(71) Applicants: Christopher Yohn, San Diego, CA (US); Philip A. Lee, Las Cruces, NM (US)

(72) Inventors: Christopher Yohn, San Diego, CA (US); Philip A. Lee, Las Cruces, NM (US)

(73) Assignee: Sapphire Energy, Inc., San Diego, CA (US)

(21) Appl. No.: 14/378,795

(22) PCT Filed: Feb. 14, 2013
(86) PCT No.: PCT/US2013/026208

§ 371 Date: Aug. 14, 2014

Related U.S. Application Data
(60) Provisional application No. 61/598,477, filed on Feb. 14, 2012.

(51) Int. Cl. C12N 15/82

(52) U.S. Cl. CPC C12N 15/8242
USPC 800/290; 435/471

(57) ABSTRACT

The present disclosure provides several novel genes that have been shown to increase the biomass yield or biomass of a photosynthetic organism. The disclosure also provides methods of using the novel genes and organisms transformed with the novel genes.

### CROSS REFERENCE TO RELATED APPLICATIONS

This application claims the benefit of U.S. Provisional patent application Ser. No. 61/598,477, filed Feb. 14, 2012, of which is herein incorporated by reference in its entirety for all purposes.

### BACKGROUND

There exists a need for increased biomass yield in algae in order to obtain more of a desired product, for example, liquid transportation fuels, biodiesel, human nutritional supplements, animal feed, fertilizer, feed stock for electricity generation, health and nutrition based products, renewable chemicals, and bioplastics.

The present disclosure provides several plant genes that have been shown to increase biomass yield, specifically EBP1 (the ErbB-3 epidermal growth factor receptor binding protein), TOR kinase, and Rubsico activase.

EBP1 (the ErbB-3 Epidermal Growth Factor Receptor Binding Protein.)

As described in Horvath, B. M., et al. (The EMBO Journal (2006) 25:4909-4029) plant EBP1 levels are tightly regulated; gene expression is highest in developing organs and correlates with genes involved in ribosome biogenesis and function. The EBP1 protein is stabilized by auxin.

Elevating or decreasing EBP1 levels in transgenic higher plants, such as Arabidopsis, results a dose-dependent increase or reduction in organ growth, respectively. During early stages of organ development, EBP1 promotes cell proliferation, influences cell-size threshold for division and shortens the period of meristematic activity. In post mitotic cells, it enhances cell expansion. EBP1 is required for expression of cell cycle genes; CyclinD3;1, ribonucleotide reductase 2 and the cyclin-dependent kinase B1;1. The regulation of these genes by EBP1 is dose and auxin dependent and might rely on the effect of EBP1 to reduce RBR1 protein levels. EBP1 is believed to be a conserved, dose-dependent regulator of cell growth that is connected to meristematic competence and cell proliferation via regulation of RBR1 levels.

TOR (Target of Rapamycin) Kinase

Plants, unlike animals, have plastic organ growth that is largely dependent on environmental information. However, so far, little is known about how this information is perceived and transduced into coherent growth and developmental decisions. Deprost, D., et al. (EMBO reports (2007) Vol., 8, No. 9, pp. 864-870) reported that the growth of Arabidopsis thaliana, a higher plant, is positively correlated with the level of expression of TOR kinase. Diminished or augmented expression of the AtTOR gene results in a dose-dependent decrease or increase, respectively, in organ and cell size, seed production and resistance to osmotic stress. Strong down regulation of AtTOR expression by inducible RNA interference also leads to a post-germinative halt in growth and development, which phenocopies the action of the plant hormone abscisic acid, to an early senescence and to a reduction in the amount of translated messenger RNA. It is believed that the AtTOR kinase is one of the contributors to the link between environmental cues and growth processes in plants.

Rubisco and Rubisco Activase (RCA)

The most abundant protein, Rubisco [ribulose-1,5-bisphosphate (RuBP) carboxylase/oxygenase; EC 4.1.1.39] catalyzes the assimilation of CO₂, by the carboxylation of ribulose-1,5-bisphosphate (RuBP) in photosynthetic carbon assimilation (Ellis, R. J. (1979) Journal of Agricultural Science 145, 31-43). However, the catalytic limitations of Rubisco compromise the efficiency of photosynthesis (Parry, M. A. J., et al. (2007) Journal of Agricultural Science 145, 31-43). Compared to other enzymes of the Calvin cycle, Rubisco has a low turnover number, meaning that relatively large amounts must be present to sustain sufficient rates of photosynthesis. Furthermore, Rubisco also catalyzes a competing and wasteful reaction with oxygen, initiating the process of photorespiration, which leads to a loss of fixed carbon and consumes energy. Although Rubisco and the photorespiratory enzymes are a major nitrogen store, and can account for more than 25% of leaf nitrogen, Rubisco activity can still be limiting.

The mechanisms involved in Rubisco regulation are described, for example, in Parry, M. A. J., et al., J. of Experimental Botany (2008) Vol. 59(7) 1569-1580), Rubisco enzymatic activity in vivo is modulated either by the carbamoylation of an essential lysine residue at the catalytic site and subsequent stabilization of the resulting carbamate by a Mg²⁺ ion, forming a catalytically active ternary complex; or through the tight binding of low molecular weight inhibitors. The CO₂ involved in active site carbonylation is distinct from CO₂ reacting with the acceptor molecule, RuBP, during catalysis. Inhibitors bind either before or after carbamylation and block the active site of the enzyme, preventing carbamylation and/or substrate binding. The removal of tightly bound inhibitors from the catalytic site of the carbamoylated and decarbarnylated forms of Rubisco requires Rubisco activase and the hydrolysis of ATP. In this way Rubisco activase ensures that the Rubisco active site is not blocked by inhibitors and so is free either to become carbamylated or to participate directly in catalysis.

The importance of Rubisco activase for complete activation of Rubisco in vivo, was first recognized during the analysis of an Arabidopsis (rca) mutant that was unable to survive under ambient CO₂ (Somerville, C. R., et al. (1982) Plant Physiology 70:381-387). Salvucci, M. E., et al. (Photosynthesis Research (1985) 7: 193-201) showed this to be due to the absence of a novel enzyme, Rubisco activase. It has subsequently been shown that Rubisco activase is essential for the activation and maintenance of Rubisco catalytic activity by promoting the removal of any tightly bound, inhibitory, sugar phosphates from the catalytic site of both the carbamylated and decarbamylated forms of Rubisco (for example, as described in Mate, C. J., et al. (1993) Plant Physiology 102:1119-1128). Rubisco activase has been detected in all plant species examined thus far and is a member of the AAA+ super family Whose members perform chaperone like functions (Spreitzer, R. J. and Salvucci, M. E. (2002) Annual Review of Plant Physiology and Plant Molecular Biology, 53:449-475).

Thermostable variants of Rubisco activase have been shown to increase biomass yield in higher plants (for example, as described in Kurek, I., et al., The Plant Cell (2007) Vol. 19:3230-3241).

Though over expression of these three proteins has been studied in higher plants, overexpression of these proteins in algae has not been studied and could result in an increase in the proteins' activity and thus an increase in biomass yield.

### SUMMARY

Described herein are several novel genes that have been shown to increase the biomass yield or biomass of a photosynthetic organism. The disclosure also provides methods of using the novel genes and organisms transformed with the novel genes.

Provided herein is a photosynthetic organism transformed with an isolated polynucleotide comprising: (a) a nucleic acid sequence of SEQ ID NO: 21, 19, 17, 20, 18, 16, 15, 61, 64, 66, 68 or 69; or (b) a nucleotide sequence with at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69; wherein the transformed photosynthetic organism's biomass is increased as compared to a biomass of an untransformed photosynthetic organism or a second transformed photosynthetic organism. In some embodiments, the increase is measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the increase is shown by the transformed photosynthetic organism having a positive selection coefficient as compared to either the untransformed photosynthetic organism or the second transformed photosynthetic organism. In some embodiments, the selection coefficient is from 0.05 to 0.10, from 0.10 to 0.5, from 0.5 to 0.75, from 0.75 to 1.0, from 1.0 to 1.5, from 1.5 to 2,0, or 2.0 to 3,0. In one embodiment, the increase is measured by growth rate. In other embodiments, the transformed photosynthetic organism has an increase in growth rate as compared to either the untransformed photosynthetic organism or the second transformed photosynthetic organism of from 5% to 10%, from 10% to 15%, from 15% to 25%, from 25% to 50%, from 50% to 100%, from 100% to 200%, or from 200% to 400%. In another embodiment, the increase is measured by an increase in carrying capacity. In one embodiment, the units of carrying capacity are mass per unit of volume or area. In another embodiment, the increase is measured by an increase in culture productivity. In yet another embodiment, the units of culture productivity are grams per meter squared per day. In some embodiments, the transformed photosynthetic organism has an increase in productivity as measured in grams per meter squared per day, as compared to either the untransformed photosynthetic organism or the second transformed photosynthetic organism of from 5% to 25%, from 25% to 50%, from 50% to 100%, from 100% to 200%, or from 200% to 400%. In yet another embodiment, the transformed photosynthetic organism is grown in an aqueous environment. In one embodiment, the transformed photosynthetic organism is a bacterium. In another embodiment, the bacterium is a cyanobacterium. In yet another embodiment, the transformed photosynthetic organism is an alga. In one embodiment, the alga is a microalga. In other embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In another embodiment, the transformed photosynthetic organism is a vascular plant.

Also provide herein is a method of increasing biomass of a photosynthetic organism, comprising: (a) transforming the photosynthetic organism with a polynucleotide, wherein the polynucleotide comprises: (i) a nucleic acid sequence of SEQ ID NO: 21, 19, 17, 20, 18, 16, 15, 61, 64, 66, 68 or 69; or (ii) a nucleotide sequence with at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69; and wherein the nucleic acid of (I) or the nucleotide of (ii) encode for a polypeptide that when expressed results in an increase in the biomass of the transformed photosynthetic organism as compared to an untransformed photosynthetic organism or a second transformed photosynthetic organism. In some embodiments, the increase is measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the increase is shown by the transformed photosynthetic organism having a positive selection coefficient as compared to either the untransformed photosynthetic organism or the second transformed photosynthetic organism. In some embodiments, the selection coefficient is from 0.05 to 0.10, from 0.10 to 0.5, from 0.5 to 0.75, from 0.75 to 1,0, from 1.0 to 1.5, from 1.5 to 2.0, or 2.0 to 3.0. In one embodiment, the increase is measured by growth rate. In other embodiments, the transformed photosynthetic organism has an increase in growth rate as compared to either the untransformed photosynthetic organism or the second transformed photosynthetic organism of from 5% to 10%, from 10% to 15%, from 15% to 25%, from 25% to 50%, from 50% to 100%, from 100% to 200%, or from 200% to 400%. In another embodiment, the increase is measured by an increase in carrying capacity. In one embodiment, the units of carrying capacity are mass per unit of volume or area. In another embodiment, the increase is measured by an increase in culture productivity. In yet another embodiment, the units of culture productivity are grams per meter squared per day. In some embodiments, the transformed photosynthetic organism has an increase in productivity as measured in grams per meter squared per day, as compared to either the untransformed photosynthetic organism or the second transformed photosynthetic organism of from 5% to 25%, from 25% to 50%, from 50% to 100%, from 100% to 200%, or from 200% to 400%. In yet another embodiment, the transformed photosynthetic organism is grown in an aqueous environment. In one embodiment, the transformed photosynthetic organism is a bacterium. In another embodiment, the bacterium is a cyanobacterium. In yet another embodiment, the transformed photosynthetic organism is an alga. In one embodiment, the alga is a microalga. In other embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus Haematococcus sp., or Desmodesmus sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In another embodiment, the transformed photosynthetic organism is a vascular plant.

Also provided herein is a photosynthetic organism transformed with an isolated polynucleotide comprising: (a) a nucleic acid sequence of SEQ ID NO: 50, 51, 52, 53, 54, 55, 56, 57, 58, or 62; or (b) a nucleotide sequence with at least 80%, at least 85%, at least 90%, at least 95%, at least 98%, or at least 99% sequence identity to the nucleic acid sequence of SEQ ID NOL 50, 51, 52, 53, 54, 55, 56, 57, 58, or 62; wherein the transformed photosynthetic organism's biomass is increased as compared to a biomass of an untransformed photosynthetic organism or a second transformed photosynthetic organism, in some embodiments, the increase is measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the increase is shown by the transformed photosynthetic organism having a positive selection coefficient as compared to either the untransformed photosynthetic organism or the second transformed photosynthetic organism. In some embodiments, the selection coefficient is from 0.05 to 0.10, from 0.10 to 0.5, from 0.5 to 0.75, from 0.75 to 1.0, front 1.0 to 1.5, from 1.5 to 2.0, or 2.0 to 3.0. In one embodiment, the increase is measured by growth rate, in other embodiments, the transformed photosynthetic organism has an increase in growth rate as compared to either the untransformed photosynthetic organism or the second transformed photosynthetic organism of from 5% to 10%, from 10% to 15%, from 15% to 25%, from 25% to 50%, from 50% to 100%, from 100% to 200%, or from 200% to 400%. In another embodiment, the increase is measured by an increase in carrying capacity. In one embodiment, the units of carrying capacity are mass per unit of volume or area. In another embodiment, the increase is measured by an increase in culture productivity. In yet another embodiment, the units of culture productivity are grams per meter squared per day. In some embodiments, the transformed photosynthetic organism has an increase in productivity as measured in grams per meter squared per day, as compared to either the untransformed photosynthetic organism or the second transformed photosynthetic organism of from 5% to 25%, from 25% to 50%, from 50% 100%, from 100% to 200%, or from 200% to 400%. In yet another embodiment, the transformed photosynthetic organism is grown in an aqueous environment. In one embodiment, the transformed photosynthetic organism is a bacterium. In another embodiment, the bacterium is a cyanobacterium. In yet another embodiment, the transformed photosynthetic organism is an alga. In one embodiment, the alga is a microalga. In other embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desinodesmus sp. In yet other embodiments, the microalga is at least one of Chlamydomonas reinhardtii, N. oceanica, N. sauna, Dunaliella salina, H. pluvalis, S. dimorphus, Dunaliella viridis, N. oculata, Dunaliella tertiolecta, S. Maximus, or A. Fusiformus. In another embodiment, the transformed photosynthetic organism is a vascular plant.

Also provided herein is a method of increasing biomass of a photosynthetic organism, comprising: (a) transforming the photosynthetic organism with a polynucleotide, wherein the polynucleotide comprises: (i) a nucleic acid sequence of SEQ ID NO: 50, 51, 52, 53, 54, 55, 56, 57, 58, or 62; or (ii) a nucleotide sequence with at least 80%, at least 85%, at least 90%, at least 95%, at least 98%, or at least 99% sequence identity to the nucleic acid sequence of SEQ ID NO: 50, 51, 52, 53, 54, 55, 56, 57, 58, or 62; and wherein the nucleic acid of (i) or the nucleotide of (ii) encode for a polypeptide that when expressed results in an increase in the biomass of the transformed photosynthetic organism as compared to an untransformed photosynthetic organism or a second transformed photosynthetic organism. In some embodiments, the increase is measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the increase is shown by the transformed photosynthetic organism having a positive selection coefficient as compared to either the untransformed photosynthetic organism or the second transformed photosynthetic organism. In some embodiments, the selection coefficient is from 0.05 to 0.10, from 0.10 to 0.5, from 0.5 to 0.75, from 0.75 to 1.0, from 1.0 to 1.5, from 1.5 to 2,0, or 2.0 to 3.0. In one embodiment, the increase is measured by growth rate. In other embodiments, the transformed photosynthetic organism has an increase in growth rate as compared to either the untransformed photosynthetic organism or the second transformed photosynthetic organism of from 5% to 10%, from 10% to 15%, from 15% to 25%, from 25% to 50%, from 50% to 100%, from 100% to 200%, or from 200% to 400%. In another embodiment, the increase is measured by an increase in carrying capacity. In one embodiment, the units of carrying capacity are mass per unit of volume or area. In another embodiment, the increase is measured by an increase in culture productivity. In yet another embodiment, the units of culture productivity are grams per meter squared per day. In some embodiments, the transformed photosynthetic organism has an increase in productivity as measured in grams per meter squared per day, as compared to either the untransformed photosynthetic organism or the second transformed photosynthetic organism of from 5% to 25%, from 25% to 50%, from 50% to 100%, from 100% to 200%, or from 200% to 400%. In yet another embodiment, the transformed photosynthetic organism is grown in an aqueous environment. In one embodiment, the transformed photosynthetic organism is a bacterium. In another embodiment, the bacterium is a cyanobacterium. In yet another embodiment, the transformed photosynthetic organism is an alga. In one embodiment, the alga is a microalga. In other embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Destnodesmus sp. In yet other embodiments, the microalga is at least one of Chlamydomonas reinhardtii, N. oceanica, N. sauna, Dunaliella salina, H. pluvalis, S. dimorphus, Dunaliella viridis, N. oculata. Dunaliella tertiolecta, S. Maximus, or A. Fusiformus. In another embodiment, the transformed photosynthetic organism is a vascular plant.

Also provided herein is a photosynthetic organism transformed with an isolated polynucleotide comprising: (a) a nucleic acid sequence of SEQ ID NO: 32, 38, 34, or 40; (b) a nucleotide sequence with at least 80%, at least 85%, at least 90%, at least 95%, at least 98%, or at least 99% sequence identity to the nucleic acid sequence of SEQ ID NO: 32, 38, 34, or 40; (c) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (d) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species; wherein the transformed photosynthetic organism's biomass is increased as compared to a biomass of an untransformed photosynthetic organism or a second transformed photosynthetic organism. In some embodiments, the nucleic acid sequence or the nucleotide sequence encodes a protein comprising, (a) an amino acid sequence of SEQ ID NO: 33 or SEQ ID NO: 39; or (b) a homolog of the amino acid sequence of (a), wherein the homolog has at least 80%, at least 85%, at least 90%, at least 95%, at least 98%, or at least 99% sequence identity to the amino acid sequence of SEQ ID NO: 33 or SEQ ID NO: 39. In some embodiments, the increase is measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the increase is show by the transformed photosynthetic organism having a positive selection coefficient as compared to either the untransformed photosynthetic organism or the second transformed photosynthetic organism. In some embodiments, the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. In one embodiment, the increase is measured by growth rate. In other embodiments, the transformed photosynthetic organism has an increase in growth rate as compared to either the untransformed photosynthetic organism or the second transformed photosynthetic organism of from 5% to 10%, from 10% to 15%, from 15% to 25%, from 25% to 50%, from 50% to 100%, from 100% to 200%, or from 200% to 400%. In another embodiment, the increase is measured by an increase in carrying capacity. In one embodiment, the units of carrying capacity are mass per unit of volume or area. In another embodiment, the increase is measured by an increase in culture productivity. In yet another embodiment, the units of culture productivity are grams per meter squared per day. In some embodiments, the transformed photosynthetic organism has an increase in productivity as measured in grams per meter squared per day, as compared to either the untransformed photosynthetic organism or the second transformed photosynthetic organism of from 5% to 25%, from 25% to 50%. from 50% to 100%, from 100% to 200%, or from 200% to 400%. In yet another embodiment, the transformed photosynthetic organism is grown in an aqueous environment. In one embodiment, the transformed photosynthetic organism is a bacterium. In another embodiment, the bacterium is a cyanobacterium. In yet another embodiment, the transformed photosynthetic organism is an alga. In one embodiment, the alga is a microalga. In other embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Scenedesmus sp., Chlorella sp., Hematococcus sp., Volvacales sp., Desmodesmus sp. In yet other embodiments, the microalga is at least one of Chlamydomonas reinhardtii, N. oceanica. N. salina, Dunaliella salina, H. pluvalis, S. dimorphus, Dunaliella viridis, N. oculata, Dunaliella tertiolecta, S. Maximus, or A. Fusiformus. In another embodiment, the transformed photosynthetic organism is a vascular plant.

Provided herein is a method of increasing biomass of a photosynthetic organism, comprising: (a) transforming the photosynthetic organism with a polynucleotide, wherein the polynucleotide comprises: (i) a nucleic acid sequence of SEQ ID NO: 32, 38, 34, or 40; (ii) a nucleotide sequence with at least 80%, at least 85%, at least 90%, at least 95%, at least 98%, or at least 99% sequence identity to the nucleic acid sequence of SEQ ID NO: 32, 38, 34, or 40; (iii) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (iv) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species; and wherein the nucleic acid of (i), (iii), or (iv), or the nucleotide sequence of (ii) encode for a polypeptide that when expressed results in an increase in the biomass of the transformed photosynthetic organism as compared to an untransformed photosynthetic organism or a second transformed photosynthetic organism. In some embodiments, the nucleic acid sequence or the nucleotide sequence encodes a protein comprising, (a) an amino acid sequence of SEQ ID NO: 33 or SEQ ID NO: 39; or (b) a homolog of the amino acid sequence of (a), wherein the homolog has at least 80%, at least 85%, at least 90%, at least 95%, at least 98%, or at least 99% sequence identity to the amino acid sequence of SEQ ID NO: 33 or SEQ ID NO: 39. In some embodiments, the increase is measured by a competition assay, with rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the increase is shown by the transformed photosynthetic organism having a positive selection coefficient as compared to either the untransformed photosynthetic organism or the second transformed photosynthetic organism. In some embodiments, the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. In one embodiment, the increase is measured by growth rate. In other embodiments, the transformed photosynthetic organism has an increase in growth rate as compared to either the untransformed photosynthetic organism or the second transformed photosynthetic organism of from 5% to 10%, from 10% to 15%, from 15% to 25%, from 25% to 50%, from 50% to 100%, from 100% to 200%, or from 200% to 400%. In another embodiment, the increase is measured by an increase in carrying capacity. In one embodiment, the units of carrying capacity are mass per unit of volume or area. In another embodiment, the increase is measured by an increase in culture productivity. In yet another embodiment, the units of culture productivity are grams per meter squared per day. In some embodiments, the transformed photosynthetic organism has an increase in productivity as measured in grams per meter squared per day, as compared to either the untransformed photosynthetic organism or the second transformed photosynthetic organism of from 5% to 25%, from 25% to 50%, from 50% to 100%, from 100% to 200%, or from 200% to 400%. In yet another embodiment, the transformed photosynthetic organism is grown in an aqueous environment. In one embodiment, the transformed photosynthetic organism is a bacterium. In another embodiment, the bacterium is a cyanobacterium. In yet another embodiment, the transformed photosynthetic organism is an alga, in one embodiment, the alga is a microalga. In other embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmiis sp. In yet other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In another embodiment, the transformed photosynthetic organism is a vascular plant.

Also provided herein is a higher plant transformed with an isolated polynucleotide comprising: (a) a nucleic acid sequence of SEQ ID NO: 21, 19, 17, 20, 18, 16, 15, 61, 64, 66, 68, 69, 50, 51, 52, 53, 54, 55, 56, 57, 58, 62, 32, 38, 34, or 40; or (b) a nucleotide sequence with at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68, 69, 50, 51., 52, 53, 54, 55, 56, 57, 58, 62, 32, 38, 34, or 40; wherein the transformed higher plant's biomass is increased as compared to a biomass of an untransformed higher plant or a second transformed higher plant. In some embodiments, the increase is measured by a competition assay, growth rate, carrying capacity, culture productivity, cell proliferation, seed yield, organ growth, or polysome accumulation. In one embodiment, the increase is measured by a competition assay. In other embodiments, the increase is shown by the transformed higher plant having a positive selection coefficient as compared to either the untransformed higher plant or the second transformed higher plant. In yet other embodiments, the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. In one embodiment, the increase is measured by growth rate. In yet other embodiments, the transformed higher plant has an increase in growth rate as compared to either the untransformed higher plant or the second transformed higher plant of from 5% to 10%, from 10% to 15%, from 15% to 25%, from 25% to 50%, from 50% to 100%, from 100% to 200%, or from 200% to 400%. In one embodiment, the increase is measured by an increase in carrying capacity. In another embodiment, the units of carrying capacity are mass per unit of volume or area. In yet another embodiment, the increase is measured by an increase in culture productivity. In another embodiment, the units of culture productivity are grams per meter squared per day. In some embodiments, the transformed higher plant has an increase in productivity as measured in grams per meter squared per day, as compared to either the untransformed higher plant or the second transformed higher plant of from 5% to 25%, from 25% to 50%, from 50% to 100%, from 100% to 200%, or from 200% to 400%. In one embodiment, the transformed higher plant is grown in an aqueous environment. In another embodiment, the higher plant is Arabidopsis thaliana. In other embodiments, the higher plant is a Brassica, Glycine, Gossypium, Medicago, Zea, Sorghum, Oryza, Triticum, or Panicum species.

Also provided herein is a codon usage table capable of being used to codon optimize a nucleic acid for expression in the nucleus of a Desmodesmus, a Chlamydomonas, a Nannochloropsis, and/or a Scenedesmus species, comprising the following data: a) for Phenylalanine: 16% codons encoding for Phenylalanine are UUU; and 84% of codons encoding for Phenylalanine are UUC; b) for Leucine: 1% of codons encoding for Leucine are UUA; 4% of codons encoding for Leucine are UUG; 5% of codons encoding for Leucine are CUU; 15% of codons encoding for Leucine are CUC; 3% of codons encoding for Leucine are CUA; and 73% of codons encoding for Leucine are CUG; c) for Isoleucine: 22% of codons encoding for Isoleucine are AUU; 75% of codons encoding for Isoleucine are AUC; and 3% of codons encoding for Isoleucine are AUA; d) for Methionine, 100% of codons encoding for Methionine are AUG; e) for Valine: 7% of codons encoding for Valine are GUU; 22% of codons encoding for Valine are GUC; 3% of codons encoding for Value are GUA; and 67% of codons encoding for Value are GUG; f) for Serine: 10% of codons encoding for Serine are UCU; 33% of codons encoding for Serine are UCC; 6% of codons encoding for Serine are UCA; 5% of codons encoding for Seville are AGU; and 46% of codons encoding for Serine are AGC; g) for Proline: 19% of codons encoding for Proline are CCU; 69% of codons encoding for Proline are CCC; and 12% of codons encoding for Proline are CCA; h) for Threonine: 10% of codons encoding for Threonine are ACU; 52% of codons encoding for Threonine are ACC; 8% of codons encoding for Threonine are ACA; and 30% of codons encoding for Threonine are ACG; i) for Alanine: 13% of codons encoding for Maniac. are GCU; 43% of codons encoding for Alanine are GCC; 8% of codons encoding for Alanine are GCA; and 35% of codons encoding for Alanine are GCG; j) for Tyrosine: 10% of codons encoding for Tyrosine are UAU; and 90% of codons encoding for Tyrosine are UAC; k) for Histidine: 100% of codons encoding for Histidine are CAC; 1) for Glutamine: 10% of codons encoding for Glutamine are CAA; and 90% of codons encoding for Glutamine are CAG; in) for Asparagine: 9% of codons encoding for Asparagine are AUU; and 91% of codons encoding for Asparagine are AAC; n) for Lysine: 5% of codons encoding for Lysine are AAA; and 95% of codons encoding for Lysine are AAG; o) for Aspartic Acid: 14% of codons encoding for Aspartic Acid are GAU; and 86% of codons encoding for Aspartic Acid are GAC; p) for Glutamic Acid: 5% of codons encoding for Glutamic Acid are GAA; and 95% of codons encoding for Glutamic Acid are GAG; q) for Cysteine: 10% of codons encoding for Cysteine are UGU; and 90% of codons encoding for Cysteine are UGC; r) for Tryptophan: 100% of codons encoding for Tryptophan are UGG; s) for Arginine: 11% of codons encoding for Arginine are CGU; 77% of codons encoding for Arginine are CGC; 4% of codons encoding for Arginine are CGA; 2% of codons encoding for Arginine are AGA; and 6% of codons encoding for Arginine are AGG; and t) for Glycine: 11% of codons encoding for Glycine are GGU; 72% of codons encoding for Glycine are GGC; 6% of codons encoding for Glycine are GGA; and 11% of codons encoding for Glycine are GGG; wherein for Serine the codon UCG should not be used, for Proline the codon CCG should not be used. for Histidine the codon CAU should not be used, and for Arginine the codon CGG should not be used. In some embodiments, the Chlamydomonas sp. is C. reinhardtii, the Nannochloropsis sp. is N. salina, or the Scenedesmus sp. is S. dimorphus.

Provided herein is an isolated polynucleotide, comprising: (a) a nucleic acid sequence of SEQ ID NO: 21, 19, 17, 20, 18, 16, 15, 61, 64, 66, 68 or 69; or (b) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69. Also provided is an organism transformed with the isolated polynucleotide and a vector comprising the isolated polynucleotide. In one embodiment, the vector further comprises a 5′ regulatory region. In another embodiment, the 5′ regulatory region further comprises a promoter. In one embodiment, the promoter is a constitutive promoter. In another embodiment, the promoter is an inducible promoter. Wherein the promoter is an inducible promoter, the inducible promoter may be a light inducible promoter, a nitrate inducible promoter, or a heat responsive promoter. In yet another embodiment, the vector further comprises a 3′ regulatory region.

Also provided herein is a photosynthetic organism transformed with an isolated polynucleotide comprising: (a) a nucleic acid sequence of SEQ ID NO: 21, 19, 17, 20, 18, 16, 15, 61, 64, 66, 68 or 69; or (b) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69 wherein the transformed organism's biomass is increased as compared to a biomass of an untransformed organism or a second transformed organism. The increase may be measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the competition assay is performed in a turbidostat and the increase is shown by the transformed organism having a positive selection coefficient as compared to either the untransformed organism or the second transformed organism. In some embodiments, the selection coefficient is at least 0.05, at least 0.10, at least 0.5, at least 0.75 at least 1.0, at least 1.5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increase in the transformed organism's biomass is measured by growth rate. In some embodiments, the transformed organism has at least a 5%, at least a 25%, at least a 50%, at least a 100%, at least a 150%, or at least a 200% increase in growth rate as compared to either the untransformed organism or the second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to either the untransformed organism or the second transformed organism. In another embodiment, the increase in the transformed organism's biomass is measured by an increase in carrying capacity. In one embodiment, the units of carrying capacity are mass per unit of volume or area. In another embodiment, the increase in the transformed organism's biomass is measured by an increase in culture productivity. In another embodiment, the units of culture productivity are grams per meter squared per day. In some embodiments, the transformed organism has at least a 5%, at least a 25%, at least: a 50%, at least a 100%, at least: a 150%, or at least a 200% increase in productivity as measured in grams per meter squared per day, as compared to either the untransformed organism or the second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to either the untransformed organism or the second transformed organism. In one embodiment, the organism is grown in an aqueous environment. In another embodiment, the organism is a vascular plant. In yet another embodiment, the organism is a non-vascular photosynthetic organism. In some embodiments, the organism is an alga or a bacterium. In one embodiment, the bacterium is a cyanobacterium. In another embodiment, the alga is a micro alga. In some embodiments, the microalga is at least one of a Chlamydomonas sp., Volvacales sp., Dunaliella sp., Scenedesmus sp., Chlorella sp., Hematococcus sp., Volvox sp., Nammochloropsis sp., Arthrospira sp., Sprirulina sp., Botryococcus sp., Haematococcus sr., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+.

Also provided herein is a method of comparing biomass of a first organism with biomass of a second organism, comprising: (a) transforming the first organism with a first polynucleotide, wherein the first polynucleotide comprises: (i) a nucleic acid sequence of SEQ ID NO: 21, 19, 17, 20, 18, 16, 15, 61, 64, 66, 68 or 69; or (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69; (b) determining the biomass of the first organism; (c) determining the biomass of the second organism; and (d) comparing the biomass of the first organism with the biomass of the second organism. In one embodiment, the second organism has been transformed with a second polynucleotide. In another embodiment, the biomass of the first organism is increased as compared to the biomass of the second organism. The increase may be measured by a competition assay, growth rate, carrying capacity, culture productivity, cell proliferation, seed yield, organ growth, or polysome accumulation. In one embodiment, the increase is measured by a competition assay. In another embodiment, the competition assay is performed in a turbidostat. In yet another embodiment, the competition assay is performed in a turbidostat and the increase in biomass of the first organism is shown by the first transformed organism having a positive selection coefficient as compared to the second organism. In other embodiments, the selection coefficient is at least 0.05, at least 0.10, at least 0.5, at least 0.75, at least 1.0, at least 1.5, or at least 2.0. In some embodiments, the selection coefficient is about 0.05, about 0.10, about 0.20, about 0.30, about 0.40, about 0.5, about 0.75, about 1.0, about 1.25, about 1.5, or about 2.0. In another embodiment, the increase in biomass of the first organism is measured by growth rate. In other embodiments, the first transformed organism has at least a 5%, at least a 25%, at least a 50%, at least a 100%, at least a 150%, or at least a 200% increase in growth rate as compared to the second organism. In some embodiments, the first transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to the second organism. In another embodiment, the increase in biomass of the first organism is measured by an increase in carrying capacity. In one embodiment, the units of carrying capacity are mass per unit of volume or area. In another embodiment, the increase in biomass of the first organism is measured by an increase in culture productivity. In one embodiment, the units of culture productivity are grams per meter squared per day. In other embodiments, the first transformed organism has at least a 5%, at least a 25%, at least a 50%, at least a 100%, at least a 150%, or at least a 200% increase in productivity as measured in grams per meter squared per day, as compared to the second organism. In some embodiments, the first transformed organism has about a 5%, about a 10%, about a 20%, about a 30%, about a 40%, about a 50%, about a 60%, about a 70%, about a 80%, about a 90%, about a 00%, about a 150%, or about a 200% increase in productivity as measured in grams per meter squared per day, as compared to the second organism. In one embodiment, the first and second organisms are grown in an aqueous environment. In another embodiment, the first and/or second organism is a vascular plant. In another embodiment, the first and/or second organism is a non-vascular photosynthetic organism. In other embodiments, the first and/or second organism is an alga or a bacterium. In one embodiment, the bacterium is a cyanobacterium. In yet another embodiment, the alga is a microalga. In some embodiments, the microalga is at least one of a Chlamydomonas sp., Volvacales sp., Dunalielia sp., Scenedesmus sp., Chlorella sp., Hematococcus sp., Volvox sp., Nannochloropsis sp. Arthrospira sp., Sprirulina sp., Botryococcus sp. Haematococcus sp. or Desmodesmus sp. In other embodiments, the microalga is at least one of Chlamydomonas reinhardtii, N. oceanica, N. salina, Dunaliella sauna, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 71 gr mt+.

Provide herein is a method of increasing biomass of an organism, comprising: (a) transforming the organism with a polynucleotide, wherein the polynucleotide comprises: (i) a nucleic acid sequence of SEQ ID NO: 21, 19, 17, 20, 18, 16, 15, 61, 64, 66, 68 or 69; or (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69; and wherein the nucleic acid of (i) or the nucleotide of (ii) encode for a polypeptide that when expressed results in an increase in the biomass of the organism. The increase may be measured by a competition assay, growth rate, carrying capacity, culture productivity, cell proliferation, seed yield, organ growth, or polysome accumulation. In one embodiment, the increase in the biomass of the organism is measured by a competition assay. In another embodiment, the competition assay is performed in a turbidostat. In yet another embodiment, the competition assay is performed in a turbidostat and the increase is shown by the transformed organism having a positive selection coefficient as compared to an untransformed organism or a second organism. In some embodiments, the selection coefficient is at least 0.05, at least 0.10, at least 0.5, at least 0.75, at least 1.0, at least 1.5, or at least 2,0. In other embodiments, the selection coefficient is about 0.05, about 0.10, about 0.20, about 0.30, about 0.40, about 0.5, about 0.75, about 1.0, about 1.25, about 1.5, or about 2.0. In another embodiment, an increase in the biomass of the organism is measured by growth rate. In some embodiments, the transformed organism has at least a 5%, at least a 25%, at least a 50%, at least a 100%, at least a 150%, or at least a 200% increase in growth rate as compared to an untransformed organism or a second organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to an untransformed organism or a second organism. In one embodiment, an increase in the biomass of the organism is measured by an increase in carrying capacity. In another embodiment, the units of carrying capacity are mass per unit of volume or area. In yet another embodiment, an increase in the biomass of the organism is measured by an increase in culture productivity, In one embodiment, the units of culture productivity are grams per meter squared per day. In some embodiments, the transformed organism has at least a 5%, at least a 25%, at least a 50%, at least a 100%, at least a 150%, or at least a 200% increase in productivity as measured in grams per meter squared per day, as compared to an untransformed organism or a second organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to an untransformed organism or a second organism. In one embodiment, the organism is grown in an aqueous environment. In another embodiment, the organism is a vascular plant. In yet another embodiment, the organism is a non-vascular photosynthetic organism. In some embodiments, the organism is an alga or a bacterium. In one embodiment, the bacterium is a cyanobacterium. In another embodiment, the alga is a microalga. In other embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In some embodiments, the microalga is at least one of Chlamydomonas reinhardtii, N. oceanica, N. salina, Dunalielia salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-169021 gr mt+.

Also provided herein is a method of screening for a protein involved in increased biomass of an organism comprising: (a) transforming the organism with a polynucleotide comprising: (i) a nucleic acid sequence of SEQ ID NO: 21, 19, 17, 20, 18, 16, 15, 61, 64, 66, 68 or 69; or (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68 or 69; wherein the nucleic acid of (i) or the nucleotide of (ii) encode for a polypeptide that when expressed results in an increase in the biomass of the organism as compared to an untransformed organism; and (b) observing a change in expression of an RNA in the transformed organism as compared to the same RNA in the untransformed organism. In one embodiment, the change is an increase in expression of the RNA in the transformed organism as compared to the same RNA in the untransformed organism. In another embodiment, the change is a decrease in expression of the RNA in the transformed organism as compared to the same RNA in the untransformed organism. In some embodiments, the change in expression of an RNA is measured by microarray, RNA-Seq, or serial analysis of gene expression (SAGE). In some embodiments, the change in expression of an RNA is at least two fold or at least four fold as compared to the untransformed organism. In one embodiment, the organism is grown in the presence of nitrogen. In another embodiment, the organism is grown in the absence of nitrogen.

Provided herein is an isolated polynucleotide, comprising: (a) a nucleic acid sequence of SEQ ID NO: 50, 51, 52, 53, 54, 55, 56, 57, 58, or 62; or (b) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 50, 51, 52, 53, 54, 55, 56, 57, 58, or 62. Also provided is an organism transformed with the isolated polynucleotide and a vector comprising the isolated polynucleotide. In one embodiment, the vector further comprises a 5′ regulatory region. In another embodiment, the 5′ regulatory region further comprises a promoter. The promoter may be a constitutive promoter or an inducible promoter. The inducible promoter may be a light inducible promoter, a nitrate inducible promoter, or a heat responsive promoter. In one embodiment, the vector further comprises a 3′ regulatory region.

Also provided herein is a photosynthetic organism transformed with an isolated polynucleotide comprising: (a) a nucleic acid sequence of SEQ ID NO: 50, 51, 52, 53, 54, 55, 56, 57, 58, or 62; or (b) nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 50, 51, 52, 53, 54, 55, 56, 57, 58, or 62; wherein the transformed organism's biomass is increased as compared to a biomass of an untransformed organism or a second transformed organism. The increase may be measured by a competition assay, growth rate, carrying capacity, culture productivity, cell proliferation, seed yield, organ growth, or polysome accumulation. In one embodiment, the increase in the transformed organism's biomass is measured by a competition assay. In another embodiment, the competition assay is performed in a turbidostat. In yet another embodiment, the competition assay is performed in a turbidostat and the increase is shown by the transformed organism having a positive selection coefficient as compared to either the untransformed organism or the second transformed organism. In some embodiments, the selection coefficient is at least 0.05, at least 0.10, at least 0.5, at least 0.75, at least 1,0, at least 1,5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increase in the transformed organism's biomass is measured by growth rate. In some embodiments, the transformed organism has at least a 5%, at least a 25%, at least a 50%, at least a 100%, at least a 150%, or at least a 200% increase in growth rate as compared to either the untransformed organism or the second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to either the untransformed organism or the second transformed organism. In one embodiment, the increase in the transformed organism's biomass is measured by an increase in carrying capacity. In one embodiment, the units of carrying capacity are mass per unit of volume or area. In one embodiment, the increase in the transformed organism's biomass is measured by an increase in culture productivity. In yet another embodiment, the units of culture productivity are grams per meter squared per day. In some embodiments, the transformed organism has at least a 5%, at least a 25%, at least a 50%, at least a 100%, at least a 150%, or at least a 200% increase in productivity as measured in grams per meter squared per day, as compared to either the untransformed organism or the second transformed organism. In other embodiments, the transformed organism has about a about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to either the untransformed organism or the second transformed organism. In one embodiment, the organism is grown in an aqueous environment. In another embodiment, the organism is a vascular plant. In yet another embodiment, the organism is a non-vascular photosynthetic organism. In some embodiments, the organism is an alga or a bacterium. In one embodiment, the bacterium is a cyanobacterium. In another embodiment, the alga is a microalga. In some embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, Dunaliella salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+.

Provided herein is a method of comparing biomass of a first organism with biomass of a second organism, comprising: (a) transforming the first organism with a first polynucleotide, wherein the first polynucleotide comprises: (i) a nucleic acid sequence of SEQ ID NO: 50, 51, 52, 53, 54, 55, 56, 57, 58, or 62; or (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 50, 51, 52, 53, 54, 55, 56, 57, 58, or 62; (b) determining the biomass of the first organism; (c) determining the biomass of the second organism; and (d) comparing the biomass of the first organism with the biomass of the second organism. In one embodiment, the second organism has been transformed with a second polynucleotide. In another embodiment, the biomass of the first organism is increased as compared to the biomass of the second organism. In some embodiments, the increase in biomass of the first organism is measured by a competition assay, growth rate, carrying capacity, culture, productivity, cell proliferation, seed yield, organ growth, or polysome accumulation. In yet another embodiment, the increase is measured by a competition assay. In another embodiment, the competition assay is performed in a turbidostat. In yet another embodiment, the competition assay is performed in a turbidostat and the increase is shown by the first transformed organism having a positive selection coefficient as compared to the second organism. In some embodiments, the selection coefficient is at least 0.05, at least 0.10, at least 0.5, at least 0.75, at least 1.0, at least 1.5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increase in the biomass of the first organism is measured by growth rate. In other embodiments, the first transformed organism has at least a 5%, at least a 25%, at least a 50%, at least a 100%, at least a 150%, or at least a 200% increase in growth rate as compared to the second organism. In other embodiments, the first transformed organism has about a 5%, about a 10%, about a 20%, about a 30%, about a 40%, about a 50%, about a 60%, about a 70%, about 80%, about a 90%, about a 100%, about a 150%, or about a 200% increase in growth rate as compared to the second organism. In one embodiment, the increase in the biomass of the first organism is measured by an increase in carrying capacity. In one embodiment, the units of carrying capacity are mass per unit of volume or area. In one embodiment, the increase in the biomass of the first organism is measured by an increase in culture productivity. In yet another embodiment, the units of culture productivity are grams per meter squared per day. In some embodiments, the first transformed organism has at least a 5%, at least a 25%, at least a 50%, at least a 100%, at least a 150%, or at least a 200% increase in productivity as measured in grams per meter squared per day, as compared to the second organism. In other embodiments, the first transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to the second organism. In yet another embodiment, the first and second organisms are grown in an aqueous environment. In other embodiments, the first and/or second organism is a vascular plant. In some embodiments, the first and/or second organism is a non-vascular photosynthetic organism. In other embodiments, the first and/or second organism is an alga or a bacterium. In one embodiment, the bacterium is a cyanobacterium. In another embodiment, the alga is a microalga. In some embodiments, the microalga is at least one of a Chlamydomonas sp. Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+.

Also provided herein is a method of increasing biomass of an organism, comprising: (a) transforming the organism with a polynucleotide, wherein the polynucleotide comprises: (i) a nucleic acid sequence of SEQ ID NO: 50, 51, 52, 53, 54, 55, 56, 57, 58, or 62; or (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 50, 51, 52, 53, 54, 55, 56, 57, 58, or 62; and wherein the nucleic acid of (i) or the nucleotide of (ii) encode for a polypeptide that when expressed results in an increase in the biomass of the organism. In some embodiments, the increase is measured by a competition assay, growth rate, carrying capacity, culture productivity, cell proliferation, seed yield, organ growth, or polysome accumulation. In one embodiment, the increase in the biomass of the organism is measured by a competition assay. In another embodiment, the competition assay is performed in a turbidostat. In yet another embodiment, the competition assay is performed in a turbidostat and the increase is shown by the transformed organism having a positive selection coefficient as compared to either an untransformed organism or a second transformed organism. In some embodiments, the selection coefficient is at least 0.05, at least 0.10, at least 0.5, at least 0.75, at least 1.0, at least 1.5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increase in the biomass of the organism is measured by growth rate. In some embodiments, the transformed organism has at least a 5%, at least a 25%, at least a 50%, at least a 100%, at least a 150%, or at least a 200% increase in growth rate as compared to either an untransformed organism or a second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to either an untransformed organism or a second transformed organism. In one embodiment, the increase in the biomass of the organism is measured by an increase in carrying capacity. In one embodiment, the units of carrying capacity are mass per unit of volume or area. In one embodiment, the increase in the biomass of the organism is measured by an increase in culture productivity. In another embodiment, the units of culture productivity are grams per meter squared per day. In some embodiments, the transformed organism has at least a 5%, at least a 25%, at least a 50%, at least a 100%, at least a 150%, or at least a 200% increase in productivity as measured in grams per meter squared per day, as compared to either an untransformed organism or a second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200%; increase in productivity as measured in grams per meter squared per day, as compared to either an untransformed organism or a second transformed organism. In one embodiment, the organism is grown in an aqueous environment. In another embodiment, the organism is a vascular plant. In yet another embodiment, the organism is a non-vascular photosynthetic organism. In other embodiments, the organism is an alga or a bacterium. In one embodiment, the bacterium is a cyanobacterium. In another embodiment, the alga is a microalga. In some embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp, Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+.

Also provided herein is a method of screening for a protein involved in increased biomass of an organism comprising: (a) transforming the organism with a polynucleotide comprising: (i) a nucleic acid sequence of SEQ ID NO: 50, 51, 52, 53, 54, 55, 56, 57, 58, or 62; or (ii) a nucleotide sequence with at least 50%, at least 60%, at least 70%, at least 75%, at least 80%, at least 85%, at least 90%, at least 95%, at least 9800, or at least 99% sequence identity to the nucleic acid sequence. of SEQ ID NO: 50, 51, 52, 53, 54, 55, 56, 57, 58, or 62; wherein the nucleic acid of (i) or the nucleotide of (ii) encode for a polypeptide that when expressed results in an increase in the biomass of the organism as compared to an untransformed organism; and (b) observing a change in expression of an RNA in the transformed organism as compared to the same RNA in the untransformed organism. In one embodiment, the change is an increase in expression of the RNA in the transformed organism as compared to the same RNA in the untransformed organism. In another embodiment, the change is a decrease in expression of the RNA in the transformed organism as compared to the same RNA in the untransformed organism. In some embodiments, the change is measured by microarray, RNA-Seq, or serial analysis of gene expression (SAGE). In other embodiments, the change is at least two fold or at least four fold as compared to the untransformed organism, in one embodiment, the organism is grown in the presence or absence of nitrogen.

Provided herein is an isolated polynucleotide, comprising: (a) a nucleic acid sequence of SEQ ID NO: 32, 38, 34, or 40; (b) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 32, 38, 34, or 40; (c) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (d) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species. Also provided is an organism transformed with the isolated polynucleotide and a vector comprising the isolated polynucleotide. In one embodiment, the vector further comprises a 5′ regulatory region. In another embodiment, the 5′ regulatory region further comprises a promoter. In another embodiment, the promoter is a constitutive promoter. In one embodiment, the promoter is an inducible promoter. Wherein the promoter is an inducible promoter, the inducible promoter may be a light inducible promoter, a nitrate inducible promoter, or a heat responsive promoter. In another embodiment, the vector further comprises a 3′ regulatory region.

Also provided herein is a photosynthetic organism transformed with an isolated polynucleotide comprising: (a) a nucleic acid sequence of SEQ ID NO: 32, 38, 34, or 40; (b) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 32, 38, 34, or 40; (c) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (d) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species; wherein the transformed organism's biomass is increased as compared to a biomass of an untransformed organism or a second. transformed organism. The increase may be measured by a competition assay, growth rate, carrying capacity, culture productivity, cell proliferation, seed yield, organ growth, or polysome accumulation. The increase in the transformed organism's biomass can be measured by a competition assay. In one embodiment, the competition assay is performed in a turbidostat. In yet another embodiment, the competition assay is performed in a turbidostat and the increase is shown by the transformed organism having a positive selection coefficient as compared to either the untransformed organism or the second transformed organism. In some embodiments, the selection coefficient is at least 0.05, at least 0.10, at least 0.5, at least 0.75, at least 1.0, at least 1.5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. The increase in the transformed organism's biomass can be measured by growth rate. In some embodiments, the transformed organism has at least a 5%, at least a 25%, at least a 50%, at least a 100%, at least a 150%, or at least a 200% increase in growth rate as compared to either the untransformed organism or the second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to either the untransformed organism or the second transformed organism. The increase in the transformed organism's biomass can be measured by an increase in carrying capacity. In one embodiment, the units of carrying capacity are mass per unit of volume or area. The increase in the transformed organism's biomass can be measured by an increase in culture productivity. In one embodiment, the units of culture productivity are grams per meter squared per day. In other embodiments, the transformed organism has at least a 5%, at least a 25%, at least a 50%, at least a 100%, at least a 150%, or at least a 200% increase in productivity as measured in grams per meter squared per day, as compared to either the untransformed organism or the second transformed organism. In some embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to either the untransformed organism or the second transformed organism. In one embodiment, the organism is grown in an aqueous environment. In another embodiment, the organism is a vascular plant. In yet another embodiment, the organism is a non-vascular photosynthetic organism. In other embodiments, the organism is an alga or a bacterium. In one embodiment, the bacterium is a cyanobacterium. In another embodiment, the alga is a microalga. In some embodiments, the microalga is at least one of a Chlamydomonas sp., Volvacales sp. Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus in one embodiment, the C. reinhardtii is wild-type strain CC-1690 21 gr mt+.

Provided herein is a method of comparing biomass of a first organism with biomass of a second organism, comprising: (a) transforming the first organism with a first polynucleotide, wherein the first polynucleotide comprises: (i) a nucleic acid sequence of SEQ ID NO: 32, 38, 34, or 40; (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 32, 38, 34, or 40; (iii) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (iv) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species; (b) determining the biomass of the first organism; (c) determining the biomass of the second organism; and (d) comparing the biomass of the first organism with the biomass of the second organism. In one embodiment, the second organism has been transformed with a second polynucleotide. In another embodiment, the biomass of the first organism is increased as compared to the biomass of the second organism. The increased biomass of the first organism may be measured by a competition assay, growth rate, carrying capacity, culture productivity, cell proliferation, seed yield, organ growth, or polysome accumulation. In one embodiment, the increased biomass of the first organism is measured by a competition assay. In one embodiment, the competition assay is performed in a turbidostat. In yet another embodiment, the competition assay is performed in a turbidostat and the increase is shown by the first transformed organism having a positive selection coefficient as compared to the second organism. In some embodiments, the selection coefficient is at least 0.05, at least 0.10, at least 0.5, at least 0.75, at least 1.0, at least 1.5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increased biomass of the first organism is measured by growth rate. In other embodiments, the first transformed organism has at least a 5%, at least a 25%, at least a 50%, at least a 100%, at least a 150%, or at least a 200% increase in growth rate as compared to the second organism. In some embodiments, the first transfomied organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to the second organism. In one embodiment, the increased biomass of the first organism is measured by an increase in carrying capacity. In another embodiment, the units of carrying capacity are mass per unit of volume or area. In one embodiment, the increased biomass of the first organism is measured by an increase in culture productivity. In another embodiment, the units of culture productivity are grams per meter squared per day. In some embodiments, the first transformed organism has at least a 5%, at least a 25%, at least a 50%, at least a 100%, at least a 150%, or at least a 200% increase in productivity as measured in grams per meter squared per day, as compared to the second organism. In other embodiments, the first transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to the second organism. In one embodiment, the first and second organisms are grown in an aqueous environment. In other embodiments, the first and/or second organism is a vascular plant. In yet other embodiments, the first and/or second organism is a non-vascular photosynthetic organism. In other embodiments, the first and/or second organism is an alga or a bacterium. In one embodiment, the bacterium is a cyanobacterium. In another embodiment, the alga is a microalga. In other embodiments, the microalga is at least one of a Chlamydomonas sp., Volvacales sp., Dunaliella sp., Scenedesmus Sp., Chlorella sp., Hematococcus sp., Volvox sp., Nannochloropsis sp., Arthrospira sp., Sprirulina sp., Botryococcus sp., Haematococcus sp., or Desmodesmus sp. In some embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+.

Also provided herein is a method of increasing biomass of an organism, comprising: (a) transforming the organism with a polynucleotide, wherein the polynucleotide comprises: (i) a nucleic acid sequence of SEQ ID NO: 32, 38, 34, or 40; (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 32, 38, 34, or 40; (iii) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (iv) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species; and wherein the nucleic acid of (i), (iii), or (iv), or the nucleotide sequence of (ii) encode for a polypeptide that when expressed results in an increase in the biomass of the organism. The increase may be measured by a competition assay, growth rate, carrying capacity, culture productivity, cell proliferation, seed yield, organ growth, or polysome accumulation. In one embodiment, the increase in the biomass of the organism is measured by a competition assay. In another embodiment, the competition assay is performed in a turbidostat. In yet another embodiment, the competition assay is performed in a turbidostat and the increase is shown by the transformed organism having a positive selection coefficient as compared to either an untransformed organism or a second transformed organism. In some embodiments, the selection coefficient is at least 0.05, at feast 0.10, at least 0.5, at least 0.75, at least 1.0, at least 1.5, or at feast 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increase in the biomass of the organism is measured by growth rate. In some embodiments, the transformed organism has at least a 5%, at least a 25%, at least a 50%, at least a 100%, at least a 150%, or at least a 200% increase in growth rate as compared to either an untransformed organism or a second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to either an untransformed organism or a second transformed organism. In one embodiment, the increase in the biomass of the organism is measured by an increase in carrying capacity. In another embodiment, the units of carrying capacity are mass per unit of volume or area. In one embodiment, the increase in the biomass of the organism is measured by an increase in culture productivity. In another embodiment, the units of culture productivity are grams per meter squared per day, some embodiments, the transformed organism has at least a 5%, at least a 25%, at least a 50%, at least a 100%, at least a 150%, or at least a 200% increase in productivity as measured in grams per meter squared per day, as compared to either an untransformed organism or a second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to either an untransformed organism or a second transformed organism. In one embodiment, the organism is grown in an aqueous environment. In another embodiment, the organism is a vascular plant. In yet another embodiment, the organism is a non-vascular photosynthetic organism. In some embodiments, the organism is an alga or a bacterium. In one embodiment, the bacterium is a cyanobacterium. In another embodiment, the alga is a microalga. In some embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata. Dunaliella tertiolecta, S. Maximus, or A. Fusiformus. In another embodiment, the C. reinhardtii is wild-type strain CC-1690 21 gr mt+.

Provided herein is a method of screening for a protein involved in increased biomass of an organism comprising: (a) transforming the organism with a polynucleotide comprising: (i) a nucleic acid sequence of SEQ ID NO: 32, 38, 34, or 40; (ii) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 32, 38, 34, or 40; (iii) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or (iv) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species; wherein the nucleic acid of (i), (iii), or (iv), or the nucleotide of (ii) encode for a polypeptide that when expressed results in an increase in the biomass of the organism as compared to an untransformed organism; and (b) observing a change in expression of an RNA in the transformed organism as compared to the same RNA in the untransformed organism. In one embodiment, the change is an increase in expression of the RNA in the transformed organism as compared to the same RNA in the untransformed organism hi another embodiment, the change is a decrease in expression of the RNA in the transformed organism as compared to the same. RNA in the untransformed organism. In some embodiments, the Change is measured by microarray, RNA-Seq, or serial analysis of gene expression (SAGE). In other embodiments, the change is at least two fold or at least four fold as compared to the untransformed organism. In other embodiments, the organism is grown in the presence or absence of nitrogen.

Also provided herein is an isolated polynucleotide encoding a protein comprising, (a) an amino acid sequence of SEQ ID NO: 33 or SEQ ID NO: 39; or (b) a homolog of the amino acid sequence of (a), wherein the homolog has at least 50%, at least 60%, at least 70%, at least 75%, at least 80%, at least 85%, at least 90%, at least 95%, at least 98%, or at least 99% sequence identity to the amino acid sequence of SEQ ID NO: 33 or SEQ ID NO: 39. Provided herein is an organism transformed with the isolated polynucleotide and an expressed protein encoded by the polynucleotide.

Provided herein is a higher plant transformed with an isolated polynucleotide comprising: (a) a nucleic acid sequence of SEQ ID NO: 21, 19, 17, 20, 18, 16, 15, 61, 64, 66, 68, 69, 50, 51, 52, 53, 54, 55, 56, 57, 58, 62, 32, 38, 34, or 40; or (b) a nucleotide sequence with at least 50%, at least 60%, at least 70%, at least 75%, at least 80%, at least 85%, at least 90%, at least 95%, at least 98%, or at least 99% sequence identity to the nucleic acid sequence of SEQ ID NO: 21, 19, 17, 20, 18, 16, 15, 61, 64, 66, 68, 69, 50, 51, 52, 53, 54, 55, 56, 57, 58, 62, 32, 38, 34, or 40; wherein the transformed organism's biomass is increased as compared to a biomass of an untransformed organism or a second transformed organism. The increase may be measured by a competition assay, growth rate, carrying capacity, culture productivity, cell proliferation, seed yield, organ growth, or polysome accumulation. In one embodiment, the increase in the transformed organism's biomass is measured by a competition assay. In one embodiment, the competition assay is performed in a turbidostat. In yet another embodiment, the competition assay is performed in a turbidostat and the increase is shown by the transformed organism having a positive selection coefficient as compared to either the untransformed organism or the second transformed organism. In some embodiments, the selection coefficient is at least 0.05, at least 0.10, at least 0.5, at least 0.75, at least 1.0, at least 1.5, or at least 2.0. In other embodiments, the selection coefficient is about 0.05, about 0.10, about 0.20, about 0.30, about 0.40, about 0.5, about 0.75, about 1.0, about 1.25, about 1.5, or about 2.0. In one embodiment, the increase in the transformed organism's biomass is measured by growth rate. In some embodiments, the transformed organism has at least a 5%, at least a 25%, at least a 50%, at least a 100%, at least a 150%, or at least a 200% increase in growth rate as compared to either the untransformed organism or the second transformed organism. In other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in growth rate as compared to either the untransformed organism or the second transformed organism. In one embodiment, the increase in the transformed organism's biomass is measured by an increase in carrying capacity. In one embodiment, the units of carrying capacity are mass per unit of volume or area. In one embodiment, the increase in the transformed organism's biomass is measured by an increase in culture productivity. In one embodiment, the units of culture productivity are grams per meter squared per day. In other embodiments, the transformed organism has at least a 5%, at least a 25%, at least a 50%, at least a 100%, at least a 150%, or at least a 200% increase in productivity as measured in grains per meter squared per day, as compared to either the untransformed organism or the second transformed organism. In some other embodiments, the transformed organism has about a 5%, about a 10%, about a 20%, about a 30%, about a 40%, about a 50%, about a 60%, about a 70%, about a 80%, about a 90%, about a 100%, about a 150%, or about a 200% increase in productivity as measured in grams per meter squared per day, as compared to either the untransformed organism or the second transformed organism. In one embodiment, the organism is grown an aqueous environment. In another embodiment, the higher plant is Arabidopsis thaliana. In other embodiments, the higher plant is a Brassica, Glycine, Gossypium, Medicago, Zen, Sorghum, Oryza, Triticum, or Panicum species.

Also provided herein is a codon usage table capable of being used to codon optimize a nucleic acid for expression in the nucleus of a Desmodesmus, a Chlamydomonas, a Nannochloropsis, and/or a Scenedesmus species, comprising the following data: a) for Phenylalanine: 16% of codons encoding for Phenylalanine are UUU; and 84% of codons encoding for Phenylalanine are UUC; b) for Leucine: 1% of codons encoding for Leucine are UUA; 4% of codons encoding for Leucine are LUG; 5% of codons encoding for Leucine are ULU; 15% of codons encoding for Leucine are CCG; 3% of codons encoding for Leucine are CUA; and 73% of codons encoding for Leucine are CUG; c) for isoleucine: 22% of codons encoding for Isoleucine are AUU; 75% of codons encoding for Isoleucine are AUC; and 3% of codons encoding for isoleucine are AUA; d) for Methionine, 100% of codons encoding for Methionine are AUG; e) for Valine: 7% of codons encoding for Valine are GUU; 22% of codons encoding for Valine are GUC; 3% of codons encoding for Valine are GUA; and 67% of codons encoding for Valine are GUG; f) for Serine: 10% of codons encoding for Serine are UCU; 33% of codons encoding for Serine are UCC; 6% of codons encoding for Serine are UCA; 5% of codons encoding for Serine are AGU; and 46% of codons encoding for Serine are AGC; g) for Proline: 19% of codons encoding for Proline are CCU; 69% of codons encoding for Proline are CCC; and 12% of codons encoding for Proline are CCA; h) for Threonine: 10% of codons encoding for Threonine are ACU; 52% of codons encoding for Threonine are ACC; 8% of codons encoding for Threonine are ACA; and 30% of codons encoding for Threonine are ACG; i) for Alanine: 13% of codons encoding for Alanine are GCU; 43% of codons encoding for Alanine are G-CC; 8% of codons encoding for Alanine are G-CA; and 35% of codons encoding for Alanine are GCG; j) for Tyrosine: 10% of codons encoding for Tyrosine are UAU; and 90% of codons encoding for Tyrosine are UAC; k) for Histidine: 100% of codons encoding for Histidine are CAC; l) for Glutamine: 10% of codons encoding for Glutamine are CAA; and 90% of codons encoding for Glutamine are CAG; in) for Asparagine: 9% of codons encoding for Asparagine are AUU; and 91% of codons encoding for Asparagine are AC; n) for Lysine: 5% of codons encoding for Lysine are AAA; and 95% of codons encoding for Lysine are AAG; o) for Aspartic Acid: 14% of codons encoding for Aspartic Acid are GAU; and 86% of codons encoding for Aspartic Acid are GAC; p) for Glutamic Acid: 5% of codons encoding for Glutamic Acid are GAA; and 95% of codons encoding for Glutamic Acid are GAG; q) for Cysteine: 10% of codons encoding for Cysteine are UGU; and 90% of codons encoding for Cysteine are UGC; r) for Tryptophan; 100% of codons encoding for Tryptophan are UGG; s) for Arginine: 11% of codons encoding for Arginine are CGU; 77% of codons encoding for Arginine are CGC; 4% of codons encoding for Arginine are CGA; 2% of codons encoding for Arginine are AGA; and 6% of codons encoding for Arginine are AGG; and t) for Glycine: 11% of codons encoding; for Glycine are GGU; 72% of codons encoding for Glycine are GGC; 6% of codons encoding for Glycine are GGA; and 11% of codons encoding for Glycine are GGG; wherein for Serine the codon UCG should not be used, for Proline the codon CCG should not be used, for Histidine the codon CAU should not be used, and for Arginine the codon CGG should not be used. In one embodiment, the Chlamydomonas sp. is C. reinhardtii. In another embodiment, the Nannochloropsis sp. is N. salina. In yet another embodiment, the Scenedesmus sp. is S. dimorphus.

### BRIEF DESCRIPTION OF THE DRAWINGS

These and other features, aspects, and advantages of the present disclosure will become better understood with regard to the following description, appended claims and accompanying figures where:

FIG. 1 shows competition data for yield genes versus wild type Chlamydomonas reinhardtii. Diamonds represent turbidostat 1, squares represent turbidostat 2, and triangles represent turbidostat 3. The y-axis is the percent of the population that is transgenic, with the balance being wild type, and the x-axis is time in weeks.

FIG. 2 shows the growth rate for several YD3 transgenic lines along with a wild type Chlamydomonas reinhardtii line.

FIG. 3 shows the growth rate for several YD5 transgenic lines along with a wild type Chlamydomonas reinhardtii line.

FIG. 4 shows the growth rate for several YD7 transgenic lines along with a wild type Chlamydomonas reinhardtii line.

FIG. 5 shows nuclear overexpression vector SENuc745. All seven nucleotide sequences (YD1-YD7) were each individually cloned into the segment of the vector entitled “YD7.”

FIG. 6 shows selection coefficients for transgenic lines over expressing YD genes (indicated on the x-axis), with each data point representing a time point from replicate turbidostats, and the mean and standard deviation indicated by the horizontal bars, Selection coefficient (s) is on the y-axis in units of day⁻¹.

FIG. 7 shows data from a 96-well micro plate growth assay measuring the growth rate of individual YD gene transformants. Each transformant was grown and analyzed in duplicate or triplicate (e.g. YD22 transformant #4=YD22-4 is represented by 2 transformants, YD27 transformant #3=YD27-3 is represented by 3 transformants). The data was analyzed by a one way am lysis of “r” (growth rate) by transformant using a Dunnet's test.

FIG. 8 shows data from a 96-well micro plate growth assay measuring the growth rate of each group of YD gene transformants. All transformants for a given YD gene (e.g. YD22-1, YD22-2, YD22-3 . . . etc.) were analyzed together. The data was analyzed by a one way analysis of r by YD gene using a Dunnet's test.

FIG. 9 shows an expression vector Senuc1728. Senuc1728 comprises a pBR322 Origin, AR4 promoter, Ble gene, PsaD terminator, aphVIII-Paro, PsaD promoter, ampicillin gene, BamHI restriction site, and an Xhol restriction site.

FIG. 10 shows an expression vector Senuc2118. Senuc2118 comprises a pBR322 Origin, AR4 promoter, Ble gene, PsaD terminator, aphVIII-Pare, PsaD promoter, ampicillin gene, BamHI restriction site, an XhoI restriction site, and a P28 transit peptide.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present inventive discovery.

As used in this specification and the appended claims, the singular forms “a”, “an” and “the” include plural reference unless the context clearly dictates otherwise

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Examples of Genes, Nucleic Acids, Proteins, and Polypeptides that can be Used in the Embodiments Disclosed Herein Include, but are not Limited to:

If an initial start codon (Met) is not present in any of the amino acid sequences disclosed herein, including sequences contained in the sequence listing, one of skill in the art would be able to include, at the nucleotide level, an initial ATG, so that the translated polypeptide would have the initial Met. If a start and/or stop codon is not present at the beginning and/or end of a coding sequence, one of skill in the art would know to insert an “ATG” at the beginning of the coding sequence and nucleotides encoding for a stop codon (any one of TAA, TAG, or TGA) at the end of the coding sequence. Several of the nucleotide sequences disclosed herein are missing an initial “ATG” and/or are missing a stop codon. Any of the disclosed nucleotide sequences can be, if desired, fused to another nucleotide sequence that when operably linked to a “control element” results in the proper translation of the encoded amino acids (for example, a fusion protein). In addition, two or more nucleotide sequences can be linked by a short peptide, for example, a viral peptide.

If an “R” appears in a nucleic acid sequence, R is A or G.

If a “Y” appears in a nucleic acid sequence, Y is C or T.

SEQ ID NO: 1 is the nucleic acid sequence of endogenous YD1 (SEQ ID NO: 22), codon-optimized for expression in the nucleus of Chlamydomonas reinhardtii.

SEQ ID NO: 2 is the nucleic acid sequence of endogenous YD2 (SEQ ID NO: 23), codon-optimized for expression in the nucleus of Chlamydomonas reinhardtii. SEQ ID NO: 2 has a deletion of three nucleic acids starting at position 997.

SEQ ID NO: 3 is the nucleic acid sequence of endogenous YD3 (SEQ ID NO: 24), codon-optimized for expression in the nucleus of Chlamydomonas reinhardtii.

SEQ ID NO: 4 is the nucleic acid sequence of endogenous YD4 (SEQ ID NO: 25), codon-optimized for expression in the nucleus of Chlamydomonas reinhardtii.

SEQ ID NO: 5 is the nucleic acid sequence of endogenous YD5 (SEQ ID NO: 26), codon-optimized for expression in the nucleus of Chlamydomonas reinhardtii. SEQ ID NO: 5 has a deletion of an “ATG” at the beginning of the sequence.

SEQ ID NO: 6 is the nucleic acid sequence of endogenous YD6 (SEQ ID NO: 27), codon-optimized for expression in the nucleus of Chlamydomonas reinhardtii. SEQ ID NO: 6 also has a CTCGAG inserted directly after the start codon.

SEQ ID NO: 7 is the nucleic acid sequence of endogenous YD7 (SEQ ID NO: 28), codon-optimized for expression in the nucleus of Chlamydomonas reinhardtii.

SEQ ID NO: 8 is the translated protein sequence of SEQ ID NO: 1.

SEQ ID NO: 9 is the translated protein sequence of SEQ ID NO: 2.

SEQ ID NO: 10 is the translated protein sequence of SEQ ID NO: 3.

SEQ ID NO: 11 is the translated protein sequence of SEQ ID NO: 4.

SEQ ID NO: 12 is the translated protein sequence of SEQ ID NO: 5.

SEQ ID NO: 13 is the translated protein sequence of SEQ ID NO: 6.

SEQ ID NO: 14 is the translated protein sequence of SEQ ID NO: 7.

SEQ ID NO: 15 is the nucleic acid sequence of SEQ ID NO: 1, without a start codon (“ATG”).

SEQ ID NO: 16 is the nucleic acid sequence of SEQ ID NO: 2, without a start codon (“ATG”).

SEQ ID NO: 17 is the nucleic acid sequence of SEQ ID NO: 3, without a start codon (“ATG”).

SEQ ID NO: 18 is the nucleic acid sequence of SEQ ID NO: 4, without a start codon (“ATG”).

SEQ ID NO: 19 is the nucleic acid sequence of SEQ ID NO: 5, without a start codon (“ATG”).

SEQ ID NO: 20 is the nucleic acid sequence of SEQ ID NO: 6, without a start codon (“ATG”), and without the CTCGAG directly after the start codon.

SEQ ID NO: 21 is the nucleic acid sequence of SEQ ID NO: 7, without a start codon (“ATG”).

SEQ ID NO: 22 is the endogenous nucleic; acid sequence of YD1.

SEQ ID NO: 23 is the endogenous nucleic acid sequence of YD2. “Y” is C or T. “R” is A or G.

SEQ ID NO: 24 is the endogenous nucleic; acid sequence of YD3.

SEQ ID NO: 25 is the endogenous nucleic; acid sequence of YD4.

SEQ ID NO: 26 is the endogenous nucleic; acid sequence of YD5.

SEQ ID NO: 27 is the endogenous nucleic acid sequence of YD6. Nucleotides 1 through 174 represent the transit peptide and starting “ATG”.

SEQ ID NO: 28 is the endogenous nucleic acid sequence of YD7. Nucleotides 1 through 99 represent the transit peptide and starting “ATG”.

SEQ ID NO: 29 is the endogenous sequence of a novel rubisco activase isolated from Scenedesmus dimorphus.

SEQ ID NO: 30 is the translated sequence of SEQ ID NO: 29.

SEQ ID NO: 31 is SEQ ID NO: 29 codon optimized for nuclear expression in a Desmodesmus species.

SEQ ID NO: 32 is SEQ ID NO: 29 without the initial “ATG.”

SEQ ID NO: 33 is SEQ ID NO: 30 without the initial “M.”

SEQ ID NO: 34 is SEQ ID NO: 31 without the initial “ATG.”

SEQ ID NO: 35 is the endogenous sequence of a novel rubisco activase isolated from a Desmodesmus species.

SEQ ID NO: 36 is the translated sequence of SEQ ID NO: 35.

SEQ ID NO: 37 is SE ID NO: 35 codon optimized for nuclear expression in a Desmodesmus species.

SEQ ID NO: 38 is SEQ ID NO: 35 without the initial “ATG.”

SEQ ID NO: 39 is SEQ ID NO: 36 without the initial “M.”

SEQ ID NO: 40 is SEQ ID NO: 37 without the initial “ATG.”

SEQ ID NO: 41 is SEQ ID NO: 23 codon optimized for nuclear expression in Scenedesmus dimorphus, with an XhoI restriction site directly before the start codon and a BamHI restriction site directly after the stop codon. Directly prior to the stop codon is an extra sequence ACGGGC. SEQ ID NO: 41 has a deletion of three nucleic acids starting at position 1003.

SEQ ID NO: 42 is SEQ ID NO: 24 codon optimized for nuclear expression in Scenedesmus dimorphus, with an XhoI restriction site directly before the start codon and a BamHI restriction site directly after the stop codon.

SEQ ID NO: 43 is a thermostable variant Rubisco activase B gene sequence (as described in Kurek, I., et al., The Plant Cell (2007) Vol. 19:3230-3241) codon optimized for nuclear expression in Scenedesmus dimorphus, with an XhoI restriction site directly before the start codon and a BamHI restriction site directly after the stop codon. The mutations made are F168L, V257I, and K310N (relative to the A. thaliana RCA1 protein sequence).

SEQ ID NO 44 is SEQ ID NO: 27 codon optimized for nuclear expression in Scenedesmus dimorphus, with an XhoI restriction site directly before the start codon and a BamHI restriction site directly after the stop codon. Directly prior to the stop codon is an extra sequence ACCGGC.

SEQ ID NO: 45 is SEQ ID NO: 27 codon optimized for chloroplast expression in Scenedesmus dimorphus, with an NdeI restriction site at the 5′ end that contains a start codon and an XbaI restriction site directly after the stop codon. Directly prior to the stop codon is an extra sequence ACTGGT. SEQ ID NO: 45 does not contain the transit peptide of SEQ ID NO: 27.

SEQ ID NO: 46 is SEQ ID NO: 28 codon optimized for nuclear expression in Scenedesmus dimorphus, with an XhoI restriction site directly before the start codon and a BamHI restriction site directly after the stop codon. Directly prior to the stop codon is an extra sequence ACCGGC.

SEQ ID NO: 47 is SEQ ID NO: 28 codon optimized for chloroplast expression in Scenedesmus dimorphus, with an NdeI restriction site at the 5′ end that contains a start codon and an XbaI restriction site directly after the stop codon. Directly prior to the stop codon is an extra sequence ACAGGT. SEQ ID NO: 47 does not contain the transit peptide of SEQ ID NO: 28.

SEQ ID NO: 48 is SEQ ID NO: 26 codon optimized for nuclear expression in Scenedesmus dimorphus, with an XhoI restriction site directly before the start codon and a BamHI restriction site directly after the stop codon. SEQ ID NO: 48 has a deletion of an “ATG” directly prior to the first “ATG”. In addition, SEQ ID NO: 48 has an extra sequences ACCGGC directly prior to the stop codon.

SEQ ID NO: 49 is SEQ ID NO: 25 codon optimized for nuclear expression in Scenedesmus dimorphus, with an XhoI restriction site directly before the start codon and a BamHI restriction site directly after the stop codon. Directly prior to the stop codon is an extra sequence ACGGGC.

SEQ ID NO: 50 is SEQ ID NO: 41 without the XhoI restriction site, the start codon, the stop codon, and the BamHI restriction site. Also the sequence “ACGGGC” is removed.

SEQ ID NO: 51 is SEQ ID NO: 42 without the XhoI restriction site, the start codon, the stop codon, and the BamHI restriction site.

SEQ ID NO: 52 is SEQ ID NO: 43 without the XhoI restriction site, the start codon, the stop codon, and the BamHI restriction site,

SEQ ID NO: 53 is SEQ ID NO: 44 without the XhoI restriction site, the start codon, the stop codon, and the BamHI restriction site, Also the sequence “ACCGGC” is removed.

SEQ ID NO: 54 is SEQ ID NO: 45 without the NdeI restriction site that contains the start codon, and without the stop codon and the XbaI restriction site. Also the sequence “ACTGGT” is removed.

SEQ ID NO: 55 is SEQ ID NO: 46 without the XhoI restriction site, the start codon, the stop codon, and the BamHI restriction site, Also the sequence “ACCGGC” is removed.

SEQ ID NO: 56 is SEQ ID NO: 47 without the NdeI restriction site that contains the start codon, and without the stop codon and the XbaI restriction site. Also the sequence “ACAGGT” is removed.

SEQ ID NO: 57 is SEQ ID NO: 48 without the XhoI restriction site., the start codon, the stop codon, and the BamHI restriction site. Also the sequence “ACCGGC” is removed.

SEQ ID NO: 58 is SEQ ID NO: 49 without the XhoI restriction site., the start codon, the stop codon, and the BamHI restriction site. Also the sequence “ACGGGC” is removed.

SEQ ID NO: 59 is SEQ ID NO: 2 with a “GYG” sequence starting at nucleotide number 997. “Y” is either C or T.

SEQ ID NO: 60 is SEQ NO: 41 with a “GYG” sequence starting at nucleotide number 1003, “Y” is either C or T.

SEQ ID NO: 61 is SEQ ID NO:59 without a start codon “ATG.”

SEQ ID NO: 62 is SEQ ID NO: 60 without an XhoI restriction site directly before the start codon, without the start codon, without the extra sequence ACGGGC prior to the stop codon, without a stop codon, and without a BamHI restriction site directly after the stop codon.

SEQ ID NO: 63 is the nucleic acid sequence of the YD3 protein (SEQ ID NO: 10) codon optimized for expression in the nucleus of C. reinhardtii. SEQ ID NO: 63 is YD41.

SEQ ID NO: 64 is the nucleic acid sequence of SEQ ID NO: 63 without the start codon and the stop codon.

SEQ ID NO: 65 is a thermostable variant Rubisco activase 13 gene sequence (as described in Kurek, I., et al., The Plant Cell (2007) Vol. 19:3230-3241) codon optimized for nuclear expression in C. reinhardtii. The mutations made are F168L, V257I, and K310N (relative to the A. thaliana RCA1 protein sequence). SEQ ID NO: 65 is YD27.

SEQ ID NO: 66 is the nucleic acid sequence of SEQ ID NO: 65 without the start codon and the stop codon.

SEQ ID NO: 67 is the nucleic acid sequence of a YD2 protein (SEQ ID NO: 70) codon optimized for expression in the nucleus of C. reinhardtii. SEQ ID NO: 67 is YD22. SEQ ID NO: 67 is lacking three nucleic acids starting at position 997.

SEQ ID NO: 68 is the nucleic acid sequence of SEQ ID NO: 67 without the start codon, without the stop codon, and without a nucleotide sequence “ACGGGC” directly before the stop codon.

SEQ ID NO: 69 is the nucleic acid sequence of SEQ ID NO: 67 without the start codon and without the stop codon.

SEQ ID NO: 70 is the translated sequence of SEQ ID NO: 67.

A number of higher plant genes have been identified as increasing biomass yield or biomass upon over expression in higher plants. This increased yield in higher plants can be manifested in phenotypes such as increased cell proliferation, increased organ or cell size and increased total plant mass, The phrases “an increase in biomass yield” and “an increase in biomass” are used interchangeably throughout the specification.

An increase in biomass yield can be defined by a number of growth measures, including, for example, a selective advantage during competitive growth, increased growth rate, increased carrying capacity, and/or increased culture productivity (as measured on a per volume or per area basis).

For example, a competition assay can be between a transgenic strain and a wild-type s i between several transgenic strains, or between several transgenic strains and a wild-type strain.

Three genes were studied, and orthologs in Chlamydomonas reinhardtii were obtained via known functional annotations and sequence identities from BLAST.

The first gene is EBP1 the ErbB-3 epidermal growth factor receptor binding protein. Overexpression of EBP1 in potato and Arabidopsis regulates plant organ growth and effects the expression of different cell cycle genes (Horvath, B. M., Z. Magyar, et al. (2006), EMBO J 25 (20): 4909-4920),

The second gene is TOR kinase. Arabidopsis growth, seed yield, osmotic stress resistance, abscisic acid (ABA) and sugar sensitivity as well as polysome accumulation are positively correlated with levels of AtTOR messenger RNA (Deprost, D. L. Yao, et al. (2007). EMBO Rep 8(9): 864-870).

The third gene is Rubisco activase. This protein regulates the activation state of Rubisco. Many plants contain two forms of RCA: the 43-kD β (short; RCA1) isoform and the 46-kD α (long; RCA2) isoform that is regulated by the redox state of the chloroplast via oxidation of two Cys residues at the C terminus portion. Additionally, overexpression of a thermotolerant version of the protein results in higher biomass and increased seed yields (Kurek, I., T. K. Chang, et al. (2007), Plant Cell 19(10): 3230-3241).

For each of these three genes, the sequences shown to increase yield in higher plants were selected for study in algae. This included EBP1 from S. tuberosum, TOR kinase from A. thaliana and Rubisco Activase (RCA2) from A. thaliana. Additional orthologs were also selected for study. First, EBP1 from A. thaliana was selected in addition to the S. tuberosum sequence. Orthologs from the published C. reinhardtii genome were identified for all three genes via published functional annotations and BLAST similarity searches.

In addition, two novel Rubisco activase genes were isolated from Scenedesmus dimorphus and a Desmodesmus species. These sequences were identified through BLAST searches using the C. reinhardtii Rubisco activase sequence as a query against a database of RNA sequences derived from these two organisms.

Lastly, a thermostable RCA variant was studied. This sequence corresponds to RCA1 from A. thaliana with three point mutations (F168L, V257I, and K310N) as described in Kurek, I., T. K. Chang, et al. (2007), Plant Cell 19(10): 3230-3241,

Host Cells or Host Organisms

Biomass useful in the methods and systems described herein can be obtained from host cells or host organisms.

A host cell can contain a polynucleotide encoding a biomass yield gene of the present disclosure. In some embodiments, a host cell is part of a multicellular organism. In other embodiments, a host cell is cultured as a unicellular organism.

Host organisms can include any suitable host, for example, a microorganism. Microorganisms which are useful for the methods described herein include, for example, photosynthetic bacteria (e.g., cyanobacteria), non-photosynthetic bacteria(e.g., E. coli) yeast (e.g., Saccharomyces cerevisiae), and algae (e.g., microalgae such as Chlamydomonas reinhardtii).

Examples of host organisms that can be transformed with a polynucleotide of interest (for example, a biomass yield gene) include vascular and non-vascular organisms. The organism can be prokaryotic or eukaryotic. The organism can be unicellular or molt cellular. A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. In some instances, a photosynthetic organism may be transformed with a construct or vector of the disclosure which renders all or part of the photosynthetic apparatus inoperable.

By way of example, a non-vascular photosynthetic microalga species (for example, C. reinhardtii, Nannochloropsis oceanic, N. salina, D. salina, H. pluvalis, S. dimorphus, D. viridis, Chlorella sp., and D. tertiolecta) can be genetically engineered to produce a polypeptide of interest, for example a protein that when expressed results in an increase in biomass. Production of such a protein in these microalgae can be achieved by engineering the microalgae to express the protein in the algal chloroplast or nucleus.

In other embodiments the host organism is a vascular plant. Non-limiting examples of such plants include various monocots and divots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annus), flax (Linum usitatissimum), corn (Zea mays), coconut (Cocos nicifera), palm (Elaeis guineensis), oil nut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

The host cell can be prokaryotic. Examples of some prokaryotic organisms of the present disclosure include, but are not limited to, cyanobacteria (e.g., Synechococcus, Synechocystis, Athrospira, Gleocapsa, Oscillatoria, and, Pseudoanabaena). Suitable prokaryotic cells include, but are not limited to, any of a variety of laboratory strains of Escherichia coli, Lactobacillus sp., Salmonella sp., and Shigella sp. (for example, as described in Carrier et al. (1992) J Immunol. 148:1176-1181; U.S. Pat. No. 6,447,784; and Sizemore et al. (1995) Science 270:299-302). Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Pseudomonas aeruginosa, Pseudomonas aeruginosa, Pseudomonas mevalonii, Rhodobacter sphaeroides, Rhodobacter capsulatus, Rhodospirillum rubrum, and Rhodococcus sp.

In some embodiments, the host organism is eukaryotic (e.g. green algae, red algae, brown algae). In some embodiments, the algae is a green algae, for example, a Chlorophycean. The algae can be unicellular or multicellular. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, and Chlamydomonas reinhardtii.

In some embodiments, eukaryotic microalgae, such as for example, a Chlamydomonas, Volvacales, Dunaliella, Nannochloropsis, Desmodesmus, Scenedemus, Chlorella, Hematococcus species, can be used in the disclosed methods.

In other embodiments, the host cell is Chlamydomonas reinhardtii, Dunaliella salina, Haematococcus pluvialis, Nannochloropsis oceania, Nannochloropsis salina, Scenedesmus dimorphus, a Chlorella species, a Spirulina species, a Desmid species, Spirulina maximus, Arthrospira fusiformis, Dunaliella viridis, or Dunaliella tertiolecta.

In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes.

In some instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants. Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae. For example, the microalgae Chlamydomonas reinhardtii may be transformed with a vector, or a linearized portion thereof, encoding one or more proteins of interest (e.g., a yield (YD) protein).

Methods for algal transformation are described in U.S. Provisional Patent Application No. 60/142,091. The methods of the present disclosure can be carried out using algae, for example, the microalga, C. reinhardtii. The use of microalgae to express a polypeptide or protein complex according to a method of the disclosure provides the advantage that large populations of the microalgae can be grown, including commercially (Cyanotech Corp.; Kailua-Kona HI), thus allowing for production and, if desired, isolation of large amounts of a desired product.

The vectors of the present disclosure may be capable of stable or transient transformation of multiple photosynthetic organisms, including, but not limited to, photosynthetic bacteria (including cyanobacteria), cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrnmesiophyta, bacillariophyta, xanthophyta, eustigmatophyta, raphidophyta, phaeophyta, and phytoplankton. Other vectors of the present disclosure are capable of stable or transient transformation of, for example, C. reinhardtii, N. oceania, N. salina, D. salina, H. pluvalis, S. dimorphus, D. viridis, or D. tertiolecta.

Examples of appropriate hosts, include but are not limited to bacterial cells, such as E. coli, Streptomyces, Salmonella typhimurium; fungal cells, such as yeast; insect cells, such as Drosophila S2 and Spodoptera Sf9; animal cells, such as CHO, COS or Bowes melanoma; adenoviruses; and plant cells. The selection of an appropriate host is deemed to be within the scope of those skilled in the art.

Polynucleotides selected and isolated as described herein are introduced into a suitable host cell. A suitable host cell is any cell which is capable of promoting recombination and/or reductive reassortment. The selected polynucleotides can be, for example, in a vector which includes appropriate control sequences. The host cell can be, for example, a higher eukaryotic cell, such as a mammalian cell, or a lower eukaryotic cell, such as a yeast cell, or the host cell can be a prokaryotic cell, such as a bacterial cell. Introduction of a construct (vector) into the host cell can be effected by, for example, calcium phosphate transfection., DEAE-Dextran mediated transfection, or electroporation.

Recombinant polypeptides, including protein complexes, can be expressed in plants, allowing for the production of crops of such plants and, therefore, the ability to conveniently produce large amounts of a desired product. Accordingly, the methods of the disclosure can be practiced using any plant, including, for example, microalga and macroalgae, (such as marine algae and seaweeds), as well as plants that grow in soil.

In one embodiment, the host cell is a plant. The term “plant” is used broadly herein to refer to a eukaryotic organism containing plastids, such as chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

The YD genes of the present disclosure can be expressed in a higher plant. For example, Arabidopsis thaliana. The YD genes can also be expressed in a Brassica, Glycine, Gossypium, Medicago, Zea, Sorghum, Oryza, Triticum, or Panicum species.

A method of the disclosure can generate a plant containing genomic DNA (for example, a nuclear and/or plastid genomic DNA) that is genetically modified to contain a stably integrated polynucleotide (for example, as described in Hager and Bock, Appl. Microbiol. Biotechnol. 54:302-310, 2000). Accordingly, the present disclosure further provides a transgenic plant, e.g. C. reinhardtii, which comprises one or more chloroplasts containing a polynucleotide encoding one or more exogenous or endogenous polypeptides, including polypeptides that can allow for secretion of fuel products and/or fuel product precursors (e.g., isoprenoids, fatty acids, lipids, triglycerides). A photosynthetic organism of the present disclosure comprises at least one host cell that is modified to generate, for example, a fuel product or a fuel product precursor.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 31., 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

Where a halophilic organism is utilized for the present disclosure, it may be transformed with any of the vectors described herein. For example, D. salina may be transformed with a vector which is capable of insertion into the chloroplast or nuclear genome and which contains nucleic acids which encode a protein (e.g., a YD protein). Transformed halophilic organisms may then be grown in high-saline environments (e.g., salt lakes, salt ponds, and high-saline media) to produce the products (e.g., lipids) of interest. Isolation of the products may involve removing a transformed organism from a high-saline environment prior to extracting the product from the organism. In instances where the product is secreted into the surrounding environment, it may be necessary to desalinate the liquid environment prior to any further processing of the product.

The present disclosure further provides compositions comprising a genetically modified host cell. A composition comprises a genetically modified host cell; and will in some embodiments comprise one or more further components, which components are selected based in part on the intended use of the genetically modified host cell. Suitable components include, but are not limited to, salts; buffers; stabilizers; protease-inhibiting agents; cell membrane- and/or cell wall-preserving compounds, e.g., glycerol and dimethylsulfoxide; and nutritional media appropriate to the cell.

Culturing of Cells or Organisms

An organism may be grown under conditions which permit photosynthesis, however, this is not a requirement (e.g., a host organism may be grown in the absence of light). In some instances, the host organism may be genetically modified in such a way that its photosynthetic capability is diminished or destroyed. In growth conditions where a host organism is not capable of photosynthesis (e.g., because of the absence of light and/or genetic modification), the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of organisms occurs usually at a temperature of about 20° C. to about 25° C., although some organisms can still grow at a temperature of up to about 35° C. Active growth is typically performed in liquid culture. If the organisms are grown in a liquid medium and are shaken or mixed, the density of the cells can be anywhere from about 1 to 5×10⁸ cells/ml at the stationary phase. For example, the density of the cells at the stationary phase for Chlamydomonas sp. can be about 1 to 5×10⁷ cells/ml; the density of the cells at the stationary phase for Nannochloropsis sp. can be about 1 to 5×10⁸ cells/ml: the density of the cells at the stationary phase for Scenedesmus sp. can be about 1 to 5×10⁷ cells/ml; and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml; Nannochloropsis sp. can be about 1×10⁸ cellsiml; Scenedesmus sp. can be about 1×10⁷ cells/ml; and Chlorella sp. can be about 1×10⁸ cells/ml. An exemplary growth rate may yield, for example, a two to twenty fold increase in cells per day, depending on the growth conditions. In addition, doubling times for organisms can be, for example, 5 hours to 30 hours. The organism can also be grown on solid media, for example, media containing about 1.5% agar, in plates or in slants.

One source of energy is fluorescent light that can be placed, for example, at a distance of about inch to about two feet from the organism. Examples of types of fluorescent lights includes, for example, cool white and daylight. Bubbling with air or CO₂ improves the growth rate of the organism. Bubbling with CO₂ can be, for example, at 1% to 5% CO₂. If the lights are turned on and off at regular intervals (for example, 12:12 or 14:10 hours of light:dark) the cells of some organisms will become synchronized.

Long term storage of organisms can be achieved by streaking them onto plates, sealing the plates with, for example, Parafilm™, and placing them in dim light at about 10° C. to about 18° C. Alternatively, organisms may be grown as streaks or stabs into agar tubes, capped, and stored at about 10° C. to about 18° C. Both methods allow for the storage of the organisms for several months.

For longer storage, the organisms can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

Organisms can be grown on a defined minimal medium (for example, high salt medium (HSM), modified artificial sea water medium (MASH), or F/2 medium) with light as the sole energy source. In other instances, the organism can be grown in a medium (for example, tris acetate phosphate (TAP) medium), and supplemented with an organic carbon source.

Organisms, such as algae, can grow naturally in fresh water or marine water. Culture media for freshwater algae can be, for example, synthetic media, enriched media, soil water media, and solidified media, such as agar. Various culture media have been developed and used for the isolation and cultivation of fresh water algae and are described in Watanabe, M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container.

Culturing techniques for algae are well known to one of skill in the an and are described, for example, in Freshwater Culture Media, In R. A. Andersen (Ed.), Algal Culturing Techniques, Elsevier Academic Press.

Because photosynthetic organisms, for example, algae, require sunlight, CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system.

In addition, in open systems there is less control over water temperature, CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system arc that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors.

Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

The raceway ponds can be operated in a continuous manner, with, for example, CO₂ and nutrients being constantly fed to the ponds, while water containing the organism is removed at the other end.

If the raceway pond is placed outdoors, there are several different ways to address the invasion of an unwanted organism. For example, the pH or salinity of the liquid in which the desired organism is in can be such that the invading organism either slows down its growth or dies.

Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such as algae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations.

On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethylene bag cultivation). A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama, et al., Appl. Micro. Biotech., 41:729-731, 1994. Additional examples of photobioreactors are described in, U.S. Appl. Publ. No. 2005/0260553, U.S. Pat. No. 5,958,761, and U.S. Pat. No. 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organisms and variations of the methods described herein are known to one of skill in the art.

Organisms can also be grown near ethanol production plants or other facilities or regions (e.g., cities and highways) generating CO₂. As such, the methods herein contemplate business methods for selling carbon credits to ethanol plants or other facilities or regions generating CO₂ while making fuels or fuel products by growing one or more of the organisms described herein near the ethanol production plant, facility, or region.

The organism of interest, grown in any of the systems described herein, can be, for example, continually harvested, or harvested one batch at a time.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Aerators, Porous Stones and Diffusers.

Nutrients that can be used in the systems described herein include, or example, nitrogen (in the form of NO₃⁻ or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Organisms can be grown in cultures, for example large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

Chlamydomonas sp., Nannochloropsis sp., Scenedesmus sp., Desmodesmus sp., and Chlorella sp. are exemplary algae that can be cultured as described herein and can grow under a wide array of conditions.

One organism that can be cultured as described herein is a commonly used laboratory species C. reinhardtii. Cells of this species are haploid, and can grow on a simple medium of inorganic salts, using photosynthesis to provide energy. This organism can also grow in total darkness if acetate is provided as a carbon source. C. reinhardtii can be readily grown at room temperature under standard fluorescent lights. In addition, the cells can be synchronized by placing them on a light-dark cycle. Other methods of culturing C. reinhardtii cells are known to one of skill in the art.

Polynucleotides and Polypeptides

Also provided are isolated polynucleotides encoding a protein, for example, a YD protein described herein. As used herein “isolated polynucleotide” means a polynucleotide that is free of one or both of the nucleotide sequences which flank the polynucleotide in the naturally-occurring genome of the organism from which the polynucleotide is derived. The term includes, for example, a polynucleotide or fragment thereof that is incorporated into a vector or expression cassette; into an autonomously replicating plasmid or virus; into the genomic DNA of a prokaryote or eukaryote; or that exists as a separate molecule independent of other polynucleotides. It also includes a recombinant polynucleotide that is part of a hybrid polynucleotide, for example, one encoding a polypeptide sequence.

The novel proteins of the present disclosure can be made by any method known in the art. The protein may be synthesized using either solid-phase peptide synthesis or by classical solution peptide synthesis also known as liquid-phase peptide synthesis. Using Val-Pro-Pro, Enalapril and Lisinopril as starting templates, several series of peptide analogs such as X-Pro-Pro, X-Ala-Pro, and X Lys-Pro, wherein X represents any amino acid residue, may be synthesized using solid-phase or liquid-phase peptide synthesis. Methods for carrying out liquid phase synthesis of libraries of peptides and oligonucleotides coupled to a soluble oligomeric support have also been described. Bayer, Ernst and Mutter, Manfred, Nature 237:512-513 (1972); Bayer, Ernst, et al., J. Am. Chem. Soc. 96:7333-7336 (1974); Bonora, urian Maria, et at., Nucleic Acids Res. 18:3155-3159 (1990). Liquid phase synthetic methods have the advantage over solid phase synthetic methods in that liquid phase synthesis methods do not require a structure present on a first reactant which is suitable for attaching the reactant to the solid phase. Also, liquid phase synthesis methods do not require avoiding chemical conditions which may cleave the bond between the solid phase and the first reactant (or intermediate product). In addition, reactions in a homogeneous solution may give better yields and more complete reactions than those obtained in heterogeneous solid phase/liquid phase systems such as those present in solid phase synthesis.

In oligomer-supported liquid phase synthesis the growing product is attached to a large soluble polymeric group. The product from each step of the synthesis can then be separated from unreacted reactants based on the large difference in size between the relatively large polymer-attached product and the unreacted reactants. This permits reactions to take place in homogeneous solutions, and eliminates tedious purification steps associated with traditional liquid phase synthesis. Oligomer-supported liquid phase synthesis has also been adapted to automatic liquid phase synthesis of peptides. Bayer, Ernst, et a Peptides: Chemistry, Structure, Biology, 426-432.

For solid-phase peptide synthesis, the procedure entails the sequential assembly of the appropriate amino acids into a peptide of a desired sequence while the end of the growing peptide is linked to an insoluble support. Usually, the carboxyl terminus of the peptide is linked to a polymer from which it can be liberated u m treatment with a cleavage reagent. In a common method, an amino acid is bound to a resin particle, and the peptide generated in a stepwise manner by successive additions of protected amino acids to produce a chain of amino acids. Modifications of the technique described by Merrifield are commonly used. See, e.g., Merrifield, J. Am. Chem. Soc. 96: 2989-93 (1964). In an automated solid-phase method, peptides are synthesized by loading the carboxy-terminal amino acid onto an organic linker (e.g., PAM, 4-oxymethylphenylacetamidomethyl), which is covalently attached to an insoluble polystyrene resin cross-linked with divinyl benzene. The terminal amine may be protected by blocking with t-butyloxycarbonyl. Hydroxyl- and carboxyl-groups are commonly protected by blocking with O-benzyl groups. Synthesis is accomplished in an automated peptide synthesizer, such as that available from Applied Biosystems (Foster City, Calif.). Following synthesis, the product may be removed from the resin, The blocking groups are removed by using hydrofluoric acid or trifluoromethyl sulfonic acid according to established methods. A routine synthesis may produce 0.5 mmole of peptide resin. Following cleavage and purification, a yield of approximately 60 to 70% is typically produced. Purification of the product peptides is accomplished by, for example, crystallizing the peptide from an organic solvent such as methyl-butyl ether, then dissolving in distilled water, and using dialysis (if the molecular weight of the subject peptide is greater than about 500 daltons) or reverse high pressure liquid chromatography (e.g., using a C¹⁸ column with 0.1% trifluoroacetic acid and acetonitrile as solvents) if the molecular weight of the peptide is less than 500 daltons. Purified peptide may be lyophilized and stored in a dry state until use, Analysis of the resulting peptides may be accomplished using the common methods of analytical high pressure liquid chromatography (HPLC) and electrospray mass spectrometry (ES-MS).

In other cases, a protein, for example, a YD protein, is produced by recombinant methods. For production of any of the proteins described herein, host cells transformed with an expression vector containing the polynucleotide encoding such a protein can be used. The host cell can be a higher eukaryotic cell, such as a mammalian cell, or a lower eukaryotic cell such as a yeast or algal cell, or the host can be a prokaryotic cell such as a bacterial cell. Introduction of the expression vector into the host cell can be accomplished by a variety of methods including calcium phosphate transfection, DEAF-dextran mediated transfection, polybrene, protoplast fusion, liposomes, direct microinjection into the nuclei, scrape loading, biolistic transformation and electroporation. Large scale production of proteins from recombinant organisms is a well-established process practiced on a commercial scale and well within the capabilities of one skilled in the art.

It should be recognized that the present disclosure is not limited to transgenic cells, organisms, and plastids containing a protein or proteins as disclosed herein, but also encompasses such cells, organisms, and plastids transformed with additional nucleotide sequences encoding enzymes involved in fatty acid synthesis. Thus, some embodiments involve the introduction of one or more sequences encoding proteins involved in fatty acid synthesis in addition to a protein disclosed herein. For example, several enzymes in a fatty acid production pathway may be linked, either directly or indirectly, such that products produced by one enzyme in the pathway, once produced, are in close proximity to the next enzyme in the pathway. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used, they can be introduced into the host cell or organism simultaneously or sequentially.

Additional embodiments provide a plastid, and in particular a chloroplast, transformed with a polynucleotide encoding a protein of the present disclosure. The protein may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in co-pending U.S. patent application Ser. No. 12/287,230 filed Oct. 6, 2008, published as US. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

One or more nucleotides of the present disclosure can also be modified such that the resulting amino acid is “substantially identical” to the unmodified or reference amino acid.

A “substantially identical” amino acid sequence is a sequence that differs from a reference sequence by one or more conservative or non-conservative amino acid substitutions, deletions, or insertions, particularly when such a substitution occurs at a site that is not the active site (catalytic domains (CDs)) of the molecule and provided that the polypeptide essentially retains its functional properties. A conservative amino acid substitution, fir example, substitutes one amino acid fir another of the same class (e.g., substitution of one hydrophobic amino acid, such as isoleucine, valine, leucine, it methionine, for another, or substitution of one polar amino acid for another, such as substitution of arginine fir lysine, glutamic acid for aspartic acid or glutamine for asparagine).

The disclosure provides alternative embodiments of the polypeptides of the invention (and the nucleic acids that encode them) comprising at least one conservative amino acid substitution, as discussed herein (e.g., conservative amino acid substitutions are those that substitute a given amino acid in a polypeptide by another amino acid of like characteristics). The invention provides polypeptides (and the nucleic acids that encode them) wherein any, some or all amino acids residues are substituted by another amino acid of like characteristics, e.g., a conservative amino acid substitution.

Conservative substitutions are those that substitute a given amino acid in a polypeptide by another amino acid of like characteristics. Examples of conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative aspects, these conservative substitutions can also be synthetic equivalents of these amino acids.

Introduction of Polynucleotide into a Host Organism or Cell

To generate a genetically modified host cell, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann, Rev. Plant. Physiol. Plant Mol. Biol. 42:205-225, 1991).

As discussed above, microprojectile mediated transformation can be used to introduce a polynucleotide into a cell (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a cell using a device such as the BIOLISTIC PD-1000 particle gun (BioRad; Hercules Calif.). Methods for the transformation using biolistic methods are well known in the art (for example, as described in Christou, Trench in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method.

The basic techniques used for transformation and expression in photosynthetic microorganisms are similar to those commonly used for E. cull, Saccharomyces cerevisiae and other species. Transformation. methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M 5, 1997, Plant Molecular Biology, Springer, N.Y.). These methods include, for example, biolistic devices (See, for example, Sanford, Trends in Biotech. (1988) 6: 299-302, U.S. Pat. No. 4,945,050; electroporation (Fromm et al., Proc. Natl. Acad. Sci. (USA) (1985) 82: 5824-5828); use of a laser beam, electroporation, microinjection or any other method capable of introducing DNA into a host cell.

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome, in some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad., Sci. USA 87:8526-8530, 1990), and can result in stable homoplasmic transformants, at a frequency of approximately one per 100 bombardments of target leaves.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded enzymes into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the enzymes. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heinle et al, (1991) Plant Mol. Biol. Rep. 9: 104; Clark et al. (1989) J Biol. Chem. 264: 17544; della-Cioppa A. (1987) Plant Physiol. 84: 965; Romer et al, (1993) Biochem. Biophys. Res. Commun. 196: 1414; and Shah et al. (1986) Seience 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad Sri, USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII, and KASIII); steroyl-ACP desaturase; thioesterases (specific thr short, medium, and long chain acyl ACP); plastid-targeted acyl transferases (e.g., glycerol-3-phosphate and acyl transferase); enzymes involved in the biosynthesis of aspartate family amino acids; phytoene synthase; gibberelic acid biosynthesis (e.g., ent-kaurene synthases 1 and 2); and carotenoid biosynthesis (e.g., lycopene synthase).

In some embodiments, an alga is transformed with a nucleic acid which encodes a YD protein of interest, and is also transformed with a gene encoding any one or more of a prenyl transferase, an isoprenoid synthase, or an enzyme capable of converting a precursor into a fuel product or a precursor of a fuel product (e.g., an isoprenoid or fatty acid).

In one embodiment, a transformation may introduce a nucleic acid into a plastid of the host alga (e.g., chloroplast). In another embodiment, a transformation may introduce a nucleic acid into the nuclear genome of the host alga. In still another embodiment, a transformation may introduce nucleic acids into both the nuclear genome and into a plastid.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized, Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s) and/or products. Protein expression screening can be performed by Western blot analysis and/or enzyme activity assays. Transporter anchor product screening may be performed by any method known in the art, for example ATP turnover assay, substrate transport assay, HPLC or gas chromatography.

The expression of the protein or enzyme can be accomplished by inserting a polynucleotide sequence (gene) encoding the protein or enzyme into the chloroplast or nuclear genome of a microalgae. The modified strain of microalgae can be made homoplasmic to ensure that the polynucleotide frill be stably maintained in the chloroplast genome of all descendents. A microalga is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein. The process of determining the plasmic state of an organism of the present disclosure involves screening transformants for the presence of exogenous nucleic acids and the absence of wild-type nucleic acids at a given locus of interest.

Vectors

Construct, vector and plasmid are used interchangeably throughout the disclosure. Nucleic acids encoding the proteins described herein, can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques know to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2nd Ed., Cold Spring Harbor Press, (1989) and Ausubel et al., Short Protocols in Molecular Biology, 2nd Ed., John Wiley & Sons (1992).

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), PI-based artificial chromosomes, yeast plasmids, yeast artificial chromosomes, and any other vectors specific for specific hosts of interest (such as E. coli and yeast). Thus, for example, a polynucleotide encoding a YD protein, can be inserted into any one of a variety of expression vectors that are capable of expressing the enzyme. Such vectors can include, for example, chromosomal, nonchromosomal and synthetic DNA sequences.

Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used. For example, vectors such as Ble2A, Arg7/2A, and SEnuc357 can be used for the expression of a protein.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSG5 (Stratagene), pSVK3, pBPV, pMSG, pET21a-d(+) vectors (Novagen), and pSVLSV40 (Pharmacia). However, any other plasmid or other vector may be used so long as it is compatible with the host cell.

The expression vector, or a linearized portion thereof, can encode one or more exogenous or endogenous nucleotide sequences. Examples of exogenous nucleotide sequences that can be transformed into a host include genes from bacteria, fungi, plants, photosynthetic bacteria or other algae. Examples of other types of nucleotide sequences that can be transformed into a host, include, but are not limited to, transporter genes, isoprenoid producing genes, genes which encode for proteins which produce isoprenoids with two phosphates (e.g., GPP synthase and/or FPP synthase), genes which encode for proteins which produce filthy acids, lipids, or triglycerides, for example, ACCases, endogenous promoters, and 5′ UTRs from the psbA, atpA, or rbcL genes. In some instances, an exogenous sequence is flanked by two homologous sequences.

Homologous sequences are, for example, those that have at least 50%, at least 55%, at least 60%, at least 65%, at least 70%, at least 75%, at least 80%, at least 85%, at least 90%, at least 95%, at least 98%, or at least 99% sequence identity to a reference amino acid sequence or nucleotide sequence, for example, the amino acid sequence or nucleotide sequence that is found in the host cell from which the protein is naturally obtained from or derived from.

A nucleotide sequence can also be homologous to a codon-optimized gene sequence. For example, a nucleotide sequence can have, for example, at least 50%, at least 55%, at least 60%, at least 65%, at least 70%, at least 75%, at least 80%, at least 85%, at least 90%, at least 95%, at least 98%, or at least 99% nucleic acid sequence identity to the codon-optimized gene sequence.

The first and second homologous sequences enable recombination of the exogenous or endogenous sequence into the genome of the host organism. The first and second homologous sequences can be at least 100, at least 200, at least 300, at least 400, at least 500, or at least 1500 nucleotides in length.

In some embodiments, about 0.5 to about 1.5 kb flanking nucleotide sequences of chloroplast genomic DNA may be used. In other embodiments about 0.5 to about 1.5 kb flanking nucleotide sequences of nuclear genomic DNA may be used, or about 2.0 to about 5.0 kb may be used.

In some embodiments, the vector may comprise nucleotide sequences that are codon-biased for expression in the organism being transformed. In another embodiment, a gene of interest, for example, a biomass yield gene, may comprise nucleotide sequences that are codon-biased for expression in the organism being transformed. In addition, the nucleotide sequence of a tag may be codon-biased er codon-optimized for expression in the organism being transformed.

A polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis. Codon bias is described in detail herein.

In some embodiments, a vector comprises a polynucleotide operably linked to one or more control elements, such as a promoter and/or a transcription terminator. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley &. Sons (1992).

A vector in some embodiments provides for amplification of the copy number of one or more polynucleotides. A vector can be, for example, an expression vector that provides for expression of a YD protein, and any one or more of a prenyl transferase, an isoprenoid synthase, or a mevalonate synthesis enzyme in a host cell, e.g., a prokaryotic host cell or a eukaryotic host cell,

A polynucleotide or polynucleotides can be contained in a vector or vectors. For example, where a second (or more) nucleic acid molecule is desired, the second nucleic acid molecule can be contained in a vector, which can, but need not be, the same vector as that containing the first nucleic acid molecule. The vector can be any vector useful for introducing a polynucleotide into a genome and can include a nucleotide sequence of genomic DNA (e.g., nuclear or plastid) that is sufficient to undergo homologous recombination with genomic DNA, for example, a nucleotide sequence comprising about 400 to about 1500 or more substantially contiguous nucleotides of genomic DNA.

A regulator or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the cytosol, nucleus, chloroplast membrane or cell membrane) can be attached to the polynucleotide encoding a protein of interest. Such signals are known in the art and have been widely reported (see, e.g., U.S. Pat. No. 5,776,689).

In a vector, a nucleotide sequence of interest is operably linked to a promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal). The promoters contemplated herein can be specific to photosynthetic organisms, non-vascular photosynthetic organisms, and vascular photosynthetic organisms (e.g., algae, flowering plants). In some instances, the nucleic acids above are inserted into a vector that comprises a promoter of a photosynthetic organism, e.g., algae. The promoter can be a constitutive promoter or an inducible promoter. A promoter typically includes necessary nucleic acid sequences near the start site of transcription, (e.g., a TATA element). Common promoters used in expression vectors include, but are not limited to, LTR or SV40 promoter, the E. coli lac or trp promoters, and the phage lambda PL promoter. Non-limiting examples of promoters are endogenous promoters such as the psbA and atpA promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

A “constitutive” promoter is, for example, a promoter that is active under most environmental and developmental conditions. Constitutive promoters can, for example, maintain a relatively constant level of transcription.

An “inducible” promoter is a promoter that is active under controllable environmental or developmental conditions. For example, inducible promoters are promoters that initiate increased levels of transcription from DNA under their control in response to some change in the environment, e.g. the presence or absence of a nutrient or a change in temperature.

Examples of inducible promoters/regulatory elements include, for example, a nitrate-inducible promoter (for example, as described in Bock et al, Plant Mal. Biol. 17:9 (1991)), or a light-inducible promoter, (for example, as described in Feinbaum et al, Mol. Gen. Genet. 226:449 (1991); and Lam and Chua, Science 248:471 (1990)), or a heat responsive promoter (for example, as described in Muller et al., Gene 111: 165-73 (1992)).

In many embodiments, a polynucleotide of the present disclosure includes a nucleotide sequence encoding a protein or enzyme of the present disclosure, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman et al. (1995) J. Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In many embodiments, a polynucleotide of the present disclosure includes a nucleotide sequence encoding a protein or enzyme of the present disclosure, where the nucleotide sequence encoding the polypeptide is operably linked to a constitutive promoter. Suitable constitutive promoters for use in prokaryotic cells are known in the art and include, but are not limited to, a sigma70 promoter, and a consensus sigma70 promoter.

Suitable promoters for use in prokaryotic host cells include, but are not limited to, a bacteriophage T7 RNA polymerase promoter; a trp promoter; a lac operon promoter; a hybrid promoter, e.g., a lac/lac hybrid promoter, a tac/trc hybrid promoter, a trp/lac promoter, a T7/lac promoter; a trc promoter; a tac promoter; an araBAD promoter; in vivo regulated promoters, such as an ssaG promoter or a related promoter (for example, as described in U.S. Patent Publication No. 200401316:37), a pagC promoter (for example, as described in Pulkkirten and Miller, J. Bacteria, 1991: 173(1): 86-9:3; and Alpuche-Aranda et al., PNAS, 1992; 89(21): 10079-83), a nirB promoter (for example, as described in Harborne et al. (1992) Mol. Micro. 6:2805-2813; Dunstan. et al, (1999) Infect, Immun. 67:5133-5141; McKelvie et al. (2004) Vaccine 22:3243-3255; and Chatfield et al. (1992) Biotechnol. 10:888-892); a sigma70 promoter, e.g., a. consensus sigma70 promoter (for example. GenBank Accession Nos. AX798980, AX798961, and AX798183); a stationary phase promoter, e.g., a dps promoter, an spv promoter; a promoter derived from the pathogenicity island SPI-2 (for example, as described in WO96/17951); an actA promoter (for example, as described in Shetron-Rama et al. (2002) Infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W. and Heinemann, U. (eds). Topics in Molecular and Structural Biology, Protein-Nucleic Acid Interaction. Macmillan, London, UK, Vol. 10, pp. 143-162); and an SP6 promoter (for example, as described in Melton et al. (1984) Nucl. Acids Res. 12:7035-7056).

In yeast, a number of vectors containing constitutive or inducible promoters may be used. For a review of such vectors see, Current Protocols in Molecular Biology, Vol. 2, 1988, Ed. Ausubel, et al., Greene Publish. Assoc. &. Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (for example, as described in Cloning in Yeast, Ch. 3, R. Rothstein In: DNA Cloning Vol. 11, A Practical Approach, Ed. DM Glover, 1986, IRL Press, Wash., D.C.). Alternatively, vectors may be used which promote integration of foreign DNA sequences into the yeast chromosome.

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that a exogenous or endogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, an IRES. Additionally, an element can be a cell compartmentalization signal (i.e., a sequence that targets a polypeptide to the cytosol, nucleus, chloroplast membrane or cell membrane). In some aspects of the present disclosure, a cell compartmentalization signal (e.g., a cell membrane targeting sequence) may be ligated to a gene and/or transcript, such that translation of the gene occurs in the chloroplast. In other aspects, a cell compartmentalization signal may be ligated to a gene such that, following translation of the gene, the protein is transported to the cell membrane. Cell compartmentalization signals are well known in the art and have been widely reported (see, e.g., U.S. Pat. No. 5,776,689).

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype.

A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacterial: 178:121, 1996; Gerdes, FEBS Lett. 389:44-47, 1996; and Jefferson, EMBO J. 6:3901-3907, 1997, fl-glucuronidase).

A selectable marker (or selectable gene) generally is a molecule that, when present or expressed in a cell, provides a selective advantage (or disadvantage) to the cell containing the marker, for example, the ability to grow in the presence of an agent that otherwise would kill the cell. The selection gene can encode for a protein necessary for the survival or growth of the host cell transformed with the vector.

A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or gowth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss. Plant Physiol. (Life Sci. Adv.) 13:143-149, 1994); neomycin phosphotransferase, which confers resistance to the aminoglycosides neomycin, kanamycin and parornycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of ttyptophart; hisD, which allows cells to utilize histinol in place of histidine (for example, as described in Hartman, Proc. Nail. Acad. Sci., USA 85:8047, 1988); mannose-6-phosphate isomerase which allows cells to utilize mannose (for example, as described in PCT Publication Application No. WO 94/20627); ornithine decarboxylase, which confers resistance to the ornithine decarboxylase inhibitor, 2-(difluoromethyl)-DL-ornithine (DEMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol, Biochem, 59:2336-2338, 1995). Additional selectable markers include those that confer herbicide resistance, for example, phosphinothricin acetyltransferase gene, which confers resistance to phosphinothricin. (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest. The promoter driving expression of the selection marker can be a constitutive or an inducible promoter.

Reporter genes geatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyhransf-erase (aadA, for example, as described in Svab and Maliga, Proc. Natl. Aced. Sci., USA 90:913-917, 1993), and the Aequorea victoria GFP (for example, as described in Sidorov et al., Plant J. 19:209-216, 1999) have been used as reporter genes (Ibr example, as described in Heifetz, Biochemie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota et al., Proc. Natl. Acad. Sci., USA 96:1840-1845, 1999), or human somatotropin (for example, as described in Staub et al., Nat. Biotechnol. 18:333-338, 2000), a potential biopharmaceutical. Several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet. 263:404-410, 2000).

In one embodiment a protein described herein is modified by the addition of an N-terminal strep tag epitope to aid in the detection of protein expression. In another embodiment, a protein described herein is modified at the C-terminus by the addition of a Flag-tag epitope to aid in the detection of protein expression, and to facilitate protein purification.

Affinity tags can be appended to proteins so that they can be purified from their crude biological source using an affinity technique. These include, for example, chitin binding protein (CBP), maltose binding protein (MBP), and glutathione-S-transferase (GST). The poly(His) tag is a widely-used protein tag; it binds to metal matrices. Some affinity tags have a dual role as a solubilization agent, such as MBP, and GST. Chromatography tags are used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include, but are not limited to, VS-tag, c-myc-tag, and HA-tag. These tags are particularly useful for western blotting and immunoprecipitation experiments, although they also find use in antibody purification. Fluorescence tags are used to give visual readout on a protein. GFP and its variants are the most commonly used fluorescence tags. More advanced applications of &FP include using it as a folding reporter (fluorescent if folded, colorless if not).

In one embodiment, any one of the YD proteins described herein can be fused at the amino-terminus to the carboxy-terminus of a highly expressed protein (fusion partner). These fusion partners may enhance the expression of the YD gene. Engineered processing sites, for example, protease, proteolytic, or tryptic processing or cleavage sites, can be used to liberate the YD protein from the fusion partner, allowing for the purification of the intended YD protein. Examples of fusion partners that can be fused to the YD gene are a sequence encoding the mammary-associated serum amyloid (M-SAA) protein, a sequence encoding the large and/or small subunit of ribulose bisphosphate carboxylase, a sequence encoding the glutathione S-transferase (GST) gene, a sequence encoding a thioredoxin (TRX) protein, a sequence encoding a maltose-binding protein (MBP), a sequence encoding any one or more of E. coli proteins NusA, NusB, NusG, or NusE, a sequence encoding a ubiqutin (Ub) protein, a sequence encoding a small ubiquitin-related modifier (SUMO) protein, a sequence encoding a cholera toxin B subunit (CTB) protein, a sequence of consecutive histidine residues linked to the 3′ end of a sequence encoding the MBP-encoding malE gene, the promoter and leader sequence of a galactokinase gene, and the leader sequence of the ampicillinase gene.

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identity vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web, at the URL “biology.duke.edu/chlamy_genome/-chloro.html.” (see “view complete genome as text file” link and “maps of the chloroplast genome” link; J. Maul, J. W. Lilly, and D. B. Stern, unpublished results; revised Jan. 28, 2002; to be published as GenBank Acc. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul., J. E., et al, (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” and “140-150 kb” link; also accessible directly on world wide web at URL “biology.duke.edu/chlam-y/chloro/chloro140.html”).

In addition, the entire nuclear genome of C. reinhardtii is described in Merchant, S. S. et al., Science (2007), 318(5848):245-250, thus facilitating one of skill in the art to select a sequence or sequences useful for constructing a vector.

For expression of the polypeptide in a host, an expression cassette or vector may be employed. The expression vector will comprise a transcriptional and translational initiation region, which may be inducible or constitutive, where the coding region is operably linked under the transcriptional control of the transcriptional initiation region, and a transcriptional and translational termination region. These control regions may be native to the gene, or may be derived from an exogenous source. Expression vectors generally have convenient restriction sites located near the promoter sequence to provide for the insertion of nucleic acid sequences encoding exogenous or endogenous proteins. A selectable marker operative in the expression host may be present.

The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausuhel et al., Short Protocols in Molecular Biology, 2^{nd} Ed., John Wiley & Sons (1992).

The description herein provides that host cells may be transformed with vectors. One of skill in the art will recognize that such transformation includes transformation with circular vectors, linearized vectors, linearized portions of a vector, or any combination of the above. Thus, a host cell comprising a vector may contain the entire vector in the cell (in either circular or linear form), or may contain a linearized portion of a vector of the present disclosure.

Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between PVC) sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

Codon Optimisation

One or more codons of an encoding polynucleotide can be “biased” or “optimized” to reflect the codon usage of the host organism. These two terms can be used interchangeably throughout the disclosure, For example, one or more codons of an encoding polynucleotide can be “biased” or “optimized” to reflect chloroplast codon usage (Table A) or nuclear codon usage (Table B) in Chlamydomonas reinhardtii. Most amino acids are encoded by two or more different (degenerate) codons, and it is well recognized that various organisms utilize certain codons in preference to others. Generally, the codon bias selected reflects codon usage of the plant (or organelle therein) which is being transformed with the nucleic acid or acids of the present disclosure. However, the codon bias need not be selected based on a particular organism in which a polynucleotide is to be expressed.

One or more codons can be modified, for example, by a method such as site directed mutagenesis, PCR using a primer that is mismatched for the nucleotide(s) to be changed such that the amplification product is biased to reflect the selected (chloroplast or nuclear) codon usage, or by the de novo synthesis of a polynucleotide sequence such that the change (bias) is introduced as a consequence of the synthesis procedure.

When codon-optimizing a specific gene sequence for expression, factors other than be codon usage may also be taken into consideration. For example, it is typical to avoid restrictions sites, repeat sequences, and potential methylation sites. Most gene synthesis companies utilize computational algorithms to optimize a DNA sequence taking into consideration these and other factors whilst maintaining the codon usage (as defined in the codon usage table) above a user-defined threshold. For example, this threshold may be set such that a codon that is used less than 10% of the time that the corresponding amino acid is present in the proteome would be avoided in the final DNA sequence.

Table A (below) shows the chloroplast codon usage for C. reinhardtii (see U.S. Patent Application Publication No. 2004/0014174, published Jan. 22, 2004).

TABLE A
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The C. reinhardtii chloroplast genome shows a high AT content and noted codon bias (for example, as described in Franklin S., et al. (2002) Plant J 30:733-744; Mayfield S. P. and Schultz J. (2004) Plant J 37:449-458).

Table B exemplifies codons that are preferentially used in Chlamydomonas nuclear genes.

TABLE B
fields: [triplet] [frequency: per thousand] ([number])
Coding GC 66.30% 1^{st} letter GC 64.80% 2^{nd} letter GC 47.90% 3^{rd} letter GC 86.21%
Nuclear Codon Usage in Chlamydomonas reinhardtii

| | | | |
|---|---|---|---|
| UUU 5.0 (2110) | UCU 4.7 (1992) | UAU 2.6 (1085) | UGU 1.4 (601) |
| UUC 27.1 (11411) | UCC 16.1 (6782) | UAC 22.8 (9579) | UGC 13.1 (5498) |
| UUA 0.6 (247) | UCA 3.2 (1348) | UAA 1.0 (441) | UGA 0.5 (227) |
| UUG 4.0 (1673) | UCG 16.1 (6763) | UAG 0.4 (183) | UGG 13.2 (5559) |
| CUU 4.4 (1869) | CCU 8.1 (3416) | CAU 2.2 (919) | CGU 4.9 (2071) |
| CUC 13.0 (5480) | CCC 29.5 (12409) | CAC 17.2 (7252) | CGC 34.9 (14676) |
| CUA 2.6 (1086) | CCA 5.1 (2124) | CAA 4.2 (1780) | CGA 2.0 (841) |
| CUG 65.2 (27420) | CCG 20.7 (8684) | CAG 36.3 (15283) | CGG 11.2 (4711) |
| AUU 8.0 (3360) | ACU 5.2 (2171) | AAU 2.8 (1157) | AGU 2.6 (1089) |
| AUC 26.6 (11200) | ACC 27.7 (11663) | AAC 28.5 (11977) | AGC 22.8 (9590) |
| AUA 1.1 (443) | ACA 4.1 (1713) | AAA 2.4 (1028) | AGA 0.7 (287) |
| AUG 25.7 (10796) | ACG 15.9 (6684) | AAG 43.3 (18212) | AGG 2.7 (1150) |
| GUU 5.1 (2158) | GCU 16.7 (7030) | GAU 6.7 (2805) | GGU 9.5 (3984) |
| GUC 15.4 (6496) | GCC 54.6 (22960) | GAC 41.7 (17519) | GGC 62.0 (26064) |
| GUA 2.0 (857) | GCA 10.6 (4467) | GAA 2.8 (1172) | GGA 5.0 (2084) |
| GUG 46.5 (19558) | GCG 44.4 (18688) | GAG 53.5 (22486) | GGG 9.7 (4087) |

Generally, the nuclear codon bias selected for purposes of the present disclosure, including, for example, in preparing a synthetic polynucleotide as disclosed herein, can reflect nuclear codon usage of an algal nucleus and includes a codon bias that results in the coding sequence containing greater than 60% G/C content.

Re-Engineering the Genome.

In addition to utilizing codon bias as a means to provide efficient translation of a polypeptide, it will be recognized that an alternative means for obtaining efficient translation of a polypeptide in an organism is to re-engineer the genome (e.g., a C. reinhardtii chloroplast or nuclear genome) for the expression of tRNAs not otherwise expressed in the genome. Such an engineered algae expressing one or more exogenous tRNA molecules provides the advantage that it would obviate a requirement to modify every polynucleotide of interest that is to be introduced into and expressed from an algal genome; instead, algae such as C. reinhardtii that comprise a genetically modified genome can be provided and utilized for efficient translation of polypeptide. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; and Komar et. al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into the genome of the host organism to complement rare or unused tRNA genes in the genome, such as a C. reinhardtii chloroplast genome.

Another Way to Codon Optimize a Sequence for Expression.

An alternative way to optimize a nucleic acid sequence for expression is to use the most frequently utilized codon (as determined by a codon usage table) for each amino acid position. This type of optimization may be referred to as ‘hot codon’ optimization. Should undesirable restriction sites be created by such a method then the next most frequently utilized codon may be substituted in a position such that the restriction site is no longer present. Table C lists the codon that would be selected for each amino acid when using this method for optimizing a nucleic acid sequence for expression in the chloroplast of C. reinhardtii.

TABLE C

| Amino acid | Codon utilized |
|---|---|
| F | TTC |
| L | TTA |
| I | ATC |
| V | GTA |
| S | TCA |
| P | CCA |
| T | ACA |
| A | GCA |
| Y | TAC |
| H | CAC |
| Q | CAA |
| N | AAC |
| K | AAA |
| D | GAC |
| E | GAA |
| C | TGC |
| R | CGT |
| G | GGC |
| W | TGG |
| M | ATG |
| STOP | TAA |

Codon Optimization for the Nucleus of a Desmodesmus, Chlamydomonas, Nannochloropsis, or Scenedesmus Species

To create a codon usage table that can be used to express a gene in the nucleus of several different species, the codon usage frequency of a number of species were analyzed. 30,000 base pairs of DNA sequence corresponding to nuclear protein coding regions for the each of the algal species Scenedesmus sp. (S. dimorphus), Desmodesimts sp. (an unknown Desmodesmus sp.), and Nannochloropsis sp. (N. salina) were used to create a unique nuclear codon usage table for each species. These tables were then compared to each other and to that of Chlamydomonas reinhardtii; the codon table for the nuclear genome of Chlamydomonas reinhardtii was used as a standard. Any codons that had very low codon usage for the other algal species but not in Chlamydomonas reinhardtii were fixed at 0 and thus should be avoided in a DNA sequence designed using this codon table (Table D). The following codons should be avoided CGG, CAT, CCG, and TCG. The codon usage table, generated is shown in Table D.

TABLE D
Nuclear Codon usage in a Chlamydomonas sp., Scenedesmus sp.,
Desmodesmus sp., and Nannochloropsis sp.
For example, in the first row, the fraction (0.16) is the percentage (16%)
of times that a codon (UUU) is used to code for F (phenylalanine).

| Triplet | a.a. | Fraction | Triplet | a.a. | Fraction | Triplet | a.a. | Fraction | Triplet | a.a. | Fraction |
|---|---|---|---|---|---|---|---|---|---|---|---|
| UUU | F | 0.16 | UCU | S | 0.1 | UAU | Y | 0.1 | UGU | C | 0.1 |
| UUC | F | 0.84 | UCC | S | 0.33 | UAC | Y | 0.9 | UGC | C | 0.9 |
| UUA | L | 0.01 | UCA | S | 0.06 | UAA | * | 0.52 | UGA | * | 0.27 |
| UUG | L | 0.04 | UCG | S | 0 | UAG | * | 0.22 | UGG | W | 1 |
| CUU | L | 0.05 | CCU | P | 0.19 | CAU | H | 0 | CGU | R | 0.11 |
| CUC | L | 0.15 | CCC | P | 0.69 | CAC | H | 1 | CGC | R | 0.77 |
| CUA | L | 0.03 | CCA | P | 0.12 | CAA | Q | 0.1 | CGA | R | 0.04 |
| CUG | L | 0.73 | CCG | P | 0 | CAG | Q | 0.9 | CGG | R | 0 |
| AUU | I | 0.22 | ACU | T | 0.1 | AAU | N | 0.09 | AGU | S | 0.05 |
| AUC | I | 0.75 | ACC | T | 0.52 | AAC | N | 0.91 | AGC | S | 0.46 |
| AUA | I | 0.03 | ACA | T | 0.08 | AAA | K | 0.05 | AGA | R | 0.02 |
| AUG | M | 1 | ACG | T | 0.3 | AAG | K | 0.95 | AGG | R | 0.06 |
| GUU | V | 0.07 | GCU | A | 0.13 | GAU | D | 0.14 | GGU | G | 0.11 |
| GUC | V | 0.22 | GCC | A | 0.43 | GAC | D | 0.86 | GGC | G | 0.72 |
| GUA | V | 0.03 | GCA | A | 0.08 | GAA | E | 0.05 | GGA | G | 0.06 |
| GUG | V | 0.67 | GCG | A | 0.35 | GAG | E | 0.95 | GGG | G | 0.11 |

(*represents stop codons)(a.a. is amino acid)

The following examples are intended to provide illustrations of the application of the present disclosure. The following examples are not intended to completely define or otherwise limit the scope of the disclosure.

One of skill in the art will appreciate that many other methods known. In the art may be substituted in lieu of the ones specifically described or referenced herein.

### Example 1

### Cloning of Biomass Yield Genes into SEnuc745 and Creation of Overexpression Cell Lines

The open reading frame (ORF) for seven biomass yield genes (described in the table below) were each codon optimized using Chlamydomonas reinhardtii nuclear codon usage tables and synthesized. The seven codon-optimized ORFs are shown in SEQ ID NOs: 1 to 7.

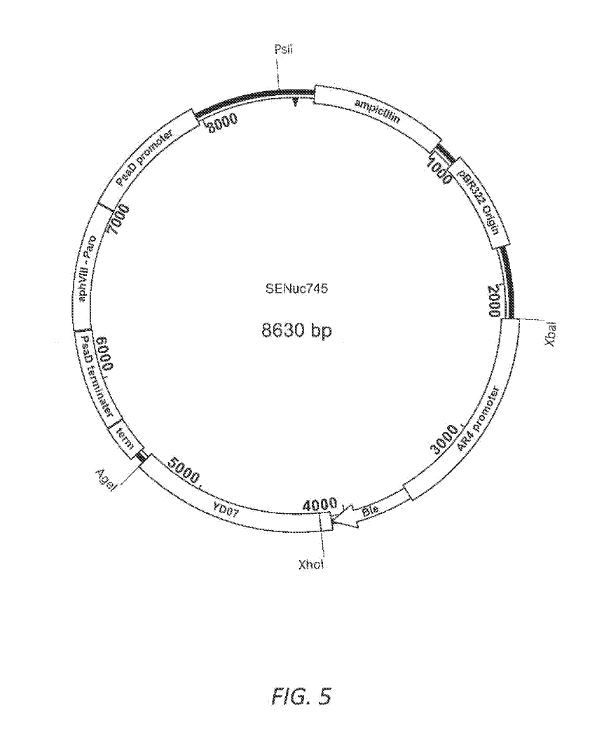

The DNA constructs (SEQ ID NOs: 1 to 7) for the seven targets were each individually cloned into nuclear overexpression vector SEnuc745 (FIG. 5) and transformed into C. reinhardtii. The resulting construct produces one RNA with a nucleotide sequence encoding a selection protein (file) and a nucleotide sequence encoding a protein of interest (any one of YD01 to YD07). The expression of the two proteins are linked by the viral peptide 2A (for example, as described in Donnelly et al., J Gen Virol (2001) vol. 82 (Pt 0.5) pp. 101:3-25). This protein sequence facilitates the expression of two polypeptides from a single mRNA. This construct also contains a cassette that confers resistance to paromomycin. The seven targets are described below in Table 1 (YD=yield gene) (YD01=YD1, YD02=YD2, and so on).

TABLE 1

| | |
|---|---|
| YD01 | AtG2, aminopeptidase/metalloexopeptidase (A. thaliana) |
| YD02 | ErbB3-binding protein 1 (EBP1) (S. tuberosum) |
| YD03 | EBP1/hypothetical protein (C. reinhardtii) |
| YD04 | Target of rapamycin (TOR) kinase (A. thaliana) |
| YD05 | TOR kinase (C. reinhardii) |
| YD06 | Rubisco activase (A. thaliana) |
| YD07 | Rubisco activase (C. reinhardtii) |

The SEnuc745 plasmid (FIG. 5) was created by using pBluescript II SK(−) (Agilent Technologies, CA) as a vector backbone. The segment labeled “AR4 Promoter” indicates a fused promoter region beginning with the C. reinhardtii Hsp70A promoter, C. reinhardtii rbeS2 promoter, and four copies of the first intron from the C. reinhardtii rbcS2 gene (Sizova et al. Gene, 277:221-229 (2001)). The gene encoding a bleomycin binding protein was fused to the 2A region of foot-and-mouth disease virus and the YD ORF was cloned in with XhoI and AgeI. A FLAG-MAT tag is contained in the vector after the AgeI restriction site and is fused to the YD ORF during the cloning process; this is followed on the construct by the Chlamydomonas reinhardtii mM rbcS2 terminator. A paromomycin resistance gene flanked by a psaD promoter and terminator in the vector allows for a secondary selection on paramomycin after transformation into an algae

Transformation DNA was prepared by digesting SENuc745 vector containing each of SEQ ID NOs: 1-7 with the restriction enzyme XbaI or Psil, followed by heat inactivation of the enzyme. For these experiments, all transformations were carried out on C. reinhardtii cc1690 (mt+) cells. Cells were grown and transformed via electroporation. Cells were grown to mid-log phase (approximately 2−6×1⁰⁶ cells/ml) in TAP media. Cells were spun down at between 2000×g and 5000×g for 5 mM. The supernatant was removed and the cells were resuspended in TAP media +40 mM sucrose. 250-1000 ng (in 1-5 μL H₂O) of transformation. DNA was mixed with 250 μL of 3×1⁰⁶ cells/mL on ice and transferred to 0.4 cm electroporation cuvettes. Electroporation was performed with the capacitance set at 25 uF, the voltage at 800 V to deliver 2000 V/cm resulting in a time constant of approximately 10-44 ms. Following electroporation, the cuvette was returned to room temperature for 5-20 min. For each transformation, cells were transferred to 10 ml of TAP media +40 mM sucrose and allowed to recover at room temperature for 12-16 hours with continuous shaking. Cells were then harvested by centrifugation at between 2000×g and 5000×g, the supernatant was discarded, and the pellet was resuspended in 0.5 ml TAP media +40 mM sucrose. The resuspended cells were then plated on solid TAP media +10 μg/mL zeocin. Algae cells were then transferred to solid TAP media +10 μg/mL paromomycin. From these cells, the YD ORF was PCR amplified and sequenced to confirm identify and completeness. As a result, overexpression cell lines for YD01 to YD07 were created.

### Example 2

### Competitive Growth Assays for Yield Genes

Twelve sequence positive., transgenic lines of 6 individual YD genes (YD1, YD3, YD4, YD6 and YD7) were grown to saturation in TAP medium in a 96-deep well block. Cells were split back 1/50 in High Salt Medium (HSM) and subsequently grown in a 5% CO₂ in air environment until cells reached early log phase. 500 ul of the transgenic lines of each individual gene were pooled into separate conical tubes. A 10 ml equal density mixture of all 6 YD transgenic lines was made based on the OD750 of each individual transgenic pool. A cell count of the equal density mixture was used to make a 19:1 wild-type C. reinhardtii to YD gene pool mixture. 2 ml of the mixture was sorted on TAP solid media and TAP solid media +10 μg/mL zeocin and 10 μg/mL paromycin. A comparison of colonies growing on TAP versus TAP selective media verified a transgenic starting population near 5%.

The mixed culture was split into biological triplicate turbidostats in a final volume equal to 60 ml. Cultures were supplemented with bubbling CO₂ at approximately 1% in air and continuously maintained at OD750=0.25 for three weeks.

Lines that possess a competitive advantage over wild type and the other transgenic lines in the pool will increase their representation in the turbidostat relative to the starting distribution.

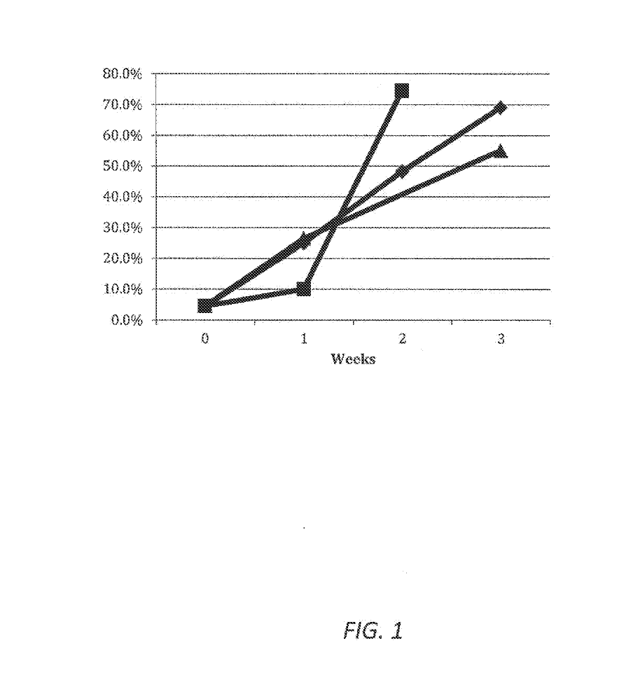

Table 2 below represents data obtained from the competition of the pool of transgenic strains vs. wild type. Once a week, colonies were sorted by FACS onto selective (TAP+10 μg/mL zeocin) and permissive (TAP) media. The number of surviving colonies were then counted and calculated as a percent of the number of colonies sorted. In each turbidostat, the “Start” line demonstrates that the 5% transgenic baseline is accurate. Samples were sorted and colonies were counted each week for three weeks. The course of the transgenic population is shown in FIG. 1. In all three turbidostats, the transgenic lines took over the culture, indicating a growth advantage over wild type. This indicates an increase in growth rate for the transgenic lines relative to the untransformed line. This increase in growth rate can be extrapolated to increased biomass, as under identical conditions and time, the transgenic line produced more cells and therefore more biomass.

TABLE 2

| | | Number of Transgenic Colonies | | | Total Number of Colonies | | |
|---|---|---|---|---|---|---|---|
| | | | Colonies | | | Colonies | |
| | | Tap + Zeo₁₀ | sorted | Percent | Tap | sorted | Percent |
| Turb 1 | Start | 36 | 960 | 3.8% | 852 | 1024 | 83.2% |
| | Week 1 | 88 | 384 | 22.9% | 353 | 384 | 91.9% |
| | Week 2 | 528 | 1152 | 45.8% | 1095 | 1152 | 95.1% |
| | Week 3 | 751 | 1152 | 65.2% | 1088 | 1152 | 94.4% |
| Turb 2 | Start | 36 | 960 | 3.8% | 852 | 1024 | 83.2% |
| | Week 1 | 36 | 384 | 9.4% | 359 | 384 | 93.5% |
| | Week 2 | 808 | 1152 | 70.1% | 1085 | 1152 | 94.2% |
| | Week 3* | 258 | 1152 | *22.4% | 1087 | 1152 | 94.4% |
| Turb 3 | Start | 36 | 960 | 3.8% | 852 | 1024 | 83.2% |
| | Week 1 | 96 | 384 | 25.0% | 363 | 384 | 94.5% |
| | Week 2 | FACS malfunctioned. No colonies sorted onto plates | | | | | |
| | Week 3 | 573 | 1152 | 49.7% | 1040 | 1152 | 90.3% |

**Turbidostat contaminated.

Colonies from the FACS sorting were lysed by boiling in 10× TE buffer and the YD ORF was amplified by PCR. Amplification products were sequenced and the final YD gene frequency of the turbidostat was determined. Six transgenes were equally represented in the starting population.

Table 3 shows the number of clones identified for each of the YD genes from the sort completed at week 2.

Table 4 shows the number of clones identified for each of the YD genes from the sort completed at week 3.

Table 5 shows the percentage of clones identified for each of the YD genes from the final sort for each of the three replicate turbidostats.

As seen in Tables 3, 4 and 5 below, YD7 is the dominant transgene present in the final population, suggesting that this transgenic line has a selective growth advantage over wild type and the other transgenic lines. This indicates an increase in growth rate for the YD07 transgenic lines relative to the untransformed line. This increase in growth rate can be extrapolated to increased biomass, as under identical conditions and time, the YD07 transgenic line produced more cells and therefore more biomass

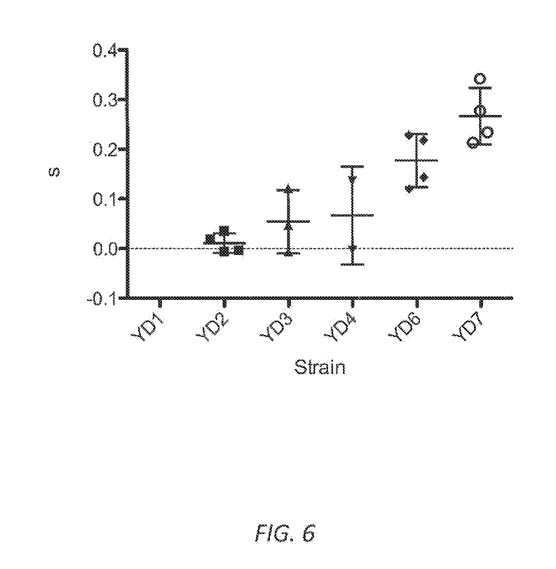

From these sequencing results, a selection coefficient can be calculated using the equation ln(r₀)=ln(rₜ)+s*t where r₀ is the ratio at time 0, rₜ is the ratio at time t and s is the selection coefficient in units of t⁻¹ (as derived from Lenski, R. E. (1991). Quantifying fitness and gene stability in microorganisms. Biotechnology (Reading, Mass), 15, 173-492.). These selection coefficients are shown in Table 6 below and in FIG. 6. Positive selection coefficients for YD07 and YD06 in all cases tested indicated an increase in growth rate for these transgenic lines relative to the untransformed line. Transgenic lines over expressing YD02, YD03 and YD04 have a positive selection coefficient in at least one case showing that these strains also have an increased growth rate relative to the untransformed line.

TABLE 3
Week 2 sequencing.

| Turbidostat 1 | Count | Turbidostat 2 | Count |
|---|---|---|---|
| YD01 | 0 | YD01 | 0 |
| YD02 | 2 | YD02 | 2 |
| YD03 | 11 | YD03 | 3 |
| YD04 | 2 | YD04 | 10 |
| YD06 | 38 | YD06 | 32 |
| YD07 | 74 | YD07 | 98 |

TABLE 4
Week 3 sequencing.

| Turbidostat 1 | Count | Turbidostat 2** | Count | Turbidostat 3 | Count |
|---|---|---|---|---|---|
| YD01 | 0 | YD01 | 7 | YD01 | 0 |
| YD02 | 2 | YD02 | 7 | YD02 | 2 |
| YD03 | 0 | YD03 | 30 | YD03 | 2 |
| YD04 | 0 | YD04 | 1 | YD04 | 0 |
| YD06 | 17 | YD06 | 33 | YD06 | 26 |
| YD07 | 64 | YD07 | 21 | YD07 | 120 |

**Turbidostat 2 was contaminated at the point of the week 3 sort.

TABLE 5

| | YD1 | YD2 | YD3 | YD4 | YD6 | YD7 |
|---|---|---|---|---|---|---|
| Turb-1 Week 3 | 0% | 2% | 0% | 0% | 20% | 77% |
| Turb-2 Week 2 | 0% | 1% | 2% | 7% | 22% | 68% |
| Turb-3 Week 3 | 0% | 1% | 1% | 0% | 17% | 80% |

TABLE 6
Selection Coefficients (day⁻¹)

| | Turb1 Week2 | Turb2 Week2 | Turb1 Week3 | Turb3 Week3 |
|---|---|---|---|---|
| YD1 | — | — | — | — |
| YD2 | −0.003 | 0.018 | 0.036 | −0.006 |
| YD3 | 0.121 | 0.048 | — | −0.006 |
| YD4 | −0.003 | 0.136 | — | — |
| YD6 | 0.217 | 0.228 | 0.144 | 0.120 |
| YD7 | 0.277 | 0.341 | 0.233 | 0.213 |

in order to better ascertain the selective advantage that lines over expressing YD07 have relative to the untransformed line, multiple one-on-one competitions were completed. Twelve sequence positive, transgenic lines of YD07 were grown to saturation in TAP medium then split back 1/50 in High Salt Medium (HSM) and subsequently gown in a 5% CO₂ in air environment until cells reached early log phase. 500 ul of the transgenic lines were pooled into conical tubes and a cell count of this mixture was used to make a 19:1 wild-type C. reinhardtii YD07 mixture 2 ml of the mixture was sorted on TAP solid media and TAP solid media +10 μg/mL zeocin and 10 μg/mL paromycin. A comparison of colonies growing on TAP versus TAP selective media verified a transgenic starting population near 5%.

The mixed culture was split into biological replicate turbidostats each in a final volume equal to 30 ml. Cultures were supplemented with bubbling CO₂ at approximately 1% in air and continuously maintained at OD750=0.25 for 11 days, Cells from the turbidostats were sorted on TAP solid media and TAP solid media +10 μg/mL zeocin and 10 μg/mL paromycin. A comparison of colonies growing on TAP versus TAP selective media indicates the final relative YD07 and wild type populations.

Lines that possess a competitive advantage over wild type will increase their representation in the turbidostat relative to the starting distribution. As shown in Table 7, the YD07 transgenic lines increased in relative abundance from 4.2% at Time 0 to between 34.2% and 91.0% at day 1. The selection coefficient (s) for these replicate experiments was calculated and is shown in Table 7.

TABLE 7
YD07 competition data

| Experiment number | Tap + Zeo | Tap | Percent | s (day⁻¹) |
|---|---|---|---|---|
| Time 0 | 21 | 502 | 4.20% | n/a |
| 7-12 | 128 | 351 | 36.5% | 0.234 |
| 7-11 | 275 | 364 | 75.5% | 0.387 |
| 7-9 | 333 | 366 | 91.0% | 0.495 |
| 16-10 | 181 | 353 | 51.3% | 0.289 |
| 16-8 | 239 | 356 | 67.1% | 0.350 |
| 16-7 | 193 | 346 | 55.8% | 0.306 |
| 32-12 | 186 | 349 | 53.3% | 0.297 |
| 32-10 | 122 | 357 | 34.2% | 0.225 |
| 34-9 | 283 | 373 | 75.9% | 0.389 |

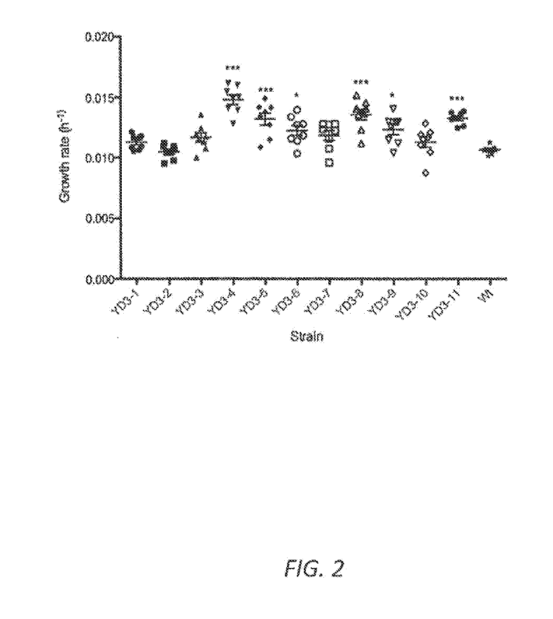

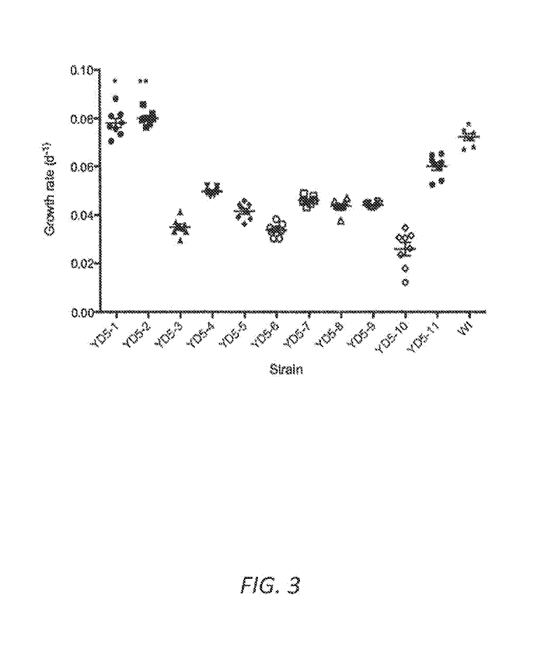

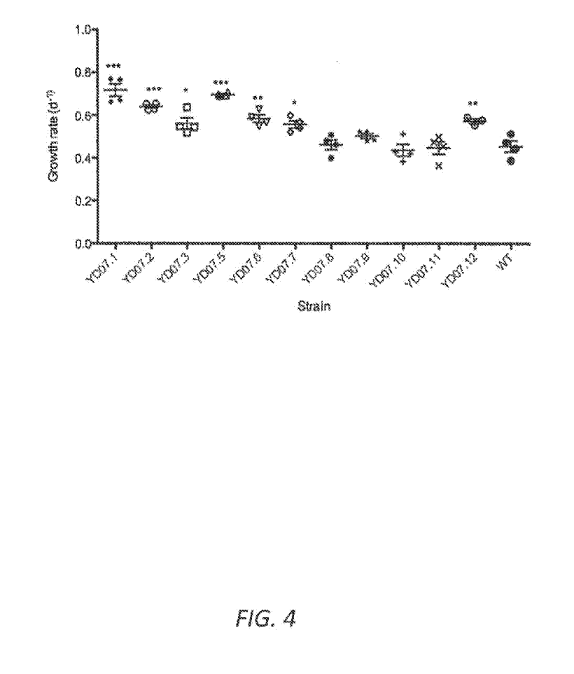

In addition to the competition growth assays described above, growth rates on 12 independent transgenic lines for three of the genes (YD3, YD5 and YD7) were determined in growth assays. Cells were grown in a 96 well plate to full saturation. Cells were then diluted into HSM media and grown overnight. From this culture, replicates of each line were diluted into HSM media in microtitre plates at OD₇₅₀=0.02. Plates were grown under light in a 5% CO₂ environment and OD750 readings were taken every 8-16 hours. Data is plotted based on the natural log of the OD. Growth rate is taken from the slope of the curve over a period of time. Growth rates for YD3, YD5 and YD7 transgenic lines along with a wild type control are shown in FIG. 2, FIG. 3, and FIG. 4, respectively.

The seven genes that resulted in increased biomass in C. reinhardtii overexpression cell lines are listed in the following Table 4 along with the Joint Genome Institute (JGI) protein ID v3 or NCBI accession number and functional annotation.

TABLE 4

| Yield Gene | Protein ID | Functional Annotation |
|---|---|---|
| YD01 | AAC14407 | EBP1 |
| YD02 | ABJ97690 | EBP1 |
| YD3 | 380918 | EBP1 |
| YD04 | NP_175425 | TOR kinase |
| YD5 | 415627 | TOR kinase |
| YD06 | NP_565913 | Robisco Activase |
| YD7 | 128745 | Rubisco Activase |

### Example 3

### Identification of Rubisco Activase from Other Algae Species

The sequence of C. reinhardtii Rubisco activase was used in a BLAST search of the transcriptome sequences of Scenedesmus dimorphus and a Desmodesmus sp. A partial protein sequence was identified from each of the two algae. These sequences were used to design oligonucleotide primers that were then used in reverse transcription and PCR amplification reactions from RNA isolated from the two algae species. Via sequencing these cloned PCR products, the full length sequences of rubisco activase from Scenedesmus dimorphus and a Desmodesmus sp. were determined (SEQ ID NO: 29 and SEQ ID NO: 35). The two genes were codon optimized for nuclear expression in a Desmodesmus sp. (SEQ ID NO: 31 and SEQ ID NO: 37). (SEQ ID NO: 31 and SEQ ID NO: 32 can also be used for nuclear expression. In Chlamydomas, Scenedesmus, or Nannochloropsis sp.)

These two genes can be expressed in any photosynthetic organism, for example, C. reinhardtii. The gene sequences can be cloned into a transformation vector (for example, as shown in FIG. 5). This vector can be transformed into C. reinhardtii to produce an increased biomass phenotype.

### Example 4

### Codon Optimization of YD2, YD3 and a Thermostable Variant of RCA

Three genes were codon optimized and expressed in the nucleus of C. reinhardtii. The three codon optimized genes are YD41 (SEQ ID NO: 63), YD27 (SEQ ID NO: 65), and YD22 (SEQ ID NO: 67). SEQ ID NO: 63 is the nucleic acid sequence of the YD3 protein (SEQ ID NO: 10) codon optimized for expression in the nucleus of C. reinhardtii (SEQ ID NO: 63 is YD41). SEQ ID NO: 63 was cloned into a vector (as described below) with an XhoI site upstream of the start codon and a BamHI site downstream of the stop codon. SEQ ID NO: 65 is a thermostable variant Rubisco activase 13 gene sequence (as described in Kurek, I., et al., The Plant Cell (2007) Vol. 19:3230-32411 codon optimized for nuclear expression in C. reinhardtii. The mutations made are F168L, V257I, and K310N (relative to the A. thaliana RCA1 protein sequence) (SEQ ID NO: 65 is YD27). SEQ ID NO: 65 was cloned into a vector (as described below) with an XhoI site upstream of the start codon and a BamHI site downstream of the stop codon. SEQ ID NO: 67 is the nucleic acid sequence of a YD2 protein (SEQ ID NO: 70) codon optimized for expression in the nucleus of C. reinhardtii (SEQ ID NO: 67 is YD22). SEQ ID NO: 67 was cloned into a vector (as described below) with an XhoI site upstream of the start codon and a BamHI site downstream of the stop codon.

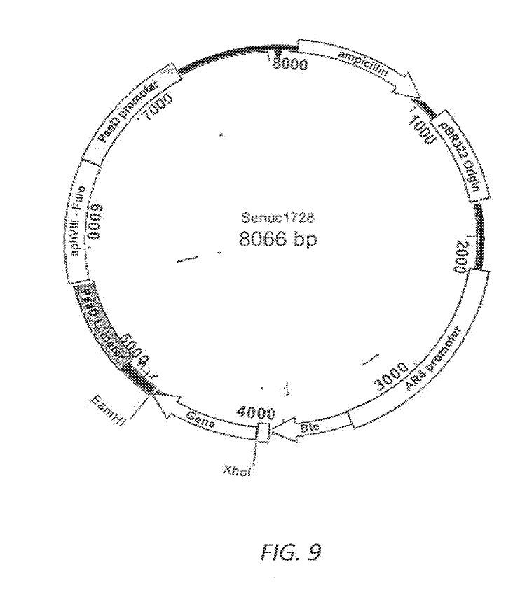

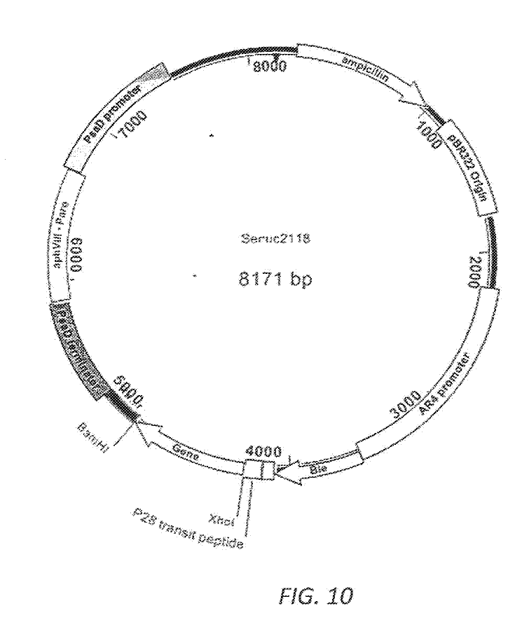

The DNA constructs (SEQ ID NOs: 63 and 67, including the XhoI and BamHI sites) for two of the three targets were each individually cloned into unclear overexpression vector SEnuc1728 (FIG. 9) and transformed into C. reinhardtii. The DNA construct (SEQ ID NO: 65 including the XhoI and BamHI sites) was cloned into nuclear overexpression vector SEnuc2118 (FIG. 10) and transformed into C. reinhardtii. SEnuc1728 and SEnuc2118 are identical in sequence, with the exception that SEnuc2118 contains a targeting peptide (P28 transit peptide) upstream of the XhoI restriction site, which will result in chloroplast targeting of the downstream peptide. The resulting constructs produces one RNA with a nucleotide sequence encoding a selection protein (Ble) and a nucleotide sequence encoding a protein of interest. The expression of the two proteins are linked by the viral peptide 2A (for example, as described in Donnelly et al., J Gen Virol (2001) vol. 82 (Pt 5) pp. 1013-25). This protein sequence facilitates the expression of two polypeptides from a single mRNA. This construct also contains a cassette that confers resistance to paromomycin.

SEnuc1728 and SEnuc2118 were created by using pBluescript II SK(−) (Agilent Technologies, CA) as a vector backbone. The segment labeled “AR4 Promoter” indicates a fused promoter region beginning with the C. reinhardtii Hsp70A promoter, C. reinhardtii rbcS2 promoter, and four copies of the first intron from the C. reinhardtii rbcS2 gene (Sizova et al. Gene, 277:221-229 (2001)). The gene encoding a bleomycin binding protein was fused to the 2A region of foot-and-mouth disease virus and the YD ORF was cloned in with XhoI and BamHI. A paromomycin resistance gene flanked by a psaD promoter and terminator in the vector allows for a secondary selection on paramomycin after transformation into an algae

Transformation DNA was prepared by digesting SEnuc1728 and SEnuc2118 containing each of SEQ NOs: 63, 65, and 67 (including the XhoI and BamHI sites) with the restriction enzyme XbaI or PsiI, followed by heat inactivation of the enzyme, SEnuc1728 has an XbaI site at nucleotides 2223-2228 and a PsiI site at nucleotides 7962-7967. SEnuc2118 has an XbaI site at nucleotides 2223-2228 and a PsiI site at nucleotides 8067-8072.

For these experiments, all transformations were carried out on C. reinhardtii cc1690 (mt+) cells. Cells were grown and transformed via electroporation. Cells were grown to mid-log phase (approximately 2−6×1⁰⁶ cells/ml) in TAP media. Cells were spun down at between 2000×g and 5000×g for 5 min. The supernatant was removed and the cells were resuspended in TAP media +40 ml)/1 sucrose. 250-1000 ng (in 1-5 μL H₂O) of transformation DNA was mixed with 250 μL of 3×1⁸ cells/mL on ice and transferred to 0.4 cm electroporation cuvettes, Electroporation was performed with the capacitance set at 25 uF, the voltage at 800 V to deliver 2000 V/cm resulting in a time constant of approximately 10-14 ms. Following electroporation, the cuvette was returned to room temperature for 5-20 min. For each transformation, cells were transferred to 10 ml of TAP media +40 mM sucrose and allowed to recover at room temperature for 12-16 hours with continuous shaking. Cells were then harvested by centrifugation at between 2000×g and 5000×g, the supernatant was discarded, and the pellet was resuspended in 0.5 ml TAP media +40 mM sucrose. The resuspended cells were then plated on solid TAP media-1-10 μg/mL zeocin. Algae cells were then transferred to solid TAP media +10 μg/mL paromomycin. From these cells, the YD ORF was PCR amplified and sequenced to confirm identify and completeness. As a result, overexpression cell lines for YD41, YD27, and YD22 were created.

### Example 5

### Microtiter Growth Assays for Yield Genes

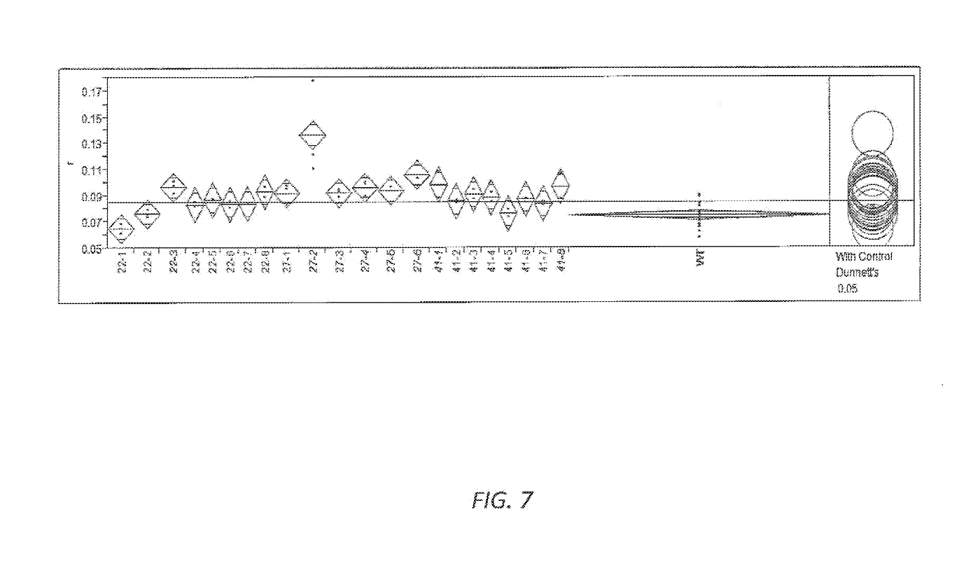

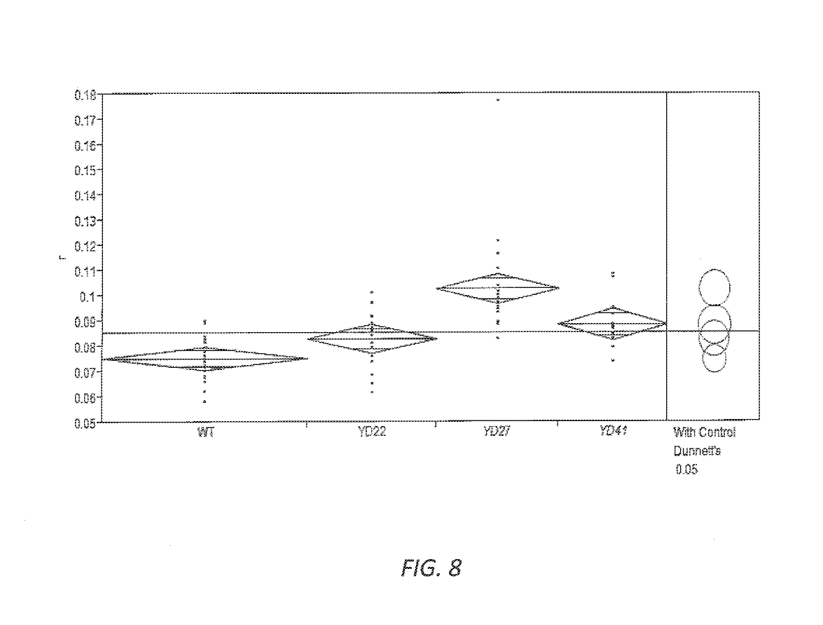

The growth rates of 22 independent transgenic lines for three of the genes (YD22, YD27 and YD41) were determined in growth assays. Cells were grown in a 96 well plate to full saturation. Cells were then diluted into HSM media and grown overnight. From this culture, replicates of each line were diluted into HSM media in microtitre plates at OD₇₅₀=0.02. Plates were grown under light in a 5% CO₂ environment and OD750 readings were taken every 6 hours. OD750 readings were plotted and an exponential curve was fit to the data. The growth rate for each transgenic line was calculated as the slope of the exponential curve at its inflection point. Growth rates for YD22, YD27 and YD41 transgenic lines along with a wild type control are were determined and the data analyzed by a Oneway analysis of “r” (growth rate) by individual YD gene transformant, or by groups of YD gene transformants as shown in FIG. 7 and FIG. 8, respectively. A Dunnet's test was also done and is shown in FIG. 7 and FIG. 8. As shown in FIG. 7, the growth rate of several individual transformants for each of YD22, YD27, and YD41 were greater than the wild type control. FIG. 8 shows that when the transformants were grouped by YD gene, all three groups had a growth rate greater than the wild type.

Dunnett's test is a statistical tool known to one skilled in the art and is described, for example, in Dunnett, C. W. (1955) “A multiple comparison procedure for comparing several treatments with a control”, Journal of the American Statistical Association, 50:1096-1121, and Dunnett, C. W. (1964) “New tables for multiple comparisons with a control”, Biometrics, 20:482-491. Dunnett's test compares group means. it is specifically designed for situations where all groups are to be pitted against one “Reference” group. It is commonly used after ANOVA has rejected the hypothesis of equality of the means of the distributions (although this is not necessary from a strictly technical standpoint). The goal of Dunnet's test is to identify groups whose means are significantly different from the mean of this reference group. It tests the null hypothesis that no group has its mean significantly different from the mean of the reference group.

How to Measure an Increase in Biomass Yield in a YD Overexpression Cell Line.

This section describes exemplary methods that can be used to determine the increase in biomass or increase in biomass yield in a cell line transformed with a YD gene.

The organism (cell line) can be grown in a flask, a plate reactor, a paddlewheel pond, or other vessel. One of skill in the art would be able to choose an appropriate vessel.

An increase in biomass or biomass yield can be measured by a competition assay, growth rate, carrying capacity, measuring culture productivity, cell proliferation, seed yield, organ growth, or polysome accumulation. These types of measurements are known to one of skill in the art.

The growth of the organism can be measured by optical density, dry weight, by total organic carbon, or by other methods known to one of skill in the art. These measurements can be, for example, fit to a growth curve to determine the maximal growth rate, the carrying capacity, and the culture productivity (for example, g/m2/day; a measurement of biomass produced per unit area/volume per unit time). These values can be compared to an untransformed cell fine or another transformed cell line, to calculate the increase in biomass yield in the YD over expressing cell line of interest.

Carrying capacity can be measured, for example, as grams per liter, grams per meter cubed, grams per meter squared, or kilograms per acre. One of skill in the art would be able to choose the most appropriate units. Any mass per unit of volume or area can be measured.

Culture productivity can be measured, for example, as grams per meter squared per day, grams per liter per day, kilograms per acre per day, or grams per meter cubed per day. One of skill in the art would be able to choose the most appropriate units.

Growth rate can be measured, for example, as per hour, per day, per generation or per week. One of skill in the art would be able to choose the most appropriate units. Any per unit time can be measured.

Analysis of RNA and Protein Expression in a YD Over Expressing Cell Line.

This section describes methods to measure expression of RNA and protein from a YD over expressing cell line. Total RNA or mRNA can be purified from the YD over expressing cell line and compared to an untransformed cell line. YD gene RNA levels can be measured by PCR, qPGR, Northern blot, microarray, RNA-Sect, serial analysis of gene expression (SAGE) or other methods known to one of skill in the art. Expression of the YD protein can be measured by Western blot, immunoprecipitation, or other methods known to one of skill in the art.

Chloroplast Expression of RCA without a Choloroblast Transit Peptide.

This section describes a method to express a YD gene from the chloroplast of a photosynthetic organism. A protein expressed by the YD gene may exert its effect in the chloroplast of the organism. This type of protein typically has a chloroplast transit peptide at the N-terminus of the protein that is cleaved upon entry into the chloroplast. The YD protein can be expressed from the chloroplast by codon optimizing the gene for chloroplast expression and removing the portion of sequence encoding the transit peptide. This gene can then be inserted into a chloroplast expression vector and transformed into the chloroplast of a photosynthetic organism.

For example, SEQ ID NO: 45 described above, is SEQ ID NO: 27 (the endogenous nucleic acid sequence of YD6) codon optimized for chloroplast expression in Scenedesmus dimorphus or C. reinhardtii.

Also, SEQ ID NO: 47 described above, is SEQ ID NO: 28 (the endogenous nucleic acid sequence of YD7) codon optimized for chloroplast expression in Scenedesmus dimorphus or C. reinhardtii.

Expression of Variant Forms of RCA.

This section describes a method to express variants of Rubisco activase. Certain modifications to this protein are known to impact the function in vivo (for example, as described in Kurek, I., et al., The Plant Cell (2007) Vol. 19:3230-3241). These modifications can be made to the coding sequence before cloning the coding sequence into a vector, optionally, the coding sequence containing the modification(s) can be codon optimized for the organism to be transformed prior to cloning into the vector. A photosynthetic organism is then transformed with the vector, and the protein of interest is expressed. Also, similar modifications can be made in orthologous positions (based on protein alignments and conservation) based on the protein sequence of other organisms.

For example, SEQ ID NO: 4:3 is a thermostable variant of Rubisco activase, codon optimized for nuclear expression in Scenedesmus dimorphus. This sequence is an RCA2 (β) or short isoform, with point mutations (F168L, V257I, and K310N) previously shown to provide thermostability in A. thaliana.

Expression of YD Genes in Other Algal Strains

This section describes a method to over express a YD gene in an alternative algae species in order to increase the biomass yield of the algae. The YD ORF (with or without modifications and/or codon optimization) can be cloned into a transformation vector, for example, as shown in FIG. 5. The vector can then be used to transform a Dunaliella sp. Scenedesmus sp., Desmodesmus sp., Nannochloropsis sp., Chlorella sp., Botryococcus sp., or Haematococcus sp., resulting in expression of the YD protein.

Alternatively, a transformation vector with nucleotide sequence elements (for example, a promoter, a terminator, and/or a UTR) specific to a host algae species can be used with the YD ORF. This alternate vector can be transformed into algae species such as a Dunaliella sp, Scenedesmus sp., Desmodesmus sp., Nannochloropsis sp., Chlorella sp., Botryococcus sp., or Haematococcus sp.

Overexpression of a YD gene in the species described herein can be used to produce a phenotype with an increased biomass yield.

For example, SEQ ID NOs: 41-49 represent nucleic acid sequences that have been codon optimized for expression in either the chloroplast and/or the nucleus of S. dimorphus. SEQ ID NOs: 41-44, 46, and 48-49 can also be used to for expression in the nucleus of a Desmodesmus sp., Nannochloropsis sp., or Chlamydomonas sp. The codon optimization table used to create these sequences is shown above in Table D.

Expression of YD Genes in Higher Plants.

This section describes a method to over express YD gene in a higher plant, such as Arabidopsis thaliana in order to change the biomass yield of the plant. The YD ORF (with or without modifications and/or codon optimization) can be cloned into a transformation vector, for example, as described in FIG. 5, a pBS SK-2×myc vector (as described in Magyar, Z. (2005) THE PLANT CELL ONLINE, 17(9), 2527-2541; doi.:10.1105/tpc.105.033761), or a pMAXY4384 vector (as described in Marek, I., et al. (2007) The Plant Cell, 19(10), 3230-3241. doi:10.1105/tpc.107.054171), and the YD protein expressed in, for example, a Brassica, Glycine, Gossypium, Medicago, Zea, Sorghum, Oryza, Triticum, or Panicum species.

Alternatively, a transformation vector with nucleotide sequence elements (for example, a promoter, a terminator, and/or a UTR) specific to a host plant species can be used with the YD ORF. This alternate vector can be transformed into higher plant species such as Brassica, Glycine, Gossypium, Medicago, Zea, Sorghum, Opyza, Triticum, or Panicum species.

Overexpression of a YD gene in any of the species disclosed herein can be used to produce a phenotype with an increased biomass yield.

It is to be understood that the present invention has been described in detail by way of illustration and example in order to acquaint others skilled in the art with the invention, its principles, and its practical application. Particular compositions and processes of the present invention are not limited to the descriptions of the specific embodiments presented, but rather the descriptions and examples should be viewed in terms of the claims that follow and their equivalents.

It is to be further understood that the specific embodiments set forth herein are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the scope of the following claims.

1-231. (canceled)

232. A method of increasing biomass of a photosynthetic organism, comprising:
(a) transforming the photosynthetic organism with a polynucleotide, wherein the polynucleotide comprises:
(i) nucleic acid sequence of SEQ ID NO: 21, 19, 17, 20, 18, 16, 15, 61, 64, 66, 68 or 69; or
(ii) a nucleotide sequence with at least 80%, at least 85%, at least 90%, at least 95%, at least 98%, or at least 99% sequence identity to nucleic acid sequence SEQ ID NO: 21, 19, 17, 20, 18, 16, 15, 61, 64, 66, 68 or 69;
and wherein the nucleic acid of (i) or the nucleotide of (ii) encode for a polypeptide that when expressed results in an increase in the biomass of the transformed photosynthetic organism as compared to an untransformed photosynthetic organism.

233. The method of claim 232, wherein:
a) the increase is measured by a competition assay, growth rate, carrying capacity, culture productivity, cell proliferation, seed yield, organ growth, or polysome accumulation;
b) the increase is measured by a competition assay;
c) the increase is measured by a competition assay and the competition assay is performed in a turbidostat;
d) the increase is shown by the transformed photosynthetic organism having a positive selection coefficient as compared to the untransformed photosynthetic organism;
e) the increase is shown by the transformed photosynthetic organism having a positive selection coefficient as compared to the untransformed photosynthetic organism and the selection coefficient is from 0.05 to 0.10, from 0.10 to 0.5, from 0.5 to 0.75, from 0.75 to 1.0, from 1.0 to 1.5, from 1.5 to 2,0, or 2.0 to 3.0;
f) the increase is measured by growth rate;
g) the increase is measured by growth rate and the transformed photosynthetic organism has an increase in growth rate as compared to the untransformed photosynthetic organism of from 5% to 10%, from 10% to 15%, from 15% to 25%, from 25% to 50%, from 50% to 100%, from 100% to 200%, or from 200% to 400%;
h) the increase is measured by an increase in carrying capacity;
i) the increase is measured by an increase in carrying capacity and the units of carrying capacity are mass per unit of volume or area;
j) the increase is measured by an increase in culture productivity;
k) the increase is measured by an increase in culture productivity and the units of culture productivity are grams per meter squared per day;
l) the increase is measured by an increase in culture productivity and the transformed photosynthetic organism has an increase in culture productivity as measured in grams per meter squared per day, as compared to the untransformed photosynthetic organism of from 5% to 25%, from 25% to 50%, from 50% to 100%, from 100% to 200%, or from 200% to 400%.

234. The method of claim 232, wherein:
a) the transformed photosynthetic organism is grown in an aqueous environment;
b) the transformed photosynthetic organism is a bacterium;
c) the transformed photosynthetic organism is a cyanobacterium;
d) the transformed photosynthetic organism is an alga;
e) the transformed photosynthetic organism is a microalga;
f) the transformed photosynthetic organism is at least one of a Chlamydomonas sp., Volvacales sp Desmid sp., Dunaliella sp., Scenedesmus sp. Chlorella sp Hematococcus sp., Volvax sp., Nannochloropsis sp., Arthrospira sp., Sprirulina sp., Botryococcus sp., Haematococcus sp., or Desmodesmus sp.;
g) the transformed photosynthetic organism is at least one of Chlamydomonas reinhardtii, N. oceanic, N. salina, Dunaliella salina, H. pluvalis, S. dimorphus, Dunaliella viridis, N. oculata, Dunaliella tertiolecta, S. Maximus, or A. Fusiformus;
h) the transformed photosynthetic organism is a vascular plant;
i) the transformed photosynthetic organism is a higher plain; or
j) the transformed photosynthetic organism is a higher plant and the higher plant is Arabidopsis thaliana, or a Brassica, Glycine, Gossypium, Medicago, Zea, Sorghum, Oryza, Triticum, or Panicum species.

235. A method of increasing biomass of a photosynthetic organism, comprising:
(a) transforming the photosynthetic organism with a polynucleotide, wherein the polynucleotide comprises:
(i) nucleic acid sequence SEQ ID NO: 50, 51, 52, 53, 54, 55, 56, 57, 58, or 62; or
(ii) a nucleotide sequence with at least 80%, at least 85%, at least 90%, at least 95%, at least 98%, or at least 99% sequence identity to nucleic acid sequence SEQ ID NO: 50, 51, 52, 53, 54, 55, 56, 57, 58, or 62;
and wherein the nucleic acid of (i) or the nucleotide of (ii) encode for a polypeptide that when expressed results in an increase in the biomass of the transformed photosynthetic organism as compared to an untransformed photosynthetic organism.

236. The method of claim 235, wherein:
a) the increase is measured by a competition assay, growth rate, carrying capacity, culture productivity, cell proliferation, seed yield, organ growth, or polysome accumulation;
b) the increase is measured by a competition assay;
c) the increase is measured by a competition assay and the competition assay is performed in a turbidostat;
d) the increase is shown by the transformed photosynthetic organism having a positive selection coefficient as compared to the untransformed photosynthetic organism;
e) the increase is shown by the transformed photosynthetic organism having a positive selection coefficient as compared to the untransformed photosynthetic organism and the selection coefficient is from 0.05 to 0.10, from 0.10 to 0.5, from 0.5 to 0.75, from 0.75 to 1.0, from 1.0 to 1.5, from 1.5 to 2,0, or 2.0 to 3.0;
f) the increase is measured by growth rate;
g) the increase is measured by growth rate and the transformed photosynthetic organism has an increase in growth rate as compared to the untransformed photosynthetic organism of from 5% to 10%, from 10% to 15%, from 15% to 25%, from 25% to 50%, from 50% to 100%, from 100% to 200%, or from 200% to 400%;
h) the increase is measured by an increase in carrying capacity;
i) the increase is measured by an increase in carrying capacity and the units of carrying capacity are mass per unit of volume or area;
j) the increase is measured by an increase in culture productivity;
k) the increase is measured by an increase in culture productivity and the units of culture productivity are grams per meter squared per day;
l) the increase is measured by an increase in culture productivity and the transformed photosynthetic organism has an increase in culture productivity as measured in grams per meter squared per day, as compared to the untransformed photosynthetic organism of from 5% to 25%, from 25% to 50%, from 50% to 100%, from 100% to 200%, or from 200% to 400%.

237. The method of claim 235, wherein:
a) the transformed photosynthetic organism is grown in an aqueous environment;
b) the transformed photosynthetic organism is a bacterium;
c) the transformed photosynthetic organism is a cyanobacterium;
d) the transformed photosynthetic organism is an alga;
e) the transformed photosynthetic organism is a microalga;
f) the transformed photosynthetic organism is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.;
g) the transformed photosynthetic organism is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus;
h) the transformed photosynthetic organism is a vascular plant;
i) the transformed photosynthetic organism is a higher plant; or
j) the transformed photosynthetic organism is a higher plant and the higher plant is Arabidopsis thaliana, or a Brassica, Glycine, Gossypium, Medicago, Zea, Sorghum, Oryza, Triticum, or Panicum species.

238. A method of increasing biomass of a photosynthetic organism, comprising:
(a) transforming the photosynthetic organism with a polynucleotide, wherein the polynucleotide comprises:
(i) nucleic acid sequence of SEQ ID NO: 32, 38, 34, or 40;
(ii) a nucleotide sequence with at least 80%, at least 85%, at least 90%, at least 95%, at least 98%, or at least 99% sequence identity to nucleic acid sequence SEQ ID NO: 32, 38, 34, or 40;
(iii) the nucleic acid sequence of SEQ ID NO: 32 or SEQ ID NO: 38 wherein the nucleic acid sequence is codon optimized for expression in the chloroplast of a Chlamydomonas, Nannochloropsis, Scenedesmus, or Desmodesmus species; or
(iv) the nucleic acid sequence of SEQ ID NO: 32 or SEQ ID NO: 38 wherein the nucleic acid sequence is codon optimized for expression in the nucleus of one or more of a Chlamydomonas, Nannochloropsis, Scenedesmus, or Desmodesmus species;
and wherein the nucleic acid of (i), (iii), or (iv), or the nucleotide sequence of (ii) encode for a polypeptide that when expressed results in an increase in the biomass of the transformed photosynthetic organism as compared to an untransformed photosynthetic organisme.

239. The method of claim 238, wherein the nucleic acid sequence or the nucleotide sequence encodes a protein comprising, (a) amino acid sequence SEQ ID NO: 33 or SEQ ID NO: 39; or (b) a homolog of the amino acid sequence of (a), wherein the homolog has at least 80%, at least 85%, at least 90%, at least 95%, at least 98%, or at least 99% sequence identity to amino acid sequence SEQ ID NO: 33 or SEQ ID NO: 39.

240. The method of claim 238, wherein:
a) the increase is measured by a competition assay, growth rate, carrying capacity, culture productivity, cell proliferation, seed yield, organ growth, or polysome accumulation;
b) the increase is measured by a competition assay;
c) the increase is measured by a competition assay and the competition assay is performed. in a turbidostat;
d) the increase is shown by the transformed photosynthetic organism having a positive selection coefficient as compared to the untransformed photosynthetic organism;
e) the increase is shown by the transformed photosynthetic organism having a positive selection coefficient as compared to the untransformed photosynthetic organism and the selection coefficient is from 0.05 to 0.10, from 0.10 to 0.5, from 0.5 to 0.75, from 0.75 to 1.0, from 1.0 to 1,5, from 1.5 to 2.0, or 2.0 to 3,0;
f) the increase is measured by growth rate;
g) the increase is measured by growth rate and the transformed photosynthetic organism has an increase in growth rate as compared to the untransformed photosynthetic organism of from 5% to 10%, from 10% to 15%, from 15% to 25%, from 25% to 50%, from 50% to 100%, from 100% to 200%, or from 200% to 400%;
h) the increase is measured by an increase in carrying capacity;
i) the increase is measured by an increase in carrying capacity and the units of carrying capacity are mass per unit of volume or area;
j) the increase is measured by an increase in culture productivity;
k) the increase is measured by an increase in culture productivity and the units of culture productivity are grams per meter squared per day;
l) the increase is measured by an increase in culture productivity and the transformed photosynthetic organism has an increase in culture productivity as measured in grams per meter squared per day, as compared to the untransformed photosynthetic organism of from 5% to 25%, from 25% to 50%, from 50% to 100%, from 100% to 200%, or from 200% to 400%.

241. The method of claim 238, wherein:
a) the transformed photosynthetic organism is grown in an aqueous environment;
b) the transformed photosynthetic organism is a bacterium;
c) the transformed photosynthetic organism is a cyanobacterium;
d) the transformed photosynthetic organism is an alga;
e) the transformed photosynthetic organism is a microalga;
f) the transformed photosynthetic organism is at least one of a Chlamydomonas sp., Volvacales sp., Desmid sp., Dunaliella sp., Scenedesmus sp. Chlorella sp., Hematococcus sp., Volvox sp., Nannochloropsis sp., Arthrospira sp., Sprirulina sp., Botryococcus sp., Haematococcus sp., or Desmodesmus sp.;
g) the transformed photosynthetic organism is at least one of Chlamydomonas reinhardtii, N. oceanica, N. salina, Dunaliella pluvalis, S. dimorphus, Dunaliella viridis, N. oculata, Dunaliella tertiolecta, S. Maximus, or A. Fusiformus;
h) the transformed photosynthetic organism is a vascular plant;
i) the transformed photosynthetic organism is a higher plant; or
j) the transformed photosynthetic organism is a higher plant and the higher plant is Arabidopsis thaliana, or a Brassica, Glycine, Gossypium, Medicago, Zea, Sorghum, Oryza, Triticum, or Panicum species.


SEQUENCE LISTING (70 sequences)

SEQ ID NO: 1 - DNA, 1176 bp, artificial sequence
  synthesized
atgagcagcgatgacgagcgtgacgagaaggagctgagcctgactagcccggaggtggtgaccaagtataagtcggcagcagagattgtgaacaaggcactccaggtggtgctcgccgagtgcaagccgaaggctaagattgtggacatctgcgagaagggcgacagcttcattaaggagcagacagcgtcgatgtacaagaactccaagaagaagatcgagcgcggcgtcgcgttcccgacatgtatttccgtcaacaacacggtcggccacttttcgcccctggcttcggatgagagcgtgctggaggatggcgacatggtgaagatcgacatgggctgccacatcgacggcttcatcgcgctggtggggcacacgcacgtgctgcaagagggccccctgtcgggccggaaggcggacgtgattgcagccgccaacaccgctgcggacgtggccctgcgcctcgtccgtcccggcaagaagaacacagacgtgaccgaggctattcagaaggtggcggctgcgtatgactgcaagatcgtggagggcgtcctgagccaccagctgaagcagcacgtgattgacggtaataaggtcgtgctctcggtgtcgagccccgagaccactgtggacgaggtggagttcgaggagaacgaggtgtacgctatcgacatcgtggcctcgaccggcgacggcaagcccaagctgctggacgagaagcaaacgaccatctacaagaaggacgagtcggtgaactaccagctgaagatgaaggcctcgcgcttcatcatcagcgagatcaagcagaacttcccccggatgcccttcacggcccgctccctggaggagaagcgcgctcgcctggggctggtcgagtgcgtgaaccacggccacctgcaaccctatccggtgctgtacgagaagcccggcgatttcgtggcgcagatcaagttcaccgtgctgctgatgcccaacggctccgaccggatcactagccataccctccaggagctgcccaagaagaccattgaggacccggagatcaagggctggctcgccctgggcattaagaagaagaagggcggcggcaagaagaagaaggcgcaaaaggccggcgagaagggcgaggcctccacggaggcggagccaatggacgcgagctcgaacgcccaggag

SEQ ID NO: 2 - DNA, 1158 bp, artificial sequence
  synthesized
atgtcggatgatgagcgtgaggagaaggagctggatctgactagccctgaggtggtgacgaagtacaagtccgccgccgagatcgtgaacaaggccctccagctggtgctgtcggagtgcaagccaaaggtgaagatcgtggacctgtgcgagaagggcgatgccttcatcaaggagcagaccgggaacatgtacaagaacgtgaagaagaagatcgagcggggcgtggccttcccgacttgtatctccgtgaacaacaccgtgtgccacttcagccctctggcgagcgacgagacgatcgtggaggagggcgacattctgaagatcgacatgggttgccacatcgacggtttcatcgcggtcgtgggtcacacccacgtgctgcacgagggcccggtcacgggccgcgccgctgacgtgatcgccgctgcgaacacggctgcggaggtggcgctgcgcctggtgcgtcccggcaagaagaactcggacgtgaccgaggccatccagaaggtcgcggctgcctacgactgcaagatcgtggagggcgtgctctcgcaccagatgaagcaattcgtgatcgacggcaacaaggtggtgctgagcgtgagcaaccccgacacccgcgtggacgaggccgagttcgaggagaacgaggtgtacagcatcgacattgtgacgagcacgggcgatggcaagcccaagctcctggacgagaagcagacaaccatctacaagcgggccgtggacaagagctacaacctgaagatgaaggcgagccgcttcattttctcggagatcaaccagaagttccccatcatgccattcaccgctcgggacctggaggagaagcgtgcccgtctgggcctggtcgagtgcgtgaaccatgagctcctgcaaccctacccggtcctgcacgagaagccgggcgacctggtggctcacattaagtttactgtgctgctgatgcccaacggcagcgaccgtgtgacatcgcacctgcaagagctgcaacccacgaagacgacggagaacgagcccgagatcaaggcgtggctggcgctccctacgaagactaagaagaagggcggtgggaagaagaagaagggcaagaagggcgacaaggtggaggaggcgtcgcaggccgagccgatggagggc

SEQ ID NO: 3 - DNA, 1161 bp, artificial sequence
  synthesized
atgagcgacgacggtagcattgagcaccaagagccaaatctgagcgtccctgaggtggtgacaaagtacaaggctgcggctgacatttgcaaccgcgccctgctcgccgtggtggaggctgcgaaggacggcgcaaaggtcgtggacctgtgccgcatgggcgaccagttcatcaacaaggagtgcgccaacatttacaagggcaaggagatcgagaagggcgtggcgttccccacctgtgtctcggctaactcgatcgtgggccatttcagccccaattcggaggatgctacggcgctgaagaacggcgatgtggtgaagattgacatgggctgccacattgacgggttcatcgccacccaggccaccaccatcgtggtgggcgatgctgcgatcagcggcaaggcagcagatgtgatcgcggcagcccgcacggccttcgatgccgcagtgcgcctgattcgcccaggcaagcacatcgcggacgtgagcgcgcctctccagaaggtggcggagagcttcggctgcaatctcgtcgagggcgtgatgagccacgagatgaagcagttcgtgattgatggctcgaagtgcatcctcaacaagcccacccccgatcagaaggtcgaggacggcgagttcgaggagaacgaggtgtacgccgtggacatcgtggtgtcgagcggcgagggtaagccgcgtgtgctggacgagaaggagacaacagtgtacaagcgggccctggaggtgacctaccagctgaagatgcaggcctcccgcgcggtgttctcgctggtgaatagcgccttcgcgacgatgcccttcaccctgcgcgcgctgctggacgaggcagcggcccagaagacggagctgaaggcgtcccagctgaagctcggcctggtggagtgcctgaaccacggcctgctgcacccgtacccggtgctgcacgagaagcccggcgaggtggtggctcagatcaagggcaccgtgctgctgatgccgaacggctccagcattattacctccgcgccgcgtcagaccgtgaccacggagaagaaggtggaggacaaggagatcctggacctcctggcaaccccaatctcggccaagtccgccaagaagaagaagaacaaggacaaggctgctgagcccgctgccgctaag

SEQ ID NO: 4 - DNA, 7443 bp, artificial sequence
  synthesized
atgagcacgtcctcgcaatcgtttgtggcaggtcgccctgcctcgatggcgagcccatcccagagccaccgcttctgtggcccgagcgccaccgcgagcggcggtgggagcttcgacacgctgaaccgcgtcattgcggatctgtgttcccgtggcaacccgaaggagggcgctcccctggccttccggaagcacgtggaggaggcggtgcgcgacctgagcggcgaggcttcgagccgcttcatggagcagctgtacgatcgtatcgcgaacctcatcgagtccacggacgtggcggagaacatgggggcgctgcgggccatcgacgagctgactgagatcggcttcggcgagaacgccacgaaggtgtcccggttcgccggctacatgcgcaccgtcttcgagctgaagcgcgacccggagatcctggtgctggcgtcccgcgtgctggggcacctggcacgggcaggcggtgcgatgacctcggacgaggtggagttccagatgaagaccgctttcgattggctgcgcgtggatcgggtcgagtaccggcggttcgcggccgtgctgatcctcaaggagatggccgagaacgcttccactgtcttcaacgtgcacgtgcctgagtttgtggacgccatctgggtggccctgcgggacccgcagctgcaagtgcgcgagcgcgcggtcgaggcgctgcgggcttgcctgcgcgtcatcgagaagcgcgagacacgctggcgtgtccagtggtattaccgcatgtttgaggccactcaggacggcctgggccgcaatgcgcccgtccacagcattcatggctccctgctggcggtgggcgagctgctgcggaacactggcgagttcatgatgtcgcgctaccgcgaggtggctgagatcgtgctccggtatctggagcaccgggatcgcctggtgcgcctgagcattacgtccctcctgccccgtattgcgcacttcctgcgcgaccgtttcgtgaccaactacctgacgatctgcatgaaccacatcctgaccgtcctgcgcatccccgccgagcgggccagcggcttcatcgctctgggcgagatggcaggcgcactggacggtgagctgattcactacctgccgaccatcatgtcccacctgcgggatgccatcgcccctcggaagggccgccccctcctggaggctgtcgcgtgcgtgggcaacatcgcgaaggcgatgggctcgaccgtggagacgcacgtgcgcgacctcctggacgtgatgttctcgtcgtccctgagcagcacgctggtggacgctctggaccagatcactatctccatcccctcgctgctgcccaccgtgcaggatcgcctcctggattgcatctccctggtcctgtcgaagtcgcactactcgcaggccaagcccccagtcaccatcgtgcgcggttcgaccgtgggcatggcccctcagagctcggacccctcgtgcagcgcgcaggtgcaactggccctccagactctggcccgcttcaactttaagggccatgatctgctggagttcgctcgcgagtccgtggtggtctacctggacgacgaggacgccgccacccgcaaggacgcggccctctgctgctgtcgcctgatcgcgaatagcctgtccggcatcacccagttcggctcgtcgcgttcgacccgtgccggcggtcggcgccgtcggctcgtggaggagatcgtggagaagctgctgcgcaccgctgtggccgacgccgatgtcaccgtgcgcaagagcatctttgtcgccctgttcgggaaccaatgcttcgacgactacctcgcgcaggccgactccctgacagccatcttcgcgtccctgaacgacgaggacctggatgtgcgcgagtacgcgatttccgtcgcgggtcgcctgtccgagaagaaccccgcgtacgtcctgccggccctccggcgccacctgatccagctgctgacgtacctggagctgagcgcggacaacaagtgccgcgaggagagcgccaagctgctgggctgcctggtgcgcaactgcgagcgcctgattctgccctacgtggccccagtccagaaggccctcgtggcacgcctgtcggagggtacaggcgtgaacgcgaacaacaacattgtgaccggggtgctggtgaccgtcggcgacctcgctcgcgtcggcggcctggccatgcggcagtacatcccggagctgatgcccctgatcgtcgaggcgctcatggacggcgctgccgtggctaagcgtgaggtggccgtgtccaccctgggccaggtggtccaatcgacgggctacgtggtgaccccgtacaaggagtacccgctgctgctgggcctcctgctcaagctgctcaagggcgacctggtgtggagcactcgccgggaggtcctgaaggtcctgggcatcatgggcgcgctggacccgcacgtgcacaagcgcaaccaacagagcctgagcggctcccacggggaggtcccacggggtacgggcgacagcggccagccgatcccaagcattgacgagctgccagtggagctgcgcccctcgttcgcgacatcggaggactactacagcactgtcgcgatcaatagcctgatgcgcattctgcgcgacgccagcctgctgtcgtaccacaagcgcgtcgtccggtccctgatgatcatcttcaagagcatgggcctgggctgcgtgccctacctgccgaaggtgctgccggagctgttccacactgtccggacttcggacgagaacctgaaggacttcatcacctggggcctcggcaccctcgtcagcatcgtccgccaacacatccgcaagtacctgcccgagctcctgagcctggtgtcggagctgtggagctcgttcaccctgcctggccccattcggcctagccgtggcctgccggtcctgcacctgctggagcatctgtgcctggctctcaacgacgagttccgtacctacctgcccgtgatcctgccgtgcttcattcaggtcctcggggacgccgagcgcttcaacgactacacctacgtgccggacatcctccacacgctggaggtgtttggcggcaccctggatgagcacatgcacctcctgctgcctgccctgatccggctcttcaaggtggacgctcccgtcgccatccggcgggatgcgatcaagacgctcacgcgtgtgatcccctgcgtccaggtcacaggccacattagcgccctggtgcaccacctgaagctcgtgctggacggcaagaacgacgagctgcgcaaggacgccgtggacgcgctgtgctgcctggcccacgcgctgggcgaggatttcaccattttcattgagtccatccacaagctgctgctcaagcaccgcctgcggcacaaggagttcgaggagatccacgcgcgctggcgccgtcgcgagcccctcatcgtggcgaccacggccactcagcagctgagccgccgcctgcctgtcgaggtgattcgcgaccccgtgatcgagaacgagattgatccgttcgaggagggcacagaccgcaaccaccaggtgaacgacggtcgcctgcgcaccgctggcgaggcgtcgcaacgcagcacgaaggaggactgggaggagtggatgcgccacttctcgatcgagctgctgaaggagagccctagcccggctctgcgcacctgcgctaagctggcgcagctccagcccttcgtgggccgtgagctgttcgctgcgggtttcgtctcgtgctgggcacaactgaacgagtcgagccagaagcagctcgtgcgttcgctggagatggccttttcctcccccaacatccctccggagatcctcgcgacgctgctgaacctggcggagtttatggagcacgacgagaagcctctgcccatcgacattcggctgctgggcgccctggcagagaagtgccgggtcttcgcgaaggccctgcactacaaggagatggagtttgagggcccccgctccaagcgcatggacgcgaaccccgtggcggtggtggaggccctcatccacatcaacaaccagctccaccagcacgaggcggcggtcggcattctgacgtacgcccagcaacacctggacgtgcagctgaaggagtcgtggtacgagaagctgcaacgctgggatgacgcgctgaaggcctacaccctgaaggcctcccagaccaccaacccccacctggtcctggaggctaccctcggccagatgcggtgcctcgcggccctggcccggtgggaggagctgaacaacctgtgcaaggagtactggtcgccggctgagccctccgcccgcctggagatggcgccaatggccgcgcaggcggcgtggaacatgggcgagtgggaccagatggcggagtatgtgagccgcctggacgacggcgacgagacgaagctgcgtggcctggcctcgcctgtgtcgagcggcgatggcagctcgaacgggaccttctttcgggcggtcctcctggtgcgccgcgctaagtacgacgaggcgcgggagtacgtggagcgcgctcgcaagtgcctggcaacagagctcgctgccctggtcctggagtcgtacgagcgggcgtactccaacatggtgcgcgtgcagcagctgtcggagctggaggaggtgatcgagtactacactctgcccgtggggaacacgatcgccgaggagcgtcgcgctctgatccgcaacatgtggacgcagcgcatccaggggtccaagcgtaacgtcgaggtgtggcaggccctcctggcggtgcgcgccctcgtgctgcctcccacggaggatgtcgagacttggctgaagttcgccagcctgtgccgcaagagcggtcgcatctcccaggccaagtccaccctgctgaagctgctccccttcgacccggaggtgtcccccgagaacatgcagtaccacggtccccctcaagtgatgctcggctacctgaagtaccagtggtccctgggcgaggagcgcaagcgcaaggaggctttcaccaagctccagatcctcacccgcgagctctcgtcggtgccacacagccagtccgacatcctggcgtcgatggtgtcgagcaagggcgccaacgtgcccctcctcgcccgcgtcaacctgaagctgggcacctggcagtgggcactgagctccggcctgaatgacggctccattcaggagatccgcgacgcgtttgacaagtccacctgttacgcaccaaagtgggcgaaggcttggcacacttgggccctgtttaacacagccgtgatgtcccactacatcagccgcggccagattgcgtcccagtacgtcgtgtccgccgtgacaggctacttctactcgatcgcgtgcgcggcgaacgctaagggcgtcgatgactcgctccaggacatcctgcggctgctgaccctgtggtttaaccacggtgcaaccgcggacgtgcagacggcgctgaagaccgggttctcgcacgtgaatatcaacacgtggctcgtggtgctgccccagatcatcgcgcgcattcactccaacaaccgcgctgtgcgcgagctgatccagagcctgctgattcggatcggcgagaatcacccgcaggcgctgatgtaccctctcctggtggcctgcaagagcattagcaacctgcgccgtgctgccgcccaggaggtggtggacaaggtccgccagcacagcggcgccctggtggaccaggcacagctggtgtcccacgagctcattcgggtggcgatcctgtggcacgagatgtggcatgaggccctggaggaggcttcccgcctgtacttcggcgagcacaacatcgagggtatgctgaaggtgctggagccgctgcacgacatgctggacgagggcgtgaagaaggactcgaccacaatccaggagcgcgccttcatcgaggcgtaccgccacgagctgaaggaggcgcacgagtgctgctgcaactacaagatcacgggtaaggacgcggagctgacccaggcgtgggacctgtactaccacgtcttcaagcgcatcgacaagcagctcgcgagcctgaccaccctggatctggagtccgtgtccccggagctgctgctgtgccgcgatctggagctggcggtgcccggcacctaccgcgcggacgcgccggtcgtcaccatctccagcttctcccgtcagctggtggtgatcacgagcaagcaacggccccggaagctcacgattcatggcaatgacggcgaggactacgccttcctgctgaagggccacgaggatctgcgccaggacgagcgcgtcatgcagctgttcggcctggtgaataccctcctggagaatagccgtaagacggcggagaaggacctgtccatccagcgctattccgtgatccccctgtcccccaacagcggcctgatcggctgggtgccgaactgcgacaccctgcaccacctcatccgcgagcaccgcgatgctcgcaagattattctgaaccaggagaacaagcacatgctgtccttcgcccctgactacgataacctcccgctgatcgcaaaggtggaggtgttcgagtacgcgctggagaacacggagggcaacgatctgagccgtgtgctgtggctgaagagccgctccagcgaggtctggctggagcgtcggacgaactacacccgcagcctcgcggtcatgagcatggtgggctacatcctgggtctgggcgaccgccacccgtccaacctgatgctgcaccgctactcgggcaagatcctgcacattgactttggcgactgcttcgaggcctccatgaaccgcgagaagtttcccgagaaggtccctttccgcctgacccggatgctggtgaaggcgatggaggtcagcggcatcgagggcaacttccgttccacatgcgagaacgtcatgcaggtcctgcggaccaacaaggactccgtgatggccatgatggaggctttcgtgcacgacccactgatcaactggcgcctgttcaacttcaacgaggtgccgcagctggccctcctgggtaacaacaacccgaacgcgcctgctgacgtggagccggacgaggaggacgaggaccccgcggacattgacctgccccaaccgcagcgcagcacccgcgagaaggagatcctccaggcggtgaacatgctgggcgatgctaatgaggtgctgaacgagcgcgccgtggtcgtgatggcccggatgtcccataagctgaccggccgggacttctccagcagcgcgatccccagcaacccaatcgctgaccacaataacctcctgggcggcgactcgcacgaggtggagcacggtctgagcgtgaaggtccaggtgcagaagctgatcaaccaggctacctcgcacgagaacctgtgccagaactacgtgggctggtgccctttctgg

SEQ ID NO: 5 - DNA, 7566 bp, artificial sequence
  synthesized
atgctgtccggtgtgggtcctgtccctacaaagcctgcgtttaaggcagggggcgacaccctgtcgcgccacctggaggagctgtgccgctccggggcgtgggagcgtcgccacaaggacggcgacaaggcgctcctggagtacatcgaggcagaggctcgcgacctgtcggtggaggccttcggccgtctgatgaccgacgtgtaccagcgtatcggcaacatgctgctgaagggtaatgacattacccgccgcatgggcggcgtgctggcgatcgacgagctgatcgacgtgaagctgtccggggacgacgccgccaagaccgctcgcctgagcgggctgctcagccgcgtcctggaggagagcgaggaccccgtgctgagcgagtccgcgtcgcacactctgggccatctggtgcggagcggtggcgccatgacgagcgacatcgtggagaaggagatccgtcggtccctggcctggtgcgacccgcgcaacgagcccaacgagtcccgccgtctgaccgcgctgctggtgctcaccgaggccgccgagagcgctccggccgtgttcaacgtgcacgtgaagagcttcatcgacgccgtgtggtttcccctccgcgatgccaagcagcacatccgggaggccgccgtgcgggcactgaaggcgtgcctgtgcctggtggagaagcgcgagacgcgctaccgcgtgcagtggtactacaagctgcacgagcagaccatgcgcgggatgaagcgcgaccaccgcaccggcgctctgccctcgcccgagtcgatccacggctcgctgctggcgctggcggagctgctgcaacacaccggggagttcatgctggcgcgctacaaggaggtcgtggagaacgtgttccggtacaaggactcgaaggagaagaacatccgccgtgcggtcatccacctgctgccccgcatggccgccttctcgccggagcgcttcgcgtccgagtacctggcacgcgccatcgcgttcctgctgattgtcctgaagaaccctcccgagcgtggcgctgcgttcgccgccctggcggacatggccgcggctctcgcacgcggctgcctgtcgcctatctacgtcgccatccgggaggcgctctcggcgccacccgccgcacgcgctgccgctcgccctcgtccggcgacctgctatgaggccctccagtgcgtgggtatgctggccgtggcgctgggtcccctgtggcgcccctacgcagcagctctggtggaggcgatggtcctgacgggcgtctcggaggtgctcgtgcaggccctgacgcaggtggccaacgcgctccctgagctgctggaggatatccagtaccaactgctggacctgctgtccctggtcctgagcaagcggcccttcaactccagcactacgcagcccaagtttgcggcgctctcggctgcgatcgcggctggggagctccagggcaacgcgctgaccaagctggcgctgcaaaccctgggcaccttcgacctgggcggcattcagctgctggagtttatgcgcgaccacattctggcgtacacggacgaccccgacaaggagatccgccaggccgcggtcctggcagcgtgcccgcgtgctggcgcagctcggagcagcctgcgcgtgcggtccctccggagcggctggcgccgcgccgccgccgctgtgtggcacactcgcgtggtggagcgctgcgtggggcgcctgctggtcgtggcggtcgccgacccctccgagcgggtgcggaaggaggtgctccgcgctctcgtggccaccaccgccctggacgactacctggcccaggccgactgcctgcgcgcgctgttcgtgggcatgaacgacgagagcgtggccgtgcgcggtctggcgatccggctggtggggcgcctggccgagcgcaaccccgcccacgtgaaccccgcactgcgcaagcacctgctccagctgctgcacgatatggagttcagcccggacaatcgcgctcgcgaggagtccgccttcctcctggaggtgctgattacagctgctgcgcggctcatcatgccttacgtcagccccatccagaaggccctggtgtcgaagctgcgtggcggctccggtcccggcattaccgtgctgtccactctgggcgccctggctgaggtgagcggcacgaccttccgccctttcatttcggaggtgatgccgctggtgatcgaggccatccaggacaacagcgacggccgtcgccgggtggtggccgtgaagaccctgggtttcattgtgagctcctgcggcaacgtgatgggcccgtacctggagtacccgcagctgctgtccgtgctgctccggatgctgcacgaggggcaccctgcccaacgccgtgaggtcatcaaggtgctgggcatcatcggggcgctcgacccgcatacgcacaagctgaaccaggcgtcgctgtccggcgagggcaagctggagaaggagggcgtgcggcccctgcgccacggtggcggcggtgcaggtggcgctgggggcggtgcgggcggtgggggcgtgggtggcggggtcgctggcgacagcaacgacggtggcatggggcctggcgacgatggcggcccaggtggcgacctgctgccctcgtccgggctggtgactagcagcgaggattactaccctacggtcgcgattaacgcgctgatgcgcgtgctccgtgatcccgcgctggcgagccaacacctcgcggtgatccgcgcgctggcggcgatcttccgtgcgctccagctgtcggtggtgccctacctgccgaaggtcctgcccatcctcctgggcgtgctgcgtgggggcgacgaggccctgcgggaggagatcctggcttccctgcgggcgctggtcggctacgtgcgtcagcacatgcgccggttcctgccggacctgacccagctggtgcacgagttttggccggctgccccgcgtacctgcctggcgctgatcgcggatctggggatggctctccgtgacgacatccgcgcgaagcccctgccgccgctcccactgctgccgcccagcagcccgcctcgtacacctcacaaccgtcaatacgtgccggagctcctgcccaagttcgtggcggtgttcagcgaggccgagcgcgctggcagctgggacctggtgcgccctgctctcggcgcgctggagtccctgggcagcgcggtggacgacagcctgcacctgctgctgccctcgatggtgcgcctgatcagcccagcggcttccagcacgcctgccgaggtgcgccgcgccgcgctgcgcagcctgcgccggctgattccccggatgcagctgggcggctacgccagcgcggtgctgcaccctctgatcaaggtgctggacggccatagcgacgagcaactgcggcgcgacgcactggacaccatctgcgccgtggccgtgtgcctgggcccggagtttgcgatcttcgtgcctacaatccgcaaggtccgcgtgcggcaccgtctgcaccatgagtggttcgaccgcctggcgggcaaggtgtgcgccgtgagccctccctgcatgagcgacgcggaggactgggagggggctgggggtgcggccagcggtgcaggcagcgctggtgctgccggtggctgggcagtggagatcgacctgctcgcccggatgcaggcggagggcggtggcgcgctcggtggccagcccccggtgccccctggccccgacggcggtccctccgctaagctcccggtgaacgccgctgtgctgcgccgcgcctgggagtcgagccaccgggtgacgaaggaggactgggccgagtggatgcgcaacttcgccgtcgagctgctgaaggagtcgcctagccctgccctgcgcgcgtgccacggtctggcgcaggtgcacccgtcgatggcgcgggagctcttcgctgccggcttcgtgagctgctgggccgagctggagcagggcctccaggagcagctggtgcgctcgctggaggccgccctggcgtccccgactattccgcccgagacagtcaccgccctgctgaacctggccgagttcatggagcacgatgacaagcgcctgcccctggacacccgcaccctgggggccctggcggagaagtgccacgcttttgcgaaggccctgcattacaaggagctggagttccagacgagcccccagtccgcgatcgaggccctgatccacatcaacaaccagctgcgccagccggaggcggcggtcggcgtgctcgcgtacgctcagaagcacctgcacatggagctgaaggagggctggtacgagaagctgtgccgctgggacgaggcactggacgcctacgagcgccggctcctgaaggaggccccaggctcgatggagtaccacaccgccctgctgggcaagatgcggtgcctggcctcgctggcggagtgggagaacctgagcaacctgtgccggacggagtggcgcaagagcgagccccacgtgcgccgcgagatggcgctcatcgccgctcacgcggcctggcacatgggcgcgtgggatgagatggccatgtacgtggacactgtggacaacccagaggcggtgggccccaactcccacacccctacgggcgccttcctgcgggccgtgctctgcgtgcgcgcgaaccaggtgtccggggcccaggcgcacgtcgagcgcacgcgggagctgatggtggccgacctggcggcgctcgtgggggagagctacgagcgtgcctacacggacatggtccgcgtccagcagctggccgagctggaggaggtctgcgcctataagcaggccctcgaccgtcgggccgcagacccaggcgggtccgaggcgcgtatcggcttcattcagcagctgtggcgtgaccgcctgcgcggcgtgcagcgccatgtggaggtgtggcagagcctgttcagcatccgctcgctggtggtgccgatggcgcaggacgtggacagctggctgaagtttgcttcgctgtgccgtaagagcggccggtcgcgccaggcgtaccggatgctgctccagctgctgcgctacaacccgatgaacatcacgcaggccggcaaccccggctacggtgcgggtagcggcgctcctcacgtgatgctggcctttctcaagcacctctggacccagggcaaccgtactgaggcgtacaaccgcattaaggacctggcctccctgaacggccgcgccttcctgcgtctcggcatctggcaatgggccatgaacgacctggacaaccctggtgtcatcgcggagaacctggccagcttccgcgctgcgacggagcacgcacccaactgggcgaaggcctggcaccaatgggccctgttcaatgtggcagtcagcgctcactaccgctgcgaccccatgcgggatgagaaccaggcggtgagccacgtgccccctgcggtgcagggcttcttccgcagcgtggcgctgggccaagccgcgggtgaccgcacgggtaacctccaggacatcctgcgcctgctgaccctgtggtttaacttcggcgcctacgctgaggtccgcgctgccctgaccgagggcttccagctggtgtcgattgacacgtggctgctggtcatcccgcagatcatcgcgcgcatccacacacataacaccgacgtgcgccagctgatccaccacctgctggtgaagatcggccgtcaccacccacaggctctgatgtacccactgctcgtcgccaccaagtcgcaatcgccggcacgccgccaggcagcctactccgtcctggagtgcatccgccagcacagcgcagcgctggtcgagcaggcccagctcgtgagcggcgagctcatccgcatggccatcctctggcacgagatgtggcacgagggcctggaggaggcctcccgcctgtactttggcgagtccaacgtcgagggtatgctgaacacgctgctgccactgcacgagatgctggagaaggctggccccaccaccctgaaggagatcgcctttgtccagtcctatggccgggagctgtccgaggcctatgagtggctgatgaagtacaaggcctcgcgcaaggaggctgagctgcaccaggcgtgggacctgtactaccatgtgttcaagcgcatcaacaagcagctgcgcagcctcaccacgctggagctgcaatacgtgagccctgcgctggtgcgcgcccaggacctggagctcgccgtgcccggcacgtatatcgccggtgagcccctggtgaccatcgccgcttttgcgccccagctgcacgtcatctcctccaagcaacgccctcgcaagctgaccatccacggcggtgacggcgcagagtacatgttcctgctgaagggtcacgaggatctgcgccaggacgagcgtgtgatgcagctgttcgggctggtgaacacaatgctggctcacgaccgcatcacggctgagcgcgacctgagcattgcgcgctacgcggtgatcccgctgagcccgaacagcggcctcattggctgggtgcctaattgcgacaccctgcacgctctgatccgcgagtaccgcgaggctcgcaagatcccgctgaactgggagcaccgcctgatgctcggcatggcccccgactacgaccacctgacggtgatccagaaggtcgaggtgttcgagtacgcgctggacagcacgtcgggcgaggacctgcacaaggtcctgtggctgaagtcgcgcaactccgaggtgtggctggaccgtcggacgaactatacgcgcagcgctgcggtgatgtcgatggtgggctacatcctgggcctgggcgatcgccacccgagcaacctgatgctggatcgctactccggcaagctgctgcacatcgacttcggcgactgcttcgaggccagcatgaaccgtgagaagttcccggagaaggtccctttccgcctgacgcgcatgatgatcaaggcgatggaggtgagcggtatcgagggcaacttccgtaccacgtgcgagaacgtgatgcgtgtgctgcgcagcaacaaggagagcgtgactgccatgctggaggcgttcgtgcacgaccccctgatcaactggcgcctgctgaacaccactgaggcagcgacggaggcggcgctggcccgcaccgacggcggtggcggcggtggtgggcacatggatggtccgggtggccacccaggcggtcgggatgctctgggcggtggtggtggcggggcaggcggcggtggtggcggcgatcccggggccatgcccagccctcctcgccgcgagactcgcgagaaggagctcaaggaggccttcgtgaacctgggggacgcaaacgaggtgctgaatacacgggccgtggaggtgatgaagcgcatgagcgacaagctgatgggccgcgactacgcgcccgagctgtgcgtgggtggcggttcgggcgcctcgggcatggagcccgacagcgtgccagcccaggtgggccgcctgatcaacatggcggtgaatcacgagaacctgtgccagtcgtacatcggctggtgcccattctgg

SEQ ID NO: 6 - DNA, 1428 bp, artificial sequence
  synthesized
atgctcgaggccgcagctgtgagcactgtcggtgcaatcaatcgggctcctctgtccctgaacggcagcggcagcggtgcggtgtccgcgccggcctccaccttcctgggcaagaaggtggtgactgtgagccggttcgcgcagtcgaacaagaagagcaacgggagcttcaaggtgctggcggtgaaggaggacaagcagaccgacggcgaccgctggcgtggcctggcctacgacacctccgacgaccagcaggacatcacccgcggcaaggggatggtcgatagcgtgtttcaggccccgatgggcaccggcacccaccacgccgtcctgtcctcctacgagtacgtctcccagggcctccgccagtacaacctggacaacatgatggacggcttctacatcgctccggccttcatggacaagctggtcgtgcatatcaccaagaactttctcaccctgcccaacatcaaggtgcccctcatcctgggcatctggggcgggaagggccagggcaagtcgttccaatgcgagctggtgatggccaagatgggcatcaacccgatcatgatgagcgcgggcgagctggagagcggcaacgccggcgagccggctaagctgatccgccagcgctaccgggaggctgccgacctcatcaagaagggtaagatgtgctgcctgttcattaacgacctggacgctggcgccgggcggatgggcggcaccacccagtacacagtgaacaaccagatggtcaacgcgaccctgatgaacatcgccgataaccccacgaacgtgcagctgcccggcatgtacaacaaggaggagaacgcccgcgtcccgatcatctgcaccggcaacgacttctccacgctgtacgctccgctgattcgcgacggccggatggagaagttctactgggcacctactcgcgaggaccggatcggcgtctgtaagggcatcttccgcaccgacaagattaaggacgaggacattgtcaccctggtggatcagttccctggccagtccatcgactttttcggcgccctccgcgctcgcgtgtacgacgacgaggtgcgcaagttcgtggagtccctgggcgtcgagaagatcggtaagcgcctggtcaactcccgcgagggccccccggtgttcgagcagccagagatgacgtacgagaagctcatggagtacgggaacatgctcgtgatggagcaggagaacgtgaagcgggtccagctggcagagacctatctgtcgcaggcggccctgggcgacgcgaacgccgatgcgattggccggggcacattttacggcaagggcgcccagcaggtcaacctgccagtgcccgagggctgcaccgacccggtggccgagaactttgaccctaccgcgcgctcggacgacggcacgtgcgtgtacaacttc

SEQ ID NO: 7 - DNA, 1224 bp, artificial sequence
  synthesized
atgcaagtgacaatgaagtcgtccgccgtgagcgggcagcgtgtgggtggcgcccgtgtggcgacccgcagcgtgcgccgcgcacaactgcaagtggtggcgagctcgcgcaagcagatgggccggtggcgcagcatcgacgcgggcgtggacgcgtcggatgatcagcaggacatcacgcgtgggcgtgagatggtcgatgacctgttccagggtggctttggcgccggcggcacccacaacgctgtgctgtcctcgcaggagtacctgagccagtcccgcgcgtccttcaacaacatcgaggacggcttctacatcagccctgcgttcctggacaagatgacaatccacatcgccaagaatttcatggacctgcccaagatcaaggtgccactgatcctgggcatttggggcggcaaggggcaaggcaagaccttccaatgcgcgctggcctacaagaagctggggattgccccaatcgtgatgagcgctggggagctggagtcgggcaacgccggcgagcctgccaagctgatccgcacgcgttaccgggaggcctccgacattatcaagaagggccggatgtgcagcctgttcatcaacgatctggatgcgggggccggccgcatgggcgacaccacgcagtacaccgtgaacaaccagatggtgaacgccaccctgatgaacattgcggacaacccaaccaacgtgcagctgccgggcgtgtacaagaacgaggagatcccccgcgtgccgatcgtgtgtaccggcaacgacttcagcacactgtacgcgccgctcatccgggatggccgcatggagaagtactattggaacccgacccgcgaggatcgcattggcgtgtgcatgggcatcttccaagaggataacgtccagcgtcgcgaggtggagaacctggtggacactttccccggccaatccatcgacttcttcggtgccctgcgggcacgcgtgtacgacgacatggtgcgccagtggatcacagacaccggcgtggacaagatcggccaacagctcgtgaacgcgcgccagaaggtggccatgcctaaggtgagcatggatctgaacgtgctgatcaagtacggtaagagcctggtggacgagcaggagaacgtgaagcgcgtccagctggccgacgcgtacctgagcggcgctgagctggcaggtcacgggggtagcagcctcccggaggcgtacagccgt

SEQ ID NO: 8 - protein, 392 aa, Arabidopsis thaliana
MSSDDERDEKELSLTSPEVVTKYKSAAEIVNKALQVVLAECKPKAKIVDICEKGDSFIKEQTASMYKNSKKKIERGVAFPTCISVNNTVGHFSPLASDESVLEDGDMVKIDMGCHIDGFIALVGHTHVLQEGPLSGRKADVIAAANTAADVALRLVRPGKKNTDVTEAIQKVAAAYDCKIVEGVLSHQLKQHVIDGNKVVLSVSSPETTVDEVEFEENEVYAIDIVASTGDGKPKLLDEKQTTIYKKDESVNYQLKMKASRFIISEIKQNFPRMPFTARSLEEKRARLGLVECVNHGHLQPYPVLYEKPGDFVAQIKFTVLLMPNGSDRITSHTLQELPKKTIEDPEIKGWLALGIKKKKGGGKKKKAQKAGEKGEASTEAEPMDASSNAQE

SEQ ID NO: 9 - protein, 386 aa, Solanum tuberosum
MSDDEREEKELDLTSPEVVTKYKSAAEIVNKALQLVLSECKPKVKIVDLCEKGDAFIKEQTGNMYKNVKKKIERGVAFPTCISVNNTVCHFSPLASDETIVEEGDILKIDMGCHIDGFIAVVGHTHVLHEGPVTGRAADVIAAANTAAEVALRLVRPGKKNSDVTEAIQKVAAAYDCKIVEGVLSHQMKQFVIDGNKVVLSVSNPDTRVDEAEFEENEVYSIDIVTSTGDGKPKLLDEKQTTIYKRAVDKSYNLKMKASRFIFSEINQKFPIMPFTARDLEEKRARLGLVECVNHELLQPYPVLHEKPGDLVAHIKFTVLLMPNGSDRVTSHLQELQPTKTTENEPEIKAWLALPTKTKKKGGGKKKKGKKGDKVEEASQAEPMEG

SEQ ID NO: 10 - protein, 387 aa, Chlamydomonas reinhardtii
MSDDGSIEHQEPNLSVPEVVTKYKAAADICNRALLAVVEAAKDGAKVVDLCRMGDQFINKECANIYKGKEIEKGVAFPTCVSANSIVGHFSPNSEDATALKNGDVVKIDMGCHIDGFIATQATTIVVGDAAISGKAADVIAAARTAFDAAVRLIRPGKHIADVSAPLQKVAESFGCNLVEGVMSHEMKQFVIDGSKCILNKPTPDQKVEDGEFEENEVYAVDIVVSSGEGKPRVLDEKETTVYKRALEVTYQLKMQASRAVFSLVNSAFATMPFTLRALLDEAAAQKTELKASQLKLGLVECLNHGLLHPYPVLHEKPGEVVAQIKGTVLLMPNGSSIITSAPRQTVTTEKKVEDKEILDLLATPISAKSAKKKKNKDKAAEPAAAK

SEQ ID NO: 11 - protein, 2481 aa, Arabidopsis thaliana
MSTSSQSFVAGRPASMASPSQSHRFCGPSATASGGGSFDTLNRVIADLCSRGNPKEGAPLAFRKHVEEAVRDLSGEASSRFMEQLYDRIANLIESTDVAENMGALRAIDELTEIGFGENATKVSRFAGYMRTVFELKRDPEILVLASRVLGHLARAGGAMTSDEVEFQMKTAFDWLRVDRVEYRRFAAVLILKEMAENASTVFNVHVPEFVDAIWVALRDPQLQVRERAVEALRACLRVIEKRETRWRVQWYYRMFEATQDGLGRNAPVHSIHGSLLAVGELLRNTGEFMMSRYREVAEIVLRYLEHRDRLVRLSITSLLPRIAHFLRDRFVTNYLTICMNHILTVLRIPAERASGFIALGEMAGALDGELIHYLPTIMSHLRDAIAPRKGRPLLEAVACVGNIAKAMGSTVETHVRDLLDVMFSSSLSSTLVDALDQITISIPSLLPTVQDRLLDCISLVLSKSHYSQAKPPVTIVRGSTVGMAPQSSDPSCSAQVQLALQTLARFNFKGHDLLEFARESVVVYLDDEDAATRKDAALCCCRLIANSLSGITQFGSSRSTRAGGRRRRLVEEIVEKLLRTAVADADVTVRKSIFVALFGNQCFDDYLAQADSLTAIFASLNDEDLDVREYAISVAGRLSEKNPAYVLPALRRHLIQLLTYLELSADNKCREESAKLLGCLVRNCERLILPYVAPVQKALVARLSEGTGVNANNNIVTGVLVTVGDLARVGGLAMRQYIPELMPLIVEALMDGAAVAKREVAVSTLGQVVQSTGYVVTPYKEYPLLLGLLLKLLKGDLVWSTRREVLKVLGIMGALDPHVHKRNQQSLSGSHGEVPRGTGDSGQPIPSIDELPVELRPSFATSEDYYSTVAINSLMRILRDASLLSYHKRVVRSLMIIFKSMGLGCVPYLPKVLPELFHTVRTSDENLKDFITWGLGTLVSIVRQHIRKYLPELLSLVSELWSSFTLPGPIRPSRGLPVLHLLEHLCLALNDEFRTYLPVILPCFIQVLGDAERFNDYTYVPDILHTLEVFGGTLDEHMHLLLPALIRLFKVDAPVAIRRDAIKTLTRVIPCVQVTGHISALVHHLKLVLDGKNDELRKDAVDALCCLAHALGEDFTIFIESIHKLLLKHRLRHKEFEEIHARWRRREPLIVATTATQQLSRRLPVEVIRDPVIENEIDPFEEGTDRNHQVNDGRLRTAGEASQRSTKEDWEEWMRHFSIELLKESPSPALRTCAKLAQLQPFVGRELFAAGFVSCWAQLNESSQKQLVRSLEMAFSSPNIPPEILATLLNLAEFMEHDEKPLPIDIRLLGALAEKCRVFAKALHYKEMEFEGPRSKRMDANPVAVVEALIHINNQLHQHEAAVGILTYAQQHLDVQLKESWYEKLQRWDDALKAYTLKASQTTNPHLVLEATLGQMRCLAALARWEELNNLCKEYWSPAEPSARLEMAPMAAQAAWNMGEWDQMAEYVSRLDDGDETKLRGLASPVSSGDGSSNGTFFRAVLLVRRAKYDEAREYVERARKCLATELAALVLESYERAYSNMVRVQQLSELEEVIEYYTLPVGNTIAEERRALIRNMWTQRIQGSKRNVEVWQALLAVRALVLPPTEDVETWLKFASLCRKSGRISQAKSTLLKLLPFDPEVSPENMQYHGPPQVMLGYLKYQWSLGEERKRKEAFTKLQILTRELSSVPHSQSDILASMVSSKGANVPLLARVNLKLGTWQWALSSGLNDGSIQEIRDAFDKSTCYAPKWAKAWHTWALFNTAVMSHYISRGQIASQYVVSAVTGYFYSIACAANAKGVDDSLQDILRLLTLWFNHGATADVQTALKTGFSHVNINTWLVVLPQIIARIHSNNRAVRELIQSLLIRIGENHPQALMYPLLVACKSISNLRRAAAQEVVDKVRQHSGALVDQAQLVSHELIRVAILWHEMWHEALEEASRLYFGEHNIEGMLKVLEPLHDMLDEGVKKDSTTIQERAFIEAYRHELKEAHECCCNYKITGKDAELTQAWDLYYHVFKRIDKQLASLTTLDLESVSPELLLCRDLELAVPGTYRADAPVVTISSFSRQLVVITSKQRPRKLTIHGNDGEDYAFLLKGHEDLRQDERVMQLFGLVNTLLENSRKTAEKDLSIQRYSVIPLSPNSGLIGWVPNCDTLHHLIREHRDARKIILNQENKHMLSFAPDYDNLPLIAKVEVFEYALENTEGNDLSRVLWLKSRSSEVWLERRTNYTRSLAVMSMVGYILGLGDRHPSNLMLHRYSGKILHIDFGDCFEASMNREKFPEKVPFRLTRMLVKAMEVSGIEGNFRSTCENVMQVLRTNKDSVMAMMEAFVHDPLINWRLFNFNEVPQLALLGNNNPNAPADVEPDEEDEDPADIDLPQPQRSTREKEILQAVNMLGDANEVLNERAVVVMARMSHKLTGRDFSSSAIPSNPIADHNNLLGGDSHEVEHGLSVKVQVQKLINQATSHENLCQNYVGWCPFW

SEQ ID NO: 12 - protein, 2522 aa, Chlamydomonas reinhardtii
MLSGVGPVPTKPAFKAGGDTLSRHLEELCRSGAWERRHKDGDKALLEYIEAEARDLSVEAFGRLMTDVYQRIGNMLLKGNDITRRMGGVLAIDELIDVKLSGDDAAKTARLSGLLSRVLEESEDPVLSESASHTLGHLVRSGGAMTSDIVEKEIRRSLAWCDPRNEPNESRRLTALLVLTEAAESAPAVFNVHVKSFIDAVWFPLRDAKQHIREAAVRALKACLCLVEKRETRYRVQWYYKLHEQTMRGMKRDHRTGALPSPESIHGSLLALAELLQHTGEFMLARYKEVVENVFRYKDSKEKNIRRAVIHLLPRMAAFSPERFASEYLARAIAFLLIVLKNPPERGAAFAALADMAAALARGCLSPIYVAIREALSAPPAARAAARPRPATCYEALQCVGMLAVALGPLWRPYAAALVEAMVLTGVSEVLVQALTQVANALPELLEDIQYQLLDLLSLVLSKRPFNSSTTQPKFAALSAAIAAGELQGNALTKLALQTLGTFDLGGIQLLEFMRDHILAYTDDPDKEIRQAAVLAACPRAGAARSSLRVRSLRSGWRRAAAAVWHTRVVERCVGRLLVVAVADPSERVRKEVLRALVATTALDDYLAQADCLRALFVGMNDESVAVRGLAIRLVGRLAERNPAHVNPALRKHLLQLLHDMEFSPDNRAREESAFLLEVLITAAARLIMPYVSPIQKALVSKLRGGSGPGITVLSTLGALAEVSGTTFRPFISEVMPLVIEAIQDNSDGRRRVVAVKTLGFIVSSCGNVMGPYLEYPQLLSVLLRMLHEGHPAQRREVIKVLGIIGALDPHTHKLNQASLSGEGKLEKEGVRPLRHGGGGAGGAGGGAGGGGVGGGVAGDSNDGGMGPGDDGGPGGDLLPSSGLVTSSEDYYPTVAINALMRVLRDPALASQHLAVIRALAAIFRALQLSVVPYLPKVLPILLGVLRGGDEALREEILASLRALVGYVRQHMRRFLPDLTQLVHEFWPAAPRTCLALIADLGMALRDDIRAKPLPPLPLLPPSSPPRTPHNRQYVPELLPKFVAVFSEAERAGSWDLVRPALGALESLGSAVDDSLHLLLPSMVRLISPAASSTPAEVRRAALRSLRRLIPRMQLGGYASAVLHPLIKVLDGHSDEQLRRDALDTICAVAVCLGPEFAIFVPTIRKVRVRHRLHHEWFDRLAGKVCAVSPPCMSDAEDWEGAGGAASGAGSAGAAGGWAVEIDLLARMQAEGGGALGGQPPVPPGPDGGPSAKLPVNAAVLRRAWESSHRVTKEDWAEWMRNFAVELLKESPSPALRACHGLAQVHPSMARELFAAGFVSCWAELEQGLQEQLVRSLEAALASPTIPPETVTALLNLAEFMEHDDKRLPLDTRTLGALAEKCHAFAKALHYKELEFQTSPQSAIEALIHINNQLRQPEAAVGVLAYAQKHLHMELKEGWYEKLCRWDEALDAYERRLLKEAPGSMEYHTALLGKMRCLASLAEWENLSNLCRTEWRKSEPHVRREMALIAAHAAWHMGAWDEMAMYVDTVDNPEAVGPNSHTPTGAFLRAVLCVRANQVSGAQAHVERTRELMVADLAALVGESYERAYTDMVRVQQLAELEEVCAYKQALDRRAADPGGSEARIGFIQQLWRDRLRGVQRHVEVWQSLFSIRSLVVPMAQDVDSWLKFASLCRKSGRSRQAYRMLLQLLRYNPMNITQAGNPGYGAGSGAPHVMLAFLKHLWTQGNRTEAYNRIKDLASLNGRAFLRLGIWQWAMNDLDNPGVIAENLASFRAATEHAPNWAKAWHQWALFNVAVSAHYRCDPMRDENQAVSHVPPAVQGFFRSVALGQAAGDRTGNLQDILRLLTLWFNFGAYAEVRAALTEGFQLVSIDTWLLVIPQIIARIHTHNTDVRQLIHHLLVKIGRHHPQALMYPLLVATKSQSPARRQAAYSVLECIRQHSAALVEQAQLVSGELIRMAILWHEMWHEGLEEASRLYFGESNVEGMLNTLLPLHEMLEKAGPTTLKEIAFVQSYGRELSEAYEWLMKYKASRKEAELHQAWDLYYHVFKRINKQLRSLTTLELQYVSPALVRAQDLELAVPGTYIAGEPLVTIAAFAPQLHVISSKQRPRKLTIHGGDGAEYMFLLKGHEDLRQDERVMQLFGLVNTMLAHDRITAERDLSIARYAVIPLSPNSGLIGWVPNCDTLHALIREYREARKIPLNWEHRLMLGMAPDYDHLTVIQKVEVFEYALDSTSGEDLHKVLWLKSRNSEVWLDRRTNYTRSAAVMSMVGYILGLGDRHPSNLMLDRYSGKLLHIDFGDCFEASMNREKFPEKVPFRLTRMMIKAMEVSGIEGNFRTTCENVMRVLRSNKESVTAMLEAFVHDPLINWRLLNTTEAATEAALARTDGGGGGGGHMDGPGGHPGGRDALGGGGGGAGGGGGGDPGAMPSPPRRETREKELKEAFVNLGDANEVLNTRAVEVMKRMSDKLMGRDYAPELCVGGGSGASGMEPDSVPAQVGRLINMAVNHENLCQSYIGWCPFW

SEQ ID NO: 13 - protein, 476 aa, Arabidopsis thaliana
MLEAAAVSTVGAINRAPLSLNGSGSGAVSAPASTFLGKKVVTVSRFAQSNKKSNGSFKVLAVKEDKQTDGDRWRGLAYDTSDDQQDITRGKGMVDSVFQAPMGTGTHHAVLSSYEYVSQGLRQYNLDNMMDGFYIAPAFMDKLVVHITKNFLTLPNIKVPLILGIWGGKGQGKSFQCELVMAKMGINPIMMSAGELESGNAGEPAKLIRQRYREAADLIKKGKMCCLFINDLDAGAGRMGGTTQYTVNNQMVNATLMNIADNPTNVQLPGMYNKEENARVPIICTGNDFSTLYAPLIRDGRMEKFYWAPTREDRIGVCKGIFRTDKIKDEDIVTLVDQFPGQSIDFFGALRARVYDDEVRKFVESLGVEKIGKRLVNSREGPPVFEQPEMTYEKLMEYGNMLVMEQENVKRVQLAETYLSQAALGDANADAIGRGTFYGKGAQQVNLPVPEGCTDPVAENFDPTARSDDGTCVYNF

SEQ ID NO: 14 - protein, 408 aa, Chlamydomonas reinhardtii
MQVTMKSSAVSGQRVGGARVATRSVRRAQLQVVASSRKQMGRWRSIDAGVDASDDQQDITRGREMVDDLFQGGFGAGGTHNAVLSSQEYLSQSRASFNNIEDGFYISPAFLDKMTIHIAKNFMDLPKIKVPLILGIWGGKGQGKTFQCALAYKKLGIAPIVMSAGELESGNAGEPAKLIRTRYREASDIIKKGRMCSLFINDLDAGAGRMGDTTQYTVNNQMVNATLMNIADNPTNVQLPGVYKNEEIPRVPIVCTGNDFSTLYAPLIRDGRMEKYYWNPTREDRIGVCMGIFQEDNVQRREVENLVDTFPGQSIDFFGALRARVYDDMVRQWITDTGVDKIGQQLVNARQKVAMPKVSMDLNVLIKYGKSLVDEQENVKRVQLADAYLSGAELAGHGGSSLPEAYSR

SEQ ID NO: 15 - DNA, 1173 bp, artificial sequence
  synthesized
agcagcgatgacgagcgtgacgagaaggagctgagcctgactagcccggaggtggtgaccaagtataagtcggcagcagagattgtgaacaaggcactccaggtggtgctcgccgagtgcaagccgaaggctaagattgtggacatctgcgagaagggcgacagcttcattaaggagcagacagcgtcgatgtacaagaactccaagaagaagatcgagcgcggcgtcgcgttcccgacatgtatttccgtcaacaacacggtcggccacttttcgcccctggcttcggatgagagcgtgctggaggatggcgacatggtgaagatcgacatgggctgccacatcgacggcttcatcgcgctggtggggcacacgcacgtgctgcaagagggccccctgtcgggccggaaggcggacgtgattgcagccgccaacaccgctgcggacgtggccctgcgcctcgtccgtcccggcaagaagaacacagacgtgaccgaggctattcagaaggtggcggctgcgtatgactgcaagatcgtggagggcgtcctgagccaccagctgaagcagcacgtgattgacggtaataaggtcgtgctctcggtgtcgagccccgagaccactgtggacgaggtggagttcgaggagaacgaggtgtacgctatcgacatcgtggcctcgaccggcgacggcaagcccaagctgctggacgagaagcaaacgaccatctacaagaaggacgagtcggtgaactaccagctgaagatgaaggcctcgcgcttcatcatcagcgagatcaagcagaacttcccccggatgcccttcacggcccgctccctggaggagaagcgcgctcgcctggggctggtcgagtgcgtgaaccacggccacctgcaaccctatccggtgctgtacgagaagcccggcgatttcgtggcgcagatcaagttcaccgtgctgctgatgcccaacggctccgaccggatcactagccataccctccaggagctgcccaagaagaccattgaggacccggagatcaagggctggctcgccctgggcattaagaagaagaagggcggcggcaagaagaagaaggcgcaaaaggccggcgagaagggcgaggcctccacggaggcggagccaatggacgcgagctcgaacgcccaggag

SEQ ID NO: 16 - DNA, 1155 bp, artificial sequence
  synthesized
tcggatgatgagcgtgaggagaaggagctggatctgactagccctgaggtggtgacgaagtacaagtccgccgccgagatcgtgaacaaggccctccagctggtgctgtcggagtgcaagccaaaggtgaagatcgtggacctgtgcgagaagggcgatgccttcatcaaggagcagaccgggaacatgtacaagaacgtgaagaagaagatcgagcggggcgtggccttcccgacttgtatctccgtgaacaacaccgtgtgccacttcagccctctggcgagcgacgagacgatcgtggaggagggcgacattctgaagatcgacatgggttgccacatcgacggtttcatcgcggtcgtgggtcacacccacgtgctgcacgagggcccggtcacgggccgcgccgctgacgtgatcgccgctgcgaacacggctgcggaggtggcgctgcgcctggtgcgtcccggcaagaagaactcggacgtgaccgaggccatccagaaggtcgcggctgcctacgactgcaagatcgtggagggcgtgctctcgcaccagatgaagcaattcgtgatcgacggcaacaaggtggtgctgagcgtgagcaaccccgacacccgcgtggacgaggccgagttcgaggagaacgaggtgtacagcatcgacattgtgacgagcacgggcgatggcaagcccaagctcctggacgagaagcagacaaccatctacaagcgggccgtggacaagagctacaacctgaagatgaaggcgagccgcttcattttctcggagatcaaccagaagttccccatcatgccattcaccgctcgggacctggaggagaagcgtgcccgtctgggcctggtcgagtgcgtgaaccatgagctcctgcaaccctacccggtcctgcacgagaagccgggcgacctggtggctcacattaagtttactgtgctgctgatgcccaacggcagcgaccgtgtgacatcgcacctgcaagagctgcaacccacgaagacgacggagaacgagcccgagatcaaggcgtggctggcgctccctacgaagactaagaagaagggcggtgggaagaagaagaagggcaagaagggcgacaaggtggaggaggcgtcgcaggccgagccgatggagggc

SEQ ID NO: 17 - DNA, 1158 bp, artificial sequence
  synthesized
agcgacgacggtagcattgagcaccaagagccaaatctgagcgtccctgaggtggtgacaaagtacaaggctgcggctgacatttgcaaccgcgccctgctcgccgtggtggaggctgcgaaggacggcgcaaaggtcgtggacctgtgccgcatgggcgaccagttcatcaacaaggagtgcgccaacatttacaagggcaaggagatcgagaagggcgtggcgttccccacctgtgtctcggctaactcgatcgtgggccatttcagccccaattcggaggatgctacggcgctgaagaacggcgatgtggtgaagattgacatgggctgccacattgacgggttcatcgccacccaggccaccaccatcgtggtgggcgatgctgcgatcagcggcaaggcagcagatgtgatcgcggcagcccgcacggccttcgatgccgcagtgcgcctgattcgcccaggcaagcacatcgcggacgtgagcgcgcctctccagaaggtggcggagagcttcggctgcaatctcgtcgagggcgtgatgagccacgagatgaagcagttcgtgattgatggctcgaagtgcatcctcaacaagcccacccccgatcagaaggtcgaggacggcgagttcgaggagaacgaggtgtacgccgtggacatcgtggtgtcgagcggcgagggtaagccgcgtgtgctggacgagaaggagacaacagtgtacaagcgggccctggaggtgacctaccagctgaagatgcaggcctcccgcgcggtgttctcgctggtgaatagcgccttcgcgacgatgcccttcaccctgcgcgcgctgctggacgaggcagcggcccagaagacggagctgaaggcgtcccagctgaagctcggcctggtggagtgcctgaaccacggcctgctgcacccgtacccggtgctgcacgagaagcccggcgaggtggtggctcagatcaagggcaccgtgctgctgatgccgaacggctccagcattattacctccgcgccgcgtcagaccgtgaccacggagaagaaggtggaggacaaggagatcctggacctcctggcaaccccaatctcggccaagtccgccaagaagaagaagaacaaggacaaggctgctgagcccgctgccgctaag

SEQ ID NO: 18 - DNA, 7440 bp, artificial sequence
  synthesized
agcacgtcctcgcaatcgtttgtggcaggtcgccctgcctcgatggcgagcccatcccagagccaccgcttctgtggcccgagcgccaccgcgagcggcggtgggagcttcgacacgctgaaccgcgtcattgcggatctgtgttcccgtggcaacccgaaggagggcgctcccctggccttccggaagcacgtggaggaggcggtgcgcgacctgagcggcgaggcttcgagccgcttcatggagcagctgtacgatcgtatcgcgaacctcatcgagtccacggacgtggcggagaacatgggggcgctgcgggccatcgacgagctgactgagatcggcttcggcgagaacgccacgaaggtgtcccggttcgccggctacatgcgcaccgtcttcgagctgaagcgcgacccggagatcctggtgctggcgtcccgcgtgctggggcacctggcacgggcaggcggtgcgatgacctcggacgaggtggagttccagatgaagaccgctttcgattggctgcgcgtggatcgggtcgagtaccggcggttcgcggccgtgctgatcctcaaggagatggccgagaacgcttccactgtcttcaacgtgcacgtgcctgagtttgtggacgccatctgggtggccctgcgggacccgcagctgcaagtgcgcgagcgcgcggtcgaggcgctgcgggcttgcctgcgcgtcatcgagaagcgcgagacacgctggcgtgtccagtggtattaccgcatgtttgaggccactcaggacggcctgggccgcaatgcgcccgtccacagcattcatggctccctgctggcggtgggcgagctgctgcggaacactggcgagttcatgatgtcgcgctaccgcgaggtggctgagatcgtgctccggtatctggagcaccgggatcgcctggtgcgcctgagcattacgtccctcctgccccgtattgcgcacttcctgcgcgaccgtttcgtgaccaactacctgacgatctgcatgaaccacatcctgaccgtcctgcgcatccccgccgagcgggccagcggcttcatcgctctgggcgagatggcaggcgcactggacggtgagctgattcactacctgccgaccatcatgtcccacctgcgggatgccatcgcccctcggaagggccgccccctcctggaggctgtcgcgtgcgtgggcaacatcgcgaaggcgatgggctcgaccgtggagacgcacgtgcgcgacctcctggacgtgatgttctcgtcgtccctgagcagcacgctggtggacgctctggaccagatcactatctccatcccctcgctgctgcccaccgtgcaggatcgcctcctggattgcatctccctggtcctgtcgaagtcgcactactcgcaggccaagcccccagtcaccatcgtgcgcggttcgaccgtgggcatggcccctcagagctcggacccctcgtgcagcgcgcaggtgcaactggccctccagactctggcccgcttcaactttaagggccatgatctgctggagttcgctcgcgagtccgtggtggtctacctggacgacgaggacgccgccacccgcaaggacgcggccctctgctgctgtcgcctgatcgcgaatagcctgtccggcatcacccagttcggctcgtcgcgttcgacccgtgccggcggtcggcgccgtcggctcgtggaggagatcgtggagaagctgctgcgcaccgctgtggccgacgccgatgtcaccgtgcgcaagagcatctttgtcgccctgttcgggaaccaatgcttcgacgactacctcgcgcaggccgactccctgacagccatcttcgcgtccctgaacgacgaggacctggatgtgcgcgagtacgcgatttccgtcgcgggtcgcctgtccgagaagaaccccgcgtacgtcctgccggccctccggcgccacctgatccagctgctgacgtacctggagctgagcgcggacaacaagtgccgcgaggagagcgccaagctgctgggctgcctggtgcgcaactgcgagcgcctgattctgccctacgtggccccagtccagaaggccctcgtggcacgcctgtcggagggtacaggcgtgaacgcgaacaacaacattgtgaccggggtgctggtgaccgtcggcgacctcgctcgcgtcggcggcctggccatgcggcagtacatcccggagctgatgcccctgatcgtcgaggcgctcatggacggcgctgccgtggctaagcgtgaggtggccgtgtccaccctgggccaggtggtccaatcgacgggctacgtggtgaccccgtacaaggagtacccgctgctgctgggcctcctgctcaagctgctcaagggcgacctggtgtggagcactcgccgggaggtcctgaaggtcctgggcatcatgggcgcgctggacccgcacgtgcacaagcgcaaccaacagagcctgagcggctcccacggggaggtcccacggggtacgggcgacagcggccagccgatcccaagcattgacgagctgccagtggagctgcgcccctcgttcgcgacatcggaggactactacagcactgtcgcgatcaatagcctgatgcgcattctgcgcgacgccagcctgctgtcgtaccacaagcgcgtcgtccggtccctgatgatcatcttcaagagcatgggcctgggctgcgtgccctacctgccgaaggtgctgccggagctgttccacactgtccggacttcggacgagaacctgaaggacttcatcacctggggcctcggcaccctcgtcagcatcgtccgccaacacatccgcaagtacctgcccgagctcctgagcctggtgtcggagctgtggagctcgttcaccctgcctggccccattcggcctagccgtggcctgccggtcctgcacctgctggagcatctgtgcctggctctcaacgacgagttccgtacctacctgcccgtgatcctgccgtgcttcattcaggtcctcggggacgccgagcgcttcaacgactacacctacgtgccggacatcctccacacgctggaggtgtttggcggcaccctggatgagcacatgcacctcctgctgcctgccctgatccggctcttcaaggtggacgctcccgtcgccatccggcgggatgcgatcaagacgctcacgcgtgtgatcccctgcgtccaggtcacaggccacattagcgccctggtgcaccacctgaagctcgtgctggacggcaagaacgacgagctgcgcaaggacgccgtggacgcgctgtgctgcctggcccacgcgctgggcgaggatttcaccattttcattgagtccatccacaagctgctgctcaagcaccgcctgcggcacaaggagttcgaggagatccacgcgcgctggcgccgtcgcgagcccctcatcgtggcgaccacggccactcagcagctgagccgccgcctgcctgtcgaggtgattcgcgaccccgtgatcgagaacgagattgatccgttcgaggagggcacagaccgcaaccaccaggtgaacgacggtcgcctgcgcaccgctggcgaggcgtcgcaacgcagcacgaaggaggactgggaggagtggatgcgccacttctcgatcgagctgctgaaggagagccctagcccggctctgcgcacctgcgctaagctggcgcagctccagcccttcgtgggccgtgagctgttcgctgcgggtttcgtctcgtgctgggcacaactgaacgagtcgagccagaagcagctcgtgcgttcgctggagatggccttttcctcccccaacatccctccggagatcctcgcgacgctgctgaacctggcggagtttatggagcacgacgagaagcctctgcccatcgacattcggctgctgggcgccctggcagagaagtgccgggtcttcgcgaaggccctgcactacaaggagatggagtttgagggcccccgctccaagcgcatggacgcgaaccccgtggcggtggtggaggccctcatccacatcaacaaccagctccaccagcacgaggcggcggtcggcattctgacgtacgcccagcaacacctggacgtgcagctgaaggagtcgtggtacgagaagctgcaacgctgggatgacgcgctgaaggcctacaccctgaaggcctcccagaccaccaacccccacctggtcctggaggctaccctcggccagatgcggtgcctcgcggccctggcccggtgggaggagctgaacaacctgtgcaaggagtactggtcgccggctgagccctccgcccgcctggagatggcgccaatggccgcgcaggcggcgtggaacatgggcgagtgggaccagatggcggagtatgtgagccgcctggacgacggcgacgagacgaagctgcgtggcctggcctcgcctgtgtcgagcggcgatggcagctcgaacgggaccttctttcgggcggtcctcctggtgcgccgcgctaagtacgacgaggcgcgggagtacgtggagcgcgctcgcaagtgcctggcaacagagctcgctgccctggtcctggagtcgtacgagcgggcgtactccaacatggtgcgcgtgcagcagctgtcggagctggaggaggtgatcgagtactacactctgcccgtggggaacacgatcgccgaggagcgtcgcgctctgatccgcaacatgtggacgcagcgcatccaggggtccaagcgtaacgtcgaggtgtggcaggccctcctggcggtgcgcgccctcgtgctgcctcccacggaggatgtcgagacttggctgaagttcgccagcctgtgccgcaagagcggtcgcatctcccaggccaagtccaccctgctgaagctgctccccttcgacccggaggtgtcccccgagaacatgcagtaccacggtccccctcaagtgatgctcggctacctgaagtaccagtggtccctgggcgaggagcgcaagcgcaaggaggctttcaccaagctccagatcctcacccgcgagctctcgtcggtgccacacagccagtccgacatcctggcgtcgatggtgtcgagcaagggcgccaacgtgcccctcctcgcccgcgtcaacctgaagctgggcacctggcagtgggcactgagctccggcctgaatgacggctccattcaggagatccgcgacgcgtttgacaagtccacctgttacgcaccaaagtgggcgaaggcttggcacacttgggccctgtttaacacagccgtgatgtcccactacatcagccgcggccagattgcgtcccagtacgtcgtgtccgccgtgacaggctacttctactcgatcgcgtgcgcggcgaacgctaagggcgtcgatgactcgctccaggacatcctgcggctgctgaccctgtggtttaaccacggtgcaaccgcggacgtgcagacggcgctgaagaccgggttctcgcacgtgaatatcaacacgtggctcgtggtgctgccccagatcatcgcgcgcattcactccaacaaccgcgctgtgcgcgagctgatccagagcctgctgattcggatcggcgagaatcacccgcaggcgctgatgtaccctctcctggtggcctgcaagagcattagcaacctgcgccgtgctgccgcccaggaggtggtggacaaggtccgccagcacagcggcgccctggtggaccaggcacagctggtgtcccacgagctcattcgggtggcgatcctgtggcacgagatgtggcatgaggccctggaggaggcttcccgcctgtacttcggcgagcacaacatcgagggtatgctgaaggtgctggagccgctgcacgacatgctggacgagggcgtgaagaaggactcgaccacaatccaggagcgcgccttcatcgaggcgtaccgccacgagctgaaggaggcgcacgagtgctgctgcaactacaagatcacgggtaaggacgcggagctgacccaggcgtgggacctgtactaccacgtcttcaagcgcatcgacaagcagctcgcgagcctgaccaccctggatctggagtccgtgtccccggagctgctgctgtgccgcgatctggagctggcggtgcccggcacctaccgcgcggacgcgccggtcgtcaccatctccagcttctcccgtcagctggtggtgatcacgagcaagcaacggccccggaagctcacgattcatggcaatgacggcgaggactacgccttcctgctgaagggccacgaggatctgcgccaggacgagcgcgtcatgcagctgttcggcctggtgaataccctcctggagaatagccgtaagacggcggagaaggacctgtccatccagcgctattccgtgatccccctgtcccccaacagcggcctgatcggctgggtgccgaactgcgacaccctgcaccacctcatccgcgagcaccgcgatgctcgcaagattattctgaaccaggagaacaagcacatgctgtccttcgcccctgactacgataacctcccgctgatcgcaaaggtggaggtgttcgagtacgcgctggagaacacggagggcaacgatctgagccgtgtgctgtggctgaagagccgctccagcgaggtctggctggagcgtcggacgaactacacccgcagcctcgcggtcatgagcatggtgggctacatcctgggtctgggcgaccgccacccgtccaacctgatgctgcaccgctactcgggcaagatcctgcacattgactttggcgactgcttcgaggcctccatgaaccgcgagaagtttcccgagaaggtccctttccgcctgacccggatgctggtgaaggcgatggaggtcagcggcatcgagggcaacttccgttccacatgcgagaacgtcatgcaggtcctgcggaccaacaaggactccgtgatggccatgatggaggctttcgtgcacgacccactgatcaactggcgcctgttcaacttcaacgaggtgccgcagctggccctcctgggtaacaacaacccgaacgcgcctgctgacgtggagccggacgaggaggacgaggaccccgcggacattgacctgccccaaccgcagcgcagcacccgcgagaaggagatcctccaggcggtgaacatgctgggcgatgctaatgaggtgctgaacgagcgcgccgtggtcgtgatggcccggatgtcccataagctgaccggccgggacttctccagcagcgcgatccccagcaacccaatcgctgaccacaataacctcctgggcggcgactcgcacgaggtggagcacggtctgagcgtgaaggtccaggtgcagaagctgatcaaccaggctacctcgcacgagaacctgtgccagaactacgtgggctggtgccctttctgg

SEQ ID NO: 19 - DNA, 7563 bp, artificial sequence
  synthesized
ctgtccggtgtgggtcctgtccctacaaagcctgcgtttaaggcagggggcgacaccctgtcgcgccacctggaggagctgtgccgctccggggcgtgggagcgtcgccacaaggacggcgacaaggcgctcctggagtacatcgaggcagaggctcgcgacctgtcggtggaggccttcggccgtctgatgaccgacgtgtaccagcgtatcggcaacatgctgctgaagggtaatgacattacccgccgcatgggcggcgtgctggcgatcgacgagctgatcgacgtgaagctgtccggggacgacgccgccaagaccgctcgcctgagcgggctgctcagccgcgtcctggaggagagcgaggaccccgtgctgagcgagtccgcgtcgcacactctgggccatctggtgcggagcggtggcgccatgacgagcgacatcgtggagaaggagatccgtcggtccctggcctggtgcgacccgcgcaacgagcccaacgagtcccgccgtctgaccgcgctgctggtgctcaccgaggccgccgagagcgctccggccgtgttcaacgtgcacgtgaagagcttcatcgacgccgtgtggtttcccctccgcgatgccaagcagcacatccgggaggccgccgtgcgggcactgaaggcgtgcctgtgcctggtggagaagcgcgagacgcgctaccgcgtgcagtggtactacaagctgcacgagcagaccatgcgcgggatgaagcgcgaccaccgcaccggcgctctgccctcgcccgagtcgatccacggctcgctgctggcgctggcggagctgctgcaacacaccggggagttcatgctggcgcgctacaaggaggtcgtggagaacgtgttccggtacaaggactcgaaggagaagaacatccgccgtgcggtcatccacctgctgccccgcatggccgccttctcgccggagcgcttcgcgtccgagtacctggcacgcgccatcgcgttcctgctgattgtcctgaagaaccctcccgagcgtggcgctgcgttcgccgccctggcggacatggccgcggctctcgcacgcggctgcctgtcgcctatctacgtcgccatccgggaggcgctctcggcgccacccgccgcacgcgctgccgctcgccctcgtccggcgacctgctatgaggccctccagtgcgtgggtatgctggccgtggcgctgggtcccctgtggcgcccctacgcagcagctctggtggaggcgatggtcctgacgggcgtctcggaggtgctcgtgcaggccctgacgcaggtggccaacgcgctccctgagctgctggaggatatccagtaccaactgctggacctgctgtccctggtcctgagcaagcggcccttcaactccagcactacgcagcccaagtttgcggcgctctcggctgcgatcgcggctggggagctccagggcaacgcgctgaccaagctggcgctgcaaaccctgggcaccttcgacctgggcggcattcagctgctggagtttatgcgcgaccacattctggcgtacacggacgaccccgacaaggagatccgccaggccgcggtcctggcagcgtgcccgcgtgctggcgcagctcggagcagcctgcgcgtgcggtccctccggagcggctggcgccgcgccgccgccgctgtgtggcacactcgcgtggtggagcgctgcgtggggcgcctgctggtcgtggcggtcgccgacccctccgagcgggtgcggaaggaggtgctccgcgctctcgtggccaccaccgccctggacgactacctggcccaggccgactgcctgcgcgcgctgttcgtgggcatgaacgacgagagcgtggccgtgcgcggtctggcgatccggctggtggggcgcctggccgagcgcaaccccgcccacgtgaaccccgcactgcgcaagcacctgctccagctgctgcacgatatggagttcagcccggacaatcgcgctcgcgaggagtccgccttcctcctggaggtgctgattacagctgctgcgcggctcatcatgccttacgtcagccccatccagaaggccctggtgtcgaagctgcgtggcggctccggtcccggcattaccgtgctgtccactctgggcgccctggctgaggtgagcggcacgaccttccgccctttcatttcggaggtgatgccgctggtgatcgaggccatccaggacaacagcgacggccgtcgccgggtggtggccgtgaagaccctgggtttcattgtgagctcctgcggcaacgtgatgggcccgtacctggagtacccgcagctgctgtccgtgctgctccggatgctgcacgaggggcaccctgcccaacgccgtgaggtcatcaaggtgctgggcatcatcggggcgctcgacccgcatacgcacaagctgaaccaggcgtcgctgtccggcgagggcaagctggagaaggagggcgtgcggcccctgcgccacggtggcggcggtgcaggtggcgctgggggcggtgcgggcggtgggggcgtgggtggcggggtcgctggcgacagcaacgacggtggcatggggcctggcgacgatggcggcccaggtggcgacctgctgccctcgtccgggctggtgactagcagcgaggattactaccctacggtcgcgattaacgcgctgatgcgcgtgctccgtgatcccgcgctggcgagccaacacctcgcggtgatccgcgcgctggcggcgatcttccgtgcgctccagctgtcggtggtgccctacctgccgaaggtcctgcccatcctcctgggcgtgctgcgtgggggcgacgaggccctgcgggaggagatcctggcttccctgcgggcgctggtcggctacgtgcgtcagcacatgcgccggttcctgccggacctgacccagctggtgcacgagttttggccggctgccccgcgtacctgcctggcgctgatcgcggatctggggatggctctccgtgacgacatccgcgcgaagcccctgccgccgctcccactgctgccgcccagcagcccgcctcgtacacctcacaaccgtcaatacgtgccggagctcctgcccaagttcgtggcggtgttcagcgaggccgagcgcgctggcagctgggacctggtgcgccctgctctcggcgcgctggagtccctgggcagcgcggtggacgacagcctgcacctgctgctgccctcgatggtgcgcctgatcagcccagcggcttccagcacgcctgccgaggtgcgccgcgccgcgctgcgcagcctgcgccggctgattccccggatgcagctgggcggctacgccagcgcggtgctgcaccctctgatcaaggtgctggacggccatagcgacgagcaactgcggcgcgacgcactggacaccatctgcgccgtggccgtgtgcctgggcccggagtttgcgatcttcgtgcctacaatccgcaaggtccgcgtgcggcaccgtctgcaccatgagtggttcgaccgcctggcgggcaaggtgtgcgccgtgagccctccctgcatgagcgacgcggaggactgggagggggctgggggtgcggccagcggtgcaggcagcgctggtgctgccggtggctgggcagtggagatcgacctgctcgcccggatgcaggcggagggcggtggcgcgctcggtggccagcccccggtgccccctggccccgacggcggtccctccgctaagctcccggtgaacgccgctgtgctgcgccgcgcctgggagtcgagccaccgggtgacgaaggaggactgggccgagtggatgcgcaacttcgccgtcgagctgctgaaggagtcgcctagccctgccctgcgcgcgtgccacggtctggcgcaggtgcacccgtcgatggcgcgggagctcttcgctgccggcttcgtgagctgctgggccgagctggagcagggcctccaggagcagctggtgcgctcgctggaggccgccctggcgtccccgactattccgcccgagacagtcaccgccctgctgaacctggccgagttcatggagcacgatgacaagcgcctgcccctggacacccgcaccctgggggccctggcggagaagtgccacgcttttgcgaaggccctgcattacaaggagctggagttccagacgagcccccagtccgcgatcgaggccctgatccacatcaacaaccagctgcgccagccggaggcggcggtcggcgtgctcgcgtacgctcagaagcacctgcacatggagctgaaggagggctggtacgagaagctgtgccgctgggacgaggcactggacgcctacgagcgccggctcctgaaggaggccccaggctcgatggagtaccacaccgccctgctgggcaagatgcggtgcctggcctcgctggcggagtgggagaacctgagcaacctgtgccggacggagtggcgcaagagcgagccccacgtgcgccgcgagatggcgctcatcgccgctcacgcggcctggcacatgggcgcgtgggatgagatggccatgtacgtggacactgtggacaacccagaggcggtgggccccaactcccacacccctacgggcgccttcctgcgggccgtgctctgcgtgcgcgcgaaccaggtgtccggggcccaggcgcacgtcgagcgcacgcgggagctgatggtggccgacctggcggcgctcgtgggggagagctacgagcgtgcctacacggacatggtccgcgtccagcagctggccgagctggaggaggtctgcgcctataagcaggccctcgaccgtcgggccgcagacccaggcgggtccgaggcgcgtatcggcttcattcagcagctgtggcgtgaccgcctgcgcggcgtgcagcgccatgtggaggtgtggcagagcctgttcagcatccgctcgctggtggtgccgatggcgcaggacgtggacagctggctgaagtttgcttcgctgtgccgtaagagcggccggtcgcgccaggcgtaccggatgctgctccagctgctgcgctacaacccgatgaacatcacgcaggccggcaaccccggctacggtgcgggtagcggcgctcctcacgtgatgctggcctttctcaagcacctctggacccagggcaaccgtactgaggcgtacaaccgcattaaggacctggcctccctgaacggccgcgccttcctgcgtctcggcatctggcaatgggccatgaacgacctggacaaccctggtgtcatcgcggagaacctggccagcttccgcgctgcgacggagcacgcacccaactgggcgaaggcctggcaccaatgggccctgttcaatgtggcagtcagcgctcactaccgctgcgaccccatgcgggatgagaaccaggcggtgagccacgtgccccctgcggtgcagggcttcttccgcagcgtggcgctgggccaagccgcgggtgaccgcacgggtaacctccaggacatcctgcgcctgctgaccctgtggtttaacttcggcgcctacgctgaggtccgcgctgccctgaccgagggcttccagctggtgtcgattgacacgtggctgctggtcatcccgcagatcatcgcgcgcatccacacacataacaccgacgtgcgccagctgatccaccacctgctggtgaagatcggccgtcaccacccacaggctctgatgtacccactgctcgtcgccaccaagtcgcaatcgccggcacgccgccaggcagcctactccgtcctggagtgcatccgccagcacagcgcagcgctggtcgagcaggcccagctcgtgagcggcgagctcatccgcatggccatcctctggcacgagatgtggcacgagggcctggaggaggcctcccgcctgtactttggcgagtccaacgtcgagggtatgctgaacacgctgctgccactgcacgagatgctggagaaggctggccccaccaccctgaaggagatcgcctttgtccagtcctatggccgggagctgtccgaggcctatgagtggctgatgaagtacaaggcctcgcgcaaggaggctgagctgcaccaggcgtgggacctgtactaccatgtgttcaagcgcatcaacaagcagctgcgcagcctcaccacgctggagctgcaatacgtgagccctgcgctggtgcgcgcccaggacctggagctcgccgtgcccggcacgtatatcgccggtgagcccctggtgaccatcgccgcttttgcgccccagctgcacgtcatctcctccaagcaacgccctcgcaagctgaccatccacggcggtgacggcgcagagtacatgttcctgctgaagggtcacgaggatctgcgccaggacgagcgtgtgatgcagctgttcgggctggtgaacacaatgctggctcacgaccgcatcacggctgagcgcgacctgagcattgcgcgctacgcggtgatcccgctgagcccgaacagcggcctcattggctgggtgcctaattgcgacaccctgcacgctctgatccgcgagtaccgcgaggctcgcaagatcccgctgaactgggagcaccgcctgatgctcggcatggcccccgactacgaccacctgacggtgatccagaaggtcgaggtgttcgagtacgcgctggacagcacgtcgggcgaggacctgcacaaggtcctgtggctgaagtcgcgcaactccgaggtgtggctggaccgtcggacgaactatacgcgcagcgctgcggtgatgtcgatggtgggctacatcctgggcctgggcgatcgccacccgagcaacctgatgctggatcgctactccggcaagctgctgcacatcgacttcggcgactgcttcgaggccagcatgaaccgtgagaagttcccggagaaggtccctttccgcctgacgcgcatgatgatcaaggcgatggaggtgagcggtatcgagggcaacttccgtaccacgtgcgagaacgtgatgcgtgtgctgcgcagcaacaaggagagcgtgactgccatgctggaggcgttcgtgcacgaccccctgatcaactggcgcctgctgaacaccactgaggcagcgacggaggcggcgctggcccgcaccgacggcggtggcggcggtggtgggcacatggatggtccgggtggccacccaggcggtcgggatgctctgggcggtggtggtggcggggcaggcggcggtggtggcggcgatcccggggccatgcccagccctcctcgccgcgagactcgcgagaaggagctcaaggaggccttcgtgaacctgggggacgcaaacgaggtgctgaatacacgggccgtggaggtgatgaagcgcatgagcgacaagctgatgggccgcgactacgcgcccgagctgtgcgtgggtggcggttcgggcgcctcgggcatggagcccgacagcgtgccagcccaggtgggccgcctgatcaacatggcggtgaatcacgagaacctgtgccagtcgtacatcggctggtgcccattctgg

SEQ ID NO: 20 - DNA, 1419 bp, artificial sequence
  synthesized
gccgcagctgtgagcactgtcggtgcaatcaatcgggctcctctgtccctgaacggcagcggcagcggtgcggtgtccgcgccggcctccaccttcctgggcaagaaggtggtgactgtgagccggttcgcgcagtcgaacaagaagagcaacgggagcttcaaggtgctggcggtgaaggaggacaagcagaccgacggcgaccgctggcgtggcctggcctacgacacctccgacgaccagcaggacatcacccgcggcaaggggatggtcgatagcgtgtttcaggccccgatgggcaccggcacccaccacgccgtcctgtcctcctacgagtacgtctcccagggcctccgccagtacaacctggacaacatgatggacggcttctacatcgctccggccttcatggacaagctggtcgtgcatatcaccaagaactttctcaccctgcccaacatcaaggtgcccctcatcctgggcatctggggcgggaagggccagggcaagtcgttccaatgcgagctggtgatggccaagatgggcatcaacccgatcatgatgagcgcgggcgagctggagagcggcaacgccggcgagccggctaagctgatccgccagcgctaccgggaggctgccgacctcatcaagaagggtaagatgtgctgcctgttcattaacgacctggacgctggcgccgggcggatgggcggcaccacccagtacacagtgaacaaccagatggtcaacgcgaccctgatgaacatcgccgataaccccacgaacgtgcagctgcccggcatgtacaacaaggaggagaacgcccgcgtcccgatcatctgcaccggcaacgacttctccacgctgtacgctccgctgattcgcgacggccggatggagaagttctactgggcacctactcgcgaggaccggatcggcgtctgtaagggcatcttccgcaccgacaagattaaggacgaggacattgtcaccctggtggatcagttccctggccagtccatcgactttttcggcgccctccgcgctcgcgtgtacgacgacgaggtgcgcaagttcgtggagtccctgggcgtcgagaagatcggtaagcgcctggtcaactcccgcgagggccccccggtgttcgagcagccagagatgacgtacgagaagctcatggagtacgggaacatgctcgtgatggagcaggagaacgtgaagcgggtccagctggcagagacctatctgtcgcaggcggccctgggcgacgcgaacgccgatgcgattggccggggcacattttacggcaagggcgcccagcaggtcaacctgccagtgcccgagggctgcaccgacccggtggccgagaactttgaccctaccgcgcgctcggacgacggcacgtgcgtgtacaacttc

SEQ ID NO: 21 - DNA, 1221 bp, artificial sequence
  synthesized
caagtgacaatgaagtcgtccgccgtgagcgggcagcgtgtgggtggcgcccgtgtggcgacccgcagcgtgcgccgcgcacaactgcaagtggtggcgagctcgcgcaagcagatgggccggtggcgcagcatcgacgcgggcgtggacgcgtcggatgatcagcaggacatcacgcgtgggcgtgagatggtcgatgacctgttccagggtggctttggcgccggcggcacccacaacgctgtgctgtcctcgcaggagtacctgagccagtcccgcgcgtccttcaacaacatcgaggacggcttctacatcagccctgcgttcctggacaagatgacaatccacatcgccaagaatttcatggacctgcccaagatcaaggtgccactgatcctgggcatttggggcggcaaggggcaaggcaagaccttccaatgcgcgctggcctacaagaagctggggattgccccaatcgtgatgagcgctggggagctggagtcgggcaacgccggcgagcctgccaagctgatccgcacgcgttaccgggaggcctccgacattatcaagaagggccggatgtgcagcctgttcatcaacgatctggatgcgggggccggccgcatgggcgacaccacgcagtacaccgtgaacaaccagatggtgaacgccaccctgatgaacattgcggacaacccaaccaacgtgcagctgccgggcgtgtacaagaacgaggagatcccccgcgtgccgatcgtgtgtaccggcaacgacttcagcacactgtacgcgccgctcatccgggatggccgcatggagaagtactattggaacccgacccgcgaggatcgcattggcgtgtgcatgggcatcttccaagaggataacgtccagcgtcgcgaggtggagaacctggtggacactttccccggccaatccatcgacttcttcggtgccctgcgggcacgcgtgtacgacgacatggtgcgccagtggatcacagacaccggcgtggacaagatcggccaacagctcgtgaacgcgcgccagaaggtggccatgcctaaggtgagcatggatctgaacgtgctgatcaagtacggtaagagcctggtggacgagcaggagaacgtgaagcgcgtccagctggccgacgcgtacctgagcggcgctgagctggcaggtcacgggggtagcagcctcccggaggcgtacagccgt

SEQ ID NO: 22 - DNA, 1176 bp, Arabidopsis thaliana
atgagttcggacgatgagagagacgagaaggagctgagtcttacctctcctgaagtcgtcaccaagtacaagagcgccgctgagatcgttaacaaggcgttacaggttgttttagctgaatgcaaaccaaaagctaagattgttgatatctgtgagaaaggagactcttttattaaagagcaaacagcaagcatgtacaagaattctaagaagaagattgagagaggtgttgcgttccctacatgcatttctgtgaacaacactgttggtcatttttcaccgcttgctagtgatgagtctgtgttggaagatggtgacatggttaaaatcgatatgggatgtcatattgatgggttcattgcccttgttggtcacacacatgttcttcaagaagggcctcttagtgggcgtaaggctgatgttatcgctgccgcaaatactgcagctgatgttgctctaaggctcgtacgtcctggaaaaaagaacactgatgtaactgaagctattcagaaggtagctgcagcttatgactgcaaaattgttgaaggtgttctttcccaccagctgaaacagcatgtgatagatggaaacaaggttgtgcttagtgtatccagccctgaaacaactgttgacgaagtggaatttgaagagaatgaagtctatgcaatagatattgtggcaagtactggtgatggcaagccaaagctattagacgagaagcaaacaactatttacaagaaagatgaaagtgttaactatcagttgaagatgaaggcctccagattcataatcagcgaaattaaacagaacttcccccgtatgccattcactgcaaggtcactggaggagaaaagggcacggcttggacttgtggagtgtgtgaaccatggtcatttgcaaccatatcctgttctttacgagaagcctggggattttgttgctcagattaagttcacagttttgctgatgccaaatggatcagataggatcacttcacatacacttcaggaacttcctaaaaagaccatcgaagaccctgagatcaaagggtggttagctttgggcatcaagaagaagaaaggtggtggaaagaagaagaaagcccaaaaggcgggagagaaaggagaggcttcaacagaggctgagccaatggacgcaagtagtaatgctcaagaa

SEQ ID NO: 23 - DNA, 1161 bp, Solanum tuberosum
atgtcggacgacgagagagaagagaaagaattggatctcacaagtcctgaggtcgtcaccaagtacaagagcgccgctgaaattgttaacaaggcgctgcagttggtgttgtccgaatgcaagccaaaagtaaagatagttgatctttgtgagaaaggggatgcctttatcaaagagcaaactggaaatatgtacaagaatgtgaagaagaagattgagagaggtgttgcatttccaacatgtatttcagttaataacaccgtgtgccatttctctccattggctagtgatgagacaatagtggaagaaggtgatatattgaagattgatatgggatgtcacattgatggatttattgcagtagttggacatacacatgttcttcacgaaggaccagttactggtagagctgctgatgtcattgcagctgctaatacagctgctgaagttgctttgagacttgtaagaccaggaaagaagaactcggatgtaacagaagctattcagaaggttgctgctgcctatgactgcaagattgtcgagggtgtattgagccatcaaatgaagcagtttgttattgatggaaacaaagttgtattgagcgtgtccaatcctgacacaagagtagatgaagcagaatttgaagagaatgaggtctactccattgatatcgtgacgagcactggtgatggaaagcccaagttgttggatgagaaacaaacaaccatctacaagagagctgtggacaaaagctataacctgaagatgaaagcctcaaggttcatcttcagtgaaatcaatcagaagttccctatcatgccatttaccgcaagggatttggaggagaagagggctcgtttgggccttgttgaatgtgttaaccatgagcttttgcagccatatcctgttctacatgagaaacctggtgatttggttgctcacattaagttcacagtgctgttaatgcccaatggatcggatagggtaacatctcatgygctccaggagcttcagcctacaaagacaacagagaatgaacctgaaatcaaggcttggctagcccttcccaccaagactaagaagaaaggyggtgggaagaaaaagaaaggaaagaaaggtgacaaggtagaagaggcatctcargctgagcctatggaagga

SEQ ID NO: 24 - DNA, 1161 bp, Chlamydomonas reinhardtii
atgtcggacgacgggtctattgagcaccaggagcccaacctcagcgtgcctgaggtggtcaccaagtacaaggcggctgcggacatctgcaaccgcgctctgctggccgtggttgaggctgccaaggatggcgctaaggtggtggacctgtgccgcatgggcgaccagttcatcaacaaggagtgcgctaacatctacaagggcaaggagatcgagaagggcgtggccttcccgacctgcgtctctgccaacagcattgttggccacttctcgcccaactcggaggatgccaccgctctgaagaacggcgatgttgtcaagattgacatgggctgccacattgacggcttcatcgccacgcaagccaccaccattgtggtcggtgacgccgccatctcgggcaaggccgcggacgtgatcgccgccgcgcgcaccgccttcgacgccgccgtgcgcttgatacgccccggcaagcacatcgccgacgtgtccgccccgctccaaaaggtggctgagtcgtttggctgcaacctggtggagggcgtgatgagccacgagatgaagcagtttgtgattgacggcagcaagtgcatcctcaacaagcccacgcccgaccagaaggtggaggacggcgagttcgaggagaacgaggtgtacgccgtggacattgtggtcagcagcggcgagggcaagccccgcgttctggacgagaaggagacgaccgtgtacaagcgcgcactggaggtgacctaccagctcaagatgcaggccagccgcgccgtgttcagcctggtcaacagcgccttcgccaccatgcccttcacgctgcgcgcactgctggacgaggcggccgcgcagaagacggagctcaaggccagccagctgaagctgggcctggtggagtgcctaaaccacggcctgctgcacccctaccccgtgctgcacgagaagcccggggaggtggtggcgcagatcaagggcacggtgctgctcatgcccaacggttcctccatcatcacctcggcgccccgccagaccgtgaccacggagaagaaggttgaggacaaggagatcctggacctgctggccacgcccatcagcgccaagagcgccaagaagaagaagaacaaggacaaggccgccgagcccgccgccgccaag

SEQ ID NO: 25 - DNA, 7443 bp, Arabidopsis thaliana
atgtctacctcgtcgcaatcttttgtggctggacggcctgcatccatggcttccccttcgcaatcgcaccgcttttgtggtccctcagccaccgcttctggtggcggaagctttgacactttgaatcgtgtcatcgctgacctttgcagccgtggtaatcctaaggagggagctcctttagcgtttaggaaacacgtagaggaagcagttcgtgatcttagtggtgaagcttcctctaggttcatggagcaattatatgacaggattgctaatttaattgagagcactgatgtggcggaaaacatgggtgcactcagagccattgatgagttgacggagattggatttggtgagaatgctactaaggtttctagatttgcgggttacatgaggactgtgttcgagttgaagcgtgatcctgaaatcttggtgcttgctagtagagttttggggcaccttgctcgggcaggtggagcaatgacttctgatgaagtggagtttcagatgaaaacagcttttgattggcttcgcgtagacagggtggaatatcgtcgtttcgccgccgttttaatattaaaggagatggccgaaaatgcttctactgtctttaacgttcatgtccctgaatttgtggatgctatctgggttgcacttagggacccccagttgcaagtgcgagaacgagctgttgaagctttgcgtgcatgccttcgtgttattgagaaaagggagactcgatggcgagtgcagtggtactatcgaatgtttgaagctacacaggatgggttgggcagaaatgctccggttcacagtattcatggttctttacttgccgtgggggagctgttgaggaatacaggtgagttcatgatgtctaggtatagagaagttgccgaaattgtcctcagataccttgaacatcgtgatcgccttgttcgccttagcatcacctcgttactgcctcgcattgctcactttctccgtgaccggtttgtgacaaactatttaacgatatgcatgaatcatattcttactgtgttaagaataccggctgaaagagccagtgggttcatcgcccttggggaaatggctggtgctttggatggtgagcttatccattatttgccgacaattatgtctcatctgcgggatgcgattgctccacgtaaaggcagacctttgcttgaagctgtggcttgtgttggtaacatcgcaaaggcaatgggatccacagtggaaactcatgttcgagatcttttagatgttatgttttcatctagtctctcttccacacttgttgacgctcttgaccagataaccatcagcattccttctttgctgccaacagtacaagatcggcttctagattgcatttcgttggttctttcaaaatcccattattctcaagcaaagcctcctgttaccattgtccgaggtagtacagtgggcatggcaccacagtcttctgaccctagttgttcagctcaagttcaactagccctgcagactcttgctcgtttcaatttcaagggacatgatcttcttgaatttgctcgggagtcagttgttgtttatttggatgatgaggatgcagccacaagaaaagatgctgctttgtgttgttgcagactaattgcaaattctctttctggcatcacacaatttggctcgagcaggtcaacacgagcaggggggagacgcaggcgccttgtggaagagattgtggaaaagcttctcaggacagccgttgcagatgctgatgtaactgttcgcaaatctatattcgttgctttatttggcaaccaatgtttcgatgattatctagcacaggctgatagtttgactgccatttttgcttccttaaatgatgaggaccttgatgttcgagaatatgccatctcagttgctggaaggttatcggaaaaaaatccagcatacgtacttccagcacttcgtcgccatcttatacagttgttgacctatcttgagctgagtgcagataacaagtgcagggaagagagtgcaaagctccttggttgtttagttcgaaattgtgaacggctcattcttccatacgtagcccctgtccaaaaggcacttgttgcgagacttagtgaaggaactggagtgaatgctaacaataatattgtcactggagttctcgtaactgttggggatcttgcaagagtgggtggcttggcaatgagacaatatattccggagctgatgcctttaattgttgaagctttaatggatggagctgctgtagcaaaacgtgaggtggctgtttctactcttggtcaagttgttcaaagtacagggtatgttgtgactccatacaaggaatacccattgttgcttgggttactcttgaaattgctgaagggtgacttagtgtggtctaccagacgagaagtgctcaaggttcttggaattatgggcgctttggatcctcatgtgcataaacgtaaccaacaaagtttatcaggatcacatggtgaagttcctcgcggcactggtgattctggtcaacctattccatcaattgatgagttacctgtcgaactccggccgtcatttgctacatctgaggattattactcaacggttgctatcaactcgcttatgcgaattcttagagatgcatcacttcttagttaccacaaaagggttgttagatctctgatgatcattttcaagtcaatgggattgggatgcgtgccttacttgccgaaggttttacctgagctttttcacactgttcgaacatctgatgagaacctgaaggacttcattacgtggggtcttgggactcttgtttccattgttcgccagcacatacgcaagtatctgccagagctgctttcattagtctctgaactatggtcatccttcaccttgcccggtcccatacgcccatcacgtggtcttccggttctgcatctactggaacatctttgcttggcacttaatgatgaattcagaacttatcttccagtcatccttccatgtttcatccaagtattaggtgacgccgagcggtttaatgattacacctatgttcctgatattctccacacactcgaagtgtttggcggaactcttgatgagcacatgcatttactccttccggcacttattcgattgtttaaagtagatgctcctgtagctataagacgcgatgccatcaaaactttgacaagagtaatcccgtgtgttcaggttactggtcatatctccgctctcgtgcatcacttgaagctagtattagatgggaagaatgatgagttgcggaaagatgctgtcgatgcactatgctgtttggctcatgcacttggagaggacttcaccatattcattgaatcaattcacaagcttttattgaagcatcgattgcggcataaagaatttgaggaaattcatgctcgctggcggagacgtgaaccattgattgtagctacaactgcaacccaacaattaagtaggcgactgccagttgaggttatcagggatcctgtaattgagaatgagatcgatcctttcgaagaaggaactgacagaaaccatcaggttaatgatggtagactacggacagctggagaagcttctcaacgcagcaccaaagaagattgggaggaatggatgagacattttagtattgaattacttaaggagtctccctctccagcattaagaacttgtgcaaaacttgctcagttgcagccatttgtcgggagagagttgtttgctgctggctttgtcagttgctgggcacagctaaacgagtctagccaaaagcagttagttaggagcttggaaatggccttttcatctccaaatatccctccagaaattttagctacactactcaatttggcagagtttatggaacatgatgagaagcctcttcccattgatattcgtcttctgggggctcttgctgaaaagtgccgtgtttttgccaaagctctgcattataaagagatggaatttgaaggtccacgatccaagaggatggatgccaacccagttgctgttgtcgaggctcttatacacataaataatcagttacaccagcatgaggctgctgtcggtatactaacctatgctcaacaacatcttgatgtgcaattaaaagaatcatggtatgagaagctgcagcgctgggacgatgcactcaaggcgtacactttgaaagcatctcaaacaacaaatcctcatcttgtattagaagccacattaggacaaatgagatgtcttgctgcacttgcacgatgggaagagctcaacaatctctgcaaagagtactggagtcctgctgagccatctgcgcgtctggaaatggcaccaatggctgcacaagctgcatggaacatgggagagtgggatcaaatggccgaatatgtgtctcggctagatgatggtgatgaaacaaagcttcggggtttagcaagcccggtttctagtggcgatgggagcagtaatggcacattcttcagggctgttctgttagttcgaagggcaaagtacgacgaggcacgcgaatatgtggaaagagctagaaaatgtcttgccacagaacttgcagcgctggttttggagagctatgagcgtgcgtacagcaatatggttcgtgttcagcagctgtcagaactagaggaggtaattgaatattatacgctgcctgtgggaaatactattgccgaagaacggagagctctaattcgtaatatgtggactcagcggattcagggatctaagcgtaatgtggaggtgtggcaagcacttttggctgtccgggcacttgtgctacctcctacagaagatgtggaaacttggctcaagtttgcctcgctttgtcgaaagagtgggaggatcagtcaggcgaaatctactctactcaagctcttaccgtttgatccagaagtatcaccagaaaacatgcaatatcacggacctccacaagtgatgcttggatacttaaaataccaatggtcacttggagaggaacgtaagcgcaaagaggcatttaccaagctgcagattctaacgagagagctctcaagtgtgccacattctcaatctgacatactggctagcatggtatctagcaagggcgcaaatgttccacttcttgcacgtgtaaatctcaaactgggaacgtggcagtgggcactttcttccggtttgaatgatgggtctattcaagaaattcgtgatgcgtttgacaaatctacttgctatgctcctaaatgggctaaagcatggcacacatgggcattattcaatacagcagtgatgtcgcattacatttcaagaggtcaaattgcttcccagtacgttgtttctgcagtcactggatatttttattctatagcatgtgcagcaaatgccaaaggagttgatgatagtttacaggacatactgcgtcttctgacattgtggttcaaccatggagctacagctgatgtccaaaccgcattgaagacaggattcagtcatgtcaacattaacacatggcttgttgtgctacctcaaatcattgctaggatacattctaataatcgtgctgtcagggaactgattcagtctcttctcatccgcataggcgaaaaccacccacaggctctgatgtatccccttctcgttgcatgtaaatcaataagcaatcttcggagagctgcggctcaagaggtggttgataaagttcgccagcacagtggtgcactcgtggatcaggcgcaacttgtatcacatgaacttatcagggttgccatactttggcatgaaatgtggcatgaagcactagaagaagctagtcgcttgtattttggtgaacataacattgaaggcatgctgaaagtacttgaacccttacatgacatgctcgacgaaggtgtaaaaaaggacagtacgaccatacaggaaagagcatttatagaggcataccgtcacgaactaaaagaggcacatgaatgctgttgcaattacaagataactgggaaagatgctgaacttacacaggcttgggatctttactatcacgttttcaaacggattgacaaacagctagccagtctcacgacattggatttggaatctgtttctcctgagttgctgctgtgccgtgacttggagctagcagttcctggaacatatcgtgcagatgcccccgtcgtgactatatcatctttttcacgccaacttgttgttataacctctaaacaaagaccaaggaaattgactattcacggaaatgacggtgaggactacgccttcttgttgaagggacatgaagatttaaggcaagatgagcgtgttatgcagctttttggtttggtgaacactttgcttgagaattccagaaaaacagccgaaaaagatctttccattcaacgctattctgtaataccactatctcccaatagtggactcatcggatgggttccgaactgcgatacccttcaccatcttattcgagagcacagagatgcaagaaagatcattcttaatcaagaaaataagcatatgttgagttttgctccagactatgacaatctaccgcttatagcaaaggttgaagtatttgagtatgctctagaaaacacagagggaaatgatctatccagggttctctggttaaaaagtcgctcgtcagaagtttggctagaaagaagaacaaactatactagaagtttagcagttatgagtatggttggttatattcttgggttaggtgatcgacacccaagtaaccttatgcttcatagatacagtggaaagatcttgcatattgattttggagattgttttgaggcttctatgaatagagagaagtttcctgaaaaggttccattccgcctgacaagaatgcttgtcaaagcaatggaagtcagtggcattgaaggaaacttccgctcaacctgcgaaaacgttatgcaagttctcagaaccaataaagatagtgtaatggcaatgatggaagcgtttgtacatgatcctttaatcaattggcgtcttttcaatttcaatgaagtcccccaattagcactgctcggtaacaacaaccccaatgctcctgctgatgttgagcctgacgaagaagatgaagatcccgctgatatagatcttcctcagcctcaaaggagtactcgagagaaggagattcttcaggctgtaaatatgcttggagatgctaatgaagttttaaatgagcgtgccgtagttgttatggcacgtatgagtcataagcttacagggcgtgatttttcttcgtctgcaattccgagcaatcccattgctgatcataataacttgctcggaggagattctcatgaagtcgaacatggtttgtctgtgaaagttcaggttcaaaaactaatcaatcaagccacttcccatgagaatctctgtcaaaactatgttgggtggtgccctttctgg

SEQ ID NO: 26 - DNA, 7569 bp, Chlamydomonas reinhardtii
atgatgctgtcgggagtgggtccggtgcccaccaaaccggctttcaaggccggtggcgacacgctctcgcggcacctggaggagctgtgccgttctggcgcatgggagcggcgccacaaggatggtgacaaagcattattggagtacatcgaggcggaggctcgggacctgtcggtggaggcttttgggcggctaatgaccgacgtgtatcagcgcatcggcaacatgctgctcaaagggaacgacatcacgcggcgcatgggtggcgtgctggcgattgacgagcttatcgatgtcaagctctctggagacgacgctgccaagacggcgcggctgtcggggctgctgtcgcgggtgctggaggagagcgaggacccggtgctcagcgagtcggcctcgcacacgctgggacacctggtgcgcagcggcggcgccatgacgtcggacatcgtggagaaggagatccgccgctcgcttgcctggtgcgacccccgcaatgagcccaatgagtcgcggcggctgactgcgctgctggtgctgacggaggcggcggagtccgcgcccgccgtgttcaacgtgcacgtcaagtcgttcattgacgcggtgtggttcccgctgcgcgacgccaagcagcatatccgcgaggcggcggtgcgggcgctcaaggcttgcctgtgcctggtggagaagcgcgagacgcgctaccgcgtgcagtggtactacaagctgcacgagcagaccatgcgcggcatgaagcgcgaccaccgcaccggcgcgcttccctcgcccgagtccatccacggctcgctgctggcgctggcggagctgctacagcacaccggcgaattcatgctggcgcgctacaaggaggttgtggagaacgtgttccgctacaaggacagcaaggagaaaaacatccgccgggcggtcatccacctgctgccgcgcatggcggccttctcgccggagcgctttgcgtcggagtacctggctcgcgccattgccttcctgctgatcgtgctgaagaacccgcccgagcgcggcgcggcgttcgcggcgctggcggacatggcggcggccctggcgcggggctgcctgtcgcccatctacgtcgccatccgggaggcgctgtcggcgccgcccgccgcgcgcgccgccgcccggccgcggcccgccacctgctacgaggccctgcagtgcgtgggcatgctggcggtggcgctgggcccgctgtggcggccctacgcggcggcgctggtggaggccatggtgctcacaggcgtgagcgaggtgctggtgcaggcgctgacgcaggtcgccaacgcgctgccggagcttctggaggacatccagtaccagctgctggacctgctgagcctggtgctcagcaagaggcccttcaacagcagcaccacgcagcccaagttcgcggccctgagtgcggccatcgcggcgggcgagctgcagggcaatgcactcaccaagctggcgctgcagacactgggcacgtttgacctgggcggcatccagcttctggagttcatgcgcgaccacatcctggcctacaccgacgaccccgacaaggagatccgccaggccgcggtgctggccgcatgcccgcgtgctggagcggcacgcagcagcctccgcgtccgcagcctccgcagcggctggcggcgcgccgccgcggctgtgtggcacacgcgcgtggtggagcgctgtgtgggccggctgctggtggtggcggtggcggaccccagtgagcgcgtgcgcaaggaggtgctgcgggcgctggtggccaccacggccctggacgactacctggcgcaggccgactgcctgcgcgcgctgttcgtgggcatgaacgacgagagcgtggcggtgcgcgggctggccatccggctggtggggcggctggcggagcgcaacccggcgcacgtgaacccggcgctgcgcaagcacctgctgcagctgctgcacgacatggagttcagccccgacaacagggccagggaggagtcggccttcctgctggaggtgctcatcaccgccgccgcccgcctcatcatgccctacgtctcgcccatccagaaggcgctggtgtccaagctgcgcggcggctcgggcccgggcataactgtgttgtccacgctgggcgcgctggctgaggtgagcggcaccacgttccgccccttcatcagcgaggtcatgccgctggtcatcgaggccattcaggacaactcggacgggcggcggcgtgtggtggccgtcaagactctgggcttcatcgtgagcagctgcggcaatgtgatgggcccctacctggagtacccacagctgctgtcggtgctgctgcgcatgctgcacgagggacaccccgcgcaacgccgggaggtcatcaaggtgctgggcatcatcggtgcgctggacccgcacacacacaagctcaaccaggccagcctgagcggggagggcaagctggagaaggagggggtgcggccgctgcggcacggcggcggcggcgcgggcggcgccggcggcggcgcaggcgggggaggcgtcggcggcggcgtggcgggcgacagcaatgacggcggcatgggccccggcgacgacggcggccccggcggcgacctgctgccctcctcgggcctggtgaccagcagcgaggactattaccccaccgtggccatcaacgcgctgatgcgggtgctgcgcgaccccgccctggcctcccagcacctggccgtcatccgggcgctggcagccatattccgcgcgctgcagctcagcgtagtgccctacctgcccaaggtcctgcccatcctgctgggcgtgctgcgcggcggcgacgaggcgctgcgtgaggagatcctggcctcgctgcgcgcgctggtgggctacgtgcggcagcacatgcgccgcttcctgcccgacctcacgcagctggtgcacgagttctggcccgccgcgccgcgcacctgcctggcgctcatagcggacctgggcatggcgctgagggacgacatacgtgccaaacccctccctcccctccctctcctgccgccctcctctcccccccgcacaccccacaacaggcagtacgtgcccgagctgctgcccaagttcgtggcggtgttcagcgaggccgagcgcgccggcagctgggacctggtgcggcccgccctgggcgccctggagagcctgggcagcgccgtggacgactcgctgcacctgctgctgccctccatggtgcggctgatcagccccgccgccagctccacgccagccgaggtgcggcgcgcggcgctgcgctcgctgcggcggctcatcccgcgcatgcagctgggcggctacgcctcggcggtgctgcacccgctcatcaaggtcctggacggccacagcgacgagcagctgcggcgtgatgcgctagacaccatctgcgccgtggccgtgtgcctggggcccgagttcgccatcttcgtgcccaccatccgcaaggtgcgtgtgcggcaccgcctgcaccacgagtggttcgaccggctggccggcaaggtgtgcgccgtgtcgccgccctgcatgtcagacgcggaggactgggagggcgccggaggcgccgcctccggcgccggctccgccggcgcagccggcggctgggccgtggagatcgacctgctcgcccgcatgcaggcggagggcggcggcgccctgggcggccagccgccggtgccgccgggtcccgacggcggcccctccgccaagctgccggtgaacgcggcggtgctgcggcgcgcctgggagagcagccaccgcgtgaccaaggaggactgggcggagtggatgcgcaacttcgcggtggagctgctcaaggagagcccctcgcccgcgctgcgcgcctgccacggcctggcgcaggtgcaccccagcatggcgcgcgagctgttcgcggcgggcttcgtgagctgctgggcggagctggagcaggggctgcaggagcagctcgtgcgcagcctggaggctgcgctggcctcccccaccatcccccccgagacggtgactgcgctgctgaacctggccgagttcatggagcacgacgacaagcgcctgcctctggacacacgcacgctgggggcgctggcggagaagtgccacgccttcgccaaggcgctgcactacaaggagctggagttccagaccagcccgcagtccgccatcgaggcgctcatccacatcaacaaccagctgcggcagccggaggcggcggtgggcgtgctggcgtacgcccagaagcacctgcacatggagctcaaggagggctggtatgagaagctgtgccgctgggacgaggcactggacgcctacgagcggaggctgctcaaggaggcgccgggcagcatggagtaccacacagcgctgctgggcaagatgcgctgcctggcctcgctggccgagtgggagaacctgtccaacctgtgccgcaccgagtggcgcaagtcggagccgcacgtgcgccgtgagatggcgctcatcgcggcgcacgcggcctggcacatgggcgcctgggacgagatggccatgtacgtggacacggtggacaaccccgaggccgtggggcccaacagccacacccccaccggcgccttcctgcgcgcggtgctgtgcgtgcgcgccaaccaggtgagcggggcgcaggcgcacgtggagcgcacccgcgagctgatggtggcggacctggcggcgctggtgggcgagagctacgagcgcgcctacacggacatggtgcgcgtgcagcagctggcggagctggaggaggtgtgcgcctacaagcaggcgctggacaggagggcagccgacccgggcggcagcgaggctcgcatcggcttcatccagcagctgtggcgcgaccggctccgcggcgtgcagcggcacgtggaggtgtggcagagcctgttctccatccgcagcctggtggtgcccatggcgcaggacgtggacagctggctcaagttcgccagcctgtgccgcaagagcggccgcagcaggcaggcctaccgcatgctgctgcagctgctgcgctacaaccctatgaacatcactcaggccggcaaccccggctacggcgccggcagcggcgcgccgcacgtgatgctggccttcctgaagcacctgtggacgcagggcaaccgcacagaggcctacaaccgcatcaaggacctggcgtcgctcaacggccgggccttcctgcggctgggcatctggcagtgggccatgaacgatctggacaacccgggtgtgattgcggagaacctggcttccttccgcgcggccaccgagcacgcgcccaattgggccaaggcctggcaccagtgggcgctgttcaatgtggcggtttcagcgcactacaggtgcgaccccatgcgggacgagaaccaggccgtgtcgcacgtgccgccggcggtgcagggcttcttccgcagcgtggcgctggggcaggcggcgggcgaccgcacaggcaacctgcaggacatcctgcggctgctgacgctgtggttcaacttcggcgcgtacgccgaggtccgcgccgcgctgacggagggcttccagctggtgtccatcgacacctggctgctggtcatcccgcagatcatcgcgcgcatccacacccacaacacagacgtgcgccagctcatccaccacctgctggtcaagatcgggcgccaccacccgcaggctctgatgtacccgctgctggtggccaccaagtcccagagcccggcccggcgccaggcggcctacagcgtgctggagtgcatccggcagcacagcgcggcgctggtggagcaggcgcagctggtcagcggcgagctcatccgcatggccatcctgtggcacgagatgtggcacgagggcctggaggaggccagccgcctgtacttcggcgagagcaatgtggagggcatgctgaacacgctgctgccgctgcacgagatgctggagaaggcggggcccaccacactcaaggaaatcgccttcgtgcagagctacggccgggagctgtcggaggcgtacgagtggctgatgaagtacaaggccagccgcaaggaggcggagctgcaccaggcctgggacctgtactaccacgtcttcaagcgcatcaacaagcagctgcgctccctaaccacgctggagctgcagtacgtcagccccgcgctggtgcgggcgcaggacctggagctggcagtgcccggcacctacattgccggggagccgctggtgaccatcgccgccttcgcgccgcagctgcacgtcatcagctccaagcagcgcccgcgcaagctcaccatacacggcggcgacggcgcggagtacatgttcctgctcaagggccacgaggacctgcgccaggacgagcgcgtgatgcagctgtttggcctggtgaacaccatgttggcgcacgaccgcatcaccgccgagcgcgacctgtccatcgcgcgctacgccgtcatcccgctgtcgcccaacagcggcctcatcggctgggtgcccaactgcgacacgctgcacgcgctcatccgggagtacagggaggcccgcaagatcccgctcaactgggagcaccgtctgatgctgggcatggcgcccgactacgaccacctgacggtcatacagaaggtggaggtgttcgagtacgcgctggactccaccagcggcgaggacctgcacaaggtgctgtggctcaagagccgcaacagcgaggtttggctggaccggcgcaccaactacacccgctccgccgccgtcatgtccatggtgggctacatcctgggcctgggcgaccgccacccctccaacctcatgctggaccgctacagcggcaagctgctgcacatcgactttggcgactgcttcgaggcgtccatgaaccgggagaagttcccggagaaggtgccgttccggctcacgcgcatgatgatcaaggccatggaggtgtcgggcatcgagggcaacttccgcaccacgtgcgagaacgtgatgcgcgtgctgcgctccaacaaggagagcgtgaccgccatgctggaggccttcgtgcacgaccccctcatcaactggcgcctgctcaacaccaccgaggcagccacggaggcggcgctggcgcgcacggacggcggcggcggcggcggtggccacatggacggccccggggggcacccggggggccgggacgcgctgggcggcggcggcggcggggcgggcggcggcggcggcggcgacccgggggccatgcccagcccgccgcggcgcgagacgcgggagaaggagctcaaggaggcgtttgtgaacctgggcgatgccaacgaggtgttgaacacgcgcgcggtggaggtgatgaagcgcatgagcgacaagctcatgggccgcgactacgcccccgagctatgtgtgggcggcggcagcggcgccagcggcatggagccggacagcgtgccggcgcaggtggggcgcctcatcaacatggcggtcaaccacgagaacctgtgccagagctacatcggctggtgccccttctgg

SEQ ID NO: 27 - DNA, 1422 bp, Arabidopsis thaliana
atggccgccgcagtttccaccgtcggtgccatcaacagagctccgttgagcttgaacgggtcaggatcaggagctgtatcagccccagcttcaaccttcttgggaaagaaagttgtaactgtgtcgagattcgcacagagcaacaagaagagcaacggatcattcaaggtgttggctgtgaaagaagacaaacaaaccgatggagacagatggagaggtcttgcctacgacacttctgatgatcaacaagacatcaccagaggcaagggtatggttgactctgtcttccaagctcctatgggaaccggaactcaccacgctgtccttagctcatacgaatacgttagccaaggccttaggcagtacaacttggacaacatgatggatgggttttacattgctcctgctttcatggacaagcttgttgttcacatcaccaagaacttcttgactctgcctaacatcaaggttccacttattttgggtatatggggaggcaaaggtcaaggtaaatccttccagtgtgagcttgtcatggccaagatgggtatcaacccaatcatgatgagtgctggagagcttgagagtggaaacgcaggagaacccgcaaagcttatccgtcagaggtaccgtgaggcagctgacttgatcaagaagggaaagatgtgttgtctcttcatcaacgatcttgacgctggtgcgggtcgtatgggtggtactactcagtacactgtcaacaaccagatggttaacgcaacactcatgaacattgctgataacccaaccaacgtccagctcccaggaatgtacaacaaggaagagaacgcacgtgtccccatcatttgcactggtaacgatttctccaccctatacgctcctctcatccgtgatggacgtatggagaagttctactgggccccgacccgtgaagaccgtatcggtgtctgcaagggtatcttcagaactgacaagatcaaggacgaagacattgtcacacttgttgatcagttccctggtcaatctatcgatttcttcggtgctttgagggcgagagtgtacgatgatgaagtgaggaagttcgttgagagccttggagttgagaagatcggaaagaggctggttaactcaagggaaggacctcccgtgttcgagcaacccgagatgacttatgagaagcttatggaatacggaaacatgcttgtgatggaacaagagaatgtcaagagagtccaacttgccgagacctacctcagccaggctgctttgggagacgcaaacgctgacgccatcggccgcggaactttctacggaaaaggagcccagcaagtaaacctgccagttcctgaagggtgtactgatcctgtggctgaaaactttgatccaacggctagaagtgacgatggaacctgtgtctacaacttt

SEQ ID NO: 28 - DNA, 1224 bp, Scenedesmus dimorphus
atgcaggtcaccatgaagagcagcgccgtcagcggccagcgcgtgggcggtgcccgcgtcgccacccgtagcgtgcgccgggcgcagctgcaggttgtggcctctagccgcaagcagatgggccgctggcggtcgatcgacgcgggcgtcgacgcgtccgatgaccagcaagacatcactcgcggccgcgagatggtggacgacctgttccagggcggcttcggtgccggcggcacccacaacgcagtgctgtccagccaggagtacctgagccagagccgcgcctcgttcaacaacattgaggacggcttctacatctcgcccgctttcctggacaagatgaccatccacattgccaagaacttcatggacctgcccaagatcaaggtgcccctcattctgggtatctggggtggcaagggccagggcaagaccttccagtgcgcgctcgcctacaagaagctgggcattgcccccatcgtcatgtccgctggtgagctggagtccggcaacgccggtgagcccgccaagctgatccgcacccgctaccgggaggcctccgacatcatcaagaagggccgcatgtgctcgctgttcatcaacgatctggacgccggtgccggccgcatgggcgacaccacccagtacaccgtgaacaaccagatggtgaacgccaccctgatgaacatcgccgacaacccgaccaacgtccagctgcccggtgtgtacaagaacgaggagatccctcgcgtgcccattgtgtgcacgggcaacgacttctccaccctgtacgcgcccctgatccgcgatggccgcatggagaagtactactggaaccccacccgcgaggaccgcatcggcgtgtgcatgggcatcttccaggaggacaacgttcagcgccgcgaggtggagaacctggtggacaccttccccggccagtccattgacttcttcggcgccctgcgtgcccgcgtgtacgacgacatggtgcgccagtggatcaccgacaccggcgtggacaagatcggccagcagctggtcaacgcccgccagaaggtggccatgcccaaggtgtccatggacctgaacgtgctgatcaagtacggcaagtcgctggtggacgagcaggagaacgtcaagcgcgtgcagctggccgatgcctacctgtcgggcgccgagctggccggccacggcggctcttccctgcccgaggcctacagccgc

SEQ ID NO: 29 - DNA, 1218 bp, Scenedesmus dimorphus
atgcagcttgcaggccagaagagcattgctgggcagcgcccatgcgctgcacgcactgccgtgaagcaggtccgcgtcgcacctgttcaggccagcaagtcccagaagggtcgctggagtgccatggatgccggcaacgaccaatccgatgaccagcaagacattgcgcgtggccgcggcatggttgacgagctgttccagggctggggcggcaccgccggcactgccaatgccatcatgaacagcagcgactacctgagccaggcagccaagaccttcaacaacattgaggatggcttctacatctctcctgctttcctggacaagatcaccatccacgtggccaagaacttcatggacctgcccaagatcaaggtgcccctcatcctgggtatctggggaggcaagggacagggtaagaccttccagtgcgcgctggccttcaagaagctgggcatcagccccatcgtgatgagcgctggtgagctggagtccggcaacgcaggagagccagccaagctgctgcgccagcgctacagggaggcgtctgaccagatcaagaagggcaagatgtgcgcgctgttcatcaacgatctggatgccggagcaggccgcatgggcgagtccacgcagtacacggtcaacaaccagatggtcaacgccacgctcatgaacattgccgacaaccccaccaacgtgcagctgccaggcgtgtacaagaacgaggagatcccccgcgtgcccatcatctgcacaggtaacgacttctctaccctgtacgcccctctgatccgtgatggccgtatggagaagtactactggaaccccacccgcgaggaccgcgtgggcgtgtgcatgggcatcttccaggaggacaaggtgtcccgtggtgaggtggaggtgctggtggacaccttccccggccagtccatcgacttcttcggagctctgcgcgcacgcgtgtacgacgacaaggtgcgtgagttcatcagcggcatcggcgtggagaacatcggcaagcgcctcatcaacagccgcgagggcaaggtcaactttgagaagcccgccatgcccctggacatcctgatcaagtacggcaagcagctggtggatgagcaggacaacgtgaagcgtgtgcagctggctgatgcctacctggcaggcgctgagctggcaggatctggcggcagctccatgccagaggcttacgcggcccag

SEQ ID NO: 30 - protein, 406 aa, Scenedesmus dimorphus
MQLAGQKSIAGQRPCAARTAVKQVRVAPVQASKSQKGRWSAMDAGNDQSDDQQDIARGRGMVDELFQGWGGTAGTANAIMNSSDYLSQAAKTFNNIEDGFYISPAFLDKITIHVAKNFMDLPKIKVPLILGIWGGKGQGKTFQCALAFKKLGISPIVMSAGELESGNAGEPAKLLRQRYREASDQIKKGKMCALFINDLDAGAGRMGESTQYTVNNQMVNATLMNIADNPTNVQLPGVYKNEEIPRVPIICTGNDFSTLYAPLIRDGRMEKYYWNPTREDRVGVCMGIFQEDKVSRGEVEVLVDTFPGQSIDFFGALRARVYDDKVREFISGIGVENIGKRLINSREGKVNFEKPAMPLDILIKYGKQLVDEQDNVKRVQLADAYLAGAELAGSGGSSMPEAYAAQ

SEQ ID NO: 31 - DNA, 1218 bp, artificial sequence
  synthesized
atgcagctcgctggtcagaagtctattgctggccaacgtccatgtgcggcgcgcaccgccgtgaagcaggtccgcgtggcccccgtccaggcgtccaagtcccagaagggccgctggtccgcgatggatgcgggcaacgaccagtctgacgaccagcaggacatcgcccgtggtcgcggcatggtggacgagctgtttcagggctggggcggtactgcggggaccgccaacgccatcatgaactctagcgactacctgagccaggcggcgaagacgttcaacaacatcgaggacggcttctacatcagccctgcgtttctggacaagatcacgatccacgtcgctaagaactttatggacctgccaaagatcaaggtgcccctgatcctgggcatctggggcgggaagggccagggcaagacgtttcagtgcgcgctggcgttcaagaagctcggcatctcccctatcgtgatgtctgccggcgagctggagtccggcaacgcgggcgagcctgcgaagctcctgcgccagcgctaccgtgaggcctccgaccagatcaagaagggtaagatgtgcgccctgttcattaacgacctggacgccggggcgggccgcatgggcgagagcacgcagtacacggtgaacaaccagatggtgaacgccactctgatgaacatcgccgacaaccccactaacgtccagctgcccggcgtgtacaagaacgaggagatcccccgcgtgcctatcatttgcaccggcaacgacttctccaccctgtacgctcccctgattcgcgacggccgtatggagaagtactactggaacccaacccgcgaggaccgcgtcggggtgtgtatgggcatcttccaggaggacaaggtgagccgtggcgaggtggaggtcctggtggacacgttccccggccagtccatcgacttcttcggggctctgcgcgctcgcgtgtacgatgacaaggtccgcgagttcatttccggcatcggcgtggagaacatcggcaagcgcctgatcaacagccgcgagggcaaggtgaacttcgagaagcccgcgatgcccctggacatcctgattaagtacggcaagcaactggtcgatgagcaggacaacgtgaagcgtgtccagctggccgacgcgtacctggccggcgcggagctggcgggttctggcggctcctccatgcctgaggcctacgcggcccag

SEQ ID NO: 32 - DNA, 1215 bp, Scenedesmus dimorphus
cagcttgcaggccagaagagcattgctgggcagcgcccatgcgctgcacgcactgccgtgaagcaggtccgcgtcgcacctgttcaggccagcaagtcccagaagggtcgctggagtgccatggatgccggcaacgaccaatccgatgaccagcaagacattgcgcgtggccgcggcatggttgacgagctgttccagggctggggcggcaccgccggcactgccaatgccatcatgaacagcagcgactacctgagccaggcagccaagaccttcaacaacattgaggatggcttctacatctctcctgctttcctggacaagatcaccatccacgtggccaagaacttcatggacctgcccaagatcaaggtgcccctcatcctgggtatctggggaggcaagggacagggtaagaccttccagtgcgcgctggccttcaagaagctgggcatcagccccatcgtgatgagcgctggtgagctggagtccggcaacgcaggagagccagccaagctgctgcgccagcgctacagggaggcgtctgaccagatcaagaagggcaagatgtgcgcgctgttcatcaacgatctggatgccggagcaggccgcatgggcgagtccacgcagtacacggtcaacaaccagatggtcaacgccacgctcatgaacattgccgacaaccccaccaacgtgcagctgccaggcgtgtacaagaacgaggagatcccccgcgtgcccatcatctgcacaggtaacgacttctctaccctgtacgcccctctgatccgtgatggccgtatggagaagtactactggaaccccacccgcgaggaccgcgtgggcgtgtgcatgggcatcttccaggaggacaaggtgtcccgtggtgaggtggaggtgctggtggacaccttccccggccagtccatcgacttcttcggagctctgcgcgcacgcgtgtacgacgacaaggtgcgtgagttcatcagcggcatcggcgtggagaacatcggcaagcgcctcatcaacagccgcgagggcaaggtcaactttgagaagcccgccatgcccctggacatcctgatcaagtacggcaagcagctggtggatgagcaggacaacgtgaagcgtgtgcagctggctgatgcctacctggcaggcgctgagctggcaggatctggcggcagctccatgccagaggcttacgcggcccag

SEQ ID NO: 33 - protein, 405 aa, Scenedesmus dimorphus
QLAGQKSIAGQRPCAARTAVKQVRVAPVQASKSQKGRWSAMDAGNDQSDDQQDIARGRGMVDELFQGWGGTAGTANAIMNSSDYLSQAAKTFNNIEDGFYISPAFLDKITIHVAKNFMDLPKIKVPLILGIWGGKGQGKTFQCALAFKKLGISPIVMSAGELESGNAGEPAKLLRQRYREASDQIKKGKMCALFINDLDAGAGRMGESTQYTVNNQMVNATLMNIADNPTNVQLPGVYKNEEIPRVPIICTGNDFSTLYAPLIRDGRMEKYYWNPTREDRVGVCMGIFQEDKVSRGEVEVLVDTFPGQSIDFFGALRARVYDDKVREFISGIGVENIGKRLINSREGKVNFEKPAMPLDILIKYGKQLVDEQDNVKRVQLADAYLAGAELAGSGGSSMPEAYAAQ

SEQ ID NO: 34 - DNA, 1215 bp, artificial sequence
  synthesized
cagctcgctggtcagaagtctattgctggccaacgtccatgtgcggcgcgcaccgccgtgaagcaggtccgcgtggcccccgtccaggcgtccaagtcccagaagggccgctggtccgcgatggatgcgggcaacgaccagtctgacgaccagcaggacatcgcccgtggtcgcggcatggtggacgagctgtttcagggctggggcggtactgcggggaccgccaacgccatcatgaactctagcgactacctgagccaggcggcgaagacgttcaacaacatcgaggacggcttctacatcagccctgcgtttctggacaagatcacgatccacgtcgctaagaactttatggacctgccaaagatcaaggtgcccctgatcctgggcatctggggcgggaagggccagggcaagacgtttcagtgcgcgctggcgttcaagaagctcggcatctcccctatcgtgatgtctgccggcgagctggagtccggcaacgcgggcgagcctgcgaagctcctgcgccagcgctaccgtgaggcctccgaccagatcaagaagggtaagatgtgcgccctgttcattaacgacctggacgccggggcgggccgcatgggcgagagcacgcagtacacggtgaacaaccagatggtgaacgccactctgatgaacatcgccgacaaccccactaacgtccagctgcccggcgtgtacaagaacgaggagatcccccgcgtgcctatcatttgcaccggcaacgacttctccaccctgtacgctcccctgattcgcgacggccgtatggagaagtactactggaacccaacccgcgaggaccgcgtcggggtgtgtatgggcatcttccaggaggacaaggtgagccgtggcgaggtggaggtcctggtggacacgttccccggccagtccatcgacttcttcggggctctgcgcgctcgcgtgtacgatgacaaggtccgcgagttcatttccggcatcggcgtggagaacatcggcaagcgcctgatcaacagccgcgagggcaaggtgaacttcgagaagcccgcgatgcccctggacatcctgattaagtacggcaagcaactggtcgatgagcaggacaacgtgaagcgtgtccagctggccgacgcgtacctggccggcgcggagctggcgggttctggcggctcctccatgcctgaggcctacgcggcccag

SEQ ID NO: 35 - DNA, 1233 bp, unknown
  Desmodesmus species
atgcagctgcaccagagcaccaccggcgtcagggcccctacagcggcaccagttcgcgcaaacaaggttgtccgtgtgaagccatgccaggcaggcaagacccagaagggcaggtggaggggcatggacgcagaccaggacgcgtctgacgaccagcaagacattgcccgcggccgtggcatggttgatgagctgttccagggctggggtggccagggaggcactgccaatgccatcctgagcagcacagactacctgagccaggctgccaagtcgttcaacaacattgaggagggcttctacatctcccctgccttcctggacaagctgaccatccacgtggcaaagaacttcatggacctgcccaagatcaaggtgcccctcatcctgggtatctggggaggaaagggtcagggtaagaccttccagtgcgctcttgcctacaagaagcttggcattgcccccattgtgatgagtgctggtgagctggagtctggcaatgcaggagagccagctaagctcatcaggcagcgctacagggaggcatcagatgtcatcaagaagggcaagatgtgctctctgttcatcaacgatctggatgccggagcaggtcgcatgggcgagtccacccagtacacagtcaacaaccagatggtgaacgccactctgatgaacattgccgacaaccccaccaacgtgcagctgccaggtgtctacaagaacgagaccatcccccgtgtgcccatcgtgtgcacaggtaacgatttctccaccctgtacgcccctctgatccgtgatggtcgtatggagaagtactactggaaccccacacgcgaggaccgtatcggtgtgtgcatgggcatcttccaggaggacgcggtcgaccgtaacgacattgaggtccatgtggacaccttccccggccagtccatcgacttcttcggtgctctgcgtgcccgcgtgtacgacgacaaggtccgtgacttcatctccggcattggtgttgagaacatcggcaagcgcctgatcaacagcagggagggcaaggtcgagttcgacaggccccaaatgaccctggatatcctgatcaagtacggaaagttcctggtggaggagcaggacaacgtcaagcgcgtgcagctggcagacgcatacctggcaggtgctgagctggccggcactggcggcagctccctgcccgagaactacaagggctccagcctcctcagccgc

SEQ ID NO: 36 - protein, 411 aa, unknown
  Desmodesmus species
MQLHQSTTGVRAPTAAPVRANKVVRVKPCQAGKTQKGRWRGMDADQDASDDQQDIARGRGMVDELFQGWGGQGGTANAILSSTDYLSQAAKSFNNIEEGFYISPAFLDKLTIHVAKNFMDLPKIKVPLILGIWGGKGQGKTFQCALAYKKLGIAPIVMSAGELESGNAGEPAKLIRQRYREASDVIKKGKMCSLFINDLDAGAGRMGESTQYTVNNQMVNATLMNIADNPTNVQLPGVYKNETIPRVPIVCTGNDFSTLYAPLIRDGRMEKYYWNPTREDRIGVCMGIFQEDAVDRNDIEVHVDTFPGQSIDFFGALRARVYDDKVRDFISGIGVENIGKRLINSREGKVEFDRPQMTLDILIKYGKFLVEEQDNVKRVQLADAYLAGAELAGTGGSSLPENYKGSSLLSR

SEQ ID NO: 37 - DNA, 1233 bp, artificial sequence
  synthesized
atgcaactccaccaatctactactggggtccgcgctcctactgctgcgcccgtgcgcgccaacaaggtcgtccgcgtgaagccttgccaggcgggtaagacccagaagggccgctggcgcggcatggacgccgaccaggacgccagcgacgaccagcaggacatcgctcgtggccgcggtatggtcgacgagctgttccaggggtgggggggccaaggcggcaccgccaacgccattctgtcctctactgactacctgagccaggccgccaagtccttcaacaacattgaggagggcttctacatcagccccgccttcctggataagctgaccatccacgtggctaagaacttcatggacctgcccaagattaaggtccctctgatcctcggcatctggggcggcaagggccaggggaagactttccagtgcgccctggcgtataagaagctgggcatcgcgcccatcgtgatgtctgcgggcgagctggagtccggcaacgccggcgagcccgccaagctgattcgccaacgttaccgcgaggcgagcgacgtgatcaagaagggcaagatgtgctctctgttcatcaacgacctggacgctggcgccggccgcatgggcgagtccacgcagtacacggtgaacaaccagatggtcaacgccaccctgatgaacattgcggacaaccccactaacgtgcagctccccggcgtgtacaagaacgagactatcccccgcgtgccaatcgtgtgcacggggaacgatttcagcaccctgtatgcgcccctgatccgtgacgggcgcatggagaagtactactggaaccccactcgcgaggaccgcattggcgtgtgcatgggcatttttcaggaggacgcggtggaccgtaacgacattgaggtgcacgtcgacaccttccccggccagagcatcgacttctttggcgccctgcgcgcgcgcgtgtacgacgacaaggtccgcgacttcatctccggcatcggcgtggagaacattggcaagcgcctgattaacagccgcgagggcaaggtggagtttgaccgcccccagatgaccctcgacatcctgatcaagtatggcaagttcctggtggaggagcaggataacgtgaagcgcgtgcagctggccgacgcgtacctggcgggcgctgagctggcgggcacgggcggctcctctctgccagagaactacaagggcagcagcctgctgtcccgc

SEQ ID NO: 38 - DNA, 1230 bp, unknown
  Desmodesmus species
cagctgcaccagagcaccaccggcgtcagggcccctacagcggcaccagttcgcgcaaacaaggttgtccgtgtgaagccatgccaggcaggcaagacccagaagggcaggtggaggggcatggacgcagaccaggacgcgtctgacgaccagcaagacattgcccgcggccgtggcatggttgatgagctgttccagggctggggtggccagggaggcactgccaatgccatcctgagcagcacagactacctgagccaggctgccaagtcgttcaacaacattgaggagggcttctacatctcccctgccttcctggacaagctgaccatccacgtggcaaagaacttcatggacctgcccaagatcaaggtgcccctcatcctgggtatctggggaggaaagggtcagggtaagaccttccagtgcgctcttgcctacaagaagcttggcattgcccccattgtgatgagtgctggtgagctggagtctggcaatgcaggagagccagctaagctcatcaggcagcgctacagggaggcatcagatgtcatcaagaagggcaagatgtgctctctgttcatcaacgatctggatgccggagcaggtcgcatgggcgagtccacccagtacacagtcaacaaccagatggtgaacgccactctgatgaacattgccgacaaccccaccaacgtgcagctgccaggtgtctacaagaacgagaccatcccccgtgtgcccatcgtgtgcacaggtaacgatttctccaccctgtacgcccctctgatccgtgatggtcgtatggagaagtactactggaaccccacacgcgaggaccgtatcggtgtgtgcatgggcatcttccaggaggacgcggtcgaccgtaacgacattgaggtccatgtggacaccttccccggccagtccatcgacttcttcggtgctctgcgtgcccgcgtgtacgacgacaaggtccgtgacttcatctccggcattggtgttgagaacatcggcaagcgcctgatcaacagcagggagggcaaggtcgagttcgacaggccccaaatgaccctggatatcctgatcaagtacggaaagttcctggtggaggagcaggacaacgtcaagcgcgtgcagctggcagacgcatacctggcaggtgctgagctggccggcactggcggcagctccctgcccgagaactacaagggctccagcctcctcagccgc

SEQ ID NO: 39 - protein, 410 aa, unknown
  Desmodesmus species
QLHQSTTGVRAPTAAPVRANKVVRVKPCQAGKTQKGRWRGMDADQDASDDQQDIARGRGMVDELFQGWGGQGGTANAILSSTDYLSQAAKSFNNIEEGFYISPAFLDKLTIHVAKNFMDLPKIKVPLILGIWGGKGQGKTFQCALAYKKLGIAPIVMSAGELESGNAGEPAKLIRQRYREASDVIKKGKMCSLFINDLDAGAGRMGESTQYTVNNQMVNATLMNIADNPTNVQLPGVYKNETIPRVPIVCTGNDFSTLYAPLIRDGRMEKYYWNPTREDRIGVCMGIFQEDAVDRNDIEVHVDTFPGQSIDFFGALRARVYDDKVRDFISGIGVENIGKRLINSREGKVEFDRPQMTLDILIKYGKFLVEEQDNVKRVQLADAYLAGAELAGTGGSSLPENYKGSSLLSR

SEQ ID NO: 40 - DNA, 1230 bp, artificial sequence
  synthesized
caactccaccaatctactactggggtccgcgctcctactgctgcgcccgtgcgcgccaacaaggtcgtccgcgtgaagccttgccaggcgggtaagacccagaagggccgctggcgcggcatggacgccgaccaggacgccagcgacgaccagcaggacatcgctcgtggccgcggtatggtcgacgagctgttccaggggtgggggggccaaggcggcaccgccaacgccattctgtcctctactgactacctgagccaggccgccaagtccttcaacaacattgaggagggcttctacatcagccccgccttcctggataagctgaccatccacgtggctaagaacttcatggacctgcccaagattaaggtccctctgatcctcggcatctggggcggcaagggccaggggaagactttccagtgcgccctggcgtataagaagctgggcatcgcgcccatcgtgatgtctgcgggcgagctggagtccggcaacgccggcgagcccgccaagctgattcgccaacgttaccgcgaggcgagcgacgtgatcaagaagggcaagatgtgctctctgttcatcaacgacctggacgctggcgccggccgcatgggcgagtccacgcagtacacggtgaacaaccagatggtcaacgccaccctgatgaacattgcggacaaccccactaacgtgcagctccccggcgtgtacaagaacgagactatcccccgcgtgccaatcgtgtgcacggggaacgatttcagcaccctgtatgcgcccctgatccgtgacgggcgcatggagaagtactactggaaccccactcgcgaggaccgcattggcgtgtgcatgggcatttttcaggaggacgcggtggaccgtaacgacattgaggtgcacgtcgacaccttccccggccagagcatcgacttctttggcgccctgcgcgcgcgcgtgtacgacgacaaggtccgcgacttcatctccggcatcggcgtggagaacattggcaagcgcctgattaacagccgcgagggcaaggtggagtttgaccgcccccagatgaccctcgacatcctgatcaagtatggcaagttcctggtggaggagcaggataacgtgaagcgcgtgcagctggccgacgcgtacctggcgggcgctgagctggcgggcacgggcggctcctctctgccagagaactacaagggcagcagcctgctgtcccgc

SEQ ID NO: 41 - DNA, 1179 bp, artificial sequence
  synthesized
ctcgagatgtctgatgatgagcgtgaggagaaggagctggatctgactagccctgaggtcgtgactaagtacaagtccgctgcggagatcgtgaacaaggccctgcagctggtgctgagcgagtgcaagcccaaggtgaagatcgtggacctgtgcgagaagggcgacgcctttatcaaggagcagacgggcaacatgtacaagaacgtgaagaagaagatcgagcgcggcgtggccttccccacttgtatcagcgtgaacaacactgtgtgccacttcagccccctggcttccgacgagacgatcgtggaggagggcgacattctgaagatcgacatgggctgccacatcgacggcttcatcgccgtggtcggccacacgcacgtgctgcacgagggccctgtgaccgggcgtgccgcggacgtgatcgccgcggccaacactgccgctgaggtggcgctccgcctggtgcgtcccggcaagaagaacagcgacgtgactgaggcgattcagaaggtggctgccgcctatgactgcaagatcgtggagggcgtgctgagccaccagatgaagcagttcgtgatcgacggtaacaaggtggtgctgtccgtcagcaacccagatacccgcgtggacgaggccgagttcgaggagaacgaggtgtacagcatcgacattgtgacctccacgggcgacgggaagcccaagctgctcgacgagaagcagacgaccatctacaagcgcgcggtggataagtcttacaacctgaagatgaaggccagccgcttcatcttctctgagatcaaccagaagttccctatcatgcccttcacggcccgcgacctggaggagaagcgcgctcgcctgggcctcgtggagtgcgtcaaccacgagctgctgcagccttaccccgtgctgcacgagaagcccggcgacctggtcgcgcacatcaagttcacggtcctgctcatgcccaacggctccgaccgcgtcacctcccacctgcaggagctgcagcccaccaagaccaccgagaacgagcccgagatcaaggcctggctggccctgcccaccaagacgaagaagaaggggggcggcaagaagaagaagggcaagaagggcgacaaggtggaggaggcgagccaggccgagcccatggaggggacgggctaaggatcc

SEQ ID NO: 42 - DNA, 1176 bp, artificial sequence
  synthesized
ctcgagatgtctgatgacggtagcattgagcaccaagagcctaacctgtctgtgcccgaggtggtgaccaagtacaaggctgcggcggatatctgcaaccgcgcgctgctggcggtggtggaggctgcgaaggacggcgccaaggtggtggacctgtgccgcatgggcgaccagttcatcaacaaggagtgcgcgaacatctacaagggcaaggagatcgagaagggcgtggcgttcccaacctgcgtgtccgcgaactctattgtgggccacttttcccccaacagcgaggacgcgaccgcgctgaagaacggtgacgtggtcaagatcgatatgggctgtcacatcgacgggttcattgctacccaggcgaccaccatcgtggtgggcgacgcggccatcagcggcaaggccgcggacgtgatcgccgctgcgcgcacggcgttcgacgccgcggtccgcctgattcgccctggcaagcacattgcggatgtgagcgctcccctccagaaggtcgctgagtccttcggctgcaacctggtggagggcgtgatgagccacgagatgaagcagttcgtgatcgacggcagcaagtgcatcctgaacaagcccacgcccgaccaaaaggtcgaggacggcgagttcgaggagaacgaggtgtacgccgtcgacatcgtggtcagcagcggcgagggcaagccccgcgtcctcgacgagaaggagactaccgtgtacaagcgcgccctggaggtcacttaccagctgaagatgcaagccagccgcgccgtgtttagcctcgtcaacagcgcgttcgctaccatgccattcaccctgcgtgcgctgctggacgaggctgccgcccaaaagaccgagctgaaggcgagccagctgaagctcggcctggtggagtgcctgaaccacggcctgctgcacccttaccccgtcctgcacgagaagcccggcgaggtggtggcccaaattaagggcaccgtgctgctgatgcctaacggctctagcatcatcaccagcgccccccgccagacggtgaccaccgagaagaaggtggaggacaaggagatcctcgacctgctggcgacgcccatcagcgcgaagagcgccaagaagaagaagaacaaggacaaggctgcggagccagcggctgccaagtaaggatcc

SEQ ID NO: 43 - DNA, 1185 bp, artificial sequence
  synthesized
ctcgagatggtgaaggaggataagcaaactgatggtgatcgttggcgtggtctggcgtacgacacctccgacgaccagcaggatattacgcgcggcaaggggatggtggattccgtgttccaggcgcccatgggcactggcacccaccacgccgtgctgagcagctacgagtacgtctcccagggcctccgtcagtacaacctggacaacatgatggacggcttctacatcgctcccgctttcatggataagctggtggtgcacatcacgaagaacttcctgacgctgcccaacatcaaggtgccactgatcctggggatctggggcggcaagggccagggcaagagcttccaatgcgagctggtgatggcgaagatgggcatcaaccccatcatgatgagcgcgggcgagctggagtccgggaacgccggcgagcccgcgaagctgatccgccagcgctaccgcgaggctgcggacctgatcaagaagggcaagatgtgctgcctgctgattaacgacctggacgcgggcgctgggcgcatgggcggcaccacgcagtacactgtgaacaaccagatggtgaacgcgacgctgatgaacatcgcggacaacccaacgaacgtgcagctgcccggtatgtataacaaggaggagaacgcccgcgtgcccatcatctgcaccggcaacgacttcagcaccctgtacgccccactgatccgcgacggccgcatggagaagttctactgggcgcccacccgcgaggaccgcatcggcatttgtaagggtattttccgcaccgacaagattaaggacgaggacatcgtgaccctcgtggaccaattccctggtcagtccatcgacttcttcggcgcgctgcgcgcccgcgtctacgacgacgaggtgcgtaagttcgtcgagtccctgggggtggagaacatcgggaagcgcctggtgaactcccgcgagggccctcctgtgttcgagcagcccgagatgacttacgagaagctgatggagtacggcaacatgctggtgatggagcaggagaacgtgaagcgcgtgcagctggctgagacttacctgtcccaggccgccctgggcgacgccaacgccgacgccatcggccgcggcaccttctacgggaagactgaggagaaggagccctccaagctggagtaaggatcc

SEQ ID NO: 44 - DNA, 1443 bp, artificial sequence
  synthesized
ctcgagatggctgcggctgtgtccaccgtgggcgctatcaaccgcgcgccactgagcctgaacggcagcggctccggcgccgtgtccgcccctgccagcacctttctggggaagaaggtggtgaccgtcagccgctttgcccagagcaacaagaagagcaacggcagcttcaaggtgctggctgtcaaggaggacaagcagacggacggggaccgctggcgcggcctggcctatgacacctccgacgatcagcaggacatcacgcgcggtaagggcatggtggacagcgtgttccaggcgcctatgggcaccggcacgcaccacgcggtcctgtccagctacgagtacgtgagccagggcctgcgccagtacaacctggacaacatgatggatggcttctacatcgctcccgcgttcatggacaagctggtcgtgcacattaccaagaacttcctcacgctgcccaacattaaggtgcccctgatcctcggcatctggggcggcaagggtcagggcaagtccttccagtgcgagctggtgatggccaagatgggcattaaccctatcatgatgagcgccggcgagctggagagcggtaacgccggcgagcccgccaagctgatccgccaacgctaccgcgaggccgcggacctcatcaagaagggcaagatgtgctgcctgttcatcaacgacctggacgcgggcgccggccgcatgggcggcaccacgcagtacacggtgaacaaccagatggtgaacgccaccctgatgaacatcgccgataaccccacgaacgtccagctccccggcatgtataacaaggaggagaacgcgcgcgtccccatcatctgcactggcaacgacttcagcaccctgtacgctcccctcattcgcgacggccgcatggagaagttctactgggcccctacccgcgaggaccgcattggcgtgtgtaagggcatttttcgcaccgacaagatcaaggacgaggacatcgtgacgctggtggaccagttcccaggccagtccatcgatttttttggcgctctgcgtgcccgcgtctacgacgacgaggtccgcaagttcgtggagtccctgggcgtggagaagatcggcaagcgcctggtcaactcccgcgagggcccacctgtcttcgagcaacccgagatgacgtacgagaagctgatggagtacggcaacatgctggtgatggagcaggagaacgtcaagcgcgtgcaactggccgagacttacctgagccaggccgcgctgggggacgctaacgctgacgcgattgggcgcggcaccttttacggcaagggcgcgcagcaggtgaacctgcccgtgccagagggctgcaccgaccctgtggcggagaactttgaccctacggcgcgcagcgacgatggcacttgcgtctacaacttcaccggctaaggatcc

SEQ ID NO: 45 - DNA, 1269 bp, artificial sequence
  synthesized
catatggctgtaaaagaagataaacaaactgatggagatcgttggcgtggtttagcttatgatacttcagatgatcagcaagatataacacgtggaaaaggtatggttgactctgttttccaagcaccaatgggcacaggtacacatcatgctgttttatcatcttatgaatatgtatctcaaggtttacgtcaatacaatttagataatatgatggatggtttctacattgcaccagcatttatggataaattagtagttcatataactaaaaacttcttaacattaccaaacataaaagtaccattaatacttggtatttggggtggtaaaggccaaggtaaatcatttcaatgtgaattagtaatggctaaaatgggtataaatcctattatgatgagtgctggtgaattagaatctggtaacgcaggtgaaccagcaaaacttattcgtcaacgttatcgtgaagctgctgaccttattaaaaaaggtaaaatgtgttgccttttcattaatgatttagatgctggtgcaggtcgtatgggtggaacaacacaatatactgttaacaatcaaatggttaatgcaacacttatgaacattgcagataatcctacaaatgttcaattacctggtatgtataacaaagaagaaaatgctcgtgttcctataatttgtactggtaatgatttttctacattatatgctccattaattcgtgatggccgtatggaaaaattctactgggcaccaactcgtgaagaccgtattggtgtatgcaaaggtatttttcgtactgacaaaatcaaagatgaagacattgtaacattagtagatcaatttccaggtcaatcaattgactttttcggtgctttacgtgctcgtgtttatgatgatgaggttcgtaaatttgtagaatctttaggagttgagaaaattggtaaacgtttagtaaattcacgtgaaggacctccagtattcgaacaaccagaaatgacatacgaaaaattaatggaatatggtaatatgttagttatggaacaagaaaatgtaaaacgtgttcaattagctgaaacatatttatctcaagcagcattaggtgacgctaacgcagatgctatcggtcgtggtacattttatggtaaaggtgctcagcaagttaatttaccagttccagagggttgcactgatccagttgctgaaaactttgatccaacagctcgttcagatgatggcacttgtgtatataacttcactggttaatctaga

SEQ ID NO: 46 - DNA, 1245 bp, artificial sequence
  synthesized
ctcgagatgcaggtgactatgaagtcctccgccgtgtccggccagcgcgtgggcggtgcgcgtgtggcgacccgctccgtgcgtcgcgcccagctccaggtggtggccagcagccgcaagcagatggggcgctggcgctccatcgacgccggcgtggacgcctccgacgatcagcaggacattacccgtggccgcgagatggtggatgacctgtttcagggcggctttggcgccggggggacccacaacgccgtcctgagctcccaggagtacctgagccagagccgcgcctccttcaacaacatcgaggacggcttctacatctcccccgcgttcctggacaagatgactattcacatcgctaagaactttatggatctgcccaagatcaaggtccccctcattctgggcatctggggcggtaagggccagggcaagaccttccagtgcgccctggcgtacaagaagctgggcatcgcccccatcgtgatgtctgccggcgagctggagagcggcaacgcgggcgagcctgcgaagctgatccgcacgcgctaccgcgaggcctccgatattatcaagaagggtcgcatgtgctccctgtttatcaacgacctggatgctggtgccggccgtatgggcgataccacccagtacaccgtgaacaaccagatggtgaacgcgacgctgatgaacattgccgacaaccccaccaacgtgcagctgcccggcgtgtacaagaacgaggagattccccgcgtccctatcgtgtgcaccggcaacgacttcagcactctgtatgcgcccctgatccgcgacggccgcatggagaagtactactggaaccccacccgcgaggaccgcattggcgtctgcatggggatcttccaggaggacaacgtccagcgccgcgaggtcgagaacctcgtggacaccttccccgggcagagcatcgactttttcggtgccctccgtgcgcgcgtctacgacgatatggtccgccagtggatcacggacacgggcgtggacaagatcggccagcagctggtcaacgcccgccagaaggtcgccatgcccaaggtcagcatggacctcaacgtcctgatcaagtacggcaagtctctggtggacgagcaggagaacgtgaagcgcgtgcagctggccgacgcttacctgtccggcgccgagctggccggccacggcggctccagcctgcccgaggcctattcccgcaccggctgaggatcc

SEQ ID NO: 47 - DNA, 1146 bp, artificial sequence
  synthesized
catatggcttcaagtcgtaaacagatgggtcgttggcgtagtattgatgctggcgtagatgcttcagatgatcaacaagatattacaagaggtcgtgaaatggtagatgacttatttcagggtggctttggtgctggtggtactcacaatgctgttttatcttcacaagaatatttatctcaatctcgtgctagtttcaataatatcgaagatggcttctacatttctcctgcatttttagacaaaatgactattcatattgctaaaaacttcatggatttacctaaaatcaaagtacctcttattttaggtatatggggtggtaaaggtcaaggtaaaacttttcaatgcgcattagcttacaaaaaacttggtattgcacctattgttatgtcagctggtgaattagaaagtggtaatgcaggtgaaccagcaaaattaatccgtacacgttatagagaagcttctgacattattaaaaaaggacgtatgtgctctttattcattaatgatttagatgctggcgctggccgtatgggtgatacaactcaatatacagtaaataaccaaatggttaatgctacattaatgaacatagctgataaccctacaaatgttcaattaccaggtgtttacaaaaatgaagaaatacctcgtgtaccaatcgtttgcacaggtaatgacttttcaacattatacgcaccattaattcgtgatggtcgtatggagaaatactattggaatcctacacgtgaagatcgtattggagtatgtatgggaatatttcaagaagataacgtacaacgtcgtgaggttgaaaacttagttgatacatttcctggacaatcaatcgatttttttggtgctcttcgtgctcgtgtatatgacgatatggttcgtcaatggatcactgatactggtgtagacaaaattggtcaacaattagtaaatgctcgtcaaaaagtagctatgccaaaagtatcaatggacttaaacgtacttatcaaatatggtaaatctttagtagatgaacaagaaaatgttaaacgtgtacaattagctgatgcttatttatctggcgcagaattagctggtcacggtggttcatctttaccagaagcatattcacgtacaggttaatctaga

SEQ ID NO: 48 - DNA, 7587 bp, artificial sequence
  synthesized
ctcgagatgctctctggcgtcggcccagtgccaaccaagcccgcgtttaaggctggcggcgacacgctgagccgccacctggaggagctgtgccgcagcggcgcgtgggagcgccgccacaaggacggcgacaaggcgctgctggagtacatcgaggcggaggcgcgcgacctgtccgtcgaggcgttcggtcgcctgatgaccgacgtgtaccagcgtatcggcaacatgctgctgaagggcaacgacatcacgcgccgcatgggcggggtgctggcgattgacgagctgatcgacgtgaagctgagcggggacgacgcggcgaagaccgcgcgtctgtctggcctgctgagccgcgtgctggaggagtctgaggacccagtcctgagcgagtccgcttcccacaccctgggccacctggtccgcagcggcggtgcgatgacctccgacatcgtggagaaggagatccgccgctctctggcttggtgcgacccacgcaacgagccaaacgagtcccgtcgcctgacggccctgctggtcctcactgaggccgccgagagcgccccagccgtctttaacgtgcacgtcaagagcttcattgacgccgtgtggtttcccctgcgcgatgctaagcagcacattcgcgaggcggccgtgcgtgccctgaaggcctgcctgtgcctggtcgagaagcgcgagactcgctaccgcgtgcagtggtactacaagctgcacgagcagacgatgcgcggcatgaagcgcgaccaccgcaccggcgccctgcccagcccagagtctatccacggcagcctgctggccctggctgagctgctgcagcacaccggcgagttcatgctggcgcgctacaaggaggtggtggagaacgtgttccgctacaaggattctaaggagaagaacatccgtcgcgccgtgatccacctgctgccccgcatggcggcgttctcccccgagcgcttcgcgtccgagtacctcgcgcgtgcgatcgctttcctgctgatcgtgctgaagaacccacctgagcgtggcgccgctttcgcggcgctcgccgacatggccgcggcgctggcccgtggctgcctcagccccatctacgtggccattcgtgaggcgctgtctgctccccctgcggctcgcgctgccgctcgcccccgtcccgcgacttgctacgaggccctgcagtgcgtgggcatgctggccgtggccctgggtcccctgtggcgcccttacgctgccgcgctggtggaggcgatggtgctgaccggcgtgtccgaggtgctggtgcaagcgctgacccaggtcgcgaacgccctccccgagctgctggaggacatccagtatcagctgctggacctgctgtccctggtcctgtccaagcgcccctttaacagcagcaccacgcagcctaagttcgcggcgctgtccgccgcgattgcggcgggtgagctgcagggcaacgccctgacgaagctcgccctgcaaaccctgggtactttcgacctgggcggcattcagctgctggagtttatgcgcgaccacatcctcgcctacaccgacgatcccgacaaggagatccgccaagccgcggtgctggccgcctgcccccgtgctggcgccgctcgcagcagcctgcgcgtgcgcagcctgcgtagcggctggcgccgtgccgcggcggccgtctggcacacccgcgtcgtggagcgctgcgtcggccgcctgctggtggtggcggtggccgacccctccgagcgcgtgcgcaaggaggtgctgcgcgctctggtggctaccacggccctggacgactacctcgcccaggctgactgcctgcgtgcgctgttcgtgggcatgaacgacgagagcgtggcggtccgcggcctggccatccgcctggtgggccgcctggccgagcgcaaccctgctcacgtcaaccccgcgctgcgcaagcacctgctgcagctcctgcacgacatggagttcagccctgacaaccgtgcccgtgaggagtccgcgtttctcctggaggtgctgatcaccgccgccgctcgcctgatcatgccctacgtgagccctatccagaaggccctggtgagcaagctgcgcggtgggagcggccctggcatcactgtgctctctactctgggcgcgctggccgaggtgagcggcaccaccttccgccccttcatttccgaggtgatgcctctcgtcatcgaggccatccaggacaactccgacgggcgtcgtcgcgtggtcgcggtgaagaccctgggcttcatcgtgtcttcctgcggcaacgtgatgggcccctatctggagtacccccagctgctgtccgtcctgctgcgcatgctgcacgaggggcaccctgcccagcgccgcgaggtgattaaggtgctgggcatcatcggcgcgctggacccccacacccacaagctcaaccaggcctccctctccggcgagggcaagctggagaaggagggcgtgcgccccctccgccacggtggtggcggcgctgggggcgcggggggtggcgctggtggcggcggcgtgggtggcggcgtggccggcgatagcaacgacggcggcatggggcctggcgatgacggcggtcctggcggggacctgctgccctccagcggcctggtgacgtcctccgaggactactaccccaccgtggccatcaacgcgctgatgcgcgtgctgcgcgaccccgctctcgcgagccagcacctggccgtgatccgcgccctggccgcgattttccgtgctctgcagctgtccgtggtgccctacctgccaaaggtcctccccatcctcctgggggtgctgcgcggtggcgacgaggcgctccgtgaggagatcctggccagcctgcgtgccctggtgggctacgtgcgtcagcacatgcgccgctttctgcccgacctgacgcagctggtgcacgagttctggcctgcggcgcctcgcacctgcctggctctgatcgccgacctgggcatggccctgcgcgacgacattcgtgctaagcccctgccacccctgcccctgctgcccccaagctctcctccccgcacgccccacaaccgccagtacgtgcccgagctgctccccaagttcgtggcggtgttttccgaggccgagcgtgccggcagctgggatctggtgcgcccagctctgggcgccctggagagcctggggagcgccgtggatgactctctgcacctgctcctgcccagcatggtgcgcctgattagcccagctgcgagctccactcccgcggaggtccgccgtgctgctctccgctctctccgccgcctcatcccccgcatgcagctgggcggctacgcgagcgccgtgctgcacccactgatcaaggtgctggacggccactccgacgagcagctgcgccgcgatgccctggacaccatctgcgcggtggccgtgtgcctgggccccgagtttgcgattttcgtgccaacgatccgcaaggtgcgcgtgcgccaccgcctccaccacgagtggtttgaccgcctcgccggcaaggtgtgcgctgtgagcccaccttgcatgagcgacgcggaggactgggagggggccggcggcgccgccagcggtgccggcagcgctggtgccgctggcgggtgggccgtggagatcgacctgctggcgcgcatgcaggccgagggtggtggggccctcggtggccagccacccgtcccacctgggcccgacggcggtccctccgccaagctgcccgtgaacgcggccgtcctccgccgtgcttgggagtccagccaccgtgtgaccaaggaggactgggccgagtggatgcgcaacttcgctgtcgagctgctgaaggagtctccctcccccgctctgcgcgcttgccacggcctggcgcaggtgcacccctccatggcccgcgagctgttcgctgccggcttcgtgtcttgctgggcggagctggagcagggcctgcaggagcagctggtgcgcagcctggaggcggcgctggcgagccctacgatcccacctgagacggtgacggcgctgctgaacctggccgagttcatggagcacgacgacaagcgcctgcccctggacacgcgcaccctgggcgccctggccgagaagtgccacgcctttgccaaggccctgcactacaaggagctggagttccagaccagcccccagagcgcgatcgaggctctgatccacattaacaaccagctgcgccagccagaggcggcggtgggcgtcctcgcctacgcgcagaagcacctgcacatggagctgaaggagggctggtacgagaagctgtgccgctgggacgaggccctggacgcttacgagcgccgcctcctgaaggaggcccctggcagcatggagtaccacaccgccctgctggggaagatgcgctgcctcgcgagcctggcggagtgggagaacctgagcaacctgtgccgtactgagtggcgtaagagcgagccccacgtgcgccgcgagatggcgctgatcgcggcccacgccgcgtggcacatgggcgcttgggacgagatggcgatgtacgtggacaccgtcgataaccccgaggcggtgggccccaactcccacacgcccaccggcgcctttctgcgcgcggtcctgtgcgtgcgcgccaaccaggtgagcggcgcccaggcgcacgtggagcgcacccgcgagctgatggtggcggacctggcggccctggtgggcgagtcctacgagcgcgcgtacacggacatggtgcgtgtgcaacagctggccgagctggaggaggtctgcgcgtacaagcaggccctcgaccgtcgcgcggctgaccctggcggcagcgaggcgcgcatcgggttcatccagcagctgtggcgtgaccgcctgcgcggcgtgcagcgccacgtggaggtgtggcagagcctcttcagcatccgcagcctggtcgtgcccatggcccaggacgtggattcttggctcaagtttgcgagcctgtgccgcaagagcggtcgcagccgccaagcctatcgcatgctgctgcagctgctgcgctacaaccccatgaacattacccaggccggcaaccctggctacggtgctggctctggcgcccctcacgtgatgctggctttcctcaagcacctgtggacccagggcaaccgcaccgaggcttacaaccgcatcaaggacctggcctccctcaacggccgcgcgtttctccgcctgggcatctggcagtgggcgatgaacgacctggacaaccccggtgtgatcgccgagaacctggcgtcctttcgtgccgccactgagcacgcccccaactgggctaaggcgtggcaccagtgggccctgttcaacgtggctgtgagcgctcactaccgctgcgaccccatgcgcgacgagaaccaggcggtgagccacgtccctccagccgtccagggctttttccgctccgtggccctgggccaagctgccggtgaccgcacgggtaacctgcaggacatcctgcgcctgctgactctctggttcaacttcggcgcgtacgctgaggtgcgcgctgccctgaccgagggcttccagctggtgagcattgacacttggctgctggtgatcccacagatcattgcgcgcatccacacgcacaacaccgacgtgcgccagctgatccaccacctgctggtgaagatcggccgccaccaccctcaggcgctgatgtaccccctgctggtcgcgaccaagagccagagcccagctcgccgccaggctgcgtatagcgtgctggagtgcatccgccagcactctgccgcgctggtcgagcaggcgcagctcgtgagcggcgagctgattcgcatggcgatcctgtggcacgagatgtggcacgagggcctggaggaggcttcccgcctgtattttggcgagagcaacgtggagggcatgctgaacaccctgctgcccctgcacgagatgctggagaaggctggtcccaccaccctgaaggagatcgcgttcgtgcagagctacgggcgcgagctctccgaggcctacgagtggctgatgaagtacaaggccagccgcaaggaggctgagctgcaccaggcctgggacctgtactaccacgtgttcaagcgcattaacaagcagctgcgctccctgaccaccctggagctgcagtacgtctccccagctctggtgcgcgcgcaggacctggagctggccgtgcccggcacgtacatcgccggggagcccctggtgacgattgccgccttcgcgccccagctccacgtgatcagctccaagcagcgtccccgcaagctgaccatccacggtggggacggcgccgagtacatgtttctgctgaagggccacgaggatctgcgccaggacgagcgcgtgatgcagctgttcggcctggtgaacactatgctggcgcacgaccgcatcaccgctgagcgtgatctgtccatcgcccgctacgccgtgatccccctgtctcctaacagcggcctgatcggctgggtcccaaactgcgacacgctccacgccctgatccgcgagtaccgcgaggctcgcaagatccctctgaactgggagcaccgcctgatgctcggcatggcgcctgactacgaccacctgactgtgatccagaaggtggaggtgttcgagtacgcgctggattccacgagcggtgaggacctgcacaaggtcctgtggctgaagtctcgcaacagcgaggtgtggctggaccgccgcaccaactacacccgcagcgctgcggtcatgagcatggtgggttacattctcggcctgggcgaccgccacccctccaacctcatgctggaccgctactccggcaagctgctgcacattgactttggcgactgcttcgaggcgagcatgaaccgcgagaagttccctgagaaggtgccctttcgtctgacgcgcatgatgatcaaggctatggaggtgagcggcatcgagggcaacttccgcaccacgtgcgagaacgtgatgcgtgtgctgcgcagcaacaaggagtccgtgaccgcgatgctggaggctttcgtccacgaccccctgatcaactggcgcctcctgaacaccactgaggctgcgaccgaggcggccctggcccgcaccgatggcggcgggggcgggggcggtcacatggatggtcctggcggtcaccccggtggccgcgacgccctgggtggcggcggtggcggtgccggcggtggcggtggcggcgacccaggcgccatgcccagccctccccgtcgtgagacgcgcgagaaggagctcaaggaggctttcgtgaacctcggcgacgccaacgaggtgctcaacacccgcgctgtggaggtcatgaagcgcatgagcgacaagctgatgggccgcgattacgctcccgagctgtgcgtcggtggtggctccggggcgtccgggatggagcctgactccgtgcccgcccaggtcggccgcctgatcaacatggcggtcaaccacgagaacctgtgccagtcttacatcggctggtgccccttttggaccggctaaggatcc

SEQ ID NO: 49 - DNA, 7464 bp, artificial sequence
  synthesized
ctcgagatgtctacgtcttctcagtcttttgtcgctggtcgtcctgcttctatggcctccccctcccagtcccaccgcttttgcggcccctccgccaccgcttctggcggcggtagcttcgacaccctgaaccgcgtgatcgcggacctgtgcagccgcggtaaccccaaggagggcgcgccactggctttccgcaagcacgtcgaggaggcggtgcgcgacctgtccggcgaggcgagcagccgcttcatggagcagctgtacgaccgcatcgccaacctgattgagtccaccgacgtggcggagaacatgggcgcgctgcgcgctatcgacgagctgacggagatcggcttcggcgagaacgccactaaggtgagccgcttcgcgggctacatgcgcactgtgttcgagctgaagcgcgaccccgagattctcgtcctggccagccgcgtgctggggcacctggctcgcgctgggggcgctatgacgagcgacgaggtggagttccagatgaagacggcgttcgactggctgcgcgtggaccgcgtggagtaccgccgctttgctgctgtgctgatcctcaaggagatggcggagaacgcgagcacggtcttcaacgtccacgtccccgagttcgtggacgccatctgggtggccctgcgcgacccacagctgcaggtgcgcgagcgcgccgtggaggccctgcgtgcctgcctgcgcgtgatcgagaagcgcgagacgcgctggcgcgtgcagtggtattaccgcatgttcgaggccactcaggacggcctgggtcgcaacgcccccgtccacagcatccacggcagcctcctggctgtcggcgagctgctgcgcaacaccggcgagttcatgatgagccgctaccgcgaggtggctgagatcgtcctgcgctatctggagcaccgcgaccgcctggtccgcctgtctatcacgtccctcctcccccgcattgcgcacttcctgcgcgaccgcttcgtcacgaactacctcaccatttgcatgaaccacatcctgactgtgctgcgcattccagccgagcgcgccagcgggttcattgctctcggcgagatggccggtgccctggacggcgagctgatccactacctccccaccatcatgagccacctgcgtgacgccattgcccctcgcaaggggcgccccctgctggaggcggtggcgtgtgtgggcaacattgccaaggccatggggagcacggtcgagactcacgtccgcgacctgctggacgtcatgttctccagcagcctgagctccactctcgtcgacgcgctcgaccagatcactatcagcatcccctccctgctgcccaccgtgcaggatcgcctgctcgattgtattagcctggtgctgtccaagagccactacagccaggccaagccccctgtgaccatcgtgcgcggcagcaccgtgggcatggcgccccagagcagcgacccctcttgcagcgcccaggtgcagctggcgctccagaccctggcgcgcttcaacttcaagggtcacgacctcctggagtttgcccgcgagtccgtcgtggtgtatctcgatgacgaggacgccgccacccgcaaggacgcggccctgtgctgctgccgcctgatcgcgaactccctcagcggcatcacccaattcgggagcagccgttccactcgcgcgggtggccgccgtcgccgcctcgtggaggagatcgtggagaagctgctgcgtactgccgtggccgacgccgacgtcaccgtgcgtaagtccattttcgtggcgctgttcggcaaccagtgctttgatgactacctggcgcaagccgactccctgactgccattttcgcgtctctgaacgacgaggacctggatgtgcgtgagtacgcgatcagcgtcgcgggccgcctgagcgagaagaaccccgcgtacgtgctgcccgccctgcgccgtcacctgatccagctgctgacctacctggagctgagcgcggacaacaagtgccgcgaggagagcgcgaagctgctgggctgcctggtgcgcaactgcgagcgtctgatcctgccctacgtggcccctgtccagaaggccctcgtggcgcgcctcagcgagggcacgggcgtcaacgctaacaacaacattgtgaccggcgtcctggtgactgtcggcgacctcgcgcgcgtgggcggcctggcgatgcgccagtatatccccgagctgatgcccctgattgtggaggccctcatggatggtgccgccgtggccaagcgcgaggtggcggtgtccaccctgggccaggtcgtgcagagcacgggctacgtggtgaccccatacaaggagtacccactgctgctgggtctgctgctcaagctgctgaagggtgacctggtgtggtctacccgtcgcgaggtgctgaaggtgctgggtattatgggtgcgctggaccctcacgtgcacaagcgcaaccagcagtccctgtccggctctcacggggaggtgccccgtggtacgggggacagcggccagcctatccctagcatcgacgagctccccgtggagctgcgccccagcttcgcgacctccgaggactactactctacggtggccattaacagcctcatgcgcatcctgcgcgacgcgagcctgctgtcctatcacaagcgcgtcgtgcgctctctgatgatcatcttcaagtctatgggcctggggtgcgtgccctatctgccaaaggtgctgcccgagctcttccacactgtgcgcaccagcgacgagaacctgaaggacttcatcacgtggggcctgggcacgctcgtgagcatcgtccgtcagcacatccgcaagtacctgcccgagctgctctccctggtgtccgagctgtggagctcctttaccctgccaggccccattcgcccctctcgcggcctgccagtgctgcacctgctggagcacctgtgcctggccctgaacgatgagttccgtacctacctgcctgtgatcctcccctgctttatccaggtcctgggcgatgccgagcgctttaacgattacacgtatgtgcccgacatcctgcacaccctggaggtgttcggtggcaccctggacgagcacatgcacctgctcctgcctgcgctgatccgcctgttcaaggtggatgcgcccgtggcgatccgccgcgacgccatcaagaccctgacccgcgtcattccctgtgtccaggtcactgggcacatctccgccctggtgcaccacctgaagctggtgctggacggcaagaacgacgagctgcgtaaggacgccgtggacgcgctgtgctgtctggcccacgccctcggcgaggatttcaccatcttcatcgagtccatccacaagctgctgctgaagcaccgcctgcgccacaaggagttcgaggagatccacgctcgctggcgccgccgtgagcccctgatcgtggcgaccacggccactcagcaactgtctcgccgcctgcccgtggaggtgatccgcgaccctgtgatcgagaacgagatcgaccccttcgaggagggcaccgaccgcaaccaccaagtgaacgacgggcgtctgcgcacggctggcgaggcctcccagcgctctaccaaggaggactgggaggagtggatgcgccacttctccatcgagctgctcaaggagtcccccagcccagcgctgcgcacctgcgcgaagctggcccagctgcagccctttgtgggccgcgagctgttcgccgcggggttcgtgagctgttgggcccagctgaacgagagcagccagaagcagctcgtccgcagcctggagatggcgttttccagccccaacatcccacccgagatcctggcgaccctgctcaacctcgcggagttcatggagcacgatgagaagcccctccccatcgacattcgcctgctgggcgccctggccgagaagtgtcgcgtcttcgccaaggctctgcactacaaggagatggagttcgagggtccccgtagcaagcgcatggacgcgaaccctgtggccgtggtggaggcgctcatccacatcaacaaccagctgcaccagcacgaggccgccgtgggcatcctgacctacgcgcagcagcacctggacgtgcagctgaaggagagctggtacgagaagctgcagcgctgggacgacgccctgaaggcgtacaccctgaaggcctcccagaccaccaacccccacctggtgctggaggctacgctgggccaaatgcgctgcctggccgccctggcccgctgggaggagctgaacaacctgtgcaaggagtactggtcccccgcggagccctccgctcgcctggagatggcgcccatggcggcccaggcggcttggaacatgggcgagtgggaccagatggcggagtacgtgagccgcctggacgacggcgacgagactaagctgcgtgggctggcgtcccccgtgtccagcggcgacggctcctccaacggcacctttttccgcgccgtgctgctggtgcgccgtgccaagtacgacgaggcgcgcgagtacgtggagcgcgctcgcaagtgtctggccaccgagctggccgcgctggtcctggagagctacgagcgcgcgtactccaacatggtccgcgtccagcagctctccgagctggaggaggtgatcgagtactacacgctgcccgtcggcaacaccatcgcggaggagcgtcgcgccctgattcgtaacatgtggacccagcgcatccagggctccaagcgcaacgtggaggtgtggcaggccctgctggctgtgcgcgctctggtgctgcctcccactgaggacgtggagacgtggctgaagttcgcttccctgtgccgcaagtccggccgcatcagccaggcgaagagcaccctcctgaagctgctgcccttcgaccctgaggtgagccccgagaacatgcagtaccacggcccaccccaggtgatgctgggctacctgaagtatcagtggtccctgggggaggagcgcaagcgcaaggaggccttcaccaagctgcagatcctgacgcgcgagctgagctccgtgccacactcccagagcgacatcctcgcgagcatggtgtccagcaagggggccaacgtgcctctcctggcgcgcgtgaacctcaagctgggcacctggcagtgggcgctgagctctggcctgaacgacggcagcattcaggagattcgcgacgccttcgacaagagcacctgctacgcgcccaagtgggccaaggcttggcacacgtgggcgctgtttaacacggctgtcatgtcccactacatttctcgcggccagatcgcttcccagtacgtggtgtccgccgtgaccggctacttttacagcatcgcctgcgcggccaacgccaagggcgtggacgatagcctgcaagacatcctgcgcctgctgaccctgtggttcaaccacggggccaccgcggacgtgcagaccgccctgaagacgggtttcagccacgtgaacattaacacctggctggtggtcctgccccagatcatcgctcgcatccacagcaacaaccgcgcggtgcgtgagctcattcagtccctgctgatccgcatcggcgagaaccacccccaggcgctgatgtaccccctgctggtggcgtgtaagagcatcagcaacctccgtcgcgccgctgctcaggaggtggtggataaggtgcgtcagcactccggcgcgctcgtggaccaggcgcagctggtgtcccacgagctcatccgcgtcgcgatcctgtggcacgagatgtggcacgaggcgctggaggaggcctcccgcctgtacttcggcgagcacaacatcgagggcatgctcaaggtgctggagcccctgcacgacatgctggacgagggcgtgaagaaggactctaccacgatccaggagcgcgcgttcatcgaggcgtaccgccacgagctgaaggaggcgcacgagtgctgctgcaactacaagatcaccggcaaggacgctgagctgacccaggcgtgggatctgtactaccacgtgttcaagcgcatcgacaagcagctggcgagcctgaccacgctggacctggagagcgtctcccccgagctgctgctgtgccgcgacctggagctcgccgtgcccggcacctaccgcgcggacgcccccgtggtgacgatcagctcctttagccgtcagctggtggtgatcacctctaagcagcgcccacgcaagctgacgattcacggcaacgacggcgaggactacgccttcctgctgaagggccacgaggatctgcgccaggacgagcgcgtgatgcagctgttcggcctggtgaacacgctcctggagaactcccgcaagaccgctgagaaggatctgtctatccagcgctacagcgtgatccccctgagccccaactccggcctgatcggctgggtgcccaactgcgacaccctgcaccacctcattcgcgagcaccgcgatgcgcgcaagattattctcaaccaggagaacaagcacatgctgagcttcgcgcccgactacgacaacctgcccctgatcgccaaggtggaggtcttcgagtacgcgctggagaacaccgagggtaacgatctgtctcgcgtgctgtggctcaagagccgcagcagcgaggtgtggctggagcgtcgcacgaactacacccgcagcctggctgtgatgtccatggtgggctacattctgggcctcggcgaccgccaccctagcaacctgatgctgcaccgctattccggcaagatcctgcacattgacttcggcgactgctttgaggcctccatgaaccgcgagaagttcccagagaaggtgcctttccgcctgacccgcatgctggtgaaggccatggaggtgagcgggatcgagggcaacttccgctccacgtgcgagaacgtgatgcaggtgctccgcaccaacaaggactccgtgatggccatgatggaggccttcgtgcacgaccccctgatcaactggcgcctgtttaacttcaacgaggtcccccagctggcgctgctgggcaacaacaacccaaacgctcccgcggacgtggagcccgacgaggaggacgaggaccctgcggacattgacctgccccaaccccagcgcagcacgcgtgagaaggagatcctgcaggccgtgaacatgctgggcgacgccaacgaggtcctgaacgagcgcgcggtcgtggtcatggcccgcatgagccacaagctgaccgggcgcgacttttcttccagcgcgatcccctccaacccaatcgcggatcacaacaacctgctcggcggcgacagccacgaggtcgagcacggcctgtccgtgaaggtgcaggtccagaagctgatcaaccaagccacctcccacgagaacctgtgccagaactacgtcggctggtgtcccttctggacgggctaaggatcc

SEQ ID NO: 50 - DNA, 1155 bp, artificial sequence
  synthesized
tctgatgatgagcgtgaggagaaggagctggatctgactagccctgaggtcgtgactaagtacaagtccgctgcggagatcgtgaacaaggccctgcagctggtgctgagcgagtgcaagcccaaggtgaagatcgtggacctgtgcgagaagggcgacgcctttatcaaggagcagacgggcaacatgtacaagaacgtgaagaagaagatcgagcgcggcgtggccttccccacttgtatcagcgtgaacaacactgtgtgccacttcagccccctggcttccgacgagacgatcgtggaggagggcgacattctgaagatcgacatgggctgccacatcgacggcttcatcgccgtggtcggccacacgcacgtgctgcacgagggccctgtgaccgggcgtgccgcggacgtgatcgccgcggccaacactgccgctgaggtggcgctccgcctggtgcgtcccggcaagaagaacagcgacgtgactgaggcgattcagaaggtggctgccgcctatgactgcaagatcgtggagggcgtgctgagccaccagatgaagcagttcgtgatcgacggtaacaaggtggtgctgtccgtcagcaacccagatacccgcgtggacgaggccgagttcgaggagaacgaggtgtacagcatcgacattgtgacctccacgggcgacgggaagcccaagctgctcgacgagaagcagacgaccatctacaagcgcgcggtggataagtcttacaacctgaagatgaaggccagccgcttcatcttctctgagatcaaccagaagttccctatcatgcccttcacggcccgcgacctggaggagaagcgcgctcgcctgggcctcgtggagtgcgtcaaccacgagctgctgcagccttaccccgtgctgcacgagaagcccggcgacctggtcgcgcacatcaagttcacggtcctgctcatgcccaacggctccgaccgcgtcacctcccacctgcaggagctgcagcccaccaagaccaccgagaacgagcccgagatcaaggcctggctggccctgcccaccaagacgaagaagaaggggggcggcaagaagaagaagggcaagaagggcgacaaggtggaggaggcgagccaggccgagcccatggagggg

SEQ ID NO: 51 - DNA, 1158 bp, artificial sequence
  synthesized
tctgatgacggtagcattgagcaccaagagcctaacctgtctgtgcccgaggtggtgaccaagtacaaggctgcggcggatatctgcaaccgcgcgctgctggcggtggtggaggctgcgaaggacggcgccaaggtggtggacctgtgccgcatgggcgaccagttcatcaacaaggagtgcgcgaacatctacaagggcaaggagatcgagaagggcgtggcgttcccaacctgcgtgtccgcgaactctattgtgggccacttttcccccaacagcgaggacgcgaccgcgctgaagaacggtgacgtggtcaagatcgatatgggctgtcacatcgacgggttcattgctacccaggcgaccaccatcgtggtgggcgacgcggccatcagcggcaaggccgcggacgtgatcgccgctgcgcgcacggcgttcgacgccgcggtccgcctgattcgccctggcaagcacattgcggatgtgagcgctcccctccagaaggtcgctgagtccttcggctgcaacctggtggagggcgtgatgagccacgagatgaagcagttcgtgatcgacggcagcaagtgcatcctgaacaagcccacgcccgaccaaaaggtcgaggacggcgagttcgaggagaacgaggtgtacgccgtcgacatcgtggtcagcagcggcgagggcaagccccgcgtcctcgacgagaaggagactaccgtgtacaagcgcgccctggaggtcacttaccagctgaagatgcaagccagccgcgccgtgtttagcctcgtcaacagcgcgttcgctaccatgccattcaccctgcgtgcgctgctggacgaggctgccgcccaaaagaccgagctgaaggcgagccagctgaagctcggcctggtggagtgcctgaaccacggcctgctgcacccttaccccgtcctgcacgagaagcccggcgaggtggtggcccaaattaagggcaccgtgctgctgatgcctaacggctctagcatcatcaccagcgccccccgccagacggtgaccaccgagaagaaggtggaggacaaggagatcctcgacctgctggcgacgcccatcagcgcgaagagcgccaagaagaagaagaacaaggacaaggctgcggagccagcggctgccaag

SEQ ID NO: 52 - DNA, 1167 bp, artificial sequence
  synthesized
gtgaaggaggataagcaaactgatggtgatcgttggcgtggtctggcgtacgacacctccgacgaccagcaggatattacgcgcggcaaggggatggtggattccgtgttccaggcgcccatgggcactggcacccaccacgccgtgctgagcagctacgagtacgtctcccagggcctccgtcagtacaacctggacaacatgatggacggcttctacatcgctcccgctttcatggataagctggtggtgcacatcacgaagaacttcctgacgctgcccaacatcaaggtgccactgatcctggggatctggggcggcaagggccagggcaagagcttccaatgcgagctggtgatggcgaagatgggcatcaaccccatcatgatgagcgcgggcgagctggagtccgggaacgccggcgagcccgcgaagctgatccgccagcgctaccgcgaggctgcggacctgatcaagaagggcaagatgtgctgcctgctgattaacgacctggacgcgggcgctgggcgcatgggcggcaccacgcagtacactgtgaacaaccagatggtgaacgcgacgctgatgaacatcgcggacaacccaacgaacgtgcagctgcccggtatgtataacaaggaggagaacgcccgcgtgcccatcatctgcaccggcaacgacttcagcaccctgtacgccccactgatccgcgacggccgcatggagaagttctactgggcgcccacccgcgaggaccgcatcggcatttgtaagggtattttccgcaccgacaagattaaggacgaggacatcgtgaccctcgtggaccaattccctggtcagtccatcgacttcttcggcgcgctgcgcgcccgcgtctacgacgacgaggtgcgtaagttcgtcgagtccctgggggtggagaacatcgggaagcgcctggtgaactcccgcgagggccctcctgtgttcgagcagcccgagatgacttacgagaagctgatggagtacggcaacatgctggtgatggagcaggagaacgtgaagcgcgtgcagctggctgagacttacctgtcccaggccgccctgggcgacgccaacgccgacgccatcggccgcggcaccttctacgggaagactgaggagaaggagccctccaagctggag

SEQ ID NO: 53 - DNA, 1419 bp, artificial sequence
  synthesized
gctgcggctgtgtccaccgtgggcgctatcaaccgcgcgccactgagcctgaacggcagcggctccggcgccgtgtccgcccctgccagcacctttctggggaagaaggtggtgaccgtcagccgctttgcccagagcaacaagaagagcaacggcagcttcaaggtgctggctgtcaaggaggacaagcagacggacggggaccgctggcgcggcctggcctatgacacctccgacgatcagcaggacatcacgcgcggtaagggcatggtggacagcgtgttccaggcgcctatgggcaccggcacgcaccacgcggtcctgtccagctacgagtacgtgagccagggcctgcgccagtacaacctggacaacatgatggatggcttctacatcgctcccgcgttcatggacaagctggtcgtgcacattaccaagaacttcctcacgctgcccaacattaaggtgcccctgatcctcggcatctggggcggcaagggtcagggcaagtccttccagtgcgagctggtgatggccaagatgggcattaaccctatcatgatgagcgccggcgagctggagagcggtaacgccggcgagcccgccaagctgatccgccaacgctaccgcgaggccgcggacctcatcaagaagggcaagatgtgctgcctgttcatcaacgacctggacgcgggcgccggccgcatgggcggcaccacgcagtacacggtgaacaaccagatggtgaacgccaccctgatgaacatcgccgataaccccacgaacgtccagctccccggcatgtataacaaggaggagaacgcgcgcgtccccatcatctgcactggcaacgacttcagcaccctgtacgctcccctcattcgcgacggccgcatggagaagttctactgggcccctacccgcgaggaccgcattggcgtgtgtaagggcatttttcgcaccgacaagatcaaggacgaggacatcgtgacgctggtggaccagttcccaggccagtccatcgatttttttggcgctctgcgtgcccgcgtctacgacgacgaggtccgcaagttcgtggagtccctgggcgtggagaagatcggcaagcgcctggtcaactcccgcgagggcccacctgtcttcgagcaacccgagatgacgtacgagaagctgatggagtacggcaacatgctggtgatggagcaggagaacgtcaagcgcgtgcaactggccgagacttacctgagccaggccgcgctgggggacgctaacgctgacgcgattgggcgcggcaccttttacggcaagggcgcgcagcaggtgaacctgcccgtgccagagggctgcaccgaccctgtggcggagaactttgaccctacggcgcgcagcgacgatggcacttgcgtctacaacttc

SEQ ID NO: 54 - DNA, 1248 bp, artificial sequence
  synthesized
gctgtaaaagaagataaacaaactgatggagatcgttggcgtggtttagcttatgatacttcagatgatcagcaagatataacacgtggaaaaggtatggttgactctgttttccaagcaccaatgggcacaggtacacatcatgctgttttatcatcttatgaatatgtatctcaaggtttacgtcaatacaatttagataatatgatggatggtttctacattgcaccagcatttatggataaattagtagttcatataactaaaaacttcttaacattaccaaacataaaagtaccattaatacttggtatttggggtggtaaaggccaaggtaaatcatttcaatgtgaattagtaatggctaaaatgggtataaatcctattatgatgagtgctggtgaattagaatctggtaacgcaggtgaaccagcaaaacttattcgtcaacgttatcgtgaagctgctgaccttattaaaaaaggtaaaatgtgttgccttttcattaatgatttagatgctggtgcaggtcgtatgggtggaacaacacaatatactgttaacaatcaaatggttaatgcaacacttatgaacattgcagataatcctacaaatgttcaattacctggtatgtataacaaagaagaaaatgctcgtgttcctataatttgtactggtaatgatttttctacattatatgctccattaattcgtgatggccgtatggaaaaattctactgggcaccaactcgtgaagaccgtattggtgtatgcaaaggtatttttcgtactgacaaaatcaaagatgaagacattgtaacattagtagatcaatttccaggtcaatcaattgactttttcggtgctttacgtgctcgtgtttatgatgatgaggttcgtaaatttgtagaatctttaggagttgagaaaattggtaaacgtttagtaaattcacgtgaaggacctccagtattcgaacaaccagaaatgacatacgaaaaattaatggaatatggtaatatgttagttatggaacaagaaaatgtaaaacgtgttcaattagctgaaacatatttatctcaagcagcattaggtgacgctaacgcagatgctatcggtcgtggtacattttatggtaaaggtgctcagcaagttaatttaccagttccagagggttgcactgatccagttgctgaaaactttgatccaacagctcgttcagatgatggcacttgtgtatataacttc

SEQ ID NO: 55 - DNA, 1221 bp, artificial sequence
  synthesized
caggtgactatgaagtcctccgccgtgtccggccagcgcgtgggcggtgcgcgtgtggcgacccgctccgtgcgtcgcgcccagctccaggtggtggccagcagccgcaagcagatggggcgctggcgctccatcgacgccggcgtggacgcctccgacgatcagcaggacattacccgtggccgcgagatggtggatgacctgtttcagggcggctttggcgccggggggacccacaacgccgtcctgagctcccaggagtacctgagccagagccgcgcctccttcaacaacatcgaggacggcttctacatctcccccgcgttcctggacaagatgactattcacatcgctaagaactttatggatctgcccaagatcaaggtccccctcattctgggcatctggggcggtaagggccagggcaagaccttccagtgcgccctggcgtacaagaagctgggcatcgcccccatcgtgatgtctgccggcgagctggagagcggcaacgcgggcgagcctgcgaagctgatccgcacgcgctaccgcgaggcctccgatattatcaagaagggtcgcatgtgctccctgtttatcaacgacctggatgctggtgccggccgtatgggcgataccacccagtacaccgtgaacaaccagatggtgaacgcgacgctgatgaacattgccgacaaccccaccaacgtgcagctgcccggcgtgtacaagaacgaggagattccccgcgtccctatcgtgtgcaccggcaacgacttcagcactctgtatgcgcccctgatccgcgacggccgcatggagaagtactactggaaccccacccgcgaggaccgcattggcgtctgcatggggatcttccaggaggacaacgtccagcgccgcgaggtcgagaacctcgtggacaccttccccgggcagagcatcgactttttcggtgccctccgtgcgcgcgtctacgacgatatggtccgccagtggatcacggacacgggcgtggacaagatcggccagcagctggtcaacgcccgccagaaggtcgccatgcccaaggtcagcatggacctcaacgtcctgatcaagtacggcaagtctctggtggacgagcaggagaacgtgaagcgcgtgcagctggccgacgcttacctgtccggcgccgagctggccggccacggcggctccagcctgcccgaggcctattcccgc

SEQ ID NO: 56 - DNA, 1125 bp, artificial sequence
  synthesized
gcttcaagtcgtaaacagatgggtcgttggcgtagtattgatgctggcgtagatgcttcagatgatcaacaagatattacaagaggtcgtgaaatggtagatgacttatttcagggtggctttggtgctggtggtactcacaatgctgttttatcttcacaagaatatttatctcaatctcgtgctagtttcaataatatcgaagatggcttctacatttctcctgcatttttagacaaaatgactattcatattgctaaaaacttcatggatttacctaaaatcaaagtacctcttattttaggtatatggggtggtaaaggtcaaggtaaaacttttcaatgcgcattagcttacaaaaaacttggtattgcacctattgttatgtcagctggtgaattagaaagtggtaatgcaggtgaaccagcaaaattaatccgtacacgttatagagaagcttctgacattattaaaaaaggacgtatgtgctctttattcattaatgatttagatgctggcgctggccgtatgggtgatacaactcaatatacagtaaataaccaaatggttaatgctacattaatgaacatagctgataaccctacaaatgttcaattaccaggtgtttacaaaaatgaagaaatacctcgtgtaccaatcgtttgcacaggtaatgacttttcaacattatacgcaccattaattcgtgatggtcgtatggagaaatactattggaatcctacacgtgaagatcgtattggagtatgtatgggaatatttcaagaagataacgtacaacgtcgtgaggttgaaaacttagttgatacatttcctggacaatcaatcgatttttttggtgctcttcgtgctcgtgtatatgacgatatggttcgtcaatggatcactgatactggtgtagacaaaattggtcaacaattagtaaatgctcgtcaaaaagtagctatgccaaaagtatcaatggacttaaacgtacttatcaaatatggtaaatctttagtagatgaacaagaaaatgttaaacgtgtacaattagctgatgcttatttatctggcgcagaattagctggtcacggtggttcatctttaccagaagcatattcacgt

SEQ ID NO: 57 - DNA, 7563 bp, artificial sequence
  synthesized
ctctctggcgtcggcccagtgccaaccaagcccgcgtttaaggctggcggcgacacgctgagccgccacctggaggagctgtgccgcagcggcgcgtgggagcgccgccacaaggacggcgacaaggcgctgctggagtacatcgaggcggaggcgcgcgacctgtccgtcgaggcgttcggtcgcctgatgaccgacgtgtaccagcgtatcggcaacatgctgctgaagggcaacgacatcacgcgccgcatgggcggggtgctggcgattgacgagctgatcgacgtgaagctgagcggggacgacgcggcgaagaccgcgcgtctgtctggcctgctgagccgcgtgctggaggagtctgaggacccagtcctgagcgagtccgcttcccacaccctgggccacctggtccgcagcggcggtgcgatgacctccgacatcgtggagaaggagatccgccgctctctggcttggtgcgacccacgcaacgagccaaacgagtcccgtcgcctgacggccctgctggtcctcactgaggccgccgagagcgccccagccgtctttaacgtgcacgtcaagagcttcattgacgccgtgtggtttcccctgcgcgatgctaagcagcacattcgcgaggcggccgtgcgtgccctgaaggcctgcctgtgcctggtcgagaagcgcgagactcgctaccgcgtgcagtggtactacaagctgcacgagcagacgatgcgcggcatgaagcgcgaccaccgcaccggcgccctgcccagcccagagtctatccacggcagcctgctggccctggctgagctgctgcagcacaccggcgagttcatgctggcgcgctacaaggaggtggtggagaacgtgttccgctacaaggattctaaggagaagaacatccgtcgcgccgtgatccacctgctgccccgcatggcggcgttctcccccgagcgcttcgcgtccgagtacctcgcgcgtgcgatcgctttcctgctgatcgtgctgaagaacccacctgagcgtggcgccgctttcgcggcgctcgccgacatggccgcggcgctggcccgtggctgcctcagccccatctacgtggccattcgtgaggcgctgtctgctccccctgcggctcgcgctgccgctcgcccccgtcccgcgacttgctacgaggccctgcagtgcgtgggcatgctggccgtggccctgggtcccctgtggcgcccttacgctgccgcgctggtggaggcgatggtgctgaccggcgtgtccgaggtgctggtgcaagcgctgacccaggtcgcgaacgccctccccgagctgctggaggacatccagtatcagctgctggacctgctgtccctggtcctgtccaagcgcccctttaacagcagcaccacgcagcctaagttcgcggcgctgtccgccgcgattgcggcgggtgagctgcagggcaacgccctgacgaagctcgccctgcaaaccctgggtactttcgacctgggcggcattcagctgctggagtttatgcgcgaccacatcctcgcctacaccgacgatcccgacaaggagatccgccaagccgcggtgctggccgcctgcccccgtgctggcgccgctcgcagcagcctgcgcgtgcgcagcctgcgtagcggctggcgccgtgccgcggcggccgtctggcacacccgcgtcgtggagcgctgcgtcggccgcctgctggtggtggcggtggccgacccctccgagcgcgtgcgcaaggaggtgctgcgcgctctggtggctaccacggccctggacgactacctcgcccaggctgactgcctgcgtgcgctgttcgtgggcatgaacgacgagagcgtggcggtccgcggcctggccatccgcctggtgggccgcctggccgagcgcaaccctgctcacgtcaaccccgcgctgcgcaagcacctgctgcagctcctgcacgacatggagttcagccctgacaaccgtgcccgtgaggagtccgcgtttctcctggaggtgctgatcaccgccgccgctcgcctgatcatgccctacgtgagccctatccagaaggccctggtgagcaagctgcgcggtgggagcggccctggcatcactgtgctctctactctgggcgcgctggccgaggtgagcggcaccaccttccgccccttcatttccgaggtgatgcctctcgtcatcgaggccatccaggacaactccgacgggcgtcgtcgcgtggtcgcggtgaagaccctgggcttcatcgtgtcttcctgcggcaacgtgatgggcccctatctggagtacccccagctgctgtccgtcctgctgcgcatgctgcacgaggggcaccctgcccagcgccgcgaggtgattaaggtgctgggcatcatcggcgcgctggacccccacacccacaagctcaaccaggcctccctctccggcgagggcaagctggagaaggagggcgtgcgccccctccgccacggtggtggcggcgctgggggcgcggggggtggcgctggtggcggcggcgtgggtggcggcgtggccggcgatagcaacgacggcggcatggggcctggcgatgacggcggtcctggcggggacctgctgccctccagcggcctggtgacgtcctccgaggactactaccccaccgtggccatcaacgcgctgatgcgcgtgctgcgcgaccccgctctcgcgagccagcacctggccgtgatccgcgccctggccgcgattttccgtgctctgcagctgtccgtggtgccctacctgccaaaggtcctccccatcctcctgggggtgctgcgcggtggcgacgaggcgctccgtgaggagatcctggccagcctgcgtgccctggtgggctacgtgcgtcagcacatgcgccgctttctgcccgacctgacgcagctggtgcacgagttctggcctgcggcgcctcgcacctgcctggctctgatcgccgacctgggcatggccctgcgcgacgacattcgtgctaagcccctgccacccctgcccctgctgcccccaagctctcctccccgcacgccccacaaccgccagtacgtgcccgagctgctccccaagttcgtggcggtgttttccgaggccgagcgtgccggcagctgggatctggtgcgcccagctctgggcgccctggagagcctggggagcgccgtggatgactctctgcacctgctcctgcccagcatggtgcgcctgattagcccagctgcgagctccactcccgcggaggtccgccgtgctgctctccgctctctccgccgcctcatcccccgcatgcagctgggcggctacgcgagcgccgtgctgcacccactgatcaaggtgctggacggccactccgacgagcagctgcgccgcgatgccctggacaccatctgcgcggtggccgtgtgcctgggccccgagtttgcgattttcgtgccaacgatccgcaaggtgcgcgtgcgccaccgcctccaccacgagtggtttgaccgcctcgccggcaaggtgtgcgctgtgagcccaccttgcatgagcgacgcggaggactgggagggggccggcggcgccgccagcggtgccggcagcgctggtgccgctggcgggtgggccgtggagatcgacctgctggcgcgcatgcaggccgagggtggtggggccctcggtggccagccacccgtcccacctgggcccgacggcggtccctccgccaagctgcccgtgaacgcggccgtcctccgccgtgcttgggagtccagccaccgtgtgaccaaggaggactgggccgagtggatgcgcaacttcgctgtcgagctgctgaaggagtctccctcccccgctctgcgcgcttgccacggcctggcgcaggtgcacccctccatggcccgcgagctgttcgctgccggcttcgtgtcttgctgggcggagctggagcagggcctgcaggagcagctggtgcgcagcctggaggcggcgctggcgagccctacgatcccacctgagacggtgacggcgctgctgaacctggccgagttcatggagcacgacgacaagcgcctgcccctggacacgcgcaccctgggcgccctggccgagaagtgccacgcctttgccaaggccctgcactacaaggagctggagttccagaccagcccccagagcgcgatcgaggctctgatccacattaacaaccagctgcgccagccagaggcggcggtgggcgtcctcgcctacgcgcagaagcacctgcacatggagctgaaggagggctggtacgagaagctgtgccgctgggacgaggccctggacgcttacgagcgccgcctcctgaaggaggcccctggcagcatggagtaccacaccgccctgctggggaagatgcgctgcctcgcgagcctggcggagtgggagaacctgagcaacctgtgccgtactgagtggcgtaagagcgagccccacgtgcgccgcgagatggcgctgatcgcggcccacgccgcgtggcacatgggcgcttgggacgagatggcgatgtacgtggacaccgtcgataaccccgaggcggtgggccccaactcccacacgcccaccggcgcctttctgcgcgcggtcctgtgcgtgcgcgccaaccaggtgagcggcgcccaggcgcacgtggagcgcacccgcgagctgatggtggcggacctggcggccctggtgggcgagtcctacgagcgcgcgtacacggacatggtgcgtgtgcaacagctggccgagctggaggaggtctgcgcgtacaagcaggccctcgaccgtcgcgcggctgaccctggcggcagcgaggcgcgcatcgggttcatccagcagctgtggcgtgaccgcctgcgcggcgtgcagcgccacgtggaggtgtggcagagcctcttcagcatccgcagcctggtcgtgcccatggcccaggacgtggattcttggctcaagtttgcgagcctgtgccgcaagagcggtcgcagccgccaagcctatcgcatgctgctgcagctgctgcgctacaaccccatgaacattacccaggccggcaaccctggctacggtgctggctctggcgcccctcacgtgatgctggctttcctcaagcacctgtggacccagggcaaccgcaccgaggcttacaaccgcatcaaggacctggcctccctcaacggccgcgcgtttctccgcctgggcatctggcagtgggcgatgaacgacctggacaaccccggtgtgatcgccgagaacctggcgtcctttcgtgccgccactgagcacgcccccaactgggctaaggcgtggcaccagtgggccctgttcaacgtggctgtgagcgctcactaccgctgcgaccccatgcgcgacgagaaccaggcggtgagccacgtccctccagccgtccagggctttttccgctccgtggccctgggccaagctgccggtgaccgcacgggtaacctgcaggacatcctgcgcctgctgactctctggttcaacttcggcgcgtacgctgaggtgcgcgctgccctgaccgagggcttccagctggtgagcattgacacttggctgctggtgatcccacagatcattgcgcgcatccacacgcacaacaccgacgtgcgccagctgatccaccacctgctggtgaagatcggccgccaccaccctcaggcgctgatgtaccccctgctggtcgcgaccaagagccagagcccagctcgccgccaggctgcgtatagcgtgctggagtgcatccgccagcactctgccgcgctggtcgagcaggcgcagctcgtgagcggcgagctgattcgcatggcgatcctgtggcacgagatgtggcacgagggcctggaggaggcttcccgcctgtattttggcgagagcaacgtggagggcatgctgaacaccctgctgcccctgcacgagatgctggagaaggctggtcccaccaccctgaaggagatcgcgttcgtgcagagctacgggcgcgagctctccgaggcctacgagtggctgatgaagtacaaggccagccgcaaggaggctgagctgcaccaggcctgggacctgtactaccacgtgttcaagcgcattaacaagcagctgcgctccctgaccaccctggagctgcagtacgtctccccagctctggtgcgcgcgcaggacctggagctggccgtgcccggcacgtacatcgccggggagcccctggtgacgattgccgccttcgcgccccagctccacgtgatcagctccaagcagcgtccccgcaagctgaccatccacggtggggacggcgccgagtacatgtttctgctgaagggccacgaggatctgcgccaggacgagcgcgtgatgcagctgttcggcctggtgaacactatgctggcgcacgaccgcatcaccgctgagcgtgatctgtccatcgcccgctacgccgtgatccccctgtctcctaacagcggcctgatcggctgggtcccaaactgcgacacgctccacgccctgatccgcgagtaccgcgaggctcgcaagatccctctgaactgggagcaccgcctgatgctcggcatggcgcctgactacgaccacctgactgtgatccagaaggtggaggtgttcgagtacgcgctggattccacgagcggtgaggacctgcacaaggtcctgtggctgaagtctcgcaacagcgaggtgtggctggaccgccgcaccaactacacccgcagcgctgcggtcatgagcatggtgggttacattctcggcctgggcgaccgccacccctccaacctcatgctggaccgctactccggcaagctgctgcacattgactttggcgactgcttcgaggcgagcatgaaccgcgagaagttccctgagaaggtgccctttcgtctgacgcgcatgatgatcaaggctatggaggtgagcggcatcgagggcaacttccgcaccacgtgcgagaacgtgatgcgtgtgctgcgcagcaacaaggagtccgtgaccgcgatgctggaggctttcgtccacgaccccctgatcaactggcgcctcctgaacaccactgaggctgcgaccgaggcggccctggcccgcaccgatggcggcgggggcgggggcggtcacatggatggtcctggcggtcaccccggtggccgcgacgccctgggtggcggcggtggcggtgccggcggtggcggtggcggcgacccaggcgccatgcccagccctccccgtcgtgagacgcgcgagaaggagctcaaggaggctttcgtgaacctcggcgacgccaacgaggtgctcaacacccgcgctgtggaggtcatgaagcgcatgagcgacaagctgatgggccgcgattacgctcccgagctgtgcgtcggtggtggctccggggcgtccgggatggagcctgactccgtgcccgcccaggtcggccgcctgatcaacatggcggtcaaccacgagaacctgtgccagtcttacatcggctggtgccccttttgg

SEQ ID NO: 58 - DNA, 7440 bp, artificial sequence
  synthesized
tctacgtcttctcagtcttttgtcgctggtcgtcctgcttctatggcctccccctcccagtcccaccgcttttgcggcccctccgccaccgcttctggcggcggtagcttcgacaccctgaaccgcgtgatcgcggacctgtgcagccgcggtaaccccaaggagggcgcgccactggctttccgcaagcacgtcgaggaggcggtgcgcgacctgtccggcgaggcgagcagccgcttcatggagcagctgtacgaccgcatcgccaacctgattgagtccaccgacgtggcggagaacatgggcgcgctgcgcgctatcgacgagctgacggagatcggcttcggcgagaacgccactaaggtgagccgcttcgcgggctacatgcgcactgtgttcgagctgaagcgcgaccccgagattctcgtcctggccagccgcgtgctggggcacctggctcgcgctgggggcgctatgacgagcgacgaggtggagttccagatgaagacggcgttcgactggctgcgcgtggaccgcgtggagtaccgccgctttgctgctgtgctgatcctcaaggagatggcggagaacgcgagcacggtcttcaacgtccacgtccccgagttcgtggacgccatctgggtggccctgcgcgacccacagctgcaggtgcgcgagcgcgccgtggaggccctgcgtgcctgcctgcgcgtgatcgagaagcgcgagacgcgctggcgcgtgcagtggtattaccgcatgttcgaggccactcaggacggcctgggtcgcaacgcccccgtccacagcatccacggcagcctcctggctgtcggcgagctgctgcgcaacaccggcgagttcatgatgagccgctaccgcgaggtggctgagatcgtcctgcgctatctggagcaccgcgaccgcctggtccgcctgtctatcacgtccctcctcccccgcattgcgcacttcctgcgcgaccgcttcgtcacgaactacctcaccatttgcatgaaccacatcctgactgtgctgcgcattccagccgagcgcgccagcgggttcattgctctcggcgagatggccggtgccctggacggcgagctgatccactacctccccaccatcatgagccacctgcgtgacgccattgcccctcgcaaggggcgccccctgctggaggcggtggcgtgtgtgggcaacattgccaaggccatggggagcacggtcgagactcacgtccgcgacctgctggacgtcatgttctccagcagcctgagctccactctcgtcgacgcgctcgaccagatcactatcagcatcccctccctgctgcccaccgtgcaggatcgcctgctcgattgtattagcctggtgctgtccaagagccactacagccaggccaagccccctgtgaccatcgtgcgcggcagcaccgtgggcatggcgccccagagcagcgacccctcttgcagcgcccaggtgcagctggcgctccagaccctggcgcgcttcaacttcaagggtcacgacctcctggagtttgcccgcgagtccgtcgtggtgtatctcgatgacgaggacgccgccacccgcaaggacgcggccctgtgctgctgccgcctgatcgcgaactccctcagcggcatcacccaattcgggagcagccgttccactcgcgcgggtggccgccgtcgccgcctcgtggaggagatcgtggagaagctgctgcgtactgccgtggccgacgccgacgtcaccgtgcgtaagtccattttcgtggcgctgttcggcaaccagtgctttgatgactacctggcgcaagccgactccctgactgccattttcgcgtctctgaacgacgaggacctggatgtgcgtgagtacgcgatcagcgtcgcgggccgcctgagcgagaagaaccccgcgtacgtgctgcccgccctgcgccgtcacctgatccagctgctgacctacctggagctgagcgcggacaacaagtgccgcgaggagagcgcgaagctgctgggctgcctggtgcgcaactgcgagcgtctgatcctgccctacgtggcccctgtccagaaggccctcgtggcgcgcctcagcgagggcacgggcgtcaacgctaacaacaacattgtgaccggcgtcctggtgactgtcggcgacctcgcgcgcgtgggcggcctggcgatgcgccagtatatccccgagctgatgcccctgattgtggaggccctcatggatggtgccgccgtggccaagcgcgaggtggcggtgtccaccctgggccaggtcgtgcagagcacgggctacgtggtgaccccatacaaggagtacccactgctgctgggtctgctgctcaagctgctgaagggtgacctggtgtggtctacccgtcgcgaggtgctgaaggtgctgggtattatgggtgcgctggaccctcacgtgcacaagcgcaaccagcagtccctgtccggctctcacggggaggtgccccgtggtacgggggacagcggccagcctatccctagcatcgacgagctccccgtggagctgcgccccagcttcgcgacctccgaggactactactctacggtggccattaacagcctcatgcgcatcctgcgcgacgcgagcctgctgtcctatcacaagcgcgtcgtgcgctctctgatgatcatcttcaagtctatgggcctggggtgcgtgccctatctgccaaaggtgctgcccgagctcttccacactgtgcgcaccagcgacgagaacctgaaggacttcatcacgtggggcctgggcacgctcgtgagcatcgtccgtcagcacatccgcaagtacctgcccgagctgctctccctggtgtccgagctgtggagctcctttaccctgccaggccccattcgcccctctcgcggcctgccagtgctgcacctgctggagcacctgtgcctggccctgaacgatgagttccgtacctacctgcctgtgatcctcccctgctttatccaggtcctgggcgatgccgagcgctttaacgattacacgtatgtgcccgacatcctgcacaccctggaggtgttcggtggcaccctggacgagcacatgcacctgctcctgcctgcgctgatccgcctgttcaaggtggatgcgcccgtggcgatccgccgcgacgccatcaagaccctgacccgcgtcattccctgtgtccaggtcactgggcacatctccgccctggtgcaccacctgaagctggtgctggacggcaagaacgacgagctgcgtaaggacgccgtggacgcgctgtgctgtctggcccacgccctcggcgaggatttcaccatcttcatcgagtccatccacaagctgctgctgaagcaccgcctgcgccacaaggagttcgaggagatccacgctcgctggcgccgccgtgagcccctgatcgtggcgaccacggccactcagcaactgtctcgccgcctgcccgtggaggtgatccgcgaccctgtgatcgagaacgagatcgaccccttcgaggagggcaccgaccgcaaccaccaagtgaacgacgggcgtctgcgcacggctggcgaggcctcccagcgctctaccaaggaggactgggaggagtggatgcgccacttctccatcgagctgctcaaggagtcccccagcccagcgctgcgcacctgcgcgaagctggcccagctgcagccctttgtgggccgcgagctgttcgccgcggggttcgtgagctgttgggcccagctgaacgagagcagccagaagcagctcgtccgcagcctggagatggcgttttccagccccaacatcccacccgagatcctggcgaccctgctcaacctcgcggagttcatggagcacgatgagaagcccctccccatcgacattcgcctgctgggcgccctggccgagaagtgtcgcgtcttcgccaaggctctgcactacaaggagatggagttcgagggtccccgtagcaagcgcatggacgcgaaccctgtggccgtggtggaggcgctcatccacatcaacaaccagctgcaccagcacgaggccgccgtgggcatcctgacctacgcgcagcagcacctggacgtgcagctgaaggagagctggtacgagaagctgcagcgctgggacgacgccctgaaggcgtacaccctgaaggcctcccagaccaccaacccccacctggtgctggaggctacgctgggccaaatgcgctgcctggccgccctggcccgctgggaggagctgaacaacctgtgcaaggagtactggtcccccgcggagccctccgctcgcctggagatggcgcccatggcggcccaggcggcttggaacatgggcgagtgggaccagatggcggagtacgtgagccgcctggacgacggcgacgagactaagctgcgtgggctggcgtcccccgtgtccagcggcgacggctcctccaacggcacctttttccgcgccgtgctgctggtgcgccgtgccaagtacgacgaggcgcgcgagtacgtggagcgcgctcgcaagtgtctggccaccgagctggccgcgctggtcctggagagctacgagcgcgcgtactccaacatggtccgcgtccagcagctctccgagctggaggaggtgatcgagtactacacgctgcccgtcggcaacaccatcgcggaggagcgtcgcgccctgattcgtaacatgtggacccagcgcatccagggctccaagcgcaacgtggaggtgtggcaggccctgctggctgtgcgcgctctggtgctgcctcccactgaggacgtggagacgtggctgaagttcgcttccctgtgccgcaagtccggccgcatcagccaggcgaagagcaccctcctgaagctgctgcccttcgaccctgaggtgagccccgagaacatgcagtaccacggcccaccccaggtgatgctgggctacctgaagtatcagtggtccctgggggaggagcgcaagcgcaaggaggccttcaccaagctgcagatcctgacgcgcgagctgagctccgtgccacactcccagagcgacatcctcgcgagcatggtgtccagcaagggggccaacgtgcctctcctggcgcgcgtgaacctcaagctgggcacctggcagtgggcgctgagctctggcctgaacgacggcagcattcaggagattcgcgacgccttcgacaagagcacctgctacgcgcccaagtgggccaaggcttggcacacgtgggcgctgtttaacacggctgtcatgtcccactacatttctcgcggccagatcgcttcccagtacgtggtgtccgccgtgaccggctacttttacagcatcgcctgcgcggccaacgccaagggcgtggacgatagcctgcaagacatcctgcgcctgctgaccctgtggttcaaccacggggccaccgcggacgtgcagaccgccctgaagacgggtttcagccacgtgaacattaacacctggctggtggtcctgccccagatcatcgctcgcatccacagcaacaaccgcgcggtgcgtgagctcattcagtccctgctgatccgcatcggcgagaaccacccccaggcgctgatgtaccccctgctggtggcgtgtaagagcatcagcaacctccgtcgcgccgctgctcaggaggtggtggataaggtgcgtcagcactccggcgcgctcgtggaccaggcgcagctggtgtcccacgagctcatccgcgtcgcgatcctgtggcacgagatgtggcacgaggcgctggaggaggcctcccgcctgtacttcggcgagcacaacatcgagggcatgctcaaggtgctggagcccctgcacgacatgctggacgagggcgtgaagaaggactctaccacgatccaggagcgcgcgttcatcgaggcgtaccgccacgagctgaaggaggcgcacgagtgctgctgcaactacaagatcaccggcaaggacgctgagctgacccaggcgtgggatctgtactaccacgtgttcaagcgcatcgacaagcagctggcgagcctgaccacgctggacctggagagcgtctcccccgagctgctgctgtgccgcgacctggagctcgccgtgcccggcacctaccgcgcggacgcccccgtggtgacgatcagctcctttagccgtcagctggtggtgatcacctctaagcagcgcccacgcaagctgacgattcacggcaacgacggcgaggactacgccttcctgctgaagggccacgaggatctgcgccaggacgagcgcgtgatgcagctgttcggcctggtgaacacgctcctggagaactcccgcaagaccgctgagaaggatctgtctatccagcgctacagcgtgatccccctgagccccaactccggcctgatcggctgggtgcccaactgcgacaccctgcaccacctcattcgcgagcaccgcgatgcgcgcaagattattctcaaccaggagaacaagcacatgctgagcttcgcgcccgactacgacaacctgcccctgatcgccaaggtggaggtcttcgagtacgcgctggagaacaccgagggtaacgatctgtctcgcgtgctgtggctcaagagccgcagcagcgaggtgtggctggagcgtcgcacgaactacacccgcagcctggctgtgatgtccatggtgggctacattctgggcctcggcgaccgccaccctagcaacctgatgctgcaccgctattccggcaagatcctgcacattgacttcggcgactgctttgaggcctccatgaaccgcgagaagttcccagagaaggtgcctttccgcctgacccgcatgctggtgaaggccatggaggtgagcgggatcgagggcaacttccgctccacgtgcgagaacgtgatgcaggtgctccgcaccaacaaggactccgtgatggccatgatggaggccttcgtgcacgaccccctgatcaactggcgcctgtttaacttcaacgaggtcccccagctggcgctgctgggcaacaacaacccaaacgctcccgcggacgtggagcccgacgaggaggacgaggaccctgcggacattgacctgccccaaccccagcgcagcacgcgtgagaaggagatcctgcaggccgtgaacatgctgggcgacgccaacgaggtcctgaacgagcgcgcggtcgtggtcatggcccgcatgagccacaagctgaccgggcgcgacttttcttccagcgcgatcccctccaacccaatcgcggatcacaacaacctgctcggcggcgacagccacgaggtcgagcacggcctgtccgtgaaggtgcaggtccagaagctgatcaaccaagccacctcccacgagaacctgtgccagaactacgtcggctggtgtcccttctgg

SEQ ID NO: 59 - DNA, 1161 bp, artificial sequence
  synthesized
atgtcggatgatgagcgtgaggagaaggagctggatctgactagccctgaggtggtgacgaagtacaagtccgccgccgagatcgtgaacaaggccctccagctggtgctgtcggagtgcaagccaaaggtgaagatcgtggacctgtgcgagaagggcgatgccttcatcaaggagcagaccgggaacatgtacaagaacgtgaagaagaagatcgagcggggcgtggccttcccgacttgtatctccgtgaacaacaccgtgtgccacttcagccctctggcgagcgacgagacgatcgtggaggagggcgacattctgaagatcgacatgggttgccacatcgacggtttcatcgcggtcgtgggtcacacccacgtgctgcacgagggcccggtcacgggccgcgccgctgacgtgatcgccgctgcgaacacggctgcggaggtggcgctgcgcctggtgcgtcccggcaagaagaactcggacgtgaccgaggccatccagaaggtcgcggctgcctacgactgcaagatcgtggagggcgtgctctcgcaccagatgaagcaattcgtgatcgacggcaacaaggtggtgctgagcgtgagcaaccccgacacccgcgtggacgaggccgagttcgaggagaacgaggtgtacagcatcgacattgtgacgagcacgggcgatggcaagcccaagctcctggacgagaagcagacaaccatctacaagcgggccgtggacaagagctacaacctgaagatgaaggcgagccgcttcattttctcggagatcaaccagaagttccccatcatgccattcaccgctcgggacctggaggagaagcgtgcccgtctgggcctggtcgagtgcgtgaaccatgagctcctgcaaccctacccggtcctgcacgagaagccgggcgacctggtggctcacattaagtttactgtgctgctgatgcccaacggcagcgaccgtgtgacatcgcacgygctgcaagagctgcaacccacgaagacgacggagaacgagcccgagatcaaggcgtggctggcgctccctacgaagactaagaagaagggcggtgggaagaagaagaagggcaagaagggcgacaaggtggaggaggcgtcgcaggccgagccgatggagggc

SEQ ID NO: 60 - DNA, 1182 bp, artificial sequence
  synthesized
ctcgagatgtctgatgatgagcgtgaggagaaggagctggatctgactagccctgaggtcgtgactaagtacaagtccgctgcggagatcgtgaacaaggccctgcagctggtgctgagcgagtgcaagcccaaggtgaagatcgtggacctgtgcgagaagggcgacgcctttatcaaggagcagacgggcaacatgtacaagaacgtgaagaagaagatcgagcgcggcgtggccttccccacttgtatcagcgtgaacaacactgtgtgccacttcagccccctggcttccgacgagacgatcgtggaggagggcgacattctgaagatcgacatgggctgccacatcgacggcttcatcgccgtggtcggccacacgcacgtgctgcacgagggccctgtgaccgggcgtgccgcggacgtgatcgccgcggccaacactgccgctgaggtggcgctccgcctggtgcgtcccggcaagaagaacagcgacgtgactgaggcgattcagaaggtggctgccgcctatgactgcaagatcgtggagggcgtgctgagccaccagatgaagcagttcgtgatcgacggtaacaaggtggtgctgtccgtcagcaacccagatacccgcgtggacgaggccgagttcgaggagaacgaggtgtacagcatcgacattgtgacctccacgggcgacgggaagcccaagctgctcgacgagaagcagacgaccatctacaagcgcgcggtggataagtcttacaacctgaagatgaaggccagccgcttcatcttctctgagatcaaccagaagttccctatcatgcccttcacggcccgcgacctggaggagaagcgcgctcgcctgggcctcgtggagtgcgtcaaccacgagctgctgcagccttaccccgtgctgcacgagaagcccggcgacctggtcgcgcacatcaagttcacggtcctgctcatgcccaacggctccgaccgcgtcacctcccacgygctgcaggagctgcagcccaccaagaccaccgagaacgagcccgagatcaaggcctggctggccctgcccaccaagacgaagaagaaggggggcggcaagaagaagaagggcaagaagggcgacaaggtggaggaggcgagccaggccgagcccatggaggggacgggctaaggatcc

SEQ ID NO: 61 - DNA, 1158 bp, artificial sequence
  synthesized
tcggatgatgagcgtgaggagaaggagctggatctgactagccctgaggtggtgacgaagtacaagtccgccgccgagatcgtgaacaaggccctccagctggtgctgtcggagtgcaagccaaaggtgaagatcgtggacctgtgcgagaagggcgatgccttcatcaaggagcagaccgggaacatgtacaagaacgtgaagaagaagatcgagcggggcgtggccttcccgacttgtatctccgtgaacaacaccgtgtgccacttcagccctctggcgagcgacgagacgatcgtggaggagggcgacattctgaagatcgacatgggttgccacatcgacggtttcatcgcggtcgtgggtcacacccacgtgctgcacgagggcccggtcacgggccgcgccgctgacgtgatcgccgctgcgaacacggctgcggaggtggcgctgcgcctggtgcgtcccggcaagaagaactcggacgtgaccgaggccatccagaaggtcgcggctgcctacgactgcaagatcgtggagggcgtgctctcgcaccagatgaagcaattcgtgatcgacggcaacaaggtggtgctgagcgtgagcaaccccgacacccgcgtggacgaggccgagttcgaggagaacgaggtgtacagcatcgacattgtgacgagcacgggcgatggcaagcccaagctcctggacgagaagcagacaaccatctacaagcgggccgtggacaagagctacaacctgaagatgaaggcgagccgcttcattttctcggagatcaaccagaagttccccatcatgccattcaccgctcgggacctggaggagaagcgtgcccgtctgggcctggtcgagtgcgtgaaccatgagctcctgcaaccctacccggtcctgcacgagaagccgggcgacctggtggctcacattaagtttactgtgctgctgatgcccaacggcagcgaccgtgtgacatcgcacgygctgcaagagctgcaacccacgaagacgacggagaacgagcccgagatcaaggcgtggctggcgctccctacgaagactaagaagaagggcggtgggaagaagaagaagggcaagaagggcgacaaggtggaggaggcgtcgcaggccgagccgatggagggc

SEQ ID NO: 62 - DNA, 1158 bp, artificial sequence
  synthesized
tctgatgatgagcgtgaggagaaggagctggatctgactagccctgaggtcgtgactaagtacaagtccgctgcggagatcgtgaacaaggccctgcagctggtgctgagcgagtgcaagcccaaggtgaagatcgtggacctgtgcgagaagggcgacgcctttatcaaggagcagacgggcaacatgtacaagaacgtgaagaagaagatcgagcgcggcgtggccttccccacttgtatcagcgtgaacaacactgtgtgccacttcagccccctggcttccgacgagacgatcgtggaggagggcgacattctgaagatcgacatgggctgccacatcgacggcttcatcgccgtggtcggccacacgcacgtgctgcacgagggccctgtgaccgggcgtgccgcggacgtgatcgccgcggccaacactgccgctgaggtggcgctccgcctggtgcgtcccggcaagaagaacagcgacgtgactgaggcgattcagaaggtggctgccgcctatgactgcaagatcgtggagggcgtgctgagccaccagatgaagcagttcgtgatcgacggtaacaaggtggtgctgtccgtcagcaacccagatacccgcgtggacgaggccgagttcgaggagaacgaggtgtacagcatcgacattgtgacctccacgggcgacgggaagcccaagctgctcgacgagaagcagacgaccatctacaagcgcgcggtggataagtcttacaacctgaagatgaaggccagccgcttcatcttctctgagatcaaccagaagttccctatcatgcccttcacggcccgcgacctggaggagaagcgcgctcgcctgggcctcgtggagtgcgtcaaccacgagctgctgcagccttaccccgtgctgcacgagaagcccggcgacctggtcgcgcacatcaagttcacggtcctgctcatgcccaacggctccgaccgcgtcacctcccacgygctgcaggagctgcagcccaccaagaccaccgagaacgagcccgagatcaaggcctggctggccctgcccaccaagacgaagaagaaggggggcggcaagaagaagaagggcaagaagggcgacaaggtggaggaggcgagccaggccgagcccatggagggg

SEQ ID NO: 63 - DNA, 1164 bp, artificial sequence
  synthesized
atgtctgatgacggtagcattgagcaccaagagcctaacctgtctgtgcccgaggtggtgaccaagtacaaggctgcggcggatatctgcaaccgcgcgctgctggcggtggtggaggctgcgaaggacggcgccaaggtggtggacctgtgccgcatgggcgaccagttcatcaacaaggagtgcgcgaacatctacaagggcaaggagatcgagaagggcgtggcgttcccaacctgcgtgtccgcgaactctattgtgggccacttttcccccaacagcgaggacgcgaccgcgctgaagaacggtgacgtggtcaagatcgatatgggctgtcacatcgacgggttcattgctacccaggcgaccaccatcgtggtgggcgacgcggccatcagcggcaaggccgcggacgtgatcgccgctgcgcgcacggcgttcgacgccgcggtccgcctgattcgccctggcaagcacattgcggatgtgagcgctcccctccagaaggtcgctgagtccttcggctgcaacctggtggagggcgtgatgagccacgagatgaagcagttcgtgatcgacggcagcaagtgcatcctgaacaagcccacgcccgaccaaaaggtcgaggacggcgagttcgaggagaacgaggtgtacgccgtcgacatcgtggtcagcagcggcgagggcaagccccgcgtcctcgacgagaaggagactaccgtgtacaagcgcgccctggaggtcacttaccagctgaagatgcaagccagccgcgccgtgtttagcctcgtcaacagcgcgttcgctaccatgccattcaccctgcgtgcgctgctggacgaggctgccgcccaaaagaccgagctgaaggcgagccagctgaagctcggcctggtggagtgcctgaaccacggcctgctgcacccttaccccgtcctgcacgagaagcccggcgaggtggtggcccaaattaagggcaccgtgctgctgatgcctaacggctctagcatcatcaccagcgccccccgccagacggtgaccaccgagaagaaggtggaggacaaggagatcctcgacctgctggcgacgcccatcagcgcgaagagcgccaagaagaagaagaacaaggacaaggctgcggagccagcggctgccaagtaa

SEQ ID NO: 64 - DNA, 1158 bp, artificial sequence; identical to SEQ ID NO: 51
  synthesized

SEQ ID NO: 65 - DNA, 1173 bp, artificial sequence
  synthesized
atggtgaaggaggataagcaaactgatggtgatcgttggcgtggtctggcgtacgacacctccgacgaccagcaggatattacgcgcggcaaggggatggtggattccgtgttccaggcgcccatgggcactggcacccaccacgccgtgctgagcagctacgagtacgtctcccagggcctccgtcagtacaacctggacaacatgatggacggcttctacatcgctcccgctttcatggataagctggtggtgcacatcacgaagaacttcctgacgctgcccaacatcaaggtgccactgatcctggggatctggggcggcaagggccagggcaagagcttccaatgcgagctggtgatggcgaagatgggcatcaaccccatcatgatgagcgcgggcgagctggagtccgggaacgccggcgagcccgcgaagctgatccgccagcgctaccgcgaggctgcggacctgatcaagaagggcaagatgtgctgcctgctgattaacgacctggacgcgggcgctgggcgcatgggcggcaccacgcagtacactgtgaacaaccagatggtgaacgcgacgctgatgaacatcgcggacaacccaacgaacgtgcagctgcccggtatgtataacaaggaggagaacgcccgcgtgcccatcatctgcaccggcaacgacttcagcaccctgtacgccccactgatccgcgacggccgcatggagaagttctactgggcgcccacccgcgaggaccgcatcggcatttgtaagggtattttccgcaccgacaagattaaggacgaggacatcgtgaccctcgtggaccaattccctggtcagtccatcgacttcttcggcgcgctgcgcgcccgcgtctacgacgacgaggtgcgtaagttcgtcgagtccctgggggtggagaacatcgggaagcgcctggtgaactcccgcgagggccctcctgtgttcgagcagcccgagatgacttacgagaagctgatggagtacggcaacatgctggtgatggagcaggagaacgtgaagcgcgtgcagctggctgagacttacctgtcccaggccgccctgggcgacgccaacgccgacgccatcggccgcggcaccttctacgggaagactgaggagaaggagccctccaagctggagtaa

SEQ ID NO: 66 - DNA, 1167 bp, artificial sequence; identical to SEQ ID NO: 52
  synthesized

SEQ ID NO: 67 - DNA, 1167 bp, artificial sequence
  synthesized
atgtctgatgatgagcgtgaggagaaggagctggatctgactagccctgaggtcgtgactaagtacaagtccgctgcggagatcgtgaacaaggccctgcagctggtgctgagcgagtgcaagcccaaggtgaagatcgtggacctgtgcgagaagggcgacgcctttatcaaggagcagacgggcaacatgtacaagaacgtgaagaagaagatcgagcgcggcgtggccttccccacttgtatcagcgtgaacaacactgtgtgccacttcagccccctggcttccgacgagacgatcgtggaggagggcgacattctgaagatcgacatgggctgccacatcgacggcttcatcgccgtggtcggccacacgcacgtgctgcacgagggccctgtgaccgggcgtgccgcggacgtgatcgccgcggccaacactgccgctgaggtggcgctccgcctggtgcgtcccggcaagaagaacagcgacgtgactgaggcgattcagaaggtggctgccgcctatgactgcaagatcgtggagggcgtgctgagccaccagatgaagcagttcgtgatcgacggtaacaaggtggtgctgtccgtcagcaacccagatacccgcgtggacgaggccgagttcgaggagaacgaggtgtacagcatcgacattgtgacctccacgggcgacgggaagcccaagctgctcgacgagaagcagacgaccatctacaagcgcgcggtggataagtcttacaacctgaagatgaaggccagccgcttcatcttctctgagatcaaccagaagttccctatcatgcccttcacggcccgcgacctggaggagaagcgcgctcgcctgggcctcgtggagtgcgtcaaccacgagctgctgcagccttaccccgtgctgcacgagaagcccggcgacctggtcgcgcacatcaagttcacggtcctgctcatgcccaacggctccgaccgcgtcacctcccacctgcaggagctgcagcccaccaagaccaccgagaacgagcccgagatcaaggcctggctggccctgcccaccaagacgaagaagaaggggggcggcaagaagaagaagggcaagaagggcgacaaggtggaggaggcgagccaggccgagcccatggaggggacgggctaa

SEQ ID NO: 68 - DNA, 1155 bp, artificial sequence; identical to SEQ ID NO: 50
  synthesized

SEQ ID NO: 69 - DNA, 1161 bp, artificial sequence
  synthesized
tctgatgatgagcgtgaggagaaggagctggatctgactagccctgaggtcgtgactaagtacaagtccgctgcggagatcgtgaacaaggccctgcagctggtgctgagcgagtgcaagcccaaggtgaagatcgtggacctgtgcgagaagggcgacgcctttatcaaggagcagacgggcaacatgtacaagaacgtgaagaagaagatcgagcgcggcgtggccttccccacttgtatcagcgtgaacaacactgtgtgccacttcagccccctggcttccgacgagacgatcgtggaggagggcgacattctgaagatcgacatgggctgccacatcgacggcttcatcgccgtggtcggccacacgcacgtgctgcacgagggccctgtgaccgggcgtgccgcggacgtgatcgccgcggccaacactgccgctgaggtggcgctccgcctggtgcgtcccggcaagaagaacagcgacgtgactgaggcgattcagaaggtggctgccgcctatgactgcaagatcgtggagggcgtgctgagccaccagatgaagcagttcgtgatcgacggtaacaaggtggtgctgtccgtcagcaacccagatacccgcgtggacgaggccgagttcgaggagaacgaggtgtacagcatcgacattgtgacctccacgggcgacgggaagcccaagctgctcgacgagaagcagacgaccatctacaagcgcgcggtggataagtcttacaacctgaagatgaaggccagccgcttcatcttctctgagatcaaccagaagttccctatcatgcccttcacggcccgcgacctggaggagaagcgcgctcgcctgggcctcgtggagtgcgtcaaccacgagctgctgcagccttaccccgtgctgcacgagaagcccggcgacctggtcgcgcacatcaagttcacggtcctgctcatgcccaacggctccgaccgcgtcacctcccacctgcaggagctgcagcccaccaagaccaccgagaacgagcccgagatcaaggcctggctggccctgcccaccaagacgaagaagaaggggggcggcaagaagaagaagggcaagaagggcgacaaggtggaggaggcgagccaggccgagcccatggaggggacgggc

SEQ ID NO: 70 - protein, 388 aa, Chlamydomonas reinhardtii
MSDDEREEKELDLTSPEVVTKYKSAAEIVNKALQLVLSECKPKVKIVDLCEKGDAFIKEQTGNMYKNVKKKIERGVAFPTCISVNNTVCHFSPLASDETIVEEGDILKIDMGCHIDGFIAVVGHTHVLHEGPVTGRAADVIAAANTAAEVALRLVRPGKKNSDVTEAIQKVAAAYDCKIVEGVLSHQMKQFVIDGNKVVLSVSNPDTRVDEAEFEENEVYSIDIVTSTGDGKPKLLDEKQTTIYKRAVDKSYNLKMKASRFIFSEINQKFPIMPFTARDLEEKRARLGLVECVNHELLQPYPVLHEKPGDLVAHIKFTVLLMPNGSDRVTSHLQELQPTKTTENEPEIKAWLALPTKTKKKGGGKKKKGKKGDKVEEASQAEPMEGTG